(19) United States
(12) Patent Application Publication
(10) Pub. No.: US 2002/0019527 A1
(43) Pub. Date: Feb. 14, 2002

(54) Substituted phenyl farnesyltransferase inhibitors

(76) Inventors: Wei-Bo Wang, Grayslake, IL (US); Michael L. Curtin, Pleasant Prairie, WI (US); Stephen A. Fakhoury, Ann Arbor, MI (US); Stephen L. Gwaltney II, Lindenhurst, IL (US); Lisa A. Hasvold, Grayslake, IL (US); Charles W. Hutchins, Green Oaks, IL (US); Qun Li, Libertyville, IL (US); Nan-Horng Lin, Vernon Hills, IL (US); Lissa Taka Jennings Nelson, Highland Park, IL (US); Steve O'Connor, Guilford, CT (US); Hing L. Sham, Vernon Hills, IL (US); Gerard M. Sullivan, Round Lake Beach, IL (US); Gary T. Wang, Niles, IL (US); Xilu Wang, Skokie, IL (US)

Correspondence Address: Gregory W. Steele, Abbott Laboratories, AP6D/2 D-377, 100 Abbott Park Road, Abbott Park, IL, 60064-6050, US

(21) Appl. No.: 09/842,391

(22) Filed: Apr. 25, 2001

Related U.S. Application Data
(60) Provisional application No. 60/200,165, filed on Apr. 27, 2000.

(51) Int. Cl. C07D 401/00

(52) U.S. Cl. 544/360

(57) ABSTRACT

Compounds of formula (I)

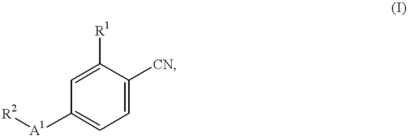

or pharmaceutically acceptable salts thereof, inhibit farnesyltransferase. Methods for making the compounds, pharmaceutical compositions containing the compounds, and methods of treatment using the compounds are disclosed.

### CROSS-REFERENCE TO RELATED APPLICATIONS

This application claims priority to U.S. provisional application 60/200,165, filed Apr. 27, 2000, which is hereby incorporated by reference.

### TECHNICAL FIELD

The instant invention provides substituted phenyl compounds which inhibit farnesyltransferase, methods for making the compounds, pharmaceutical compositions containing the compounds, and methods of treatment using the compounds.

### BACKGROUND OF THE INVENTION

Ras oncogenes are the most frequently identified activated oncogenes in human tumors, and transformed protein Ras is involved in the proliferation of cancer cells. The Ras must be farnesylated by farnesyl pyrophosphate before this proliferation can occur, and farnesylation of Ras by farnesyl pyrophosphate is effected by protein farnesyltransferase. Inhibition of protein farnesyltransferase, and thereby farnesylation of the Ras protein, blocks the ability of transformed cells to proliferate.

Activation of Ras and related proteins which are farnesylated also partially mediates smooth muscle cell proliferation (Circulation, 1-3: 88 (1993)). Inhibition of protein isoprenyl transferases, and thereby farnesylation of the Ras protein, also aids in the prevention of intimal hyperplasia associated with restenosis and atherosclerosis, a condition which compromises the success of angioplasty and surgical bypass for obstructive vascular lesions.

Because of this pivotal role played by farnesyltransferase in tumor formation and metastasis, compounds such as those reported in WO 97/36897, WO 97/36881, WO 97/36875, WO 97/36901, WO 99/17777, WO 99/18096, WO 99/20609, WO 99/27928, WO 99/27933, WO 99/27929, WO 99/28313, and WO 99/28314 have been the subject of current research.

However, there is still an ongoing need for farnesyltransferase inhibitors with modified or improved profiles of activity.

### SUMMARY OF THE INVENTION

In its principle embodiment, therefore, the instant invention discloses compounds of formula (I)

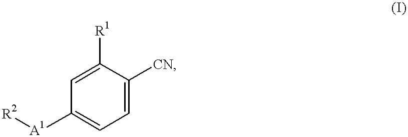

or pharmaceutically acceptable salts thereof, wherein

A¹ is L¹-M¹-L² or alkylene, wherein the alkylene can be optionally substituted with one, two, or three substituents independently selected from the group consisting of amino, hydroxyl, oxo, and -Q¹-Q²-R³;

with the proviso that when A¹ is methylene, the methylene is substituted;

L¹and L² are independently absent or alkylene, wherein the alkylenes defining L¹ and L² can be optionally substituted with one or two substituents independently selected from the group consisting of alkyl, cycloalkyl, cycloalkylalkyl, aryl, arylalkyl, heteroaryl, heteroarylalkyl, and oxo;

with the proviso that at least one of L¹ or L² is present;

M¹ is selected from the group consisting of O, N(R⁴), N(R⁵)SO₂, SO₂N(R⁵), N(R⁵)C(O), C(O)N(R⁵), OC(O), C(O)O, C(O), N(R⁵)C(O)O, OC(O)N(R⁵), OC(O)O, N(R⁵)C(O)N(R⁵), and S(O)ₜ, wherein t is zero, one, or two; wherein, for the groups defining M¹, the left ends are attached to L¹ and the right ends are attached to L²;

Q¹ is absent or selected from the group consisting of O, N(R⁴), N(R⁵)C(O), N(R⁵)SO₂, and S(O)ₜ;

Q² is absent or selected from the group consisting of alkylene, alkenylene, and alkynylene;

R¹ is selected from the group consisting of halo, cycloalkyl, aryl, and heteroaryl;

R² is a heteroaryl selected from the group consisting of imidazolyl, pyrazolyl, pyrrolyl, thienyl, triazolyl, pyridyl, and thiazolyl;

R³ is selected from the group consisting of alkyl, cycloalkyl, aryl, heteroaryl, and heterocycloalkyl;

R⁴ is selected from the group consisting of hydrogen, alkyl, alkenyl, alkynyl, alkanoyl, alkylsulfonyl, a nitrogen protecting group, aminosulfonyl, aryl, arylalkyl, aryloyl, arylsulfonyl, cycloalkyl, cycloalkylalkyl, cycloalkyloyl, cycloalkylsulfonyl, heteroaryl, heteroarylalkyl, heteroaryloyl, heteroarylsulfonyl, heterocycloalkyl, heterocycloalkylalkyl, heterocycloalkyloyl, and heterocycloalkylsulfonyl; and

R⁵ is selected from the group consisting of hydrogen, alkyl, aryl, arylalkyl, cycloalkyl, cycloalkylalkyl, heteroaryl, heteroarylalkyl, heterocycloalkyl, and heterocycloalkylalkyl.

In another embodiment, the instant invention discloses compounds of formula (II)

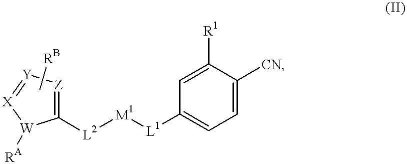

or a pharmaceutically acceptable salt thereof, wherein

R^{A} is absent or selected from the group consisting of hydrogen, optionally substituted alkyl, alkoxycarbonyl, and a nitrogen protecting group;

R^{B} is absent or selected from the group consisting of optionally substituted alkyl, alkoxy, alkanoyl, alkanoyloxy, alkoxycarbonyl, alkylsulfonyl, amino, aminosulfonyl, azido, carboxamido, carboxyl, cyano, halo, hydroxyl, perfluoroalkyl, and perfluoroalkoxy;

W is C(H)═C(H), X is N, and Y and Z are C(H); or

W is C(H)═N or N═C(H), wherein each group is drawn with its left end attached to X and its right end attached to the carbon substituted with L²; and X, Y and Z are C(H); or

W is N or S, one of X, Y, or Z is C(H), and the remainder are C(H) or N;

with the proviso that R^{A} is present when and only when W is N.

In a preferred embodiment of compounds of formula (II) are compounds wherein

M¹ is O;

L¹ is optionally substituted alkylene;

L² is optionally substituted alkylene;

W and Y are N; and

X and Z are C(H).

Compounds which support this embodiment include, but are not limited to,

4-(((4-cyanophenyl)(1-methyl-1H-imidazol-5-yl)methoxy)methyl)-2-(1-naphthyl)-benzonitrile,

4-((2-(4-cyanophenyl)-1-(1-methyl-1H-imidazol-5-yl)ethoxy)methyl)-2-(1-naphthyl)benzonitrile,

4-((1-(1-methyl-1H-imidazol-5-yl)-3-phenylpropoxy)methyl)-2-(1-naphthyl)-benzonitrile,

4-(((1-methyl-1H-imidazol-5-yl)(phenyl)methoxy)methyl)-2-(1-naphthyl)-benzonitrile, and

4-((1-(1-methyl-1H-imidazol-5-yl)-2-phenylethoxy)methyl)-2-(1-naphthyl)-benzonitrile.

In another preferred embodiment of compounds of formula (II) are compounds wherein

M¹ is O;

L¹ is optionally substituted alkylene;

L² is optionally substituted alkylene;

W is N═C(H); and

X, Y, and Z are C(H).

Compounds which support this embodiment include, but are not limited to,

Example 516,

Example 517,

Example 518,

Example 519, and

Example 521.

In another preferred embodiment of compounds of formula (II) are compounds wherein

M is O;

L¹ is optionally substituted alkylene;

L² is optionally substituted alkylene;

W is S;

Y is N; and

X and Z are C(H).

Compounds which support this embodiment include, but are not limited to,

Example 775,

Example 776,

Example 777,

Example 778, and

Example 780.

In another preferred embodiment of compounds of formula (II) are compounds wherein

M¹is N(R⁴);

W is N;

Y is N; and

X and Z are C(H).

Compounds which support this embodiment include, but are not limited to,

5-((benzyl((1-methyl-1H-imidazol-5-yl)methyl)amino)methyl)-2′-methyl(1,1′-biphenyl)-2-carbonitrile,

4-(((4-cyanobenzyl)((1-methyl-1H-imidazol-5-yl)methyl)amino)methyl)-2-(1-naphthyl)benzonitrile,

4-(((4-chlorobenzyl)((1-methyl-1H-imidazol-5-yl)methyl)amino)methyl)-2-(1-naphthyl)benzonitrile,

4-((((1-methyl-1H-imidazol-5-yl)methyl)(4-(trifluoromethoxy)benzyl)amino)-methyl)-2-(1-naphthyl)benzonitrile,

4-(((4-cyanobenzyl)(1H-imidazol-5-ylmethyl)amino)methyl)-2-(1-naphthyl)-benzonitrile,

5-(((2-cyclohexylethyl)((1-methyl-1H-imidazol-5-yl)methyl)amino)methyl)-2′-methyl(1,1′-biphenyl)-2-carbonitrile,

4-(((2-cyclohexylethyl)((1-methyl-1H-imidazol-5-yl)methyl)amino)methyl)-2-(1-naphthyl)benzonitrile,

4-(((cyclohexylmethyl)((1-methyl-1H-imidazol-5-yl)methyl)amino)methyl)-2-(1-naphthyl)benzonitrile,

4-(((4-cyanobenzyl)((1-methyl-1H-imidazol-5-yl)methyl)amino)methyl)-2-(8-quinolinyl)benzonitrile,

4-(((3,4-dichlorobenzyl)((1-methyl-1H-imidazol-5-yl)methyl)amino)methyl)-2-(1-naphthyl)benzonitrile,

4-cyano-N-(4-cyanobenzyl)-N-((1-methyl-1H-imidazol-5-yl)methyl)-3-(1-naphthyl)benzamide,

4-((((1-methyl-1H-imidazol-5-yl)methyl)(4-(trifluoromethyl)benzyl)amino)methyl)-2-(1-naphthyl)benzonitrile,

4-(((4-cyano-3-(1-naphthyl)benzyl)((1-methyl-1H-imidazol-5-yl)methyl)amino)-methyl)benzoic acid,

N-(4-(((4-cyano-3-(1-naphthyl)benzyl)((1-methyl-1H-imidazol-5-yl)methyl)amino)-methyl)phenyl)acetamide,

4-((((1-methyl-1H-imidazol-5-yl)methyl)(4-(methylsulfonyl)benzyl)amino)methyl)-2-(1-naphthyl)benzonitrile,

4-cyano-N-(4-cyano-3-(1-naphthyl)benzyl)-N-((1-methyl-1H-imidazol-5-yl)-methyl)benzamide,

3,4-dichloro-N-(4-cyano-3-(1-naphthyl)benzyl)-N-((1-methyl-1H-imidazol-5-yl)-methyl)benzamide,

4-chloro-N-(4-cyano-3-(1-naphthyl)benzyl)-3-fluoro-N-((1-methyl-1H-imidazol-5-yl)methyl)benzamide,

5,6-dichloro-N-(4-cyano-3-(1-naphthyl)benzyl)-N-((1-methyl-1H-imidazol-5-yl)-methyl)nicotinamide,

4-cyano-N-(4-cyanobenzyl)-N-((1-methyl-1H-imidazol-5-yl)methyl)-3-(8-quinolinyl)-benzamide,

4-(((2-hydroxy-5-(trifluoromethoxy)benzyl)((1-methyl-1H-imidazol-5-yl)methyl)-amino)methyl)-2-(1-naphthyl)benzonitrile,

methyl 6-(((4-cyano-3-(1-naphthyl)benzyl)((1-methyl-1H-imidazol-5-yl)methyl)-amino)-methyl)nicotinate,

ethyl 4-((4-cyano-3-(1-naphthyl)benzyl)((1-methyl-1H-imidazol-5-yl)methyl)-amino)-1-piperidinecarboxylate,

2′-methyl-5-(((1-methyl-1H-imidazol-5-yl)methyl)amino)(1,1′-biphenyl)-2-carbonitrile,

5-(benzyl((1-methyl-1H-imidazol-5-yl)methyl)amino)-2′-methyl(1,1′-biphenyl)-2-carbonitrile,

4-(methyl((1-methyl-1H-imidazol-5-yl)methyl)amino)-2-(1-naphthyl)benzonitrile,

4-(allyl((1-methyl-1H-imidazol-5-yl)methyl)amino)-2-(1-naphthyl)benzonitrile,

5-((4-cyanobenzyl)((1-methyl-1H-imidazol-5-yl)methyl)amino)-2′-methyl(1,1′-biphenyl)-2-carbonitrile,

4-(((1-methyl-1H-imidazol-5-yl)methyl)(3-phenylpropyl)amino)-2-(1-naphthyl)benzonitrile,

4-((4-cyanobenzyl)((1-methyl-1H-imidazol-5-yl)methyl)amino)-2-(1-naphthyl)-benzonitrile,

4-(benzyl((1-methyl-1H-imidazol-5-yl)methyl)amino)-2-(1-naphthyl)benzonitrile,

4-(hexyl((1-methyl-1H-imidazol-5-yl)methyl)amino)-2-(1-naphthyl)benzonitrile,

4-(((1-methyl-1H-imidazol-5-yl)methyl)amino)-2-(1-naphthyl)benzonitrile,

N-(4-cyano-3-(1-naphthyl)phenyl)-N-((1-methyl-1H-imidazol-5-yl)methyl)-benzamide,

N-(6-cyano-2′-methyl(1,1′-biphenyl)-3-yl)-N-((1-methyl-1H-imidazol-5-yl)methyl)-benzamide,

5-((3-cyanobenzyl)((1-methyl-1H-imidazol-5-yl)methyl)amino)-2′-methyl(1,1′-biphenyl)-2-carbonitrile,

4-((((1-methyl-1H-imidazol-5-yl)(phenyl)methyl)amino)methyl)-2-(1-naphthyl)benzonitrile,

4-(((1-(1-methyl-1H-imidazol-5-yl)-2-phenylethyl)amino)methyl)-2-(1-naphthyl)-benzonitrile,

4-(((1-(1-methyl-1H-imidazol-5-yl)-3-phenylpropyl)amino)methyl)-2-(1-naphthyl)-benzonitrile,

4-(((2-(4-cyanophenyl)-1-(1-methyl-1H-imidazol-5-yl)ethyl)amino)methyl)-2-(1-naphthyl)benzonitrile,

4-((3-chlorobenzyl)((1-methyl-1H-imidazol-5-yl)methyl)amino)-2-(1-naphthyl)-benzonitrile,

4-(benzyl(1H-imidazol-5-ylmethyl)amino)-2-(1-naphthyl)benzonitrile,

4-((3-cyanobenzyl)((1-methyl-1H-imidazol-5-yl)methyl)amino)-2-(1-naphthyl)-benzonitrile,

N-(4-cyano-3-(1-naphthyl)phenyl)-N-((1-methyl-1H-imidazol-5-yl)methyl)benzene-sulfonamide,

methyl 4-((4-cyano((1-methyl-1H-imidazol-5-yl)methyl)-3-(1-naphthyl)anilino)-methyl)benzoate,

4-((4-cyano((1-methyl-1H-imidazol-5-yl)methyl)-3-(1-naphthyl)anilino)methyl)-benzoic acid,

5-(benzyl(1H-imidazol-5-ylmethyl)amino)-2′-methyl(1,1′-biphenyl)-2-carbonitrile,

methyl 3-((4-cyano((1-methyl-1H-imidazol-5-yl)methyl)-3-(1-naphthyl)anilino)-methyl)benzoate,

4-(((4-cyanophenyl)(1-methyl-1H-imidazol-5-yl)methyl)amino)-2-(1-naphthyl)-benzonitrile, and

6-(((4-cyano-3-(1-naphthyl)benzyl)((1-methyl-1H-imidazol-5-yl)methyl)amino)-methyl)nicotinonitrile.

In another preferred embodiment of compounds of formula (II) are compounds wherein

M¹ is N(R⁴);

W is N═C(H); and

X, Y and Z are C(H).

Compounds which support this embodiment include, but are not limited to,

2′-methyl-5-((3-pyridinylamino)methyl)(1,1′-biphenyl)-2-carbonitrile,

5-((benzyl(3-pyridinylmethyl)amino)methyl)-2′-methyl(1,1′-biphenyl)-2-carbonitrile,

2′-methyl-5-((3-pyridinylmethyl)amino)(1,1′-biphenyl)-2-carbonitrile,

5-(benzyl(3-pyridinylmethyl)amino)-2′-methyl(1,1′-biphenyl)-2-carbonitrile,

Example 322,

Example 328,

Example 329,

Example 363,

Example 364,

Example 365,

Example 390,

Example 450,

Example 467,

Example 468,

Example 469,

Example 470,

Example 471,

Example 472,

Example 473,

Example 474,

Example 475,

Example 482,

Example 483,

Example 484,

Example 485,

Example 490,

Example 491,

Example 492,

Example 493,

Example 494,

Example 495,

Example 496,

Example 497,

Example 498,

Example 499,

Example 500,

Example 520,

Example 522,

Example 523,

Example 524,

Example 527,

Example 528,

Example 529,

Example 530,

Example 531,

Example 532,

Example 548, and

Example 549.

In another preferred embodiment of compounds of formula (II) are compounds wherein

M¹ is N(R⁴);

W is S;

Y is N; and

X and Z are C(H).

Compounds which support this embodiment include, but are not limited to,

Example 578,

Example 580,

Example 586,

Example 587,

Example 620,

Example 621,

Example 622,

Example 646,

Example 706,

Example 723,

Example 724,

Example 725,

Example 726,

Example 727,

Example 728,

Example 729,

Example 730,

Example 731,

Example 738,

Example 739,

Example 740,

Example 741,

Example 753,

Example 754,

Example 755,

Example 756,

Example 757,

Example 758,

Example 759,

Example 779,

Example 781,

Example 782,

Example 783,

Example 786,

Example 787,

Example 788,

Example 788,

Example 789,

Example 790,

Example 791,

Example 807, and

Example 808.

In another embodiment, the instant invention discloses compounds of formula (III)

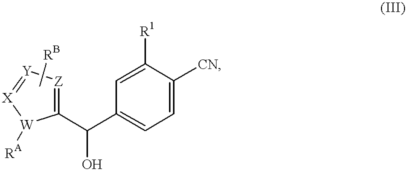

or a pharmaceutically acceptable salt thereof, wherein

R^{A} is absent or selected from the group consisting of hydrogen, optionally substituted alkyl, alkoxycarbonyl, and a nitrogen protecting group;

R^{B} is absent or selected from the group consisting of optionally substituted alkyl, alkoxy, alkanoyl, alkanoyloxy, alkoxycarbonyl, alkylsulfonyl, amino, aminosulfonyl, azido, carboxamido, carboxyl, cyano, halo, hydroxyl, perfluoroalkyl, and perfluoroalkoxy; and

W is C(H)═C(H), X is N, and Y and Z are C(H); or

W is C(H)═N or N═C(H), wherein each group is drawn with its left end attached to X and its right end attached to the carbon substituted with L²; and X, Y and Z are C(H); or

W is N or S, one of X, Y, or Z is C(H), and the remainder are C(H) or N;

with the proviso that R^{A} is present when and only when W is N.

In a preferred embodiment of compounds of formula (III) are compounds wherein

W is N;

Y is N; and

X and Z are C(H).

A compound which supports this embodiment includes, but is not limited to,

5-(hydroxy(1-methyl-1H-imidazol-5-yl)methyl)-2′-methyl(1,1′-biphenyl)-2-carbonitrile.

In another preferred embodiment of compounds of formula (III) are compounds wherein

W is S;

Y is N; and

X and Z are C(H).

A compound which supports this embodiment includes, but is not limited to,

5-(hydroxy(1,3-thiazol-5-yl)methyl)-2′-methyl(1,1′-biphenyl)-2-carbonitrile.

In another preferred embodiment of compounds of formula (III) are compounds wherein

W is N═C(H); and

X, Y, and Z are C(H).

A compound which supports this embodiment includes, but is not limited to,

5-(hydroxy(3-pyridinyl)methyl)-2′-methyl(1,1′-biphenyl)-2-carbonitrile,

In another embodiment, the instant invention discloses compounds of formula (IV)

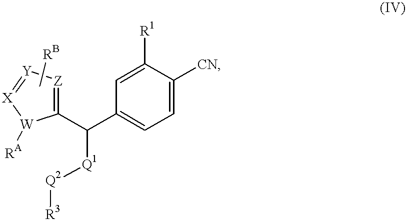

or a pharmaceutically acceptable salt thereof, wherein

Q² is absent or alkylene;

R^{A} is absent or selected from the group consisting of hydrogen, optionally substituted alkyl, alkoxycarbonyl, and a nitrogen protecting group;

R^{B} is absent or selected from the group consisting of optionally substituted alkyl, alkoxy, alkanoyl, alkanoyloxy, alkoxycarbonyl, alkylsulfonyl, amino, aminosulfonyl, azido, carboxamido, carboxyl, cyano, halo, hydroxyl, perfluoroalkyl, and perfluoroalkoxy; and

W is C(H)═C(H), X is N, and Y and Z are C(H); or

W is C(H)═N or N═C(H), wherein each group is drawn with its left end attached to X and its right end attached to the carbon substituted with L²; and X, Y and Z are C(H); or

W is N or S, one of X, Y, or Z is C(H), and the remainder are C(H) or N;

with the proviso that R^{A} is present when and only when W is N.

In a preferred embodiment of compounds of formula (IV) are compounds wherein

Q¹ is O;

W is N;

Y is N; and

X and Z are C(H).

Compounds which support this embodiment include, but are not limited to,

2′-methyl-5-((1-methyl-1H-imidazol-5-yl)(phenoxy)methyl)(1,1′-biphenyl)-2-carbonitrile,

5-((benzyloxy)(1-methyl-1H-imidazol-5-yl)methyl)-2′-methoxy(1,1′-biphenyl)-2-carbonitrile,

5-((benzyloxy)(1-methyl-1H-imidazol-5-yl)methyl)-3′-phenyl(1,1′-biphenyl)-2-carbonitrile,

(2-(9-anthryl)-4-((benzyloxy)(1-methyl-1H-imidazol-5-yl)methyl)benzonitrile,

5-((benzyloxy)(1-methyl-1H-imidazol-5-yl)methyl)-2′-isopropyl(1,1′-biphenyl)-2-carbonitrile,

4-((benzyloxy)(1-methyl-1H-imidazol-5-yl)methyl)-2-(1,2-dihydro-5-acenaphth-ylenyl)benzonitrile,

5-((benzyloxy)(1-methyl-1H-imidazol-5-yl)methyl)-2′-chloro(1,1′-biphenyl)-2-carbonitrile,

5-((benzyloxy)(1-methyl-1H-imidazol-5-yl)methyl)-2′-methyl(1,1′-biphenyl)-2-carbonitrile,

4-((cyclohexylmethoxy)(1-methyl-1H-imidazol-5-yl)methyl)-2-(1-naphthyl)benzonitrile,

4-(((4-cyanobenzyl)oxy)(1-methyl-1H-imidazol-5-yl)methyl)-2-(8-quinolinyl)-benzonitrile,

4-(((4-cyanobenzyl)oxy)(1-methyl-1H-imidazol-5-yl)methyl)-2-(4-quinolinyl)-benzonitrile,

4-(((4-cyanobenzyl)oxy)(1-methyl-1H-imidazol-5-yl)methyl)-2-(5-quinolinyl)-benzonitrile,

4-(((4-cyanobenzyl)oxy)(1-methyl-1H-imidazol-5-yl)methyl)-2-(5-isoquinolinyl)-benzonitrile,

4-(((4-cyanobenzyl)oxy)(1H-imidazol-5-yl)methyl)-2-(1-naphthyl)benzonitrile,

4-((1-methyl-1H-imidazol-5-yl)((4-nitrobenzyl)oxy)methyl)-2-(1-naphthyl)-benzonitrile,

4-(((4-cyanobenzyl)oxy)(1-methyl-1H-imidazol-5-yl)methyl)-2-iodobenzonitrile,

4-(((3-chloro-4-cyanobenzyl)oxy)(1-methyl-1H-imidazol-5-yl)methyl)-2-(1-naphthyl)benzonitrile,

4-(((4-cyano-3-iodobenzyl)oxy)(1-methyl-1H-imidazol-5-yl)methyl)-2-(1-naphthyl)-benzonitrile,

methyl 4-(((4-cyano-3-(1-naphthyl)phenyl)(1-methyl-1H-imidazol-5-yl)methoxy)-methyl)benzoate,

4-((1-methyl-1H-imidazol-5-yl)((4-(trifluoromethyl)benzyl)oxy)methyl)-2-(1-naphthyl)benzonitrile,

4-(((4-chlorobenzyl)oxy)(1-methyl-1H-imidazol-5-yl)methyl)-2-(1-naphthyl)-benzonitrile,

4-((1-methyl-1H-imidazol-5-yl)((4-(trifluoromethoxy)benzyl)oxy)methyl)-2-(1-naphthyl)benzonitrile,

4-((1-methyl-1H-imidazol-5-yl)((3-(trifluoromethyl)benzyl)oxy)methyl)-2-(1-naphthyl)benzonitrile,

4-(((4-cyano-3-(1-naphthyl)phenyl)(1-methyl-1H-imidazol-5-yl)methoxy)methyl)-benzoic acid,

4-(((4-cyano-3-(1-naphthyl)phenyl)(1-methyl-1H-imidazol-5-yl)methoxy)methyl)-N,N-dimethylbenzamide,

4-(((2,4-dichlorobenzyl)oxy)(1-methyl-1H-imidazol-5-yl)methyl)-2-(1-naphthyl)-benzonitrile,

4-((1-methyl-1H-imidazol-5-yl)((4-(methylsulfonyl)benzyl)oxy)methyl)-2-(1-naphthyl)benzonitrile,

4-(((2,6-dichloro-4-pyridinyl)methoxy)(1-methyl-1H-imidazol-5-yl)methyl)-2-(1-naphthyl)benzonitrile,

4-(((3-bromo-4-cyanobenzyl)oxy)(1-methyl-1H-imidazol-5-yl)methyl)-2-(1-naphthyl)benzonitrile,

6-(((4-cyano-3-(1-naphthyl)phenyl)(1-methyl-1H-imidazol-5-yl)methoxy)methyl)-nicotinonitrile,

4-(((4-cyano-3-fluorobenzyl)oxy)(1-methyl-1H-imidazol-5-yl)methyl)-2-(1-naphthyl)benzonitrile,

5-((benzyloxy)(1-methyl-1H-imidazol-5-yl)methyl)(1,1′-biphenyl)-2-carbonitrile,

4-((benzyloxy)(1-methyl-1H-imidazol-5-yl)methyl)-2-(1-naphthyl)benzonitrile,

4-((benzyloxy)(1-methyl-1H-imidazol-5-yl)methyl)-2-(3-thienyl)benzonitrile,

5-((benzyloxy)(1-methyl-1H-imidazol-5-yl)methyl)-3′-methyl(1,1′-biphenyl)-2-carbonitrile,

4-((benzyloxy)(1-methyl-1H-imidazol-5-yl)methyl)-2-(2-naphthyl)benzonitrile,

5-((benzyloxy)(1-methyl-1H-imidazol-5-yl)methyl)-4′-methyl(1,1′-biphenyl)-2-carbonitrile,

5-((benzyloxy)(1-methyl-1H-imidazol-5-yl)methyl)-2′-phenyl(1,1′-biphenyl)-2-carbonitrile,

5-((benzyloxy)(1-methyl-1H-imidazol-5-yl)methyl)-2′,5′-dimethyl(1,1′-biphenyl)-2-carbonitrile,

4-(((4-cyanobenzyl)oxy)(1-methyl-1H-imidazol-5-yl)methyl)-2-(1-naphthyl)-benzonitrile,

4-(((2-methoxy-5-nitrobenzyl)oxy)(1-methyl-1H-imidazol-5-yl)methyl)-2-(1-naphthyl)-benzonitrile,

5-((benzyloxy)(1-methyl-1H-imidazol-5-yl)methyl)-2′-ethyl(1,1′-biphenyl)-2-carbonitrile,

5-((benzyloxy)(1-methyl-1H-imidazol-5-yl)methyl)-2′,3′-dimethyl(1,1′-biphenyl)-2-carbonitrile,

4-((benzyloxy)(1-methyl-1H-imidazol-5-yl)methyl)-2-cyclohexylbenzonitrile,

4-((benzyloxy)(1-methyl-1H-imidazol-5-yl)methyl)-2-(5,6,7,8-tetrahydro-1-naphthalenyl)benzonitrile,

4-((benzyloxy)(1-methyl-1H-imidazol-5-yl)methyl)-2-(2-methyl-1-naphthyl)-benzonitrile,

2-(1-anthryl)-4-((benzyloxy)(1-methyl-1H-imidazol-5-yl)methyl)benzonitrile,

4-((benzyloxy)(1-methyl-1H-imidazol-5-yl)methyl)-2-(4-isoquinolinyl)benzonitrile,

4-((benzyloxy)(1-(ethoxymethyl)-1H-imidazol-5-yl)methyl)-2-(1-naphthyl)-benzonitrile,

4-(((4-cyanobenzyl)oxy)(1-(ethoxymethyl)-1H-imidazol-5-yl)methyl)-2-(1-naphthyl)benzonitrile,

5-(((4-cyanobenzyl)oxy)(1-methyl-1H-imidazol-5-yl)methyl)-2′-phenyl(1,1′-biphenyl)-2-carbonitrile,

4-(((4-cyanobenzyl)oxy)(1-(3-hydroxypropyl)-1H-imidazol-5-yl)methyl)-2-(1-naphthyl)benzonitrile,

4-(((4-fluoro-3-(1-naphthyl)phenyl)(1-methyl-1H-imidazol-5-yl)methoxy)methyl)-benzonitrile,

5-(((3-cyanobenzyl)oxy)(1-methyl-1H-imidazol-5-yl)methyl)-2′-methyl(1,1′-biphenyl)-2-carbonitrile,

5-(((4-(tert-butyl)benzyl)oxy)(1-methyl-1H-imidazol-5-yl)methyl)-2′-methyl(1,1′-biphenyl)-2-carbonitrile,

5-(((4-cyanobenzyl)oxy)(1-methyl-1H-imidazol-5-yl)methyl)-2′-methyl(1,1′-biphenyl)-2-carbonitrile,

5-(((3-iodobenzyl)oxy)(1-methyl-1H-imidazol-5-yl)methyl)-2′-methyl(1,1′-biphenyl)-2-carbonitrile,

5-(((4-fluorobenzyl)oxy)(1-methyl-1H-imidazol-5-yl)methyl)-2′-methyl(1,1′-biphenyl)-2-carbonitrile,

5-(((4-bromobenzyl)oxy)(1-methyl-1H-imidazol-5-yl)methyl)-2′-methyl(1,1′-biphenyl)-2-carbonitrile,

5-(((3-chlorobenzyl)oxy)(1-methyl-1H-imidazol-5-yl)methyl)-2′-methyl(1,1,′-biphenyl)-2-carbonitrile,

(2′-methyl-5-((1-methyl-1H-imidazol-5-yl)((4-nitrobenzyl)oxy)methyl)(1,1′-biphenyl)-2-carbonitrile,

5-(((2-methoxy-5-nitrobenzyl)oxy)(1-methyl-1H-imidazol-5-yl)methyl)-2′-methyl-(1,1′-biphenyl)-2-carbonitrile,

(2′-methyl-5-((1-methyl-1H-imidazol-5-yl)((3-(trifluoromethyl)benzyl)oxy)methyl)-(1,1′-biphenyl)-2-carbonitrile,

5-(((2,6-dichlorobenzyl)oxy)(1-methyl-1H-imidazol-5-yl)methyl)-2′-methyl(1,1′-biphenyl)-2-carbonitrile,

5-(((3,4-dichlorobenzyl)oxy)(1-methyl-1H-imidazol-5-yl)methyl)-2′-methyl(1,1′-biphenyl)-2-carbonitrile,

5-(((2-cyanobenzyl)oxy)(1-methyl-1H-imidazol-5-yl)methyl)-2′-methyl(1,1′-biphenyl)-2-carbonitrile,

(2′-methyl-5-(((4-methylbenzyl)oxy)(1-methyl-1H-imidazol-5-yl)methyl)(1,1′-biphenyl)-2-carbonitrile,

(2′-methyl-5-(((3-methylbenzyl)oxy)(1-methyl-1H-imidazol-5-yl)methyl)(1,1′-biphenyl)-2-carbonitrile,

5-(((2,5-difluorobenzyl)oxy)(1-methyl-1H-imidazol-5-yl)methyl)-2′-methyl(1,1′-biphenyl)-2-carbonitrile,

methyl 4-(((6-cyano-2′-methyl(1,1′-biphenyl)-3-yl)(1-methyl-1H-imidazol-5-yl)methoxy)-methyl)benzoate,

5-(((3,5-difluorobenzyl)oxy)(1-methyl-1H-imidazol-5-yl)methyl)-2′-methyl(1,1′-biphenyl)-2-carbonitrile,

5-(((2-chlorobenzyl)oxy)(1-methyl-1H-imidazol-5-yl)methyl)-2′-methyl(1,1′-biphenyl)-2-carbonitrile,

5-(((4-chlorobenzyl)oxy)(1-methyl-1H-imidazol-5-yl)methyl)-2′-methyl(1,1′-biphenyl)-2-carbonitrile,

5-(((3-methoxybenzyl)oxy)(1-methyl-1H-imidazol-5-yl)methyl)-2′-methyl(1,1′-biphenyl)-2-carbonitrile,

(2′-methyl-5-(((2-methylbenzyl)oxy)(1-methyl-1H-imidazol-5-yl)methyl)(1,1′-biphenyl)-2-carbonitrile,

5-(((3-fluorobenzyl)oxy)(1-methyl-1H-imidazol-5-yl)methyl)-2′-methyl(1,1′-biphenyl)-2-carbonitrile,

5-(((2,6-dichloro-4-pyridinyl)methoxy)(1-methyl-1H-imidazol-5-yl)methyl)-2′-methyl(1,1′-biphenyl)-2-carbonitrile,

5-(((2-fluorobenzyl)oxy)(1-methyl-1H-imidazol-5-yl)methyl)-2′-methyl(1,1′-biphenyl)-2-carbonitrile,

(2′-methyl-5 -((1-methyl-1H-imidazol-5-yl)((4-(trifluoromethyl)benzyl)oxy)-methyl)(1,1′-biphenyl)-2-carbonitrile,

5-(((3,5-dimethylbenzyl)oxy)(1-methyl-1H-imidazol-5-yl)methyl)-2′-methyl(1,1′-biphenyl)-2-carbonitrile,

5-(((4-fluoro-2-(trifluoromethyl)benzyl)oxy)(1-methyl-1H-imidazol-5-yl)methyl)-2′-methyl-(1,1′-biphenyl)-2-carbonitrile,

(2′-methyl-5-((1-methyl-1H-imidazol-5-yl)((2-nitrobenzyl)oxy)methyl)(1,1′-biphenyl)-2-carbonitrile,

(2′-methyl-5-((1-methyl-1H-imidazol-5-yl)((3-(trifluoromethoxy)benzyl)oxy)-methyl)(1,1′-biphenyl)-2-carbonitrile,

4-(((6-cyano-2′-methyl(1,1′-biphenyl)-3-yl)(1-methyl-1H-imidazol-5-yl)methoxy)-methyl)-6-methylisophthalonitrile,

5-(((2′-cyano(1,1′-biphenyl)-4-yl)methoxy)(1-methyl-1H-imidazol-5-yl)methyl)-2′-methyl(1,1′-biphenyl)-2-carbonitrile,

methyl 3-(((6-cyano-2′-methyl(1,1′-biphenyl)-3-yl)(1-methyl-1H-imidazol-5-yl)methoxy)-methyl)benzoate,

5-(((3,4-difluorobenzyl)oxy)(1-methyl-1H-imidazol-5-yl)methyl)-2′-methyl(1,1′-biphenyl)-2-carbonitrile,

(2′-methyl-5-((1-methyl-1H-imidazol-5-yl)((3,4,5-trimethoxybenzyl)oxy)methyl)-(1,1′-biphenyl)-2-carbonitrile,

(2′-methyl-5-((1-methyl-1H-imidazol-5-yl)(8-quinolinylmethoxy)methyl)(1,1′-biphenyl)-2-carbonitrile,

5-(((3,5-dimethoxybenzyl)oxy)(1-methyl-1H-imidazol-5-yl)methyl)-2′-methyl(1,1′-biphenyl)-2-carbonitrile,

(2′-methyl-5-((1-methyl-1H-imidazol-5-yl)((4-(methylsulfonyl)benzyl)oxy)-methyl)(1,1′-biphenyl)-2-carbonitrile,

5-(((6-chloro-1,3-benzodioxol-5-yl)methoxy)(1-methyl-1H-imidazol-5-yl)methyl)-2′-methyl(1,1′-biphenyl)-2-carbonitrile,

5-(((4-isopropylbenzyl)oxy)(1-methyl-1H-imidazol-5-yl)methyl)-2′-methyl(1,1′-biphenyl)-2-carbonitrile,

5-(((3,4-dimethylbenzyl)oxy)(1-methyl-1H-imidazol-5-yl)methyl)-2′-methyl(1,1′-biphenyl)-2-carbonitrile,

5-(((4-(benzyloxy)benzyl)oxy)(1-methyl-1H-imidazol-5-yl)methyl)-2′-methyl(1,1′-biphenyl)-2-carbonitrile,

5-(((6-fluoro-4H-1,3-benzodioxin-8-yl)methoxy)(1-methyl-1H-midazol-5-yl)methyl)-2′-methyl(1,1′-biphenyl)-2-carbonitrile,

5-(((2,4-dichlorobenzyl)oxy)(1-methyl-1H-imidazol-5-yl)methyl)-2′-methyl(1,1′-biphenyl)-2-carbonitrile,

5-(((3,5-dimethylbenzyl)oxy)(1-methyl-1H-imidazol-5-yl)methyl)-2′-methyl(1,1′-biphenyl)-2-carbonitrile,

5-(((5-(tert-butyl)-1,2,4-oxadiazol-3-yl)methoxy)(1-methyl-1H-imidazol-5-yl)methyl)-2′-methyl(1,1′-biphenyl)-2-carbonitrile,

5-(((4-iodobenzyl)oxy)(1-methyl-1H-imidazol-5-yl)methyl)-2′-methyl(1,1′-biphenyl)-2-carbonitrile,

5-(((1,1′-biphenyl)-4-ylmethoxy)(1-methyl-1H-imidazol-5-yl)methyl)-2′-methyl(1,1′-biphenyl)-2-carbonitrile,

5-(((2-(4-chlorophenyl)-1,3-thiazol-4-yl)methoxy)(1-methyl-1H-imidazol-5-yl)-methyl)-2′-methyl(1,1′-biphenyl)-2-carbonitrile,

5-(((5-(2-methoxyphenyl)-1,2,4-oxadiazol-3-yl)methoxy)(1-methyl-1H-imidazol-5-yl)methyl)-2′-methyl(1,1′-biphenyl)-2-carbonitrile,

5-(((4-chloro-2-nitrobenzyl)oxy)(1-methyl-1H-imidazol-5-yl)methyl)-2′-methyl(1,1′-biphenyl)-2-carbonitrile,

methyl 5-(((6-cyano-2′-methyl(1,1′-biphenyl)-3-yl)(1-methyl-1H-imidazol-5-yl)-methoxy)methyl)-2-furoate,

2′-methyl-5-((1-methyl-1H-imidazol-5-yl)((5-(4-(trifluoromethyl)phenyl)-1,2,4-oxadiazol-3-yl)methoxy)methyl)(1,1′-biphenyl)-2-carbonitrile,

methyl 8-(((6-cyano-2′-methyl(1,1′-biphenyl)-3-yl)(1-methyl-1H-imidazol-5-yl)methoxy)methyl)-4H-1,3-benzodioxine-6-carboxylate,

(2′-methyl-5-((1-methyl-1H-imidazol-5-yl)((6-nitro-4H-1,3-benzodioxin-8-yl)methoxy)methyl)(1,1′-biphenyl)-2-carbonitrile,

2′-methyl-5-((1-methyl-1H-imidazol-5-yl)((5-(3-(trifluoromethyl)phenyl)-1,2,4-oxadiazol-3-yl)methoxy)methyl)(1,1′-biphenyl)-2-carbonitrile,

5-(((5-acetyl-2-methoxybenzyl)oxy)(1-methyl-1H-imidazol-5-yl)methyl)-2′-methyl(1,1′-biphenyl)-2-carbonitrile,

2′-methyl-5-((1-methyl-1H-imidazol-5-yl)((5-phenyl-1,2,4-oxadiazol-3-yl)-methoxy)methyl)(1,1′-biphenyl)-2-carbonitrile,

5-(((5-(4-methoxyphenyl)-1,2,4-oxadiazol-3-yl)methoxy)(1-methyl-1H-imidazol-5-yl)methyl)-2′-methyl(1,1′-biphenyl)-2-carbonitrile,

5-(((5-(3-methoxyphenyl)-1,2,4-oxadiazol-3-yl)methoxy)(1-methyl-1H-imidazol-5-yl)methyl)-2′-methyl(1,1′-biphenyl)-2-carbonitrile,

2′-methyl-5-((1-methyl-1H-imidazol-5-yl)((2-(4-(trifluoromethyl)phenyl)-1,3-thiazol-4-yl)methoxy)methyl)(1,1′-biphenyl)-2-carbonitrile,

2′-methyl-5-((1-methyl-1H-imidazol-5-yl)((5-methyl-3-isoxazolyl)methoxy)methyl)-(1,1′-biphenyl)-2-carbonitrile,

(2′-methyl-5-((1-methyl-1H-imidazol-5-yl)((2-methyl-1-naphthyl)methoxy)methyl)-(1,1′-biphenyl)-2-carbonitrile,

(2′-methyl-5-((1-methyl-1H-imidazol-5-yl)((2,3,5,6-tetramethylbenzyl)oxy)methyl)-(1,1′-biphenyl)-2-carbonitrile,

(2′-methyl-5-((1-methyl-1H-imidazol-5-yl)((4-(trifluoromethoxy)benzyl)oxy)-methyl)(1,1′-biphenyl)-2-carbonitrile,

5-(((5,6-dichloro-3-pyridinyl)methoxy)(1-methyl-1H-imidazol-5-yl)methyl)-2′-methyl(1,1′-biphenyl)-2-carbonitrile,

5-(((3-chloro-5-(trifluoromethyl)-2-pyridinyl)methoxy)(1-methyl-1H-imidazol-5-yl)-methyl)-2′-methyl(1,1′-biphenyl)-2-carbonitrile,

2′-methyl-5-((1-methyl-1H-imidazol-5-yl)(2-naphthylmethoxy)methyl)(1,1′-biphenyl)-2-carbonitrile,

5-(((3-bromobenzyl)oxy)(1-methyl-1H-imidazol-5-yl)methyl)-2′-methyl(1,1′-biphenyl)-2-carbonitrile,

5-(((2-bromobenzyl)oxy)(1-methyl-1H-imidazol-5-yl)methyl)-2′-methyl(1,1′-biphenyl)-2-carbonitrile,

5-(((2,6-difluorobenzyl)oxy)(1-methyl-1H-imidazol-5-yl)methyl)-2′-methyl(1,1′-biphenyl)-2-carbonitrile,

5-(((2-fluoro-4-(trifluoromethyl)benzyl)oxy)(1-methyl-1H-imidazol-5-yl)methyl)-2′-methyl(1,1′-biphenyl)-2-carbonitrile,

4-(((6-cyano-2′-methyl(1,1′-biphenyl)-3-yl)(1-methyl-1H-imidazol-5-yl)methoxy)-methyl)-benzamide,

4-(((6-cyano-2′-methyl(1,1′-biphenyl)-3-yl)(1-methyl-1H-imidazol-5-yl)methoxy)-methyl)-N-methylbenzamide,

4-(((6-cyano-2′-methyl(1,1′-biphenyl)-3-yl)(1-methyl-1H-imidazol-5-yl)methoxy)-methyl)-N,N-dimethylbenzamide,

5-(((4-cyanobenzyl)oxy)(1-methyl-1H-imidazol-5-yl)methyl)-2′-formyl(1,1′-biphenyl)-2-carbonitrile,

5-(((4-cyanobenzyl)oxy)(1-methyl-1H-imidazol-5-yl)methyl)-2′-(trifluoromethyl)-(1,1′-biphenyl)-2-carbonitrile,

2′,4′-dichloro-5-(((4-cyanobenzyl)oxy)(1-methyl-1H-imidazol-5-yl)methyl)(1,1′-biphenyl)-2-carbonitrile,

2-(1-benzothien-2-yl)-4-(((4-cyanobenzyl)oxy)(1-methyl-1H-imidazol-5-yl)methyl)-benzonitrile,

5-(((4-cyanobenzyl)oxy)(1-methyl-1H-imidazol-5-yl)methyl)-2′-(hydroxymethyl)-(1,1′-biphenyl)-2-carbonitrile,

2′-cyano-5′-(((4-cyanobenzyl)oxy)(1-methyl-1H-imidazol-5-yl)methyl)(1,1′-biphenyl)-2-carboxylic acid,

4-(((3,4-dichlorobenzyl)oxy)(1-methyl-1H-imidazol-5-yl)methyl)-2-(8-quinolinyl)-benzonitrile,

4-(((3-fluoro-4-(trifluoromethyl)benzyl)oxy)(1-methyl-1H-imidazol-5-yl)methyl)-2-(8-quinolinyl)benzonitrile,

4-(((4-fluoro-3-(trifluoromethyl)benzyl)oxy)(1-methyl-1H-imidazol-5-yl)methyl)-2-(8-quinolinyl)benzonitrile,

4-(((4-cyano-3-(8-quinolinyl)phenyl)(1-methyl-1H-imidazol-5-yl)methoxy)methyl)-benzoic acid,

6-(((4-cyano-3-(8-quinolinyl)phenyl)(1-methyl-1H-imidazol-5-yl)methoxy)methyl)-nicotinamide,

6-(((4-cyano-3-(8-quinolinyl)phenyl)(1-methyl-1H-imidazol-5-yl)methoxy)methyl)-nicotinic acid,

4-(((3-chloro-5-(trifluoromethyl)-2-pyridinyl)methoxy)(1-methyl-1H-imidazol-5-yl)methyl)-2-(8-quinolinyl)benzonitrile,

6-(((4-cyano-3-(8-quinolinyl)phenyl)(1-methyl-1H-imidazol-5-yl)methoxy)methyl)-nicotinonitrile,

5-(((3,4-dichlorobenzyl)oxy)(1-methyl-1H-imidazol-5-yl)methyl)-2′-(trifluoromethyl)(1,1′-biphenyl)-2-carbonitrile,

5-(((3-fluoro-4-(trifluoromethyl)benzyl)oxy)(1-methyl-1H-imidazol-5-yl)methyl)-2′-(trifluoromethyl)(1,1′-biphenyl)-2-carbonitrile,

5-(((4-fluoro-3-(trifluoromethyl)benzyl)oxy)(1-methyl-1H-imidazol-5-yl)methyl)-2′-(trifluoromethyl)(1,1′-biphenyl)-2-carbonitrile,

6-(((6-cyano-2′-(trifluoromethyl)(1,1′-biphenyl)-3-yl)(1-methyl-1H-imidazol-5-yl)-methoxy)methyl)nicotinonitrile,

4-(((3-cyanobenzyl)oxy)(1-methyl-1H-imidazol-5-yl)methyl)-2-(1-naphthyl)-benzonitrile,

4-(((4-bromobenzyl)oxy)(1-methyl-1H-imidazol-5-yl)methyl)-2-(1-naphthyl)-benzonitrile,

4-(((6-cyano-2′-methyl(1,1′-biphenyl)-3-yl)(1-methyl-1H-imidazol-5-yl)methoxy)-methyl)-benzoic acid,

4-((1-methyl-1H-imidazol-5-yl)((3-chlorobenzyl)oxy)methyl)-2-(1-naphthyl)-benzonitrile,

5-(((4-cyano-3-(1-naphthyl)phenyl)(1-methyl-1H-imidazol-5-yl)methoxy)methyl)-2-pyridinecarbonitrile,

4-((1-methyl-1H-imidazol-5-yl)((4-azidobenzyl)oxy)methyl)-2-(1-naphthyl)-benzonitrile,

methyl 6-(((6-cyano-2′-(trifluoromethyl)(1,1′-biphenyl)-3-yl)(1-methyl-1H-imidazol-5-yl)methoxy)methyl)nicotinate,

5-(((4-cyanobenzyl)oxy)(1-methyl-1H-imidazol-5-yl)methyl)-2′,3′-dimethyl(1,1′-biphenyl)-2-carbonitrile,

2′,3′-dichloro-5-(((4-cyanobenzyl)oxy)(1-methyl-1H-imidazol-5-yl)methyl)(1,1′-biphenyl)-2-carbonitrile,

6-(((2′,3′-dichloro-6-cyano(1,1′-biphenyl)-3-yl)(1-methyl-1H-imidazol-5-yl)methoxy)-methyl)nicotinonitrile,

6-(((6-cyano-2′,3′-dimethyl(1,1′-biphenyl)-3-yl)(1-methyl-1H-imidazol-5-yl)-methoxy)methyl)nicotinonitrile, and

4-((4-cyanophenoxy)(1-methyl-1H-imidazol-5-yl)methyl)-2-(1-naphthyl)-benzonitrile.

In another preferred embodiment of compounds of formula (IV) are compounds wherein

Q¹ is O;

W is N═C(H); and

X, Y, and Z are C(H).

Compounds which support this embodiment include, but are not limited to,

6-(((4-cyano-3-(1-naphthyl)phenyl)(3-pyridinyl)methoxy)methyl)nicotinonitrile,

Example 296,

Example 297,

Example 298,

Example 299,

Example 300,

Example 301,

Example 302,

Example 303,

Example 306,

Example 310,

Example 311,

Example 331,

Example 332,

Example 344,

Example 345,

Example 346,

Example 348,

Example 349,

Example 350,

Example 351,

Example 352,

Example 353,

Example 354,

Example 355,

Example 357,

Example 358,

Example 359,

Example 360,

Example 362,

Example 366,

Example 368,

Example 369,

Example 370,

Example 371,

Example 372,

Example 373,

Example 374,

Example 375,

Example 376,

Example 377,

Example 378,

Example 379,

Example 380,

Example 381,

Example 382,

Example 383,

Example 384,

Example 389,

Example 391,

Example 392,

Example 393,

Example 394,

Example 395,

Example 396,

Example 397,

Example 398,

Example 399,

Example 400,

Example 401,

Example 402,

Example 403,

Example 404,

Example 405,

Example 406,

Example 407,

Example 408,

Example 409,

Example 410,

Example 411,

Example 412,

Example 413,

Example 414,

Example 415,

Example 416,

Example 417,

Example 418,

Example 419,

Example 420,

Example 421,

Example 422,

Example 423,

Example 424,

Example 425,

Example 426,

Example 427,

Example 428,

Example 429,

Example 430,

Example 431,

Example 432,

Example 433,

Example 434,

Example 435,

Example 436,

Example 437,

Example 438,

Example 439,

Example 440,

Example 441,

Example 442,

Example 443,

Example 444,

Example 445,

Example 446,

Example 447,

Example 448,

Example 449,

Example 451,

Example 453,

Example 454,

Example 455,

Example 456,

Example 457,

Example 458,

Example 459,

Example 460,

Example 461,

Example 462,

Example 463,

Example 464,

Example 465,

Example 466,

Example 476,

Example 477,

Example 478,

Example 479,

Example 480,

Example 481,

Example 503,

Example 504,

Example 505,

Example 506,

Example 507,

Example 508,

Example 509,

Example 510,

Example 511,

Example 512,

Example 513,

Example 514,

Example 525,

Example 526,

Example 533,

Example 534,

Example 535,

Example 537,

Example 538,

Example 539,

Example 540,

Example 541,

Example 542, and

Example 547.

In another preferred embodiment of compounds of formula (IV) are compounds wherein

Q is O;

W is S;

Y is N; and

X, and Z are C(H).

Compounds which support this embodiment include, but are not limited to,

5-((benzyloxy)(1,3-thiazol-5-yl)methyl)-2′-methyl(1,1′-biphenyl)-2-carbonitrile,

4-(((4-cyanobenzyl)oxy)(1,3-thiazol-5-yl)methyl)-2-(1-naphthyl)benzonitrile,

6-(((4-cyano-3-(1-naphthyl)phenyl)(1,3-thiazol-5-yl)methoxy)methyl)nicotinonitrile,

Example 552,

Example 553,

Example 554,

Example 555,

Example 556,

Example 557,

Example 558,

Example 559,

Example 563,

Example 567,

Example 568,

Example 589,

Example 590,

Example 602,

Example 603,

Example 604,

Example 606,

Example 607,

Example 608,

Example 609,

Example 610,

Example 611,

Example 612,

Example 613,

Example 615,

Example 616,

Example 617,

Example 618,

Example 619,

Example 623,

Example 625,

Example 626,

Example 627,

Example 628,

Example 629,

Example 630,

Example 631,

Example 632,

Example 633,

Example 634,

Example 635,

Example 636,

Example 637,

Example 638,

Example 639,

Example 640,

Example 645,

Example 647,

Example 648,

Example 649,

Example 650,

Example 651,

Example 652,

Example 653,

Example 654,

Example 655,

Example 656,

Example 657,

Example 658,

Example 659,

Example 660,

Example 661,

Example 662,

Example 663,

Example 664,

Example 665,

Example 666,

Example 667,

Example 668,

Example 669,

Example 670,

Example 671,

Example 672,

Example 673,

Example 674,

Example 675,

Example 676,

Example 677,

Example 678,

Example 679,

Example 680,

Example 681,

Example 682,

Example 683,

Example 684,

Example 685,

Example 686,

Example 687,

Example 688,

Example 689,

Example 690,

Example 691,

Example 692,

Example 693,

Example 694,

Example 695,

Example 696,

Example 697,

Example 698,

Example 699,

Example 700,

Example 701,

Example 702,

Example 703,

Example 704,

Example 705,

Example 707,

Example 709,

Example 710,

Example 711,

Example 712,

Example 713,

Example 714,

Example 715,

Example 716,

Example 717,

Example 718,

Example 719,

Example 720,

Example 721,

Example 722,

Example 732,

Example 733,

Example 734,

Example 735,

Example 736,

Example 737,

Example 762,

Example 763,

Example 764,

Example 765,

Example 766,

Example 767,

Example 768,

Example 769,

Example 770,

Example 771,

Example 772,

Example 773,

Example 784,

Example 785,

Example 792,

Example 793,

Example 794,

Example 796,

Example 797,

Example 798,

Example 799,

Example 800,

Example 801, and

Example 806.

In another preferred embodiment of compounds of formula (IV) are compounds wherein

Q¹ is N(R⁴);

W is N;

Y is N; and

X and Z are C(H).

Compounds which support this embodiment include, but are not limited to,

(2′-methyl-5-((1-methyl-1H-imidazol-5-yl)((4-nitrobenzyl)amino)methyl)(1,1′-biphenyl)-2-carbonitrile,

4-(((4-cyanobenzyl)amino)(1-methyl-1H-imidazol-5-yl)methyl)-2-(1-naphthyl)-benzonitrile,

5-(((1-benzoyl-4-piperidinyl)amino)(1-methyl-1H-imidazol-5-yl)methyl)-2′-methyl-(1,1′-biphenyl)-2-carbonitrile,

4-((l1-methyl-1H-imidazol-5-yl)((4-(methylsulfonyl)benzyl)amino)methyl)-2-(1-naphthyl)benzonitrile,

5-((benzylamino)(1-methyl-1H-imidazol-5-yl)methyl)-2′-methyl(1,1′-biphenyl)-2-carbonitrile,

5-(((cyclohexylmethyl)amino)(1-methyl-1H-imidazol-5-yl)methyl)-2′-methyl(1,1′-biphenyl)-2-carbonitrile,

5-(((4-cyanobenzyl)amino)(1-methyl-1H-imidazol-5-yl)methyl)-2′-methyl(1,1′-biphenyl)-2-carbonitrile,

5-((((6-cyano-2′-methyl(1,1′-biphenyl)-3-yl)methyl)amino)(1-methyl-1H-imidazol-5-yl)methyl)-2′-methyl(1,1′-biphenyl)-2-carbonitrile,

5-((ethyl(4-nitrobenzyl)amino)(1-methyl-1H-imidazol-5-yl)methyl)-2′-methyl(1,1′-biphenyl)-2-carbonitrile,

5-(((4-cyanobenzyl)(ethyl)amino)(1-methyl-1H-imidazol-5-yl)methyl)-2′-methyl-(1,1′-biphenyl)-2-carbonitrile,

4-(((4-cyanobenzyl)(methyl)amino)(1-methyl-1H-imidazol-5-yl)methyl)-2-(1-naphthyl)benzonitrile,

4-((butyl(4-cyanobenzyl)amino)(1-methyl-1H-imidazol-5-yl)methyl)-2-(1-naphthyl)-benzonitrile,

4-((1-methyl-1H-imidazol-5-yl)(phenethylamino)methyl)-2-(1-naphthyl)benzonitrile,

4-(((3-bromo-4-cyanobenzyl)amino)(1-methyl-1H-imidazol-5-yl)methyl)-2-(1-naphthyl)benzonitrile,

4-(((3-chloro-4-cyanobenzyl)amino)(1-methyl-1H-imidazol-5-yl)methyl)-2-(1-naphthyl)benzonitrile,

4-(((1-(4-cyanophenyl)ethyl)amino)(1-methyl-1H-imidazol-5-yl)methyl)-2-(1-naphthyl)benzonitrile,

4-(((4-cyano-3-iodobenzyl)amino)(1-methyl-1H-imidazol-5-yl)methyl)-2-(1-naphthyl)benzonitrile,

methyl 4-((((4-cyano-3-(1-naphthyl)phenyl)(1-methyl-1H-imidazol-5-yl)methyl)-amino)methyl)benzoate,

4-((((4-cyano-3-(1-naphthyl)phenyl)(1-methyl-1H-imidazol-5-yl)methyl)amino)-methyl)benzoic acid,

4-(((4-chlorobenzyl)amino)(1-methyl-1H-imidazol-5-yl)methyl)-2-(1-naphthyl)-benzonitrile,

4-(((3,4-dichlorobenzyl)amino)(1-methyl-1H-imidazol-5-yl)methyl)-2-(1-naphthyl)-benzonitrile,

4-((((4-cyano-3-(1-naphthyl)phenyl)(1-methyl-1H-imidazol-5-yl)methyl)amino)-methyl)-N-methylbenzamide,

ethyl 4-(((4-cyano-3-(1-naphthyl)phenyl)(1-methyl-1H-imidazol-5-yl)methyl)-amino)-1-piperidinecarboxylate,

6-((((4-cyano-3-(1-naphthyl)phenyl)(1-methyl-1H-imidazol-5-yl)methyl)amino)-methyl)nicotinonitrile,

methyl 6-((((4-cyano-3-(1-naphthyl)phenyl)(1-methyl-1H-imidazol-5-yl)methyl)-amino)methyl)nicotinate,

N-(4-((((4-cyano-3-(1-naphthyl)phenyl)(1-methyl-1H-imidazol-5-yl)methyl)amino)-methyl)phenyl)acetamide,

benzyl 4-(((4-cyano-3-(1-naphthyl)phenyl)(1-methyl-1H-imidazol-5-yl)methyl)-amino)-1-piperidinecarboxylate,

4-(((1-benzyl-4-piperidinyl)amino)(1-methyl-1H-imidazol-5-yl)methyl)-2-(1-naphthyl)benzonitrile,

tert-butyl 4-(((4-cyano-3-(1-naphthyl)phenyl)(1-methyl-1H-imidazol-5-yl)methyl)-amino)-1-piperidinecarboxylate,

4-(((1-benzoyl-4-piperidinyl)amino)(1-methyl-1H-imidazol-5-yl)methyl)-2-(1-naphthyl)benzonitrile,

4-((((4-cyano-3-(1-naphthyl)phenyl)(1-methyl-1H-imidazol-5-yl)methyl)amino)-methyl)benzamide,

4-((1-methyl-1H-imidazol-5-yl)(((1-methyl-2-oxo-1,2-dihydro-4-pyridinyl)methyl)-amino)methyl)-2-(1-naphthyl)benzonitrile,

4-((4-cyanoanilino)(1-methyl-1H-imidazol-5-yl)methyl)-2-(1-naphthyl)benzonitrile, and

4-((3-cyanoanilino)(1-methyl-1H-imidazol-5-yl)methyl)-2-(1-naphthyl)benzonitrile.

In another preferred embodiment of compounds of formula (IV) are compounds wherein

Q¹ is N(R⁴);

W is N═C(H);

X, Y, and Z are C(H).

Compounds which support this embodiment include, but are not limited to,

Example 304,

Example 305,

Example 308,

Example 309,

Example 312,

Example 313,

Example 314,

Example 315,

Example 316,

Example 317,

Example 318,

Example 319,

Example 320,

Example 321,

Example 323,

Example 324,

Example 325,

Example 326,

Example 327,

Example 330,

Example 333,

Example 334,

Example 335,

Example 336,

Example 337,

Example 338,

Example 339,

Example 340,

Example 341,

Example 342,

Example 343,

Example 452,

Example 544, and

Example 545.

In another preferred embodiment of compounds of formula (IV) are compounds wherein

Q¹ is N(R⁴);

W is S;

Y is N; and

X and Z are C(H).

Compounds which support this embodiment include, but are not limited to,

Example 561,

Example 562,

Example 565,

Example 566,

Example 569,

Example 570,

Example 571,

Example 572,

Example 573,

Example 574,

Example 575,

Example 576,

Example 577,

Example 579,

Example 581,

Example 582,

Example 583,

Example 584,

Example 585,

Example 588,

Example 591,

Example 592,

Example 593,

Example 594,

Example 595,

Example 596,

Example 597,

Example 598,

Example 599,

Example 600,

Example 601,

Example 708,

Example 747,

Example 748,

Example 749,

Example 750,

Example 751,

Example 752,

Example 803, and

Example 804.

In another preferred embodiment of compounds of formula (IV) are compounds wherein

Q¹ is S(O)ₜ, wherein t is zero, one, or two;

W is N;

Y is N; and

X and Z are C(H).

Compounds which support this embodiment include, but are not limited to,

4-(((4-cyanobenzyl)sulfanyl)(1-methyl-1H-imidazol-5-yl)methyl)-2-(1-naphthyl)-benzonitrile, and

4-(((4-cyanobenzyl)sulfonyl)(1-methyl-1H-imidazol-5-yl)methyl)-2-(1-naphthyl)-benzonitrile.

In another preferred embodiment of compounds of formula (IV) are compounds wherein

Q¹ is S(O)ₜ, wherein t is zero, one, or two;

W is N═C(H); and

X, Y, and Z are C(H).

Compounds which support this embodiment include, but are not limited to,

Example 347, and

Example 356.

In another preferred embodiment of compounds of formula (IV) are compounds wherein

Q¹ is S(O)ₜ, wherein t is zero, one, or two;

W is S;

Y is N; and

X and Z are C(H).

Compounds which support this embodiment include, but are not limited to,

Example 605, and

Example 614.

In another preferred embodiment of compounds of formula (IV) are compounds wherein

Q¹ is N(R⁵)SO₂;

W is N;

Y is N; and

X and Z are C(H).

A compound which supports this embodiment includes, but is not limited to, 4-cyano-N-((4-cyano-3-(1-naphthyl)phenyl)(1-methyl-1H-imidazol-5-yl)methyl)-benzenesulfonamide.

In another preferred embodiment of compounds of formula (IV) are compounds wherein

Q¹ is N(R⁵)SO²;

W is N═C(H); and

X, Y, and Z are C(H).

A compound which supports this embodiment includes, but is not limited to,

Example 543.

In another preferred embodiment of compounds of formula (IV) are compounds wherein

Q¹ is N(R⁵)SO₂;

W is S;

Y is N; and

X and Z are C(H).

A compound which supports this embodiment includes, but is not limited to,

Example 802.

In another preferred embodiment of compounds of formula (IV) are compounds wherein

Q¹ is absent;

W is N;

Y is N; and

X and Z are C(H).

Compounds which support this embodiment include, but are not limited to,

(2′-methyl-5-((1-methyl-1H-imidazol-5-yl)(3-oxo-4-(3-(trifluoromethoxy)phenyl)-1-piperazinyl)methyl)(1,1′-biphenyl)-2-carbonitrile, and

tert-butyl 1-((4-cyano-3-(1-naphthyl)phenyl)(1-methyl-1H-imidazol-5-yl)methyl)-4-piperidinylcarbamate.

In another preferred embodiment of compounds of formula (IV) are compounds wherein

Q¹ is absent;

W is N═C(H); and

X, Y, and Z are C(H).

Compounds which support this embodiment include, but are not limited to,

Example 307, and

Example 546.

In another preferred embodiment of compounds of formula (IV) are compounds wherein

Q¹ is absent;

W is S;

Y is N; and

X and Z are C(H).

Compounds which support this embodiment include, but are not limited to,

Example 564, and

Example 805.

In another embodiment, the instant invention discloses compounds of formula (V)

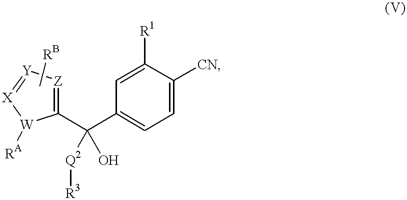

or a pharmaceutically acceptable salt thereof, wherein

R^{A} is absent or selected from the group consisting of hydrogen, optionally substituted alkyl, alkoxycarbonyl, and a nitrogen protecting group;

R^{B} is absent or selected from the group consisting of optionally substituted alkyl, alkoxy, alkanoyl, alkanoyloxy, alkoxycarbonyl, alkylsulfonyl, amino, aminosulfonyl, azido, carboxamido, carboxyl, cyano, halo, hydroxyl, perfluoroalkyl, and perfluoroalkoxy; and

W is C(H)═C(H), X is N, and Y and Z are C(H); or

W is C(H)═N or N═C(H), wherein each group is drawn with its left end attached to X and its right end attached to the carbon substituted with L²; and X, Y and Z are C(H); or

W is N or S, one of X, Y, or Z is C(H), and the remainder are C(H) or N;

with the proviso that R^{A} is present when and only when W is N.

In a preferred embodiment of compounds of formula (V) are compounds wherein

W is N;

Y is N; and

X and Z are C(H).

Compounds which support this embodiment include, but are not limited to,

5-(1-hydroxy-1-(1-methyl-1H-imidazol-5-yl)-3-phenyl-2-propynyl)-2′-methyl(1,1′-biphenyl)-2-carbonitrile,

5-(1-hydroxy-1-(1-methyl-1H-imidazol-5-yl)-3-phenylpropyl)-2′-methyl(1,1′-biphenyl)-2-carbontrile, and

4-(1-hydroxy-1-(1-methyl-1H-imidazol-5-yl)-3-phenyl-2-propynyl)-2-(1-naphthyl)benzonitrile.

In another preferred embodiment of compounds of formula (V) are compounds wherein

W is N═C(H); and

X, Y, and Z are C(H).

Compounds which support this embodiment include, but are not limited to,

Example 385,

Example 386, and

Example 387.

In another preferred embodiment of compounds of formula (V) are compounds wherein

W is S;

Y is N; and

X and Z are C(H).

Compounds which support this embodiment include, but are not limited to,

Example 641,

Example 642, and

Example 643.

In another embodiment, the instant invention discloses compounds of formula (VI)

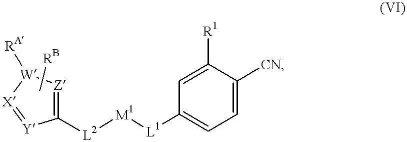

or pharmaceutically acceptable salts thereof, wherein

W′ is N or S; and

one of X′, Y′, or Z′ is C(H), and the remainder are C(H) or N;

R^{A′} is absent or selected from the group consisting of hydrogen, optionally substituted alkyl, alkoxycarbonyl, hydroxyl, and a nitrogen protecting group; and

R^{B} is absent or selected from the group consisting of optionally substituted alkyl, alkoxy, alkanoyl, alkanoyloxy, alkoxycarbonyl, alkylsulfonyl, amino, aminosulfonyl, azido, carboxamido, carboxyl, cyano, halo, hydroxyl, perfluoroalkyl, and perfluoroalkoxy;

with the proviso that R^{A} is present when and only when W′ is N.

In another embodiment, the instant invention discloses compounds of formula (VII)

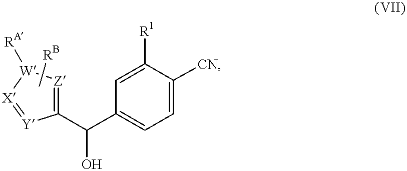

or pharmaceutically acceptable salts thereof, wherein

W′ is N or S; and

one of X′, Y′, or Z′ is C(H), and the remainder are C(H) or N;

R^{A′} is absent or selected from the group consisting of hydrogen, optionally substituted alkyl, alkoxycarbonyl, hydroxyl, and a nitrogen protecting group; and

R^{B} is absent or selected from the group consisting of optionally substituted alkyl, alkoxy, alkanoyl, alkanoyloxy, alkoxycarbonyl, alkylsulfonyl, amino, aminosulfonyl, azido, carboxamido, carboxyl, cyano, halo, hydroxyl, perfluoroalkyl, and perfluoroalkoxy;

with the proviso that R^{A} is present when and only when W′ is N.

In another embodiment, the instant invention discloses compounds of formula (VIII)

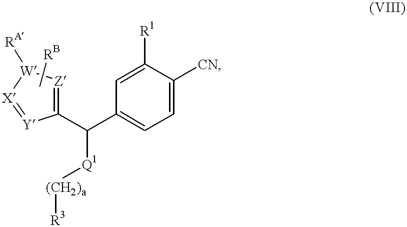

or pharmaceutically acceptable salts thereof, wherein

a is zero to six;

W′ is N or S; and

one of X′, Y′, or Z′ is C(H), and the remainder are C(H) or N;

R^{A′} is absent or selected from the group consisting of hydrogen, optionally substituted alkyl, alkoxycarbonyl, hydroxyl, and a nitrogen protecting group; and

R^{B} is absent or selected from the group consisting of optionally substituted alkyl, alkoxy, alkanoyl, alkanoyloxy, alkoxycarbonyl, alkylsulfonyl, amino, aminosulfonyl, azido, carboxamido, carboxyl, cyano, halo, hydroxyl, perfluoroalkyl, and perfluoroalkoxy;

with the proviso that R^{A′} is present when and only when W′ is N.

In a preferred embodiment of compounds of formula (VIII) are compounds wherein

Q¹ is O;

W′ is N;

Y′ is N; and

X′ and Z′ are C(H).

A compound which supports this embodiment includes, but is not limited to,

4-(((4-cyanobenzyl)oxy)(1-trityl-1H-imidazol-4-yl)methyl)-2-(1-naphthyl)benzonitrile.

In another preferred embodiment of compounds of formula (VIII) are compounds wherein

Q is O;

W′ is S; and

X′, Y′, and Z′ are C(H).

Compounds which support this embodiment include, but are not limited to,

5-((benzyloxy)(3-thienyl)methyl)-2′-methyl(1,1′-biphenyl)-2-carbonitrile, and

6-(((4-cyano-3-(1-naphthyl)phenyl)(3-thienyl)methoxy)methyl)nicotinonitrile.

In another embodiment, the instant invention discloses compounds of formula (IX)

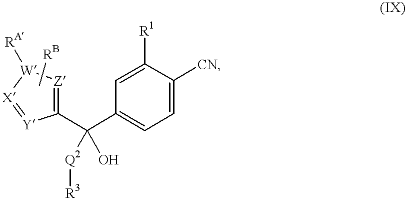

or pharmaceutically acceptable salts thereof, wherein

W′ is N or S; and

one of X′, Y′, or Z′ is C(H), and the remainder are C(H) or N;

R^{A′} is absent or selected from the group consisting of hydrogen, optionally substituted alkyl, alkoxycarbonyl, hydroxyl, and a nitrogen protecting group; and

R^{B} is absent or selected from the group consisting of optionally substituted alkyl, alkoxy, alkanoyl, alkanoyloxy, alkoxycarbonyl, alkylsulfonyl, amino, aminosulfonyl, azido, carboxamido, carboxyl, cyano, halo, hydroxyl, perfluoroalkyl, and perfluoroalkoxy;

with the proviso that R^{A′} is present when and only when W′ is N.

In another embodiment, the instant invention discloses compounds of formula (X)

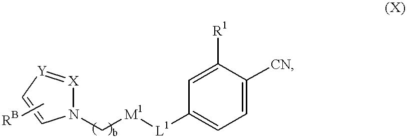

or pharmaceutically acceptable salts thereof, wherein

b is two to six;

R^{B} is absent or selected from the group consisting of optionally substituted alkyl, alkoxy, alkanoyl, alkanoyloxy, alkoxycarbonyl, alkylsulfonyl, amino, aminosulfonyl, azido, carboxamido, carboxyl, cyano, halo, hydroxyl, perfluoroalkyl, and perfluoroalkoxy; and

one of X and Y is C(H) and the other is C(H) or N.

In another embodiment, the instant invention discloses compounds of formula (XI)

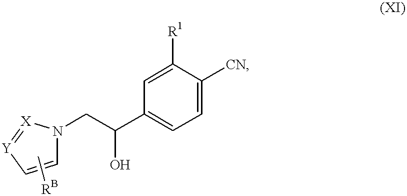

or pharmaceutically acceptable salts thereof, wherein

R^{B} is absent or selected from the group consisting of optionally substituted alkyl, alkoxy, alkanoyl, alkanoyloxy, alkoxycarbonyl, alkylsulfonyl, amino, aminosulfonyl, azido, carboxamido, carboxyl, cyano, halo, hydroxyl, perfluoroalkyl, and perfluoroalkoxy; and

one of X and Y is C(H) and the other is C(H) or N.

In another embodiment, the instant invention discloses compounds of formula (XII)

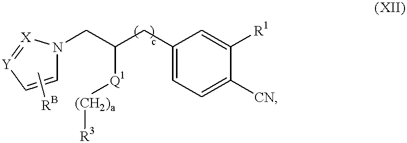

or pharmaceutically acceptable salts thereof, wherein

a is zero to six;

c is zero to two;

R^{B} is absent or selected from the group consisting of optionally substituted alkyl, alkoxy, alkanoyl, alkanoyloxy, alkoxycarbonyl, alkylsulfonyl, amino, aminosulfonyl, azido, carboxamido, carboxyl, cyano, halo, hydroxyl, perfluoroalkyl, and perfluoroalkoxy; and

one of X and Y is C(H) and the other is C(H) or N.

In a preferred embodiment of compounds of formula (XII) are compounds wherein

c is zero;

X is C(H);

Y is N; and

Q¹ is O.

A compound which supports this embodiment includes, but is not limited to,

5-(1-(benzyloxy)-2-(1H-imidazol-1-yl)ethyl)-2′-methyl(1,1′-biphenyl)-2-carbonitrile.

In another embodiment, the instant invention discloses compounds of formula (XIII)

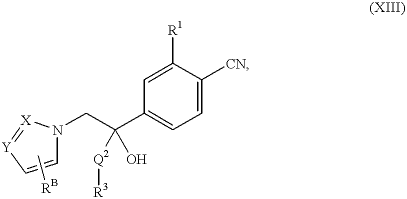

or pharmaceutically acceptable salts thereof, wherein

R^{B} is absent or selected from the group consisting of optionally substituted alkyl, alkoxy, alkanoyl, alkanoyloxy, alkoxycarbonyl, alkylsulfonyl, amino, aminosulfonyl, azido, carboxamido, carboxyl, cyano, halo, hydroxyl, perfluoroalkyl, and perfluoroalkoxy; and

one of X and Y is C(H) and the other is C(H) or N.

In another embodiment, the instant invention discloses compounds of formula (XIV)

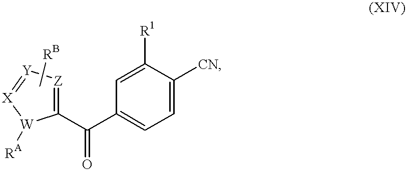

or a pharmaceutically acceptable salt thereof, wherein

R^{A} is absent or selected from the group consisting of hydrogen, optionally substituted alkyl, alkoxycarbonyl, and a nitrogen protecting group;

R^{B} is absent or selected from the group consisting of optionally substituted alkyl, alkoxy, alkanoyl, alkanoyloxy, alkoxycarbonyl, alkylsulfonyl, amino, aminosulfonyl, azido, carboxamido, carboxyl, cyano, halo, hydroxyl, perfluoroalkyl, and perfluoroalkoxy; and

W is C(H)═C(H), X is N, and Y and Z are C(H); or

W is C(H)═N or N═C(H), wherein each group is drawn with its left end attached to X and its right end attached to the carbon substituted with L²; and X, Y and Z are C(H); or

W is N or S, one of X, Y, or Z is C(H), and the remainder are C(H) or N;

with the proviso that R^{A} is present when and only when W is N.

In a preferred embodiment of compounds of formula (XIV) are compounds wherein

W is N;

Y is N; and

X and Z are C(H).

Compounds which support this embodiment include, but are not limited to,

(2′-methyl-5-((1-methyl-1H-imidazol-5-yl)carbonyl)(1,1′-biphenyl)-2-carbonitrile,

4-((1-methyl-1H-imidazol-5-yl)carbonyl)-2-(8-quinolinyl)benzontrile, and

5-((1-methyl-1H-midazol-5-yl)carbonyl)-2′-(trifluoromethyl)(1,1′-biphenyl)-2-carbonitrile.

In another preferred embodiment of compounds of formula (XIV) are compounds wherein

W is N═C(H); and

X, Y, and Z are C(H).

Compounds which support this embodiment are

Example 367,

Example 501, and

Example 502.

In another preferred embodiment of compounds of formula (XIV) are compounds wherein

W is S; and

X, Y, and Z are C(H).

Compounds which support this embodiment are

Example 624,

Example 760, and

Example 761.

In another embodiment, the instant invention discloses a method for inhibiting farnesyltransferase comprising administering a pharmaceutically acceptable amount of a compound of formula (I).

In another embodiment, the instant invention discloses a method for inhibiting farnesyltransferase comprising administering a pharmaceutically acceptable amount of a compound of formula (II).

In another embodiment, the instant invention discloses a method for inhibiting farnesyltransferase comprising administering a pharmaceutically acceptable amount of a compound of formula (III).

In another embodiment, the instant invention discloses a method for inhibiting farnesyltransferase comprising administering a pharmaceutically acceptable amount of a compound of formula (IV).

In another embodiment, the instant invention discloses a method for inhibiting farnesyltransferase comprising administering a pharmaceutically acceptable amount of a compound of formula (V).

In another embodiment, the instant invention discloses a method for inhibiting farnesyltransferase comprising administering a pharmaceutically acceptable amount of a compound of formula (VI).

In another embodiment, the instant invention discloses a method for inhibiting farnesyltransferase comprising administering a pharmaceutically acceptable amount of a compound of formula (VII).

In another embodiment, the instant invention discloses a method for inhibiting farnesyltransferase comprising administering a pharmaceutically acceptable amount of a compound of formula (VIII).

In another embodiment, the instant invention discloses a method for inhibiting farnesyltransferase comprising administering a pharmaceutically acceptable amount of a compound of formula (IX).

In another embodiment, the instant invention discloses a method for inhibiting farnesyltransferase comprising administering a pharmaceutically acceptable amount of a compound of formula (X).

In another embodiment, the instant invention discloses a method for inhibiting farnesyltransferase comprising administering a pharmaceutically acceptable amount of a compound of formula (XI).

In another embodiment, the instant invention discloses a method for inhibiting farnesyltransferase comprising administering a pharmaceutically acceptable amount of a compound of formula (XII).

In another embodiment, the instant invention discloses a method for inhibiting farnesyltransferase comprising administering a pharmaceutically acceptable amount of a compound of formula (XIII).

In another embodiment, the instant invention discloses a method of inhibiting farnesyltransferase comprising administering a pharmaceutically acceptable amount of a compound of formula (XIV).

In another embodiment, the instant invention discloses a method for treating cancer in a mammal in recognized need of such treatment comprising administering to the mammal a pharmaceutically acceptable amount of a compound of formula (I).

In another embodiment, the instant invention discloses a method for treating cancer in a mammal in recognized need of such treatment comprising administering to the mammal a pharmaceutically acceptable amount of a compound of formula (II).

In another embodiment, the instant invention discloses a method for treating cancer in a mammal in recognized need of such treatment comprising administering to the mammal a pharmaceutically acceptable amount of a compound of formula (III).

In another embodiment, the instant invention discloses a method for treating cancer in a mammal in recognized need of such treatment comprising administering to the mammal a pharmaceutically acceptable amount of a compound of formula (IV).

In another embodiment, the instant invention discloses a method for treating cancer in a mammal in recognized need of such treatment comprising administering to the mammal a pharmaceutically acceptable amount of a compound of formula (V).

In another embodiment, the instant invention discloses a method for treating cancer in a mammal in recognized need of such treatment comprising administering to the mammal a pharmaceutically acceptable amount of a compound of formula (VI).

In another embodiment, the instant invention discloses a method for treating cancer in a mammal in recognized need of such treatment comprising administering to the mammal a pharmaceutically acceptable amount of a compound of formula (VII).

In another embodiment, the instant invention discloses a method for treating cancer in a mammal in recognized need of such treatment comprising administering to the mammal a pharmaceutically acceptable amount of a compound of formula (VIII).

In another embodiment, the instant invention discloses a method for treating cancer in a mammal in recognized need of such treatment comprising administering to the mammal a pharmaceutically acceptable amount of a compound of formula (IX).

In another embodiment, the instant invention discloses a method for treating cancer in a mammal in recognized need of such treatment comprising administering to the mammal a pharmaceutically acceptable amount of a compound of formula (X).

In another embodiment, the instant invention discloses a method for treating cancer in a mammal in recognized need of such treatment comprising administering to the mammal a pharmaceutically acceptable amount of a compound of formula (XI).

In another embodiment, the instant invention discloses a method for treating cancer in a mammal in recognized need of such treatment comprising administering to the mammal a pharmaceutically acceptable amount of a compound of formula (XII).

In another embodiment, the instant invention discloses a method for treating cancer in a mammal in recognized need of such treatment comprising administering to the mammal a pharmaceutically acceptable amount of a compound of formula (XIII).

In another embodiment, the instant invention discloses a method for treating cancer in a mammal in recognized need of such treatment comprising administering to the mammal a pharmaceutically acceptable amount of a compound of formula (XIV).

In another embodiment, the instant invention discloses a compound of formula (I) in combination with a pharmaceutically acceptable carrier.

In another embodiment, the instant invention discloses a compound of formula (II) in combination with a pharmaceutically acceptable carrier.

In another embodiment, the instant invention discloses a compound of formula (III) in combination with a pharmaceutically acceptable carrier.

In another embodiment, the instant invention discloses a compound of formula (IV) in combination with a pharmaceutically acceptable carrier.

In another embodiment, the instant invention discloses a compound of formula (V) in combination with a pharmaceutically acceptable carrier.

In another embodiment, the instant invention discloses a compound of formula (VI) in combination with a pharmaceutically acceptable carrier.

In another embodiment, the instant invention discloses a compound of formula (VII) in combination with a pharmaceutically acceptable carrier.

In another embodiment, the instant invention discloses a compound of formula (VIII) in combination with a pharmaceutically acceptable carrier.

In another embodiment, the instant invention discloses a compound of formula (IX) in combination with a pharmaceutically acceptable carrier.

In another embodiment, the instant invention discloses a compound of formula (X) in combination with a pharmaceutically acceptable carrier.

In another embodiment, the instant invention discloses a compound of formula (XI) in combination with a pharmaceutically acceptable carrier.

In another embodiment, the instant invention discloses a compound of formula (XII) in combination with a pharmaceutically acceptable carrier.

In another embodiment, the instant invention discloses a compound of formula (XIII) in combination with a pharmaceutically acceptable carrier.

In another embodiment, the instant invention discloses a compound of formula (XIV) in combination with a pharmaceutically acceptable carrier.

### DETAILED DESCRIPTION OF THE INVENTION

The instant invention provides substituted phenyl farnesyltransferase inhibitors. As used in the specification, the following terms have the meanings indicated.

The term “alkanoyl,” as used herein, refers to an alkyl group, as defined herein, or a substituted alkyl group, as defined herein, attached to the parent molecular group through a carbonyl, as defined herein.

The term “alkoxy,” as used herein, refers to an alkyl group, as defined herein, or a substituted alkyl group, as defined herein, attached to the parent molecular group through an oxygen atom.

The term “alkoxycarbonyl,” as used herein, refers to an ester group; e.g., an alkoxy group as defined herein, attached to the parent molecular group through a carbonyl, as defined herein.

The term “alkenyl,” as used herein, refers to a monovalent straight or branched chain hydrocarbon radical having from two to six carbons and at least one carbon-carbon double bond.

The term “alkenylene,” as used herein, refers to a divalent straight or branched chain hydrocarbon radical having from two to six carbons and at least one carbon-carbon double bond.

The term “alkyl,” as used herein, refers to a saturated, monovalent straight or branched chain hydrocarbon having from one to six carbons.

The term “alkylene,” as used herein, refers to a divalent straight or branched chain saturated hydrocarbon diradical having from one to six carbons.

The term “alkylsulfonyl,” as used herein, refers to an alkyl group, as defined herein, or a substituted alkyl group, as defined herein, attached to the parent molecular group through a sulfonyl group, as defined herein.

The term “alkynyl,” as used herein, refers to a monovalent straight or branched chain hydrocarbon group having from two to six carbons and at least one carbon-carbon triple bond.

The term “alkynylene,” as used herein, refers to a divalent straight or branched chain hydrocarbon group having from two to six carbons and at least one carbon-carbon triple bond.

The term “amino,” as used herein, refers to —NH₂ or derivatives thereof formed by independent replacement of one or both hydrogen atoms thereon with a substituent or substituents independently selected from the group consisting of alkyl, alkenyl, alkynyl, aryl, arylalkyl, cycloalkyl, cycloalkylalkyl, heteroaryl, heteroarylalkyl, and an amino protecting group.

The term “aminosulfonyl,” as used herein, refers to an amino group, as defined herein, attached to the parent molecular group through a sulfonyl group, as defined herein.

The terms “amino protecting group,” or “nitrogen protecting group,” as used herein, refer to selectively introducible and removable groups which protect amino groups against undesirable side reactions during synthetic procedures. Examples of amino protecting groups include methoxycarbonyl, ethoxycarbonyl, trichloroethoxycarbonyl, benzyloxycarbonyl (Cbz), chloroacetyl, trifluoroacetyl, phenylacetyl, formyl, acetyl, benzoyl, tert-butoxycarbonyl (Boc), para-methoxybenzyloxycarbonyl, isopropoxycarbonyl, phthaloyl, succinyl, benzyl, diphenylmethyl, triphenylmethyl (trityl), methanesulfonyl, para-toluenesulfonyl, trimethylsilyl, triethylsilyl, triphenylsilyl, and the like. Preferred nitrogen protecting groups of the instant invention are benzyloxycarbonyl (Cbz), formyl, acetyl, methoxycarbonyl, ethoxycarbonyl, benzoyl, tert-butoxycarbonyl (Boc), and triphenylmethyl (trityl).

The term “aryl,” as used herein, refers to groups containing at least one aromatic, carbocyclic ring. Aryl groups of the instant invention are exemplified by phenyl, naphthyl, dihydronaphthyl, tetrahydronaphthyl, indanyl, indenyl, anthracenyl, acenaphthylenyl, dihydroacenaphthylenyl, and the like. The aryl groups of the instant invention can be optionally substituted with one, two, three, four, or five radicals independently selected from the group consisting of optionally substituted alkyl, alkenyl, alkynyl, alkoxy, alkanoyl, alkanoyloxy, alkoxycarbonyl, alkylsulfonyl, amino, aminosulfonyl, azido, carboxamido, carboxyl, cyano, halo, hydroxyalkyl, hydroxyl, nitro, perfluoroalkyl, perfluoroalkoxy, oxo, thioalkoxy, phenyl, heteroaryl selected from the group consisting of furanyl, thienyl, pyrrolyl, oxazolyl, thiazolyl, imidazolyl, isoxazolyl, isothiazolyl, oxadiazolyl, oxadiazolyl, triazolyl, thiadiazolyl, pyridyl, pyridazinyl, pyrimidinyl, pyrazinyl, and triazinyl, and heterocycloalkyl selected from the group consisting of tetrahydrofuranyl, piperidinyl, piperazinyl, morpholinyl, and thiomorpholinyl. The phenyl, the heteroaryl, and the heterocycloalkyl groups optionally substituting the aryl groups of the instant invention are attached to the aryl groups through either a covalent bond, an alkyl group, an oxygen atom, or a carbonyl group, as defined herein. The phenyl, the heteroaryl, and the heterocycloalkyl groups optionally substituting the aryl groups of the instant invention can also be further substituted with one, two, or three substituents independently selected from the group consisting of alkyl, alkoxy, carboxyl, azido, carboxaldehyde, halo, hydroxyl, perfluoroalkyl, and perfluoroalkoxy.

The term “arylalkyl,” as used herein, refers to an aryl group, as defined herein, attached to the parent molecular group through an alkyl group, as defined herein.

The term “arylsulfonyl,” as used herein, refers to an aryl group, as defined herein, attached to the parent molecular group through a sulfonyl group, as defined herein.

The term “aryloyl,” as used herein, refers to an aryl group, as defined herein, attached to the parent molecular group through a carbonyl group, as defined herein.

The term “azido,” as used herein, refers to —N₃.

The term “carbonyl,” as used herein, refers to —C(O)—.

The term “carboxamido,” as used herein, refers to an amide; e.g., an amino group attached to the parent molecular group through a carbonyl group, as defined herein.

The term “carboxyl,” as used herein, refers to —CO₂H or a derivative thereof formed by replacement of the hydrogen atom thereon by a carboxyl protecting group.

The term “carboxyl protecting group,” as used herein, refers to selectively introducible and removable groups which protect carboxyl groups against undesirable side reactions during synthetic procedures and includes all conventional carboxyl protecting groups. Examples of carboxyl groups include methyl, ethyl, n-propyl, isopropyl, 1,1-dimethylpropyl, n-butyl, tert-butyl, phenyl, naphthyl, benzyl, diphenylmethyl, triphenylmethyl (trityl), para-nitrobenzyl, para-methoxybenzyl, acetylmethyl, benzoylmethyl, para-nitrobenzoylmethyl, para-bromobenzoylmethyl, 2-tetrahydropyranyl 2-tetrahydrofuranyl, 2,2,2-trichloroethyl cyclopropyl, cyclobutyl, cyclopentyl, cyclohexyl, methoxymethyl, methoxyethoxymethyl, arylalkoxyalkyl benzyloxymethyl 1,1-dimethyl-2-propenyl, 3-methyl-3-butenyl, allyl, and the like. Preferred carboxyl protecting groups of the instant invention are alkyl and arylalkyl.

The term “cyano,” as used herein, refers to —CN.

The term “cycloalkyl,” as used herein, refers to a monovalent saturated cyclic hydrocarbon group of three to seven carbons. The cycloalkyl groups of the instant invention can be optionally substituted with one, two, three, or four substituents independently selected from the group consisting of alkyl, amino, alkoxy, alkoxycarbonyl, carboxaldehyde, carboxyl, halo, hydroxyl, phenyl, heteroaryl, heterocycloalkyl, and oxo. The phenyl, the heteroaryl, and the heterocycloalkyl groups optionally substituting the cycloalkyl groups of the instant invention can also be further substituted with one, two, or three substituents independently selected from the group consisting of alkyl, alkoxy, carboxyl, azido, carboxaldehyde, halo, hydroxyl, perfluoroalkyl, and perfluoroalkoxy.

The term “cycloalkylalkyl,” as used herein, refers to a cycloalkyl group, as defined herein, attached to the parent molecular group through an alkyl group, as defined herein.

The term “cycloalkyloyl,” as used herein, refers to a cycloalkyl group, as defined herein, attached to the parent molecular group through a carbonyl group, as defined herein.

The term “cycloalkylsulfonyl,” as used herein, refers to a cycloalkyl group, as defined herein, attached to the parent molecular group through a sulfonyl group, as defined herein.

The terms “halo” or “halide,” as used herein, refer to F, Cl, Br, or I.

The term “heteroaryl,” as used herein, refers to cyclic, aromatic five- and six-membered groups, wherein at least one atom is selected from the group consisting of nitrogen, oxygen, and sulfur, and the remaining atoms are carbon. The five-membered rings have two double bonds, and the six-membered rings have three double bonds. Heteroaryls of the instant invention are exemplified by furanyl, thienyl, pyrrolyl, oxazolyl, thiazolyl, imidazolyl, isoxazolyl, isothiazolyl, oxadiazolyl, oxadiazolyl, triazolyl, thiadiazolyl, pyridyl, pyridazinyl, pyrimidinyl, pyrazinyl, pyrazolyl, triazinyl, and the like. The heteroaryl groups of the instant invention are connected to the parent molecular group through a carbon atom in the ring or, as exemplified by imidazole and pyrazolyl, through either a carbon atom or nitrogen atom in the ring. The heteroaryl groups of the instant invention can be optionally substituted with one, two, or three radicals independently selected from the group consisting of optionally substituted alkyl, alkenyl, alkynyl, alkoxy, alkanoyl, alkanoyloxy, alkoxycarbonyl, alkylsulfonyl, amino, aminosulfonyl, azido, carboxamido, carboxyl, cyano, halo, hydroxyalkyl, hydroxyl, nitro, perfluoroalkyl, perfluoroalkoxy, oxo, thioalkoxy, a nitrogen protecting group, phenyl, and a heterocycloalkyl selected from the group consisting of tetrahydrofuranyl, piperidinyl, piperazinyl, morpholinyl, and thiomorpholinyl. The phenyl and the heterocycloalkyl groups optionally substituting the heteroaryl groups of the instant invention are attached to the heteroaryl through either a covalent bond, an alkyl group, an oxygen, or a carbonyl group, as defined herein. The phenyl and the heterocycloalkyl groups optionally substituting the heteroaryl groups of the instant invention can also be further substituted with one, two, or three substituents independently selected from the group consisting of alkyl, alkoxy, carboxyl, azido, carboxaldehyde, halo, hydroxyl, perfluoroalkyl, and perfluoroalkoxy. The heteroaryl groups of the instant invention can also be fused to a phenyl ring, in which case the heteroaryl group can be connected to the parent molecular group through either the heteroaryl part or the phenyl part of the fused ring system. Heteroaryl groups of this type are exemplified by quinolinyl, isoquinolinyl, benzodioxolyl, benzodioxinyl, and the like.

The term “heteroarylalkyl,” as used herein, refers to a heteroaryl group, as defined herein, attached to the parent molecular group through an alkyl group, as defined herein.

The term “heteroaryloyl,” as used herein, refers to a heteroaryl group, as defined herein, attached to the parent molecular group through a carbonyl group, as defined herein.

The term “heteroarylsulfonyl,” as used herein, refers to a heteroaryl group, as defined herein, attached to the parent molecular group through a sulfonyl group, as defined herein.

The term “heterocycloalkyl,” as used herein, refers to cyclic, non-aromatic, four-, five-, six-, or seven-membered groups containing at least one atom selected from the group consisting of oxygen, nitrogen, and sulfur. The four-membered rings have zero double bonds, the five-membered rings have zero or one double bonds, and the six- and seven-membered rings have zero, one, or two double bonds. Heterocycloalkyl groups of the instant invention are exemplified by dihydropyridinyl, imidazolinyl, morpholinyl, piperazinyl, pyrrolidinyl, pyrazolidinyl, tetrahydropyridinyl, piperidinyl, thiomorpholinyl, 1,3-dioxolanyl, 1,4-dioxanyl, 1,3-dioxanyl. The heterocycloalkyl groups of the instant invention can be attached through a carbon atom or nitrogen atom in the ring. The heterocyalkalkyls of the instant invention can be optionally substituted one, two, or three substituents independently selected from the group consisting of optionally substituted alkyl, alkoxy, alkanoyl, alkanoyloxy, alkoxycarbonyl, alkylsulfonyl, amino, aminosulfonyl, azido, carboxamido, carboxyl, cyano, halo, hydroxyalkyl, hydroxyl, a nitrogen protecting group, perfluoroalkyl, perfluoroalkoxy, oxo, phenyl, and heteroaryl selected from the group consisting of furanyl, thienyl, pyrrolyl, oxazolyl, thiazolyl, imidazolyl, isoxazolyl, isothiazolyl, oxadiazolyl, oxadiazolyl, triazolyl, thiadiazolyl, pyridyl, pyridazinyl, pyrimidinyl, pyrazinyl, pyrazolyl, and triazinyl. The phenyl and the heteroaryl groups optionally substituting the heterocycloalkyl groups of the instant invention can be attached through a covalent bond, an alkyl group, an oxygen atom, or a carbonyl group. The phenyl and the heteroaryl groups optionally substituting the heterocycloalkyl groups of the instant invention can also be further substituted with one, two, or three substituents independently selected from the group consisting of alkyl, alkoxy, carboxyl, azido, carboxaldehyde, halo, hydroxyl, perfluoroalkyl, and perfluoroalkoxy. The term “heterocycloalkyl” also includes bicyclic groups in which the heterocycloalkyl ring is fused to a phenyl group, in which case the heterocycloalkyl group can be connected to the parent molecular group through either the heterocycloalkyl part or the phenyl part of the fused ring system. Heterocycloalkyl groups of this type are exemplified by 1,3-benzodioxanyl, 1,3-benzodioxolyl, 2,4-dihydro-2H-1,4-benzoxazinyl, and the like.

The term “heterocycloalkylalkyl,” as used herein, refers to a heterocycloalkyl group, as defined herein, attached to the parent molecular group through an alkyl group, as defined herein.

The term “heterocycloalkyloyl,” as used herein, refers to a heterocycloalkyl group, as defined herein, attached to the parent molecular group through a carbonyl group, as defined herein.

The term “heterocycloalkylsulfonyl,” as used herein, refers to a heterocycloalkyl group, as defined herein, attached to the parent molecular group through a sulfonyl group, as defined herein.

The term “hydroxyalkyl,” as used herein, refers to a hydroxyl group attached to the parent molecular group through an alkyl group, as defined herein.

The term “hydroxyl,” as used herein, refers to —OH or a derivative thereof formed by replacement of the hydrogen atom thereon with a hydroxyl protecting group.

The term “hydroxyl protecting group,” as used herein, refers to selectively introducible and removable groups which protect hydroxyl groups against undesirable side reactions during synthetic procedures. Examples of hydroxyl protecting groups include benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl, isopropoxycarbonyl, diphenylmethoxycarbonyl, 2,2,2-trichloroethoxycarbonyl, 2-(trimethylsilyl)ethoxycarbonyl, 2-furfuryloxycarbonyl, allyloxycarbonyl, acetyl, formyl, chloroacetyl, trifluoroacetyl, methoxyacetyl, phenoxyacetyl, benzoyl, methyl, tert-butyl, 2,2,2-trichloroethyl, 2-trimethylsilylethyl, 1,1-dimethyl-2-propenyl, 3-methyl-3-butenyl, allyl, benzyl, para-methoxybenzyldiphenylmethyl, triphenylmethyl (trityl), tetrahydrofuryl methoxymethyl, methylthiomethyl, benzyloxymethyl, 2,2,2-trichloroethoxymethyl, 2-(trimethylsilyl)ethoxymethyl, methanesulfonyl, para-toluenesulfonyl, trimethylsilyl, triethylsilyl, triisopropylsilyl, and the like. Preferred hydroxyl protecting groups for the instant invention are acetyl, benzyl (Bn), benzoyl (Bz), and tert-butyl.

The term “oxo,” as used herein, refers to a group formed by the replacement of two hydrogen atoms on the same carbon atom with a single oxygen atom.

The term “perfluoroalkoxy,” as used herein, refers to a perfluoroalkyl group attached to the parent group through an oxygen atom.

The term “perfluoroalkyl,” as used herein, refers to an alkyl group in which all of the hydrogen atoms have been replaced with fluoride atoms.

The compounds of the instant invention can exist as pharmaceutically acceptable salts. The term “pharmaceutically acceptable salt,” as used herein, refers to salts or zwitterionic forms of the compounds of the instant invention which are water or oil-soluble or dispersible, which are suitable for treatment of diseases without undue toxicity, irritation, and allergic response, which are commensurate with a reasonable benefit/risk ratio, and which are effective for their intended use. The salts can be prepared during the final isolation and purification of the compounds or separately by reacting an amino group with a suitable acid. Representative acid addition salts include acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, formate, fumarate, hydrochloride, hydrobromide, hydroiodide, 2-hydroxyethansulfonate (isethionate), lactate, maleate, mesitylenesulfonate, methanesulfonate, naphthylenesulfonate, nicotinate, 2-naphthalenesulfonate, oxalate, pamoate, pectinate, persulfate, 3-phenylpropionate, picrate, pivalate, propionate, succinate, tartrate, thiocyanate, trichloroacetic, trifluoroacetic, phosphate, glutamate, bicarbonate, para-toluenesulfonate, and undecanoate. Also, amino groups in the compounds of the instant invention can be quaternized with as methyl, ethyl, propyl, and butyl chlorides, bromides and iodides; dimethyl, diethyl, dibutyl, and diamyl sulfates; decyl, lauryl, myristyl, and stearyl chlorides, bromides, and iodides; benzyl and phenethyl bromides. Examples of acids which can be employed to form pharmaceutically acceptable acid addition salts include inorganic acids such as hydrochloric, hydrobromic, sulphuric, and phosphoric and organic acids such as oxalic, maleic, succinic, and citric.

Basic addition salts can be prepared during the final isolation and purification of the compounds by reacting a carboxyl group with a suitable base such as the hydroxide, carbonate, or bicarbonate of a metal cation or with ammonia or an organic primary, secondary or tertiary amine. Pharmaceutically acceptable salts cations based on lithium, sodium, potassium, calcium, magnesium, and aluminum and nontoxic quaternary ammonia and amine cations such as ammonium, tetramethylammonium, tetraethylammonium, methylamine, dimethylamine, trimethylamine, triethylamine, diethylamine, ethylamine, tributlyamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

The compounds of the instant invention can also exist as pharmaceutically acceptable prodrugs. The term “pharmaceutically acceptable prodrug,” as used herein, refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the instant invention.

The term “prodrug,” as used herein, represents compounds which are rapidly transformed in vivo to parent compounds of formulas (I)-(XIII), for example, by hydrolysis in blood.

The term “substituted alkyl,” as used herein, refers to an alkyl group substituted with one, two, or three substituents independently selected from the group consisting of alkoxy, alkanoyloxy, alkoxycarbonyl, alkoxy, alkoxyalkoxy, amino, carboxaldehyde, cycloalkyl, cyano, halo, hydroxyl, oxo, phenyl, heterocycloalkyl, and heteroaryl.

The term “sulfonyl,” as used herein, refers to —SO₂—.

Asymmetric centers exist in the compounds of the instant invention. The instant invention contemplates stereoisomers and mixtures thereof. Individual stereoisomers of compounds are prepared by synthesis from starting materials containing the chiral centers or by preparation of mixtures of enantiomeric products followed by separation such as conversion to a mixture of diastereomers followed by separation or recrystallization, chromatographic techniques, or direct separation of the enantiomers on chiral chromatographic columns. Starting compounds of particular stereochemistry are either commercially available or are made by the methods described below and resolved by techniques well-known in the art.

Tautomers can exist in the compounds of the instant invention. The instant invention contemplates tautomers due to proton shifts from one atom to another atom of the same molecule generating two distinct compounds which are in equilibrium with each other.

The term “tautomer” as used herein refers to a proton shift from one atom of a molecule to another atom of the same molecule to provide two or more structurally distinct compounds which are in equilibrium with each other.

According to methods of treatment, the compounds of the instant invention can be useful for the prevention of metastases from the tumors described above either when used alone or in combination with radiotherapy and/or other chemotherapeutic treatments conventionally administered to patients for treating cancer. When using the compounds of the instant invention for chemotherapy, the specific therapeutically effective dose level for any particular patient will depend upon factors such as the disorder being treated and the severity of the disorder; the activity of the particular compound used; the specific composition employed; the age, body weight, general health, sex, and diet of the patient; the time of administration; the route of administration; the rate of excretion of the compound employed; the duration of treatment; and drugs used in combination with or coincidently with the compound used. For example, when used in the treatment of solid tumors, compounds of the instant invention can be administered with chemotherapeutic agents such as alpha inteferon, COMP (cyclophosphamide, vincristine, methotrexate, and prednisone), etoposide, mBACOD (methortrexate, bleomycin, doxorubicin, cyclophosphamide, vincristine, and dexamethasone), PRO-MACE/MOPP (prednisone, methotrexate (w/leucovin rescue), doxorubicin, cyclophosphamide, taxol, etoposide/mechlorethamine, vincristine, prednisone, and procarbazine), vincristine, vinblastine, angioinhibins, TNP-470, pentosan polysulfate, platelet factor 4, angiostatin, LM-609, SU-101, CM-101, Techgalan, thalidomide, SP-PG, and the like. For example, a tumor may be treated conventionally with surgery, radiation or chemotherapy and a compound of the instant invention with subsequent compound adminsteration of the compound to extend the dormancy of micrometastases and to stabilize and inhibit the growth of any residual primary tumor.

The compounds of the instant invention can be administered orally, parenterally, osmotically (nasal sprays), rectally, vaginally, or topically in unit dosage formulations containing carriers, adjuvants, diluents, vehicles, or combinations thereof. The term “parenteral” includes infusion as well as subcutaneous, intravenous, intramuscular, and intrasternal injection.

Parenterally adminstered aqueous or oleaginous suspensions of the compounds of the instant invention can be formulated with dispersing, wetting, or suspending agents. The injectable preparation can also be an injectable solution or suspension in a diluent or solvent. Among the acceptable diluents or solvents employed are water, saline, Ringer's solution, buffers, dilute acids or bases, dilute amino acid solutions, monoglycerides, diglycerides, fatty acids such as oleic acid, and fixed oils such as monoglycerides or diglycerides.

The chemotherapeutic effect of parenterally administered compounds can be prolonged by slowing their absorption. One way to slow the absorption of a particular compound is adminstering injectable depot forms comprising suspensions of crystalline, amorphous, or otherwise water-insoluble forms of the compound. The rate of absorption of the compound is dependent on its rate of dissolution which is, in turn, dependent on its physical state. Another way to slow absorption of a particular compound is administering injectable depot forms comprising the compound as an oleaginous solution or suspension. Yet another way to slow absorption of a particular compound is administering injectable depot forms comprising microcapsule matrices of the compound trapped within liposomes, microemulsions, or biodegradable polymers such as polylactide-polyglycolide, polyorthoesters or polyanhydrides. Depending on the ratio of drug to polymer and the composition of the polymer, the rate of drug release can be controlled.

Transdernal patches also provide controlled delivery of the compounds. The rate of absorption can be slowed by using rate controlling membranes or by trapping the compound within a polymer matrix or gel. Conversely, absorption enhancers can be used to increase absorption.

Solid dosage forms for oral administration include capsules, tablets, pills, powders, and granules. In these solid dosage forms, the active compound can optionally comprise diluents such as sucrose, lactose, starch, talc, silicic acid, aluminum hydroxide, calcium silicates, polyamide powder, tableting lubricants, and tableting aids such as magnesium stearate or microcrystalline cellulose. Capsules, tablets and pills can also comprise buffering agents; and tablets and pills can be prepared with enteric coatings or other release-controlling coatings. Powders and sprays can also contain excipients such as talc, silicic acid, aluminum hydroxide, calcium silicate, polyamide powder, or mixtures thereof. Sprays can additionally contain customary propellants such as chlorofluorohydrocarbons or substitutes therefor.

Liquid dosage forms for oral administration include emulsions, microemulsions, solutions, suspensions, syrups, and elixirs comprising inert diluents such as water. These compositions can also comprise adjuvants such as wetting, emulsifying, suspending, sweetening, flavoring, and perfuming agents.

Topical dosage forms include ointments, pastes, creams, lotions, gels, powders, solutions, sprays, inhalants, and transdermal patches. The compound is mixed under sterile conditions with a carrier and any needed preservatives or buffers. These dosage forms can also include excipients such as animal and vegetable fats, oils, waxes, paraffins, starch, tragacanth, cellulose derivatives, polyethylene glycols, silicones, bentonites, silicic acid, talc and zinc oxide, or mixtures thereof. Suppositories for rectal or vaginal administration can be prepared by mixing the compounds of the instant invention with a suitable nonirritating excipient such as cocoa butter or polyethylene glycol, each of which is solid at ordinary temperature but fluid in the rectum or vagina. Ophthalmic formulations comprising eye drops, eye ointments, powders, and solutions are also contemplated as being within the scope of the instant invention.

The total daily dose of the compounds of the instant invention administered to a host in single or divided doses can be in amounts from about 0.1 to about 200 mg/kg body weight or preferably from about 0.25 to about 100 mg/kg body weight. Single dose compositions can contain these amounts or submultiples thereof to make up the daily dose.

Determination of Biological Activity

### Farnesyltransferase Inhibition

Farnesyltransferase (FTase) or geranylgeranyltransferase I (GGTase I) fractions were isolated from bovine brains and purified by a series of methods which separate FTase from GGTase I and GGTase I from GGTase II. The methods involved a partial purification of all three enzymes by precipitation from a beef brain homogenate with 30% to 50% saturated (NH)₂SO₄ followed by chromatography on DEAE Sepharose. A Hydrophobic Interaction Chromatography (SHC) media, Fractogel-Phenyl (EM Industries) was used to separate Flase from GGTase; and chromatography of each enzyme on MonoQ (Pharmacia) resulted in further purification of the enzymes. The catalytic purity of each enzyme was assayed separately with substrate acceptor proteins specific for that enzyme. After quickly freezing in liquid nitrogen, the various prenyl transferases were stored at −80° C.

Bovine Frase was assayed at 37° C. for 30 minutes in a volume of 100 μL containing 44 mM HEPES, pH 7.4, 26 mM MgCl₂, 4.4 mM DTT, 18 mM KCl, 0.009% Triton X-100, 256 nM [³H]-farnesyl pyrophosphate, triammonium salt ([³H]-FPP, 759 GBq/mmol, New England Nuclear), 100 nM biotin-K-ras peptide (American Peptide lo Company), and Frase (12.5 μg/mL total protein). Reactions are initiated by the addition of Frase and stopped by the addition of 75 μL of a 1.43 mg/mL suspension of streptavidin SPA (Scintillation Proximity Assay) beads (Amersham) in 0.2M sodium phosphate, pH 4, containing 1.5M MgCl₂, 0.5% BSA and 0.05% sodium azide. The quenched reactions stood for 1 hour before analysis in a Packard TopCount scintillation counter. Purified compounds were dissolved in 100% ethanol and diluted 10-fold into the assay. The percent inhibition of the compounds of the instant invention at 10⁻⁶ M was then measured.

The percent inhibition of representative compounds of the instant invention are shown in Table 1.

TABLE 1
Inhibitory Potencies of Representative Compounds

| | % Inhibition at | | % Inhibition at |
|---|---|---|---|
| Example | 10⁻⁶M | Example | 10⁻⁶M |
| 4 | 88 | 142 | 94 |
| 5 | 91 | 143 | 97 |
| 6 | 61 | 144 | 96 |
| 7 | 92 | 145 | 97 |
| 8 | 100 | 146 | 93 |
| 9 | 64 | 147 | 96 |
| 10 | 94 | 148 | 95 |
| 11 | 94 | 149 | 93 |
| 12 | 100 | 150 | 93 |
| 13 | 100 | 151 | 94 |
| 14 | 100 | 152 | 89 |
| 15 | 100 | 153 | 92 |
| 16 | 100 | 154 | 95 |
| 17 | 100 | 155 | 92 |
| 18 | 100 | 156 | 92 |
| 19 | 100 | 157 | 97 |
| 23 | 100 | 158 | 95 |
| 24 | 100 | 159 | 91 |
| 25 | 100 | 160 | 96 |
| 26 | 100 | 161 | 96 |
| 27 | 100 | 162 | 94 |
| 28 | 100 | 163 | 84 |
| 29 | 100 | 164 | 99 |
| 30 | 100 | 168 | 96 |
| 31 | 100 | 169 | 70 |
| 32 | 93 | 170 | 85 |
| 33 | 100 | 171 | 75 |
| 34 | 100 | 172 | 85 |
| 35 | 100 | 173 | 80 |
| 36 | 100 | 175 | 75 |
| 37 | 100 | 177 | 77 |
| 38 | 100 | 178 | 90 |
| 39 | 100 | 179 | 93 |
| 40 | 100 | 180 | 95 |
| 41 | 100 | 181 | 93 |
| 42 | 100 | 182 | 89 |
| 43 | 100 | 183 | 90 |
| 44 | 100 | 184 | 95 |
| 45 | 100 | 185 | 85 |
| 46 | 100 | 186 | 85 |
| 47 | 100 | 187 | 93 |
| 48 | 100 | 188 | 92 |
| 49 | 62 | 189 | 95 |
| 50 | 100 | 190 | 90 |
| 51 | 100 | 191 | 99 |
| 52 | 100 | 193 | 91 |
| 53 | 100 | 194 | 97 |
| 54 | 100 | 195 | 98 |
| 55 | 100 | 196 | 90 |
| 56 | 100 | 197 | 93 |
| 57 | 100 | 198 | 94 |
| 58 | 100 | 199 | 89 |
| 59 | 100 | 200 | 99 |
| 60 | 96 | 201 | 99 |
| 61 | 100 | 202 | 92 |
| 62 | 100 | 203 | 86 |
| 63 | 100 | 204 | 96 |
| 64 | 100 | 205 | 90 |
| 65 | 100 | 206 | 88 |
| 66 | 100 | 207 | 91 |
| 67 | 100 | 208 | 95 |
| 68 | 100 | 209 | 93 |
| 69 | 100 | 210 | 48 |
| 70 | 100 | 211 | 30 |
| 71 | 100 | 212 | 10 |
| 72 | 100 | 213 | 41 |
| 73 | 100 | 214 | 20 |
| 74 | 100 | 215 | 29 |
| 75 | 100 | 216 | 27 |
| 76 | 100 | 217 | 71 |
| 77 | 100 | 225 | 50 |
| 80 | 12 | 226 | 100 |
| 81 | 14 | 227 | 100 |
| 82 | 87 | 228 | 100 |
| 85 | 46 | 229 | 100 |
| 86 | 86 | 230 | 100 |
| 87 | 89 | 231 | 100 |
| 90 | 90 | 232 | 100 |
| 91 | 94 | 233 | 100 |
| 92 | 53 | 234 | 100 |
| 93 | 58 | 235 | 100 |
| 100 | 83 | 236 | 93 |
| 105 | 82 | 237 | 100 |
| 109 | 100 | 240 | 95 |
| 110 | 96 | 241 | 98 |
| 111 | 100 | 242 | 98 |
| 112 | 100 | 243 | 82 |
| 113 | 90 | 244 | 90 |
| 114 | 99 | 245 | 90 |
| 115 | 89 | 246 | 90 |
| 116 | 99 | 247 | 95 |
| 117 | 90 | 248 | 98 |
| 118 | 90 | 249 | 98 |
| 119 | 93 | 250 | 99 |
| 120 | 90 | 251 | 99 |
| 121 | 96 | 256 | 94 |
| 122 | 94 | 260 | 90 |
| 123 | 97 | 262 | 100 |
| 124 | 90 | 263 | 100 |
| 125 | 97 | 264 | 100 |
| 126 | 92 | 265 | 100 |
| 127 | 93 | 266 | 100 |
| 128 | 94 | 267 | 100 |
| 129 | 93 | 268 | 100 |
| 130 | 97 | 269 | 100 |
| 131 | 95 | 270 | 100 |
| 132 | 94 | 271 | 100 |
| 133 | 97 | 273 | 100 |
| 134 | 93 | 274 | 100 |
| 135 | 94 | 275 | 100 |
| 136 | 95 | 276 | 100 |
| 137 | 96 | 277 | 95 |
| 138 | 92 | 278 | 99 |
| 139 | 97 | 279 | 99 |
| 140 | 94 | 280 | 98 |
| 141 | 95 | 281 | 98 |
| | | 289 | 75 |

Representative compounds of the instant invention were also tested for cardiovascular liability (see Journal of Cardiovascular Pharmacology, 607-618: 37 (2001)). Example 291 was shown to possess an improved electrophysiological profile.

As shown by the data in Table 1, the compounds of the instant invention, including but not limited to those specified in the examples, are useful for the treatment of diseased caused or exascerbated by farnesyltransferase. As farnesyltransferase inhibitors, these compounds are useful in the treatment of both primary and metastatic solid tumors and carcinomas of the breast; colon; rectum; lung; oropharynx; hypopharynx; esophagus; stomach; pancreas; liver; gallbladder; bile ducts; small intestine; urinary tract (kidney, baldder, and urothelium); female genital tract (cervix, uterus, and ovaries); male genital tract (prostate, seminal vesicles, and testes); endocrine glands (thyroid, adrenal, and pituitary); skin (hemangiomas, melanomas, and sarcomas); tumors of the brain, nerves, and eyes; meninges (astrocytomas, gliomas, glioblastomas, retinoblastomas, neuromas, neuroblastomas, and meningiomas); solid tumors arising from hematopoietic malignancies (leukemias and chloromas); plasmacytomas; plaques; tumors of mycosis fungoides; cutaneous T-cell lymphoma/leukemia; lymphomas including Hodgkin's and non-Hodgkin's lymphomas; prophylaxis of autoimmune diseases (rheumatoid, immune and degenerative arthritis); ocular diseases (diabetic retinopathy, retinopathy of prematurity, corneal graft rejection, retrolental fibroplasia, neovascular glaucoma, rubeosis, retinal neovascularization due to macular degeneration, and hypoxia); skin diseases (psoriasis, hemagiomas and capillary proliferation within atherosclerotic plaques).

Synthetic Methods

The compounds and processes of the instant invention will be better understood in connection with the following synthetic schemes which illustrate methods by which the compounds can be prepared. The compounds of the instant invention can be prepared by a variety of synthetic routes. Representative procedures are shown below in Schemes 1-19. The groups a, b, c, A¹, L¹, L², M¹, Q¹, Q², R¹, R^{b}R¹, R², R³, R⁴, R⁵, W, W′, X, X′, Y, Y′, Z, and Z′ are defined above, and the groups M^{1p}, Q^{1p}, and Q^{2p} are defined below. It will be readily apparent to one of ordinary skill in the art that the compounds can be synthesized by substitution of the appropriate reactants and agents in the syntheses shown below. It will also be apparent to one skilled in the art that the selective protection and deprotection steps, as well as the order of the steps themselves, can be carried out in varying order, depending on the nature of a, b, c, A¹, L¹, L², M¹, Q¹, Q², R^{a}, R^{b}R¹, R², R³, R⁴, R⁵, W, W′, X, X′, Y, Y′, Z, and Z′ to successfully complete the syntheses of compounds of the instant invention.

Abbreviations which have been used in the descriptions of the schemes and the examples that follow are: OAc for acetate; PyBop for benzotriazol-1-yl-oxy-tris- (pyrrolidino)phosphoniumhexafluorophosphate; DMAP for 4-(N,N-dimethylamino)pyridine; DME for dimethoxyethane; DMF for N,N-dimethylformamide; DMSO for dimethylsulfoxide; EDC for 1-(3-(dimethylamino)propyl)-3-ethylcarbodiimide hydrochloride; HOBt for 1-hydroxybenzotriazole hydrate; HPLC for high pressure liquid chromatography; LDA for lithium diisopropylamide; MTBE for methyl tert-butyl ether; TEA for triethylamine; TFA for trifluoroacetic acid; and THF for tetrahydrofuran.

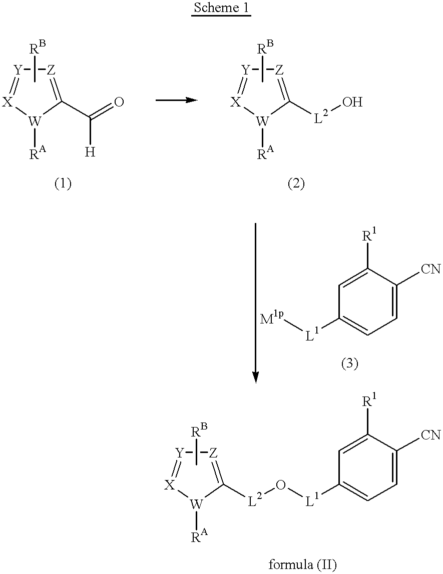

As shown in Scheme 1, compounds of formula (1) can be converted to compounds of formula (2), wherein L² is optionally substituted alkylene, by treatment of the former with an organometallic nucleophile in a solvent such as THF, dioxane, MTBE, or diethyl ether. Representative organometallic nucleophiles include Grignard reagents, organolithium reagents, organozinc reagents, and organocadmium reagents. The reaction temperature is about −78 ° C. to about 35° C. and depends on the method chosen. Reaction times are typically about 0.5 to about 4 hours. Compounds of formula (1) can be converted to compounds of formula (2), wherein L² is alkylene, by treatment of the former with a reducing agent in a solvent such as THF, dioxane, or diethyl ether. Representative reducing agents include LiAlH₄ and NaBH₄. The reaction temperature is about −78° C. to about 35° C. and depends on the method chosen. Reaction times are typically about 0.5 to about 4 hours. Compounds of formula (2) can be converted to compounds of formula (II) by treatment of the former with compounds of formula (3), wherein M^{1p} is an M¹ precursor such as halo, in the presence of silver(I) oxide in a solvent such as dichloromethane, carbon tetrachloride, or chloroform. The reaction temperature is about 20 ° C. to about 40° C. and depends on the method chosen. Reaction times are typically about 6 to about 48 hours.

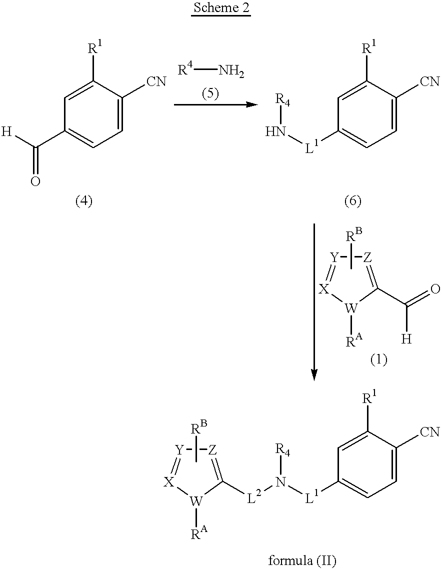

As shown in Scheme 2, compounds of formula (4) can be converted to compounds of formula (6) by treatment of the former with compounds of formula (5) in the presence of a reducing agent such as sodium triacetoxyborohydride, sodium cyanoborohydride, sodium borohydride, or borane-pyridine in a solvent such as 1,2-dichloroethane, dichloromethane, chloroform, or carbon tetrachloride. The reaction temperature is about 0° C. to about 40° C. and depends on the method chosen. Reaction times are typically about 6 to about 24 hours. Compounds of formula (6) can be converted to compounds of formula (II) by condensation of the former with compounds of formula (1) as described in Scheme 1.

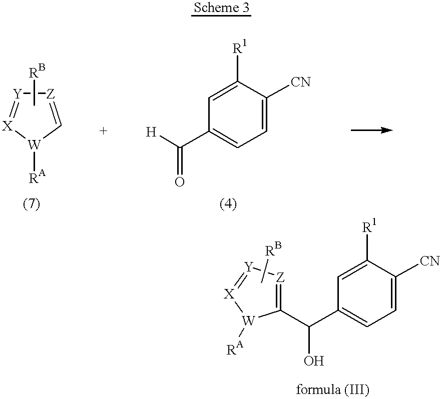

As shown in Scheme 3, compounds of formula (7) can be converted to compounds of formula (III) by sequential treatment of the former with a base such as tert-butyllithium, n-butyllithium, and lithium hexamethyldisilazide and compounds of formula (4) in a solvent such as THF, MTBE, or diethyl ether. The reaction temperature is about −78° C. to about 0° C. and depends on the method chosen. Reaction times are typically about 0.5 to about 2 hours. Compounds of formula (III) can be oxidized to compounds of formula (IIIa) by treatment of the same with an oxidizing agent such as manganese dioxide, potassium permanganate, potassium dichromate, or Jones reagent in a solvent such as dioxane, acetone, THF, or dichloromethane. The reaction temperature is about 0° C. to about 100° C. and depends on the method chosen. Reaction times are typically about 0.5 to about 12 hours.

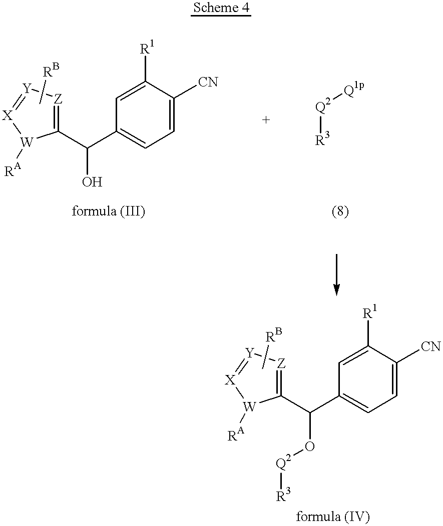

As shown in Scheme 4, compounds of formula (III) can be treated with compounds of formula (8), wherein Q^{1p} is a Q¹ precursor such as halo, under the conditions described in Scheme 1 to provide compounds of formula (IV).

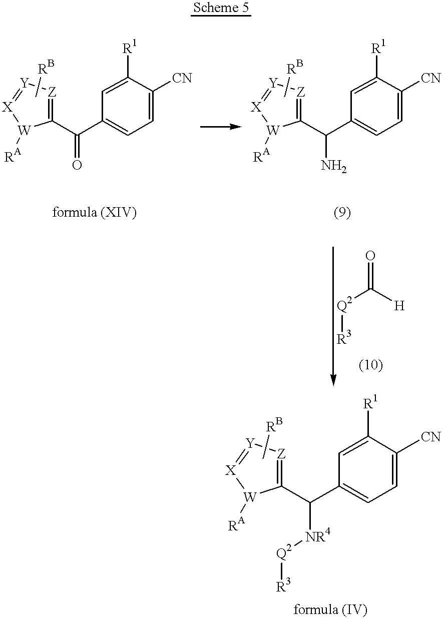

As shown in Scheme 5, compounds of formula (XIV) can be converted to compounds of formula (9) by sequential treatment of the former with a chlorinating agent such as SOCl₂, PPh₃/CCl₄, PCl₅, or PPh₃/NCS and ammonium hydroxide in a solvent such as dichloromethane, carbon tetrachloride, or chloroform. The reaction temperature is about −10° C. to about 25° C. and depends on the method chosen. Reaction times are typically about 1 to about 12 hours. Conversion of compounds of formula (9) to compounds of formula (IV) can be accomplished by the methods described in Scheme 2.

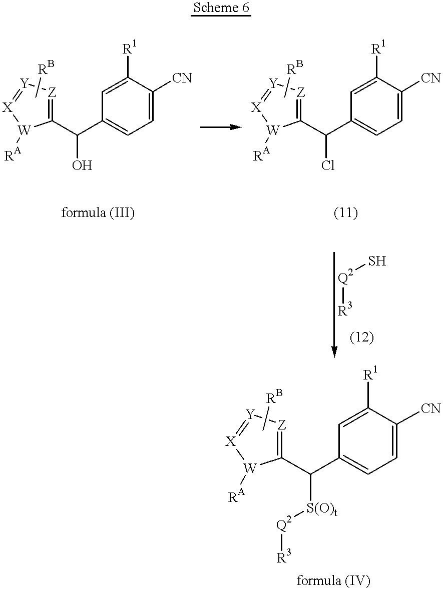

As shown in Scheme 6, compounds of formula (III) can be converted to compounds of formula (11) by treatment of the former with the chlorinating agent in a solvent such as dichloromethane, carbon tetrachloride, or chloroform. The reaction temperature is about −10° C. to about 25° C. and depends on the method chosen. Reaction times are typically about 1 to about 12 hours. Conversion of compounds of formula (11) to compounds of formula (IV), wherein t is 0, can be accomplished by treatment of the former with compounds of formula (12) in the presence of a base such as triethylamine, diisopropylethylamine, or pyridine in a solvent such as dichloromethane, carbon tetrachloride, or chloroform. The reaction temperature is about 20° C. to about 35° C. and depends on the method chosen. Reaction times are typically about 12 to about 24 hours. Conversion of compounds of formula (IV), wherein t is 0, to compounds of formula (IV), wherein, t is 1 or 2, can be accomplished by treatment of the former with an oxidizing agent such as m-CPBA, hydrogen peroxide, NaIO₄, and NaOCl in a solvent such as dichloromethane, carbon tetrachloride, and chloroform. The reaction temperature is about 20° C. to about 40° C. and depends on the method chosen. Reaction times are typically about 12 to about 72 hours.

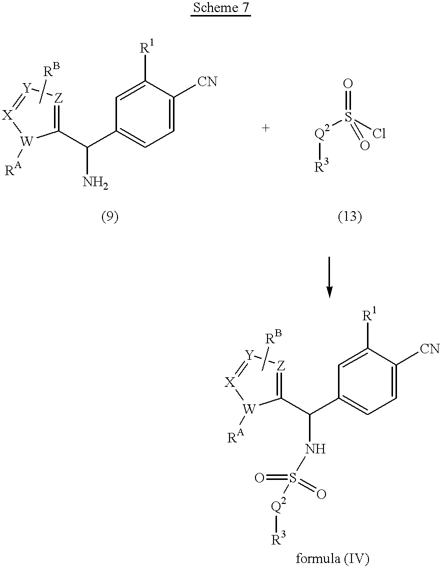

As shown in Scheme 7, compounds of formula (9) can be converted to compounds of formula (IV) by treatment of the former with compounds of formula (13) in the presence of a base such as DMAP, triethylamine, diisopropylethylamine, pyridine, or mixtures thereof in a solvent such as dichloromethane, chloroform, or carbon tetrachloride. The reaction temperature is about 20° C. to about 40° C. and depends on the method chosen. Reaction times are typically about 6 hours to about 24 hours.

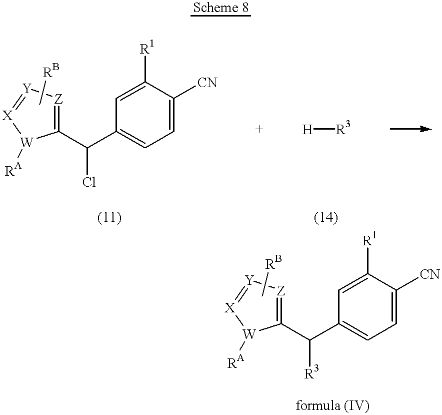

As shown in Scheme 8, compounds of formula (11) can be converted to compounds of formula (IV) by treatment of the former with compounds of formula (14), wherein R³ is an alcohol, thiol, or a primary or secondary amine, in the presence of a base such as diisopropylethylamine, pyridine, or triethylamine in a solvent such as dichloromethane, carbon tetrachloride, or chloroform. The reaction temperature is about 30° C. to about 100° C. and depends on the method chosen. Reaction times are typically about 1 to about 12 hours.

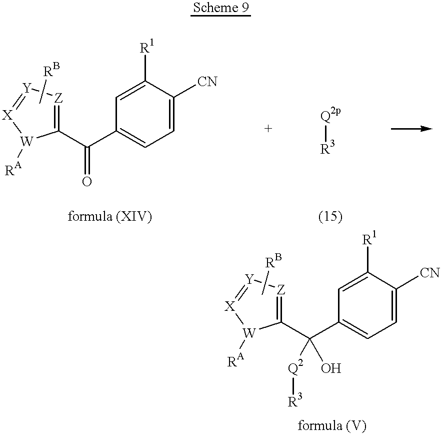

As shown in Scheme 9, compounds of formula (15), wherein Q^{2p} is an alkynyl Q² precursor, can be treated sequentially with a base such as tert-butyllithium, n-butyllithium, LDA, or lithium hexamethyldisilazide and compounds of formula (XIV) to provide compounds of formula (V), wherein Q² is alkynylene, in a solvent such as THF, MTBE, dioxane, or diethyl ether. The reaction temperature is about −78° C. to about 25° C. and depends on the method chosen. Reaction times are typically about 0.5 to about 24 hours. Compounds of formula (V), wherein Q² is alkynyl, can be intraconverted to compounds of formula (V), wherein Q² is alkylene or alkenylene, by hydrogenation in the presence of palladium catalysts such as Pd/BaSO₄, Pd/CaCO₃, and Pd/C in a solvent such as methanol, ethanol, or isopropanol. The reaction temperature is about 25° C. to about 40° C. and depends on the method chosen. Reaction times are typically about 2 to about 32 hours.

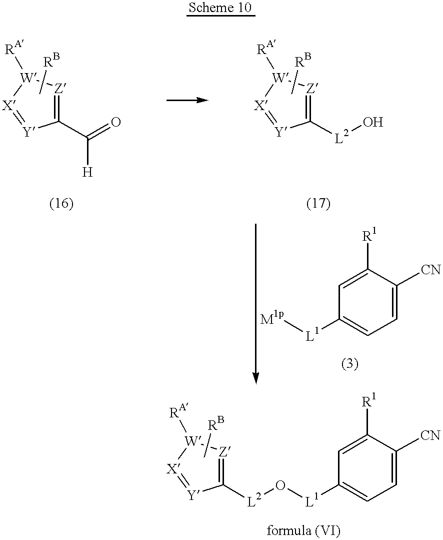

As shown in Scheme 10, compounds of formula (16) can be converted to compounds of formula (17) and subsequently to compounds of formula (VI) by the methods described in Scheme 1.

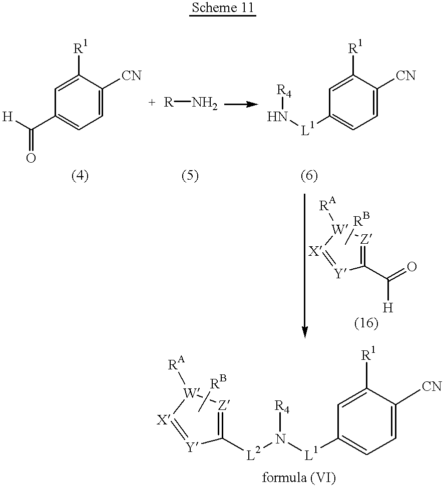

As shown in Scheme 11, compounds of formula (4) can be converted to compounds of formula (6) and subsequently to compounds of formula (VI) by the methods described in Scheme 2.

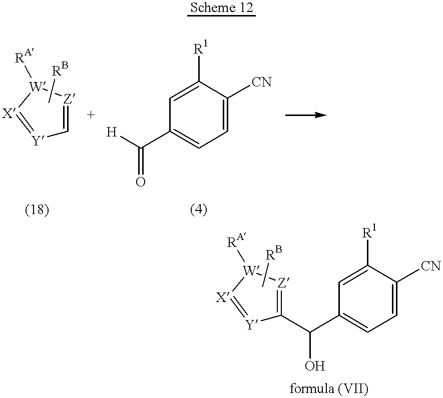

As shown in Scheme 12, compounds of formula (18) can be converted to compounds of formula (VII) by the methods described in Scheme 3.

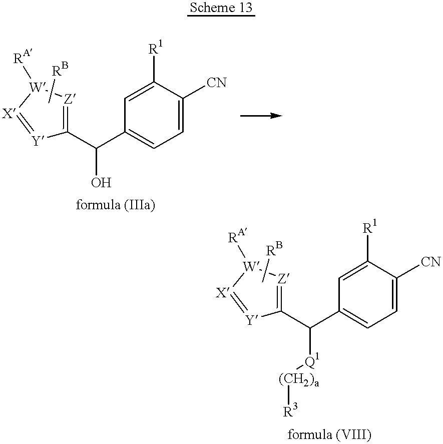

As shown in Scheme 13, compounds of formula (IIIa) can be converted to compounds of formula (VIII) by the methods described in Schemes 4 through 8.

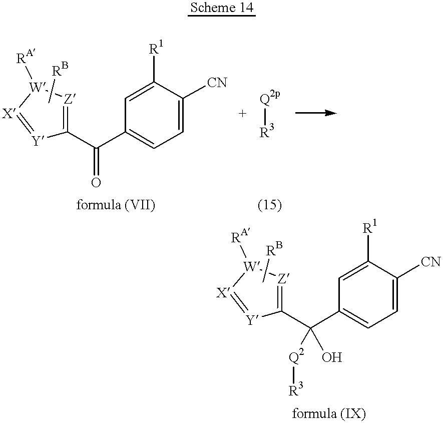

As shown in Scheme 14, compounds of formula (VII) can be converted to compounds of formula (IX) by treatment with compounds of formula (15) under the conditions described in Scheme 9.

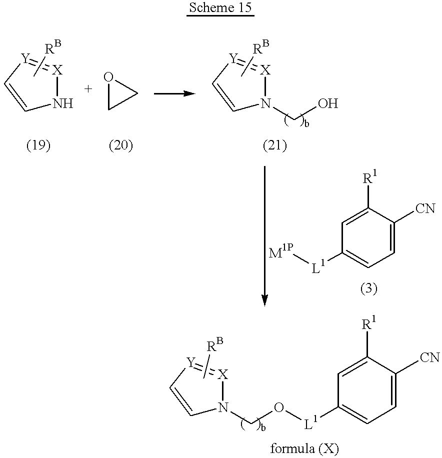

As shown in Scheme 15, compounds of formula (19) can be reacted with oxirane to provide compounds of formula (21), wherein b is 2, which can be converted to compounds of formula (X) by treatment with compounds of formula (3) under the conditions described in Scheme 1.

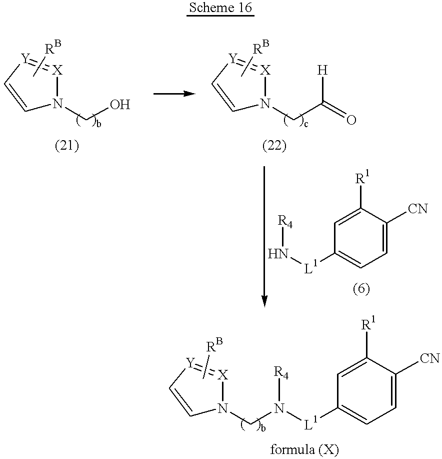

As shown in Scheme 16, compounds of formula (21) can be converted to compounds of formula (22) by treatment with an oxidizing agent such as Dess-Martin periodinane, MnO₂, PCC, and K₂Cr₂O₇ in a solvent such as these reactions include dichloromethane, chloroform, and carbon tetrachloride. The reaction temperature is about 0° C. to about 35° C. and depends on the method chosen. Reaction times are typically about 0.5 to about 12 hours. Compounds of formula (22) can be condensed with compounds of formula (6) to provide compounds of formula (X) using the conditions described in Scheme 2.

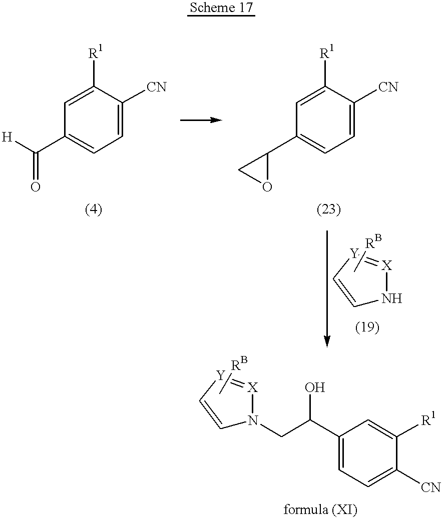

As shown in Scheme 17, compounds of formula (4) can be converted to compounds of formula (23) by treatment of the former with a sulfonium ylide such as trimethylsulfonium iodide in the presence of a base such as potassium hydroxide or sodium hydroxide in a solvent such as DMSO, DMF, or mixtures thereof. The reaction temperature is about 25° C. to about 80° C. and depends on the method chosen. Reaction times are typically about 1 to about 6 hours. Compounds of formula (23) can be converted to compounds of formula compounds of formula (XI) by treatment of the former with catalytic base such as DMAP, pyridine, or diisopropylethylamine and compounds of formula (19) in solvents such as methanol, ethanol, or isopropanol. The reaction temperature is about 35° C. to about 100° C. and depends on the method chosen. Reaction times are typically about 2 to about 24 hours.

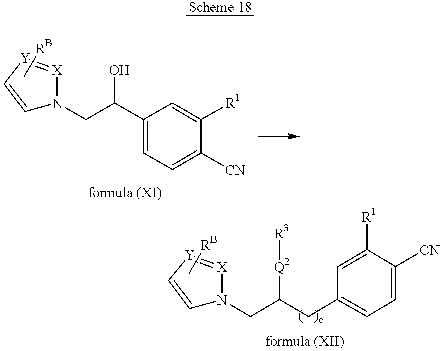

As shown in Scheme 18, compounds of formula (XI) can be converted to compounds of formula (XII) using the conditions described in Schemes 4 through 8.

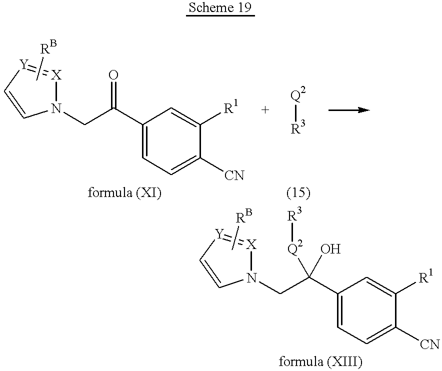

As shown in Scheme 19, compounds of formula (XI) can be converted to compounds of formula (XIII) by treatment of the former with compounds of formula (15) under the conditions described in Scheme 9.

The instant invention will now be described in connection with other particularly preferred embodiments of Schemes 1-19, which are not intended to limit its scope. On the contrary, the instant invention covers all alternatives, modifications, and equivalents which are included within the scope of the claims. Thus, the following examples will illustrate an especially preferred practice of the instant invention, it being understood that the examples are for the purposes of illustration of certain preferred embodiments and are presented to provide what is believed to be the most useful and readily understood description of its procedures and conceptual aspects.

It will be evident to one skilled in the art that the instant invention is not limited to the forgoing examples, and that it can be embodied in other specific forms without departing from the essential attributes thereof. Thus, it is desired that the examples be considered as illustrative and not restrictive, reference being made to the claims, and that all changes which come within the meaning and range of equivalency of the claims be embraced therein.

### EXAMPLE 1

### 5-((benzyloxy)(6-fluoro-2′methyl(1,1′-biphenyl)-3-yl)methyl)-1-methyl-1H-imidazole hydrochloride

### EXAMPLE 1A

### 6-fluoro-2′-methyl(1′-biphenyl)-3-carbaldehyde

A mixture of 3-bromo-4-fluorobenzaldehyde (1.1 g, 5.9 mmol), 2-methylphenylboronic acid (9.05 mg, 6.6 mmol), palladium(II) acetate (23 mg, 6.6 mmol), 2M Na₂CO₃ (14 mL), and triphenylphosphine (102 mg, 0.39 mmol) in toluene (13 mL) was heated to 100° C. for 90 minutes with vigorous stirring, and cooled to room temperature to provide two separate layers. The organic layer was concentrated, and the concentrate was purified by flash column chromatography on silica gel with 95:5/hexanes:ethyl acetate to provide the desired product. MS (DCI/NH₃) m/z 214 (M+H)⁺ and 232 (M+NH₄)⁺; ¹H NMR (300 MHz, CDCl₃) δ 10.0 (s, 1H), 8.95 (m, 1), 8.83 (dd, 1H), 7.40-7.15 (m, 5H), 2.2 (s, 3H).

### EXAMPLE 1B

### (6-fluoro-2′-methyl(1,1′-biphenyl)-3-yl)(1-methyl-1H-imidazol-5-yl)methanol

A solution of Example 87F (471.3 mg, 2.4 mmol) in THF (5 mL) at −75° C. was treated with 1.7M tert-butyllithium in pentane (1.7 mL, 2.88 mmol), stirred for 15 minutes, treated with Example 1A (514 mg, 2.4 mmol) in THF (5 mL), stirred for 1 hour, warmed to 0° C. for 20 minutes, treated sequentially with methanol (3 mL) and 1M tetrabutylammonium fluoride in THF (2.4 mL, 2.4 mmol), warmed to room temperature, stirred for 18 hours, poured into water (50 mL), and extracted with ethyl acetate. The extract was washed sequentially with saturated NaHCO₃, water, and brine, dried (Na₂SO₄), filtered, and concentrated. The concentrate was purified by flash column chromatography on silica gel with 96.5:2.5:1 to 89:10:1 ethyl acetate/methanol/ concentrated ammonium hydroxide to provide the desired product. MS (DCI/NH₃) m/z 297 (M+H)⁺; ¹H NMR (300 MHz, CD₃OD) δ 7.6 (s, 1H), 7.45 (m, 1H), 7.35-7.10 (m, 5H), 6.55 (s, 1H), 5.90 (s, 1H), 3.65 (s, 3H), 2.15 (s, 3H), 1.90 (s, 1H).

### EXAMPLE 1C

### 5-((benzyloxy)(6-fluoro-2′methyl(1,1′-biphenyl)-3-yl)methyl)-1-methyl-1H-imidazole hydrochloride

A solution of Example 1B (133 mg, 0.45 mmol) in DMF (1 mL) at −3° C. was treated with a 60% oily sodium hydride (28 mg, 0.68 mmol), stirred for 1 hour, treated with (bromomethyl)benzene (60 μL, 0.5 mmol), stirred for 18 hours at room temperature, treated with water, and extracted with ethyl acetate. The extract was washed with brine, dried (Na₂SO₄), filtered, and concentrated. The concentrate was purified by preparative HPLC with 4:1/CH₃CN:0. 1% aqueous TFA to 0.1% aqueous TFA. The appropriate fractions were combined and concentrated. The concentrate was treated with saturated NaHCO₃, and the resulting solution was extracted with ethyl acetate. The extract was dried (Na₂SO₄), filtered, and concentrated. The concentrate was dissolved in 4M HCl in dioxane (2 mL), and the resulting solution was stirred for 2 hours and concentrated. This concentrate was dissolved in water and lyophilized to provide the desired product.

MS (ESI(+)) m/z 387 (M+H)⁺; ¹H NMR (300 MHz, DMSO-d₆) δ 9.1 (s, 1H), 7.6-7.2 (m, 12H), 5.95 (s, 1H), 4.55 (q, 2H), 3.75 (s, 3H), 2.15 (s, 3H); Anal. calcd for C₂₅H₂₄ClFN₂O·1.25H₂O: C, 67.41; H, 6.00; N, 6.29. Found: C, 67.48; H, 5.85; N 6.13.

### EXAMPLE 2

### benzyl (2′-methyl(1,1′-biphenyl)-3-yl)(1-methyl-1H-imidazol-5-yl)methyl ether, hydrochloride

### EXAMPLE 2A

### 2′-methyl(1,1′-biphenyl)-3-carbaldehyde

The desired product was prepared by substituting 3-bromobenzaldehyde for 3-bromo-4-fluorobenzaldehyde in Example 1A. MS (DCI/NH₃) m/z 214 (M+NH₄)⁺; ¹H NMR (300 MHz, CDCl₃) δ 10.1 (s, 1H), 7.85 (m, 2H), 7.6 (m, 2H), 7.35-7.2 (m, 4H), 2.25 (s, 3H).

### EXAMPLE 2B

### (2′-methyl(1,1′-biphenyl)-3-yl)(1-methyl-1H-imidazol-5-yl)methanol

The desired product was prepared by substituting Example 2A for Example 1A in Example 1B. MS (DCI/NH₃) m/z 279 (M+H)⁺; ¹H NMR (300 MHz, CD₃OD) δ 7.6 (s, 1H), 7.5-7.1 (m, 7H), 6.55 (s, 1H), 5.95 (s, 1H), 3.7 (s, 3H), 2.2 (s, 3H).

### Example 2C

### benzyl (2′-methyl(1,1′-biphenyl)-3-yl)(1-methyl-1H-imidazol-5-yl)methyl ether hydrochloride

The desired product was prepared by substituting Example 2B for Example 1B in Example 1C, and purified by flash column chromatography on silica gel with 95:5:0.1/ethyl acetate:methanol:concentrated ammonium hydroxide. The appropriate fractions were concentrated, and the concentrate was dissolved in 4M HCl in dioxane (1.5 mL), stirred for 3 hours, and concentrated. The concentrate was treated with water and lyophilized to provide the desired product.

MS (ESI(+)) m/z 369 (M+H)⁺; ¹H NMR (300 MHz, DMSO-d₆) δ 9.1 (s, 1H), 7.8-7.2 (m, 13H), 5.95 (s, 1H), 4.6 (q, 2H), 3.75 (s, 3H), 2.2 (s, 3H); Anal. calcd for C₂₅H₂₅ClN₂O·1.35H₂O: C, 69.95; H, 6.50; N, 6.53. Found: C, 69.89, H, 6.23; N, 6.78.

### EXAMPLE 3

### 5-((benzyloxy)(6-chloro-2′-methyl(1,1′-biphenyl)-3-yl)methyl)-1-methyl-1H-imidazole hydrochloride

### EXAMPLE 3A

### 4-chloro-3-iodobenzoic acid

A solution of 3-amino-4-chlorobenzoic acid (8.6 g, 50 mmol) in 2:1 3M HCl/acetone (150 mL) at −3° C. was treated dropwise with sodium nitrite (3.8 g, 55 mmol) in water (30 mL), stirred for 30 minutes, treated with potassium iodide (14.5 g, 87.5 mmol) in water (50 mL), stirred for 15 minutes at 0° C. and at room temperature for 2 hours, and treated with water (500 mL) and excess NaHCO₃ to provide a solid. The solid was collected by filtration and recrystalized from 20% methanol/water to provide the desired product.

MS (DCI/NH₃) m/z 282 (M+H)⁺; ¹H NMR (300 MHz, DMSO-d₆) δ 13.35 (br s, 1H), 8.4 (d, 1H), 7.95 (dd, 1H), 7.7 (d, 1H).

### EXAMPLE 3B

### 4-chloro-3-iodo-N-methoxy-N-methylbenzamide

A mixture of Example 3A (2.82 g, 10 mmol), EDC (2.11 g, 11 mmol), HOBt (1.68 g, 11 mmol), and N,O-dimethylhydroxylamine hydrochloride (1.26 g, 13 mmol) in DMF (30 mL) was stirred until all of the reagents dissolved, treated with triethylamine (2.54 mL, 18 mmol), stirred for 3 days at room temperature, treated with 1:1/ethyl acetate:water, stirred for 1 hour, poured into water, and extracted with ethyl acetate. The extract was washed sequentially with 2M Na₂CO₃, water, and brine, dried (Na₂SO₄), filtered, and concentrated. The concentrate was purified by flash column chromatography on silica gel with 3:1/hexanes:ethyl acetate to provide the desired product.

MS (DCI/NH₃) m/z 343 (M+NH₄)⁺; ¹H NMR (300 MHz, CDCl₃) δ 8.2 (d, 1H), 7.65 (dd, 1H), 7.5 (s, 1H), 3.55 (s, 3H), 3.35 (s, 3H).

### EXAMPLE 3C

### 6-chloro-N-methoxy-N,2′-dimethyl(1,1′-biphenyl)-3-carboxamide

A mixture of Example 3B (2.61 g, 8.02 mmol), 2-methylphenylboronic acid (1.20 g, 8.82 mmol), (1,1′-bis(diphenylphosphino)ferrocene)dichloropalladium (II) (196 mg, 0.24 mmol), CsF (2.44 g, 16.14 mmol), and DME (40 mL) was heated to reflux for 18 hours, cooled to room temperature, treated with diethyl ether, filtered through diatomaceous earth (Celite®), and concentrated. The concentrate was purified by flash column chromatography on silica gel with 7:3/hexanes:ethyl acetate to provide the desired product.

MS (DCI/NH₃) m/z 307 (M+N₄)⁺; ¹H NMR (300 MHz, CDCl₃) δ 7.7-7.1 (m, 7H), 3.55 (s, 3H), 3.35 (s, 3H), 2.1 (s, 3H).

### EXAMPLE 3D

### 6-chloro-2′-methyl(1,1′-biphenyl)-3-carbaldehyde

A solution of Example 3C (1.167 g, 4 mmol) in THF (10 mL) at −10° C. was treated dropwise with 1M lithium aluminum hydride in THF (4.4 mL, 3.3 mmol), stirred for 2 hours, treated sequentially with THF/water (1 mL:0.17 mL), 4M NaOH (0.17 mL), and water (0.5 mL), warmed to room temperature, and extracted with 1: I/ethyl acetate:hexanes. The extract was dried (Na₂SO₄), filtered through a pad of silica gel, and concentrated to provide material of sufficient purity for subsequent use without further purification.

MS (DCI/NH₃) m/z 230 (M+H)⁺; ¹H NMR (300 MHz, CDCl₃) δ 10.0 (s, 1H), 7.9-7.1 (m, 7H), 2.1 (s, 3H).

### EXAMPLE 3E

### (6-chloro-2′-methyl(1,1′-biphenyl)-3-yl)(1-methyl-1H-imidazol-5-yl)methanol

The desired product was prepared by substituting Example 3D for Example 1A in Example 1B.

MS (DCI/NH₃) m/z 313 (M+H)⁺; ¹H NMR (300 MHz, CDCl₃) δ 7.5-7.0 (m, 7H), 6.7 (s, 1H), 5.9 (s, 1H), 3.6 (d, 3H), 2.1 (d, 3H).

### EXAMPLE 3F

### 5-((benzyloxy)(6-chloro-2′-methyl(1,1′-biphenyl)-3-yl)methyl)-1-methyl-1H-imidazole hydrochloride

The desired product was prepared by substituting Example 3E for Example 1B in Example 1C.

MS (ESI(+)) m/z 403 (M+H)⁺; ¹H NMR (300 MHz, DMSO-d₆) δ 9.1 (s, 1H), 7.7 (dd, 1H), 7.5 (dt, 1H), 7.4-7.1 (m, 10H), 5.95 (s, 1H), 4.6 (q, 2H), 3.75 (d, 3H), 2.1 (s, 3H); Anal. calcd for C₂₅H₂₄Cl₂N₂O·1.05H₂O: C, 65.52; H, 5.74; N, 6.11. Found: C, 65.49; H, 5.77; N, 6.18.

### EXAMPLE 4

### 2′-methyl-5-((1-methyl-1H-imidazol-5-yl)(phenoxy)methyl)(1,1′-biphenyl)-2-carbonitrile hydrochloride

### EXAMPLE 4A

### 5-(hydroxy(1-methyl-1H-imidazol-5-yl)methyl)-2′-methyl(1,1′-biphenyl)-2-carbonitrile

The desired product was prepared by substituting Example 86I for Example 1A in Example 1B.

MS (DCI/NH₃) m/z 304 (M+H)⁺; ¹H NMR (300 MHz, CD₃OD) δ 7.85 (dd, 1H), 7.6-7.1 (m, 6H), 6.55 (s, 1H), 6.0 (s, 1H), 3.7 (s, 3H).

### EXAMPLE 4B

### 2′-methyl-5-((1-methyl-1H-imidazol-5-yl)(phenoxy)methyl)(1,1′-biphenyl)-2-carbonitrile

A solution of Example 4A (106 mg, 0.35 mmol), phenol (38.5 mg, 0.35 mmol), and triphenylphosphine (139.2 mg, 0.525 mmol) in THF (2 mL) was treated with diethyl azodicarboxylate (90 μL, 0.525 mmol), stirred for 24 hours, poured into water, and extracted with ethyl acetate. The extract was washed with brine, dried (Na₂SO₄), filtered, and concentrated. The concentrate was purified by flash column chromatography on silica gel with 96.5:2.5:1/ethyl acetate:methanol:concentrated ammonium hydroxide to provide the desired product.

MS (ESI(+)) m/z 380 (M+H)⁺; ¹H NMR (300 MHz, CDCl₃) δ 7.8-6.9 (m, 12H), 6.7 (s, 1H), 6.35 (s, M1), 3.6 (s, 3H).

### EXAMPLE 4C

### 2′-methyl-5-((1-methyl-1H-imidazol-5-yl)(phenoxy)methyl)(1,1′-biphenyl)-2-carbonitrile hydrochloride

A solution of Example 4B (82 mg) in 4M HCl in dioxane (2 mL) was stirred for 2 hours and concentrated. The concentrate was treated with water (2 mL) and lyophilized. The product was purified by HPLC with continuous 20% to 100% :0.1% TFA/water: CH₃CN. The appropriate fractions were combined, adjusted to pH 7-8 with NaHCO₃, and extracted with ethyl acetate. The extract was dried (NaSO₄), filtered, and concentrated. The concentrate was dissolved in 4M HCl in dioxane (0.5 mL), stirred for 3 hours, and concentrated. The concentrate was treated with water and lyophilized to provide the desired product.

MS (ESI(+)) m/z 380 (M+H)⁺; ¹H NMR (300 MHz, DMSO-d₆) δ 9.1 (s, 1H), 8.05 (d, 2H), 7.75-6.95 (m, 13H), 3.8 (s, 3H), 3.55 (s, 1H), 2.0 (s, 3H); Anal. calcd for C₂₅H₂₂ClN₃O·2.6H₂O: C, 64.76; H, 5.93; N, 9.06. Found: C, 64.90; H, 5.40; N, 7.60.

### EXAMPLE 5

### 5-((benzyloxy)(1-methyl-1H-imidazol-5-yl)methyl)-2′-methoxy(1,1′-biphenyl)-2-carbonitrile hydrochloride

### EXAMPLE 5A

### ethyl 6-cyano-2′-methoxy(1,1′-biphenyl)-3-carboxylate

The desired product was prepared by substituting ethyl 3-bromo-4-cyanobenzoate and 2-methoxyphenylboronic acid for 3-bromo-4-fluorobenzaldehyde and 2-methylphenylboronic acid, respectively, in Example 1A.

MS (DCI/NH₃) m/z 299 (M+NH₄)⁺; ¹H NMR (300 MHz, CDCl₃) δ 8.1 (m, 2H), 7.8 (d, 1H), 7.45 (m, 1H), 7.25 (dd, 1H), 7.05 (m, 2H), 4.4 (q, 2H), 3.8 (s, 3H), 1.4 (t, 3H).

### EXAMPLE 5B

### 5 -(hydroxymethyl)-2′-methoxy(1,1′-biphenyl)-2-carbonitrile

A solution of Example 5A (389 mg, 1.38 mmol) in THF (3 mL) was treated sequentially with calcium chloride (312 mg, 2.76 mmol), absolute ethanol (4 mL), and sodium borohydride (209 mg, 5.52 mmol), stirred for 48 hours, treated with water (1 mL) and 2M HCl (2 mL) to break up any solid, and extracted with diethyl ether. The extract was washed with brine, dried (Na₂SO₄), filtered, and concentrated. The product was purified by flash column chromatography on silica gel with 1: 1 ethyl/acetate:hexanes to provide the desired product.

MS (DCI/NH₃) m/z 357 (M+NH₄)⁺; ¹H NMR (300 MHz, CD₃OD) δ 7.75 (d, 1H), 7.45 (m, 3H), 7.25 (dd, 1H), 7.15-7.0 (m, 2H), 4.7 (s, 2H), 3.8 (s, 3H).

### EXAMPLE 5C

### 5-formyl-2′-methoxy(1,1′-biphenyl)-2-carbonitrile

A solution of oxalyl chloride (0.25 mL, 2.76 mmol) in dichloromethane (2 mL) at −78 ° C. was treated with DMSO (0.4 mL, 5.52 mmol), stirred for 20 minutes, treated with Example 5B (331 mg, 1.38 mmol) in dichloromethane (3 mL), stirred for 3 hours at −78 ° C., treated with triethylamine (0.77 mL, 5.52 mmol), warmed to room temperature, poured into diethyl ether (20 mL), washed sequentially with water, saturated NaHCO₃, water, and brine, dried (Na₂SO₄), filtered, and concentrated to provide material of sufficient purity for subsequent use without further purification.

MS (DCI/NH₃) m/z 255 (M+NH₄)⁺; ¹H NMR (300 MHz, CDCl₃) δ 10.1 (s, 1H), 7.95 (m, 3H), 7.45 (dt, 1H), 7.3 (dd, 1H), 7.15-7.0 (m, 2H), 3.85 (s, 3H).

### EXAMPLE 5D

### 5-(hydroxy(l -methyl-1H-imidazol-5-yl)methyl)-2′-methoxy(1,1′-biphenyl)-2-carbonitrile

The desired product was prepared by substituting Example 5C for Example 1A in Example 1B.

MS (DCI/NH₃) m/z 320 (M+H)⁺; ¹H NMR (300 MHz, CD₃OD) δ 7.8 (d, 1H), 7.7-7.4 (m, 3H), 7.3-6.9 (m, 3H), 6.6 (s, 1H), 6.0 (s, 1H), 3.8 (s, 3H), 3.7 (s, 3H).

### EXAMPLE 5E

### 5-((benzyloxy)(1-methyl-1H-imidazol-5-yl)methyl)-2′-methoxy(1,1′-biphenyl)-2-carbonitrile hydrochloride

A mixture of Example 5D (113 mg, 0.35 mmol), silver(I) oxide (91 mg, 0.39 mmol), (bromomethyl)benzene (0.05 mL, 0.42 mmol), and dichloromethane (15 mL) was stirred for 36 hours in darkness, filtered through a pad of diatomaceous earth (Celite®), and concentrated. The concentrate was purified by flash column chromatography on silica gel with 95:5:1/ethyl acetate:methanol:concentrated ammonium hydroxide. The appropriate fractions were concentrated, and the concentrate was dissolved in 4M HCl in dioxane (1 mL), stirred for 3 hours, and concentrated. The concentrate was treated with water and lyophilized to provide the desired product.

MS (ESI(+)) m/z 410 (M+H)⁺; ¹H NMR (300 MHz, DMSO-d₆) δ 9.0 (s, 1H), 8.0-7.0 (m, 12H), 6.0 (s, 1H), 4.55 (q, 2H), 3.75 (s, 3H), 3.65 (s, 3H).

### EXAMPLE 6

### 5-((benzyloxy)(1-methyl-1H-imidazol-5-yl)methyl)-3′-(phenyl)(1,1′-biphenyl)-2-carbonitrile hydrochloride

### EXAMPLE 6A

### 3-(dihydroxyboryl)-1,1′-biphenyl

A solution of 1.7M tert-butyllithium in pentane (12.6 mL, 21.5 mmol) in diethyl ether (65 mL) at −78 ° C. was treated with 3-bromo-1,1′-biphenyl (2 g, 8.6 mmol) in diethyl ether (20 mL), stirred for 1 hour, treated with triisopropylborate (5 mL, 21.5 mmol), warmed to room temperature over 1 hour, poured into 2M NaOH (200 mL), stirred for 15 minutes, cooled, adjusted to pH 1 with concentrated HCl, and extracted with diethyl ether and ethyl acetate. The extract was washed with brine, dried (Na₂SO₄), filtered, and concentrated to provide material of sufficient purity for subsequent use without further purification.

MS (DCI/NH₃) m/z 198 (M+H)⁺.

### EXAMPLE 6B

### ethyl 6-cyano-3′-(phenyl)(1,1′-biphenyl)-3-carboxylate

The desired product was prepared by substituting 3-bromo-4-cyanoethylbenzoate and Example 6A for 3-bromo-4-fluorobenzaldehyde and 2-methoxyphenylboronic acid, respectively, in Example 1A, and purified by flash column chromatography on silica gel with 95:5/hexanes:ethyl acetate.

MS (DCI/NH₃) m/z 345 (M+NH₄)⁺; ¹H NMR (300 MHz, CDCl₃) δ 8.25 (d, 1H), 7.9-7.3 (m, 10H), 4.45 (q, 2H), 1.4 (t, 3H).

### EXAMPLE 6C

### 5-(hydroxymethyl)-3′-(phenyl)(1,1′-biphenyl)-2-carbonitrile

The desired product was prepared by substituting Example 6B for Example 5A in Example 5B.

MS (DCI/NH₃) m/z 303 (M+NH₄)⁺; ¹H NMR (300 MHz, CD₃OD) δ 8.1-7.3 (m, 12H), 4.8 (s, 2H).

### EXAMPLE 6D

### 5-formyl-3′-(phenyl)(1,1′-biphenyl)-2-carbonitrile

The desired product was prepared by substituting Example 6C for Example 5B in Example 5C, and purified by flash column chromatography on silica gel with 7:3/hexanes:ethyl acetate.

MS (DCI/NH₃) m/z 301 (M+NH₄)⁺; ¹H NMR (300 MHz, CDCl₃) δ 10.15 (s, 1H), 8.1-7.35 (m, 12H).

### EXAMPLE 6E

### 5-(hydroxy(1-methyl-1H-imidazol-5-yl)methyl)-3′-(phenyl)(1,1′-biphenyl)-2-carbonitrile

The desired product was prepared by substituting Example 6D for Example 1A in Example 1B.

¹H NMR (300 MHz, CDCl₃) δ 7.8-7.3 (m, 12H), 6.75 (s, 1H), 6.0 (s, 1H), 3.6 (s, 3H), 3.4-3.0 (brs, 1H).

### EXAMPLE 6F

### 5-((benzyloxy)(1-methyl-1H-imidazol-5-yl)methyl)-3-(phenyl)(1,1-biphenyl)-2-carbonitrile hydrochloride

The desired product was prepared by substituting Example 6E for Example 6D in Example 6E.

MS (ESI(+)) m/z 456 (M+H)⁺; ¹H NMR (300 MHz, DMSO-d₆) δ 9.1 (s, 1H), 8.1-7.3 (m, 12H), 6.1 (s, 1H), 4.6 (q, 2H), 3.8 (s, 3H); Anal. calcd for C₃₁H₂₆ClN₃O·2.3H₂O: C, 69.56; H, 5.80; N, 7.85. Found: C, 69,43; H, 5.50; N, 8.32.

### EXAMPLE 7

### (2-(9-anthryl)-4-((benzyloxy)(1-methyl-1H-imidazol-5-yl)methyl)benzonitrile hydrochloride

### EXAMPLE 7A

### 9-anthrylboronic acid

The desired product was prepared by substituting 9-bromoanthracene for 3-bromo-1,1′-biphenyl in Example 6A, and purified by flash column chromatography on silica gel with 9:1/hexanes:ethyl acetate to 7:3/hexanes:ethyl acetate.

MS (DCI/NH₃) m/z 268 (M+2NH₄)⁺.

### EXAMPLE 7B

### ethyl 3-(9-anthryl)-4-cyanobenzoate

The desired product was prepared by substituting ethyl 3-bromo-4-cyanobenzoate and Example 7A for 3-bromo-4-fluorobenzaldehyde and 2-methoxyphenylboronic acid, respectively, in Example 7A, and purified by flash column chromatography on silica gel with 9:1/hexanes:ethyl acetate.

MS (DCI/NH₃) m/z 369 (M+NH₄)⁺; ¹H NMR (300 MHz, CDCl₃) δ 8.6 (s, 1H), 8.35 (dd, 1H), 8.25-7.95 (m, 3H), 7.85-7.3 (m, 6H), 4.4 (q, 2H), 1.4 (t, 3H).

### EXAMPLE 7C

### 2-(9-anthryl)-4-(hydroxymethyl)benzonitrile

The desired product was prepared by substituting Example 7B for Example 5A in Example 5B.

MS (DCI/NH₃) m/z 327 (M+NH₄)⁺; ¹H NMR (300 MHz, CD₃OD) δ 8.65 (s, 1H), 8.17 (s, 1H), 8.12 (s, 1H), 8.0 (d, 1H), 7.8-7.6 (m, 2H), 7.6-7.4 (m, 6H), 4.8 (s, 2H).

### EXAMPLE 7D

### 2-(9-anthryl)-4-formylbenzonitrile

The desired product was prepared by substituting Example 7C for Example 5B in Example 5C, and purified by flash column chromatography on silica gel with 7:3/hexanes:ethyl acetate.

MS (DCI/NH₃) m/z 325 (M+NH₄)⁺; 1H NMR (300 MHz, CDCl₃) δ 10.2 (s, 1H), 8.15 (s, 1H), 8.3-7.8 (m, 5H), 7.6-7.35 (m, 6H).

### EXAMPLE 7E

### 2-(9-anthryl)-4-(hydroxy(1-methyl-1H-imidazol-5-yl)methyl)benzonitrile

The desired product was prepared by substituting Example 7D for Example 1A in Example 1B and purified by flash column chromatography on silica gel with 95:5:1/ethyl acetate:methanol:concentrated ammonium hydroxide.

MS (DCI/NH₃) m/z 390 (M+H)⁺.

### EXAMPLE 7F

### 2-(9-anthryl)-4-((benzyloxy)(1-methyl-1H-imidazol-5-yl)methyl)benzonitrile hydrochloride

The desired product was prepared by substituting Example 7E and dichloromethane for Example 1B and DMF, respectively, in Example 1C.

MS (ESI(+)) m/z 480 (M+H)⁺; ¹H NMR (300 MHz, DMSO-d₆) δ 8.8 (s, 1H), 8.3-8.2 (m, 2H), 7.9 (dd, 1H), 7.7-7.3 (m, 14H), 6.1 (s, 1H), 4.65 (q, 2H), 3.8 (s, 3H).

### EXAMPLE 8

### 5-((benzyloxy)(1-methyl-1H-imidazol-5-yl)methyl)-2′-isopropyl(1,1′-biphenyl)-2-carbonitrile hydrochloride

### EXAMPLE 8A

### 2-isopropylphenylboronic acid

A mixture of magnesium (720 mg, 30 mmol) and diethyl ether (15 mL) was treated with a small aliquot of 1-bromo-2-isopropylbenzene, stirred for 30 minutes, treated dropwise with 2-bromoisopropylbenzene (4.978 g, 25 mmol) in diethyl ether (10 mL), stirred at reflux for 1 hour, cooled to room temperature, added to a solution of triisopropylborate (6.4 mL, 27.5 mmol) in diethyl ether (15 mL) at −78° C., warmed to room temperature, treated with 4M NaOH (10 mL), stirred for 10 minutes, poured into water, washed with diethyl ether, adjusted to pH 1 with concentrated HCl, and extracted with diethyl ether. The extract was dried (Na₂SO₄), filtered, and concentrated to provide material of sufficient purity for subsequent use without further purification.

MS (DCI/NH₃) m/z 182 (M+NH₄)⁺.

### EXAMPLE 8B

### ethyl 6-cyano-2′-isopropyl(1,1′-biphenyl)-3-carboxylate

The desired product was prepared by substituting ethyl 3-bromo-4-cyanobenzoate and Example 8A for 3-bromo-4-fluorobenzaldehyde and 2-methoxyphenylboronic acid, respectively, in Example 1A, and purified by flash column chromatography on silica gel with 9:1/hexanes:ethyl acetate.

MS (DCI/NH₃) m/z 311 (M+NH₄)⁺; ¹H NMR (300 MHz, CDCl₃) δ 8.1 (dd, 1H), 8.05 (d, 1H), 7.8 (d, 1H), 7.48 (s, 1H), 7.42 (s, 1H), 7.25 (m, 1H), 7.15 (d, 1H), 4.4 (q, 2H), 2.7 (sept., 1H), 1.4 (t, 3H), 1.25 (dd, 6H).

### EXAMPLE 8C

### 5-(hydroxymethyl)-2′-isopropyl(1,1′-biphenyl)-2-carbonitrile

The desired product was prepared by substituting Example 8B for Example 5A in Example 5B.

MS (DCI/NH₃) m/z 269 (M+NH₄)⁺; ¹H NMR (300 MHz, CD₃OD) δ 7.8 (d,1H), 7.6-7.35 (m, 4H), 7.25 (dt, 1H), 7.1 (d, 1H), 4.7 (s, 2H), 2.7 (sept., 1H), 1.15 (dd, 6H).

### EXAMPLE 8D

### 5-formyl-2′-isopropyl(1,1′-biphenyl)-2-carbonitrile

The desired product was prepared by substituting Example 8C for Example 5B in Example 5C.

MS (DCI/NH₃) m/z 267 (M+NH₄)⁺; ¹H NMR (300 MHz, CDCl₃) δ 10.1 (s, 1H), 8.0-7.8 (m, 3H), 7.48 (s, 1H), 7.45 (s, 1H), 7.3 (m, 1H), 7.15 (d, 1H), 2.7 (sept., 1H), 1.2 (dd, 6H).

### EXAMPLE 8E

### 5-(hydroxy(1-methyl-1H-imidazol-5-yl)methyl)-2′-isopropyl(1,1′-biphenyl)-2-carbonitrile

The desired product was prepared by substituting Example 8D for Example 1A in Example 1B, and purified by flash column chromatography on silica gel with 95:5:1/ethyl acetate:methanol:concentrated ammonium hydroxide.

MS (DCI/NH₃) m/z 332 (M+H)⁺; ¹H NMR (300 MHz, CDCl₃) δ 7.8-6.9 (m, 7H), 6.75 (s, 1H), 6.0 (s, 1H), 3.6 (s, 3H), 2.7 (sept., 1H), 1.1 (dd, 6H).

### EXAMPLE 8F

### 5-((benzyloxy)(1-methyl-1H-imidazol-5-yl)methyl)-2′-isopropyl(1,1′-biphenyl)-2-carbonitrile hydrochloride

The desired product was prepared by substituting Example 8E for Example 5D in Example 5E, and purified by flash column chromatography on silica gel with 95:5:1/ethyl acetate:methanol:concentrated ammonium hydroxide.

MS (ESI(+)) m/z 422 (M+H)⁺; ¹H NMR (300 MHz, DMSO-d₆) δ 9.1 (s, 1H), 8.05 (dd, 1H), 7.7-7.1 (m, 11H), 6.1 (s, 1H), 4.6 (q, 2H), 3.75 (s, 3H), 2.65 (sept., 1H), 1.05 (dd, 6H); Anal. calcd for C₂₈H₂₈ClN₃O·0.85H₂O: C, 71.05; H, 6.32; N, 8.88. Found: C, 71.15; H, 6.36; N, 8.01.

### EXAMPLE 9

### 4-((benzyloxy)(1-methyl-1H-imidazol-5-yl)methyl)-2-(1,2-dihydro-5-acenaphthylenyl)benzonitrile hydrochloride

### EXAMPLE 9A

### 1,2-dihydro-5-acenaphthylenylboronic acid

The desired product was prepared by substituting 5-bromo-1,2-dihydroacenaphthylene for 3-bromo-1,1′-biphenyl in Example 6A.

MS (DCI/NH₃) m/z 216 (M+NH₄)⁺; ¹H NMR (300 MHz, CD₃OD) δ 7.55 (dd, 2H), 7.45 (t, 1H), 7.25 (dd, 2H), 3.4 (s, 4H).

### EXAMPLE 9B

### ethyl 4-cyano-3-(1,2-dihydro-5-acenaphthylenyl)benzoate

The desired product was prepared by substituting ethyl 3-bromo-4-cyanobenzoate and Example 9A for 3-bromo-4-fluorobenzaldehyde and 2-methoxyphenylboronic acid, respectively, in Example 1A, and purified by flash column chromatography on silica gel with 3:1/hexanes:ethyl acetate.

MS (DCI/NH₃) m/z 345 (M+NH₄)⁺; ¹H NMR (300 MHz, CDCl₃) δ 8.25 (d, 1H), 8.15 (dd, 1H), 7.9 (d, 1H), 7.5-7.25 (m, 5H), 4.4 (q, 2H), 3.5 (s, 4H), 1.4 (t, 3H).

### EXAMPLE 9C

### 2-(1,2-dihydro-5-acenaphthylenyl)-4-(hydroxymethyl)benzonitrile

The desired product was prepared by substituting Example 9B for Example 5A in Example 5B.

MS (DCI/NH₃) m/z 303 (M+NH₄)⁺; ¹H NMR (300 MHz, CD₃OD) δ 7.6 (t, 2H), 7.45-7.25 (m, 5H), 7.35 (d, 1H), 4.75 (s, 2H), 3.45 (s, 4H).

### EXAMPLE 9D

### 2-(1,2-dihydro-5-acenaphthylenyl)-4-formylbenzonitrile

The desired product was prepared by substituting Example 9C for Example 5B in Example 5C, and purified by flash column chromatography on silica gel with 7:3/hexanes:ethyl acetate.

MS (DCI/NH₃) m/z 301 (M+NH₄)⁺; ¹H NMR (300 MHz, CDCl₃) δ 10.15 (s, 1H), 8.1 (d, 1H), 8.0 (t, 2H), 7.5-7.25 (m, 5H), 3.45 (s, 4H).

### EXAMPLE 9E

### 2-(1,2-dihydro-5-acenaphthylenyl)-4-(hydroxy(1-methyl-1H-imidazol-5-yl)methyl)benzonitrile

The desired product was prepared by substituting Example 9D for Example 1A in Example 1B, and purified by flash column chromatography on silica gel with 95:5:1/ethyl acetate:methanol:concentrated ammonium hydroxide.

MS (DCI/NH₃) m/z 366 (M+H)⁺; ¹H NMR (300 MHz, CDCl₃) δ 7.85 (d, 1H), 7.65 (s, 1H), 7.55 (m, 1H), 7.5-7.3 (m, 5H), 6.75 (s, 1H), 6.0 (s, 1H), 3.7 (s, 1H), 3.6 (s, 3H), 3.45 (s, 4H).

### EXAMPLE 9F

### 4-((benzyloxy)(1-methyl-1H-imidazol-5-yl)methyl)-2-(1,2-dihydro-5-acenaphthylenyl)benzonitrile hydrochloride

The desired product was prepared by substituting Example 9E for Example 5D in Example 5E, and purified by flash column chromatography on silica gel with 95:5: 1/ethyl acetate:methanol:concentrated ammonium hydroxide.

MS (ESI(+)) m/z 456 (M+H)⁺; ¹H NMR (300 MHz, DMSO-d₆) δ 9.1 (s, 1H), 8.2-7.2 (m, 13H), 6.1 (s, 1H), 4.6 (q, 2H), 3.8 (s,3H), 3.5 (s,4H); Anal. calcd for C₃₁H₂₆ClN₃O·1.5H₂O: C, 71.61; H 5.64; N, 8.08. Found: C, 71.62; H, 5.35; N, 8.26.

### EXAMPLE 10

### 5-((benzyloxy)(1-methyl-1H-imidazol-5-yl)methyl)-2′-chloro(1,1′-biphenyl)-2-carbonitrile hydrochloride

### EXAMPLE 10A

### 2′-chloro-6-(methoxycarbonyl)(1,1′-biphenyl)-3-carboxylic acid

The desired product was prepared by substituting dimethyl 2-iodoterephthalate and 2-chlorophenylboronic acid and for Example 3B and 2-methylphenylboronic acid, respectively, in Example 3C to provide material of sufficient purity for use without further purification.

### EXAMPLE 10B

### 2′-chloro-6-(methoxycarbonyl)(1,1′-biphenyl)-3-carboxylic acid

A solution of Example 10A in THF (100 mL) was treated with 1M LiOH (33 mL), stirred for 4 days, concentrated, treated with water, and adjusted to pH 1 with 4M HCl to precipitate a first crop of desired product. This first crop was recrystallized from 1:1 ethanol/water and filtered. The filtrate was concentrated to remove the ethanol and extracted with ethyl acetate. The extract was washed water and brine, dried (Na₂SO₄), filtered, and concentrated to provide a second crop material of sufficient purity for subsequent use without further purification.

¹H NMR (300 MHz, CDCl₃) δ 8.2 (dd, 1H), 8.1 (d, 1H), 8.05 (d, 1H), 7.45 (mn, 1H), 7.35 (m, 2H), 7.25 (m, 1H), 3.7 (s, 3H).

### EXAMPLE 10C

### methyl 2′-chloro-5-(hydroxymethyl)(1,1′-bighenyl)-2-carboxylate

A solution of Example 10B (6.29 g, 21.64 mmol) in THF (30 mL) at 0° C. was treated with 10M boranedimethylsulfide in THF (4.4 mL, 43.28 mmol), stirred for 24 hours, treated with additional borane-dimethylsulfide (2 mL), stirred for 24 hours, treated dropwise with 4:1/THF:water (25 mL), stirred for 1 hour, and treated with 3M HCl (50 mL) to form two separate layers. The layers were separated, and the aqueous layer was extracted with ethyl acetate. The extract was washed sequentially with 2M Na₂CO₃, water, and brine, dried (Na₂SO₄), filtered, and concentrated. The product was purified by flash column chromatography on silica gel with 3:1 to 3:2/hexanes:ethyl acetate to provide the desired product.

MS (DCI/NH₃) m/z 294 (M+NH₄)⁺.

### EXAMPLE 10D

### methyl 2′-chloro-5-formyl(1,1′-biphenyl)-2-carboxylate

The desired product was prepared by substituting Example 10C for Example 5B in Example 5C, and purified by flash column chromatography on silica gel with 75:25/hexanes:ethyl acetate.

MS (DCI/NH₃) m/z 292 (M+NH₄)⁺; ¹H NMR (300 MHz, CDCl₃) δ 10.1 (s, 1H), 8.15 (dd, 1H), 8.0 (dd, 2H), 7.8 (d, 1H), 7.5 (m, 1H), 7.35 (m, 1H), 3.7 (s, 3H).

### EXAMPLE 10E

### methyl 2′-chloro-5-(hydroxy(1-methyl-1H-imidazol-5-yl)methyl)(1,1′-biphenyl)-2-carboxylate

The desired product was prepared by substituting Example 10D for Example 1A in Example 1B, and purified by flash column chromatography on silica gel with 95:5: 1/ethyl acetate:methanol:concentrated ammonium hydroxide.

MS (DCI/NH₃) m/z 356 (M+H)⁺; ¹H NMR (300 MHz, CD₃OD) δ 8.0 (dd, 1H), 7.6-6.9 (m, 6H), 6.6 (s, 1H), 6.0 (s, 1H), 3.65 (s, 3H), 3.6 (s, 3H).

### EXAMPLE 10F

### methyl 5-((benzyloxy)(1-methyl-1H-imidazol-5-yl)methyl)-2′-chloro(1,1′-biphenyl)-2-carboxylate

The desired product was prepared by substituting Example 10E for Example 5D in Example 5E, and purified by flash column chromatography on silica gel with 95:5:1/ethyl acetate:methanol:concentrated ammonium hydroxide.

MS (DCI/NH₃) m/z 447 (M+H)⁺; ¹H NMR (300 MHz, CDCl₃) δ 8.05 (dd, 1H), 7.6-7.1 (m, 11H), 6.9 (s, 1H), 5.6 (s, 1H), 4.55 (s, 2H), 3.65 (s, 3H), 3.45 (s, 3H).

### EXAMPLE 10G

### 5-((benzyloxy)(1-methyl-1H-imidazol-5-yl)methyl)-2′-chloro(1,1′-biphenyl)-2-carboxylic acid

A solution of Example 10F (835 mg, 1.87 mmol) in methanol (10 mL) was treated with 4M NaOH, heated to reflux for 4 hours, cooled, concentrated, poured into 0.5M H₃PO₄ in diethyl ether, and extracted with 4:10/chloroform:isopropyl alcohol. The extract was dried (Na₂SO₄), filtered, and concentrated to provide material of sufficient purity for subsequent use without further purification.

MS (DCI/NH₃) m/z 433 (M+H)⁺; ¹H NMR (300 MHz, CDCl₃) δ 8.1 (s, 1H), 7.7-7.2 (m, 11H), 6.85 (s, 1H), 5.6 (s, 1H), 4.55 (s, 2H), 3.45 (s, 3H).

### EXAMPLE 10H

### 5-((benzyloxy)(1-methyl-1H-imidazol-5-yl)methyl)-2′-chloro(1,1′-biphenyl)-2-carboxamide

A slurry of Example 10G (794 mg, 1.83 mmol), EDC (385 mg, 2.01 mmol), and HOBt (271 mg, 2.01 mmol) in DMF (4 mL) was stirred until a clear solution resulted, treated with concentrated ammonium hydroxide (0.62 mL, 9.15 mmol), stirred for 24 hours, treated with ethyl acetate, washed sequentially with 0.5M H₃PO₄, saturated NaHCO₃, and brine, dried (Na₂SO₄), filtered, and concentrated to provide the desired product of sufficient purity for subsequent use without further purification.

¹H NMR (300 MHz, CD₃OD) δ 7.95 (s, 1H), 7.8-7.2 (m, 11H), 6.65 (s, 1H), 5.75 (s, 1H), 4.55 (s, 2H), 3.55 (s, 3H), 3.0 (s, 1H), 2.85 (s, 1H).

### EXAMPLE 10I

### 5-((benzyloxy)(1-methyl-1H-imidazol-5-yl)methyl)-2′-chloro(1,1′-biphenyl)-2-carbonitrile hydrochloride

A solution of Example 10H (519 mg, 1.20 mmol) in THF (2.5 mL) at 0° C. was treated with triethylamine (1 mL, 7.08 mmol), stirred for 10 minutes, treated with trifluoroacetic anhydride (0.5 mL, 3.60 mmol), stirred for 40 minutes, warmed to room temperature, stirred for 1 hour, poured onto ice, treated with concentrated ammonium hydroxide/THF until a clear solution formed, poured into water, and extracted with diethyl ether. The extract was washed with brine, and the washes were back-extracted with diethyl ether. The extract was dried (Na₂SO₄), filtered, and concentrated. The product was purified by flash column chromatography on silica gel with 95:5: 1/ethyl acetate:methanol:concentrated ammonium hydroxide. The appropriate fractions were concentrated, and the concentrate was dissolved in 4M HCl in dioxane (1 mL), stirred for 3 hours, and concentrated. The concentrate was treated with water and lyophilized to provide the desired product.

MS (ESI(+)) m/z 414 (M+H)⁺; ¹H NMR (300 MHz, DMSO-d₆) δ 9.2 (s, 1H), 8.1 (d 1H), 7.8-7.45 (m, 5H), 7.4-7.2 (m, 6H), 6.1 (s, 1H), 4.6 (q, 2H), 3.75 (s, 3H); Anal. calcd for C₂₅H₂₁Cl₂N₃O·0.7H₂O·0.35TFA: C, 61.38; H, 4.56; N, 8.36. Found: C, 61.47; H, 4.62; N, 8.09.

### EXAMPLE 11

### 5-((benzyloxy)(1-methyl-1H-imidazol-5-yl)methyl)-2′-methyl(1,1′-biphenyl)-2-carbonitrile hydrochloride

The desired product was prepared by substituting Example 4A for Example 1B in Example 1C.

MS (ESI(+)) m/z 394 (M+H)⁺; ¹H NMR (300 MHz, DMSO-d₆) δ 9.05 (s, 1H), 8.05 (d, 1H), 7.7 (dd, 1H), (d, 1H), 7.4-7.2 (m, 1H), 6.05 (s, 1H), 4.6 (q, 2H), 3.75 (s, 3H), 2.15 (s, 3H); Anal. calcd for C₂₆H₂₄ClN₃O·0.75H₂O: C, 70.42; H, 5.80; N, 9.48. Found: C, 70.44; H, 5.86; N, 8.90.

### EXAMPLE 12

### (2′-methyl-5-((1-methyl-1H-imidazol-5-yl)((4-nitrobenzyl)amino)methyl)(1,1′-biphenyl)-2-carbonitrile

### EXAMPLE 12A

### 5-(amino(1-methyl-1H-imidazol-5-yl)methyl)-2′-methyl(1,1′-biphenyl)-2-carbonitrile

A suspension of Example 4A (0.3 g, 1.0 mmol) in dichloromethane (3 mL) was cooled to 0° C., treated with a solution of thionyl chloride (240 mg, 2.0 mmol) in dichloromethane (2 mL), stirred 30 minutes, warmed to room temperature, stirred 4 hours, cooled to 0° C., treated with concentrated ammonium hydroxide (5 mL), warmed to room temperature, stirred for 16 hours, and concentrated. The concentrate was treated with ethyl acetate, washed with water and brine, dried (Na₂SO₄), filtered, and concentrated. The concentrate was purified by flash column chromatography on silica gel with 9:1/dichloromethane:methanol to provide the desired product.

### EXAMPLE 12B

### (2′-methyl-5-((1-methyl-1H-imidazol-5-yl)((4-nitrobenzyl)amino)methyl)(1,1′-biphenyl)-2-carbonitrile

A solution of Example 12A (100 mg, 0.33 mmol) in 1,2-dichloroethane (2 mL) was treated with 4-nitrobenzaldehyde (94 mg, 0.62 mmol) and acetic acid (150 mg, 2.5 mmol), stirred for 30 minutes, treated with sodium triacetoxyborohydride (265 mg, 1.25 mmol), and stirred for 16 hours. The mixture was diluted with ethyl acetate, washed sequentially with saturated NaHCO₃, water, and brine, dried (MgSO₄), filtered, and concentrated. The concentrate was treated with dichloromethane (5 mL) and a solution of 4M HCl in dioxane (1 mL), stirred for 30 minutes, and concentrated. The concentrate was treated with ethyl acetate, washed sequentially with saturated NaHCO₃, water, and brine, dried (MgSO₄), filtered, and concentrated. The concentrate was purified by flash column chromatography on silica gel with dichloromethane then 98:2/dichloromethane: methanol to provide the desired product.

MS (ESI(+)) m/z 438 (M+H)⁺; ¹H NMR (300 MHz, CDCl₃) δ 8.20 (d, 2H), 7.78 (d, 1H), 7.50-7.29 (m, 8H), 7.20-7.17 (m, 1H), 6.88 (s, 1H), 4.95 (s, 1H), 3.89 (abq, 2H), 3.53 (s, 3H), 2.17 (s, 3H), 2.05 (s, 1H).

### EXAMPLE 13

### 4-(((4-cyanobenzyl)amino)(1-methyl-1H-imidazol-5-yl)methyl)-2-(1-naphthyl)benzonitrile dihydrochloride

### EXAMPLE 13A

### 4-(amino(1-methyl-1H-imidazol-5-yl)methyl)-2-(1-naphthyl)benzonitrile

A suspension of Example 89D (0.5 g, 1.48 mmol) in dichloromethane (10 mL) at 0° C. was treated with thionyl chloride (0.65 mL, 8.85 mmol), stirred for 30 minutes, warmed to room temperature, stirred for 1.5 hours, and concentrated. The concentrate was treated with dichloromethane (3 mL), and the resulting solution was added to a solution of concentrated ammonium hydroxide (10 mL) at 0° C. This solution was stirred for 30 minutes, warmed to room temperature, stirred for 2 hours, and concentrated. The concentrate was treated with ethyl acetate, washed with water and brine, dried (Na₂SO₄), filtered, and concentrated. The concentrate was purified by flash column chromatography on silica gel with 9:1/dichloromethane:methanol to provide the desired product.

MS (ESI(+)) m/z 339 (M+H)⁺; ¹H NMR (300 MHz, CDCl₃) δ 7.95 (dd, 2H), 7.84 (d, 1H), 7.57-7.42 (m, 8H), 6.86 (d, 1H), 5.32 (d, 1H), 3.59 (d, 3H).

### EXAMPLE 13B

### 4-(((4-cyanobenzyl)amino)(1-methyl-1H-imidazol-5-yl)methyl)-2-(1-naphthyl)benzonitrile dihydrochloride

The desired product was prepared by substituting 4-formylbenzonitrile and Example 13A for 4-nitrobenzaldehyde and Example 12A, respectively, in Example 12B. The purified concentrate was dissolved in dichloromethane, treated with 1M HCl in diethyl ether, and concentrated to provide the desired product.

MS (ESI(+)) m/z 454 (M+H)⁺; ¹H NMR (400 MHz, CDCl₃) δ 7.96-7.93 (m, 2H), 7.83 (d, 1H), 7.60-7.38 (m, 12H), 6.89-6.88 (m, 1H), 4.96 (d, 1H), 3.90-3.80 (m, 2H), 3.53 (d, 3H).

### EXAMPLE 14

### 4-((cyclohexylmethoxy)(1-methyl-1H-imidazol-5-yl)methyl)-2-(1-naphthyl)benzonitrile hydrochloride

A suspension of Example 89D (68 mg, 0.2 mmol) in dichloromethane (4 mL) at 0° C. was treated with thionyl chloride (48 mg, 0.4 mmol), stirred for 30 minutes, warmed to room temperature, stirred for 1.5 hours, treated with cyclohexylmethanol and N,N-diisopropylethylamine, warmed to 35° C., stirred for 16 hours, and concentrated. The concentrate was treated with ethyl acetate, washed with water and brine, dried (MgSO₄), filtered, and concentrated. The concentrate was purified by flash column chromatography on silica gel with dichloromethane then 98:2/dichloromethane:methanol, treated with dichloromethane and 1M HCl in diethyl ether, and concentrated to provide the desired product.

MS (ESI(+)) m/z 436 (M+H)⁺; ¹H NMR (500 MHz, CDCl₃) δ 7.98-7.91 (m, 2H), 7.84-7.82 (m, 1H), 7.60-7.40 (m, 8H), 6.81 (d, 1H), 5.50 (d, 1H), 3.52 (d, 3H), 3.29 (d, 2H), 1.77- 1.63 (m, 6H), 1.27-1.10 (m, 3H), 0.99-0.92 (m, 2H).

### EXAMPLE 15

### (2′-methyl-5-((1-methyl-1H-imidazol-5-yl)(3-oxo-4-(3-(trifluoromethoxy)phenyl)-1-piperazinyl)methyl) (1,1′-biphenyl)-2-carbonitrile dihydrochloride

### EXAMPLE 15A

### tert-butyl 2-oxoethylcarbamate

A solution of tert-butyl allylcarbamate (5.0 g, 31 mmol) in dichloromethane (200 mL) and methanol (25 mL) at −78° C. was treated with ozone until green, treated with zinc (4.0 g, 61.0 mmol) and acetic acid (4 mL), stirred for 16 hours, filtered through a pad of silica gel, and concentrated to provide the desired product of sufficient purity for subsequent use without further purification.

### EXAMPLE 15B

### tert-butyl 2-(3-(trifluoromethoxy)anilino)ethylcarbamate

A solution of 3-(trifluoromethoxy)aniline (4.45 g, 25 mmol) in 1,2-dichloroethane (100 mL) was treated with Example 15A (4.0 g, 25 mmol) and acetic acid (9.0 g, 150 mmol), stirred for 30 minutes, treated with sodium triacetoxyborohydride (15.9 g, 75 mmol), stirred for 16 hours, and concentrated. The residue was treated with ethyl acetate, washed sequentially with saturated NaHCO₃, water, and brine, dried (MgSO₄), filtered, and concentrated. The concentrate was purified by flash column chromatography on silica gel with 4:1/hexanes:ethyl acetate to provide the desired product.

¹H NMR (300 MHz, CDCl₃) δ 7.14 (t, 1H), 6.55-6.48 (m, 2H), 6.40 (s, 1H), 4.78 (s, 1H), 4.31 (s, 1H), 3.42-3.36 (m, 2H), 3.28-3.22 (m, 2H), 1.45 (s, 9H).

### EXAMPLE 15C

### tert-butyl 2-((chloroacetyl)-3-(trifluoromethoxy)anilino)ethylcarbamate

A solution of Example 15B (1.5 g, 4.68 mmol) in ethyl acetate (20 mL) at 0° C. was treated with chloroacetyl chloride (0.38 mL, 5.6 mmol) and saturated NaHCO₃ (20 mL), and stirred for 2 hours to provide two layers. The layers were separated, and the aqueous layer was extracted with ethyl acetate. The extract was dried (MgSO₄), filtered, and concentrated to provide the desired product of sufficient purity for subsequent use without further purification.

MS (ESI(+)) m/z 397 (M+H)⁺; ¹H NMR (300 MHz, CDCl₃) δ 7.51 (t, 1H), 7.31-7.28 (m, 2H), 7.20 (s, 1H), 4.88-4.87 (m, 1H), 3.87-3.81 (m, 4H), 3.39-3.32 (m, 2H), 1.41 (s, 9H).

### EXAMPLE 15D

### tert-butyl 3-oxo-4-(3-(trifluoromethoxy)phenyl)-1-piperazinecarboxylate

A solution of Example 15C (1.7 g, 4.4 mmol) in DMF (10 mL) at 0° C. was treated with cesium carbonate (1.4 g, 4.3 mmol), warmed to room temperature, stirred for 16 hours, treated with ethyl acetate, washed with water and brine, dried (MgSO₄), filtered, and concentrated. The concentrate was purified by flash column chromatography on silica gel with 10:1 to 2:1/hexanes:ethyl acetate to provide the desired product.

¹H NMR (300 MHz, CDCl₃) δ 7.44 (t, 1H), 7.28-7.13 (m, 3H), 4.84-4.74 (m, 4H), 4.27 (s, 2H), 1.50 (s, 9H).

### EXAMPLE 15E

### 1-(3-(trifluoromethoxy)phenyl)-2-piperazinone hydrochloride

A solution of Example 15D (1.2 g, 3.3 mmol) in ethyl acetate (10 mL) at room temperature was treated with 1M HCl in diethyl ether (20 mL), stirred for 30 minutes, and concentrated to provide 0.98 g of the desired product of sufficient purity for subsequent use without further purification.

### EXAMPLE 15F

### (2′-methyl-5-((1-methyl-1H-imidazol-5-yl)(3-oxo-4-(3-(trifluoromethoxy)phenyl)-1-piperazinyl)methyl) (1,1′-biphenyl)-2-carbonitrile dihydrochloride

A suspension of Example 4A (30 mg, 0.1 mmol) in dichloromethane (2 mL) at 0° C. was treated with thionyl chloride (0.15 mL, 2.05 mmol), stirred for 30 minutes, warmed to room temperature, stirred for 3.5 hours, and concentrated. The concentrate was treated with a solution of Example 15E (35 mg, 0.12 mmol) in acetonitrile (2 mL) and N,N-diisopropylethylamine(100 μL, 0.57 mmol), warmed to 80° C., stirred for 3 hours, diluted with ethyl acetate, washed sequentially with saturated NaHCO₃, water, and brine, dried (MgSO₄), filtered, and concentrated. The concentrate was purified by flash column chromatography on silica gel with dichloromethane then 95:5/dichloromethane:methanol. The appropriate fractions were dissolved in dichloromethane, treated with 1M HCl in diethyl ether, and concentrated to provide the desired product.

MS (ESI(+)) m/z 546 (M+H)⁺; ¹H NMR (400 MHz, CDCl₃) δ 7.76 (d, 1H), 7.54 (dd, 1H), 7.47 (d, 1H), 7.44-7.12 (m, 9H), 7.08 (s, 1H), 4.73 (s, 1H), 3.76-3.67 (m, 2H), 3.62 (s, 3H), 3.33 (q, 2H), 2.97-2.82 (m, 2H), 2.16 (d, 3H).

### EXAMPLE 16

### 5-(((1-benzoyl-4-piperidinyl)amino)(1-methyl-1H-imidazol-5-yl)methyl)-2′-methyl(1,1′-biphenyl)-2-carbonitrile dihydrochloride

The desired product was prepared by substituting 1-benzoyl-4-piperidinone for 4-nitrobenzaldehyde in Example 12B. The purified concentrate was dissolved in dichloromethane, treated with 1M HCl in diethyl ether, and concentrated to provide the desired product.

MS (ESI(+)) m/z 490 (M+H)⁺; ¹H NMR (400 MHz, CDCl₃) δ 7.73 (d, 1H), 7.43 (dd, 1H), 7.41-7.24 (m, 10H), 7.18 (d, 1H), 6.72 (s, 1H), 5.11 (s, 1H), 4.51 (s, 1H), 3.76-3.70 (m, 1H), 3.57 (s, 3H), 2.95 (s, 2H), 2.75-2.70 (m, 1H), 2.17 (s, 3H), 1.99-1.65 (m, 3H), 1.50-1.26 (m, 2H).

### EXAMPLE 17

### 4-((1-methyl-1H-imidazol-5-yl)((4-(methylsulfonyl)benzyl amino)methyl)-2-(1-naphthyl)benzonitrile dihydrochloride

The desired product was prepared by substituting 4-(methylsulfonyl)-benzaldehyde for 4-nitrobenzaldehyde in Example 12B. The purified concentrate was dissolved in dichloromethane, treated with 1M HCl in diethyl ether, and concentrated to provide the desired product.

MS (ESI(+)) m/z 507 (M+H)⁺; ¹H NMR (400 MHz, CDCl₃) δ 7.94 (dd, 2H), 7.88-7.82 (m, 3H), 7.59-7.38 (m, 10H), 6.88 (d, 1H), 4.97 (s, 1H), 3.92-3.83 (m, 2H), 3.54 (d, 3H), 3.01 (s, 3H), 2.25 (s, 1H).

### EXAMPLE 18

### 4-(((4-cyanobenzyl)oxy)(1-methyl-1H-imidazol-5-yl)methyl)-2-(8-quinolinyl)benzonitrile dihydrochloride

The desired product was prepared by substituting 8-quinolinylboronic acid for Example 43B in Example 43C.

MS (ESI(+)) m/z 456 (M+H)⁺; 1H NMR (400 MHz, CDCl₃) δ 8.85-8.83 (m, 1H), 8.22 (dd, 1H), 7.93 (dd, 1H) 7.83 (d, 1H), 7.75 (dd, 1H), 7.66-7.61 (m, 4H), 7.57-7.54 (m, 1H), 7.46-7.42 (m, 4H), 7.01 (s, 1H), 5.70 (s, 1H), 4.67-4.60 (m, 2H), 3.50 (s, 3H).

### EXAMPLE 19

### 4-(((4-cyanobenzyl)oxy)(1-methyl-1H-imidazol-5-yl)methyl)-2-(4-quinolinyl)benzonitrile dihydrochloride

### EXAMPLE 19A

### 4-iodoquinoline

A solution of 4-chloroquinoline (5.0 g, 30.56 mmol) in 2-butanone (40 mL) at room temperature was treated with sodium iodide (23 g, 153 mmol) and 47% hydriodic acid (20 mL), heated to reflux for 8 hours, cooled to room temperature, adjusted to pH 7 with saturated NaHCO₃, and extracted with ethyl acetate. The extract was concentrated, and the concentrate was purified by flash column chromatography on silica gel with 4:1/hexanes:ethyl acetate to provide the desired product.

MS (ESI(+)) m/z 256 (M+H)⁺; ¹H NMR (300 MHz, CDCl₃) δ 8.46 (d, 1H), 8.06-8.00 (m, 3H), 7.79-7.73 (m, 1H), 7.66-7.61 (m, 1H).

### EXAMPLE 19B

### 4-quinolinylboronic acid

The desired product was prepared by substituting Example 19A for Example 43A in Example 43B.

¹H NMR (300 MHz, DMSO-d₆) δ 8.86 (d, 1H), 8.75 (s, 1H), 8.25 (dd, 1H), 8.25 (dd, 1H), 8.00 (dd, 1H), 7.76-7.70 (m, 1H), 7.62-7.56 (m, 1H).

### EXAMPLE 19C

### 4-(((4-cyanobenzyl)oxy)(1-methyl-1H-imidazol-5-yl)methyl)-2-(4-quinolinyl)benzonitrile dihydrochloride

The desired product was prepared by substituting Example 19B for Example 43B in Example 43C. The purified concentrate was dissolved in dichloromethane, treated with 1M HCl in diethyl ether, and concentrated to provide the desired product.

MS (ESI(+)) m/z 456 (M+H)⁺; ¹H NMR (400 MHz, CDCl₃) δ 9.03 (dd, 1H), 8.23 (d, 1H), 7.90 (d, 1H), 7.80-7.75 (m, 1H), 7.67-7.32 (m, 10H), 6.98 (d, 1H), 5.70 (d, 1H), 4.63 (abq, 2H), 3.46 (s, 3H).

### EXAMPLE 20

### 5-((5-(hydroxymethyl)-1H-imidazol-1-yl)methyl)-2′-methyl(1,1′-biphenyl)-2-carbonitrile

### EXAMPLE 20A

### 5-(bromomethyl)-2′-methyl(1,1′-biphenyl)-2-carbonitrile

The desired product was prepared by substituting Example 86H for Example 61A in Example 61B.

¹H NMR (300 MHz, CDCl₃) δ 7.72 (d, 1H), 7.47 (dd, 1H), 7.40 (d, 1H), 7.39-7.26 (m, 3H), 7.22-7.18 (m, 1H), 4.50 (s, 2H), 2.20 (s, 3H).

### EXAMPLE 20B

### (1-trityl-1H-imidazol-4-yl)methanol

A solution of 1H-imidazol-5-ylmethanol hydrochloride (1.37 g, 10.2 mmol) and triethylamine (3.55 mL, 25.5 mmol) in DMF (7 mL) at room temperature was treated with a solution of triphenylmethyl chloride (3.0 g, 10.7 mmol) in DMF (14 mL), stirred for 3 days, poured into ice water, and filtered. The filter cake was washed with ice water and dried in a vacuum oven for 16 hours to provide the desired product of sufficient purity for subsequent use without further purification.

¹H NMR (300 MHz, CDCl₃) δ 7.45-7.34 (m, 10H), 7.11-7.07 (m, 6H), 6.72-6.71 (m, 1H), 4.86 (t, 1H), 4.33 (d, 2H).

### EXAMPLE 20C

### (1-trityl-1H-imidazol-4-yl)methyl acetate

A solution of Example 20B (3.5 g, 10.3 mmol) in pyridine (20 mL) at room temperature was treated with acetic anhydride (2.0 mL, 21.2 mmol), stirred for 2 days, cooled, treated with ethyl acetate, washed sequentially with saturated NaHCO₃, water, and brine, dried (MgSO₄), filtered, and concentrated to provide the desired product of sufficient purity for subsequent use without further purification.

¹H NMR (300 MHz, CDCl₃) δ 7.42 (d, 1H), 7.35-7.31 (m, 9H), 7.15-7.10 (m, 6H), 6.88-6.87 (m, 1H), 5.01 (s, 2H), 2.07 (s, 3H).

### EXAMPLE 20D

### (1-((6-cyano-2′-methyl(1,1′-biphenyl)-3-yl)methyl)-1H-imidazol-5-yl)methyl acetate hydrobromide

A solution of Example 20C (2.39 g, 6.25 mmol) in ethyl acetate (15 mL) at 60° C. was treated with Example 20A (1.79 g, 6.25 mmol), stirred for 16 hours, cooled to room temperature, and filtered. The filtrate was reheated to 60° C., stirred for 16 hours, cooled to room temperature, and filtered a second time. The combined solids were dissolved in methanol (20 mL), heated to 60° C., stirred for 6 hours, cooled to room temperature, filtered, and washed with hexanes to provide the desired product of sufficient purity for subsequent use without further purification.

### EXAMPLE 20E

### 5-((5-(hydroxymethyl)-1H-imidazol-1-yl)methyl)-2′-methyl(1,1′-biphenyl)-2-carbonitrile

A solution of Example 20D in 3:1 THF/water at 0° C. was treated with lithium hydroxide monohydrate (840 mg, 19.1 mmol), stirred for 2 hours, and extracted with ethyl acetate. The extract was concentrated, and the concentrate was purified by flash column chromatography on silica gel with 9:1/dichloromethane:methanol to provide the desired product.

MS (ESI(+)) m/z 304 (M+H)⁺; ¹H NMR (300 MHz, CDCl₃) δ 7.72 (d, 1H), 7.53 (s, 1H), 7.37-7.09 (m, 6H), 7.01 (s, 1H), 5.34 (s, 2H), 4.52 (s, 2H), 2.14 (s, 3H).

### EXAMPLE 21

### 2′-methyl-5-((5-((3-oxo-4-(3-(trifluoromethoxy)phenyl)-1-piperazinyl)methyl)-1H-imidazol-1-yl)methyl) (1,1′-biphenyl)-2-carbonitrile dihydrochloride

### EXAMPLE 21A

### 5-((5-formyl-1H-imidazol-1-yl)methyl)-2′-methyl(1,1′-biphenyl)-2-carbonitrile

The desired product was prepared by substituting Example 20E for Example 37A in Example 37B.

MS (ESI(+)) m/z 302 (M+H)⁺; ¹H NMR (300 MHz, CDCl₃) δ 9.76-9.75 (m, 1H), 7.88 (s, 1H), 7.79 (s, 1H), 7.72 (d, 1H), 7.38-7.12 (m, 6H), 5.61 (s, 2H), 2.13 (s, 3H).

### EXAMPLE 21B

### 2′-methyl-5-((5-((3-oxo-4-(3-(trifluoromethoxy)phenyl)-1-piperazinyl)methyl)-1H-imidazol-1-yl)methyl) (1,1′-biphenyl)-2-carbonitrile dihydrochloride

The desired product was prepared by substituting Example 21A and Example 15E for 4-nitrobenzaldehyde and Example 12A, respectively, in Example 12B. The purified concentrate was dissolved in dichloromethane, treated with 1M HCl in diethyl ether, and concentrated to provide the desired product.

MS (ESI(+)) m/z 546 (M+H)⁺; ¹H NMR (300 MHz, CDCl₃) δ 7.74 (d, 1H), 7.60 (s, 1H), 7.44-7.11 (m, 9H), 7.06 (s, 2H), 5.38 (s, 2H), 3.52 (dd, 2H), 3.44 (s, 2H), 3.28 (s, 2H), 2.74 (dd, 2H), 2.13 (s, 3H).

### EXAMPLE 22

### 2′-methyl-5 -((5-(((1-methyl-2-oxo-1,2-dihydro-6-quinolinyl)amino)methyl)-1H-imidazol-1-yl)methyl) (1,1′-biphenyl)-2-carbonitrile dihydrochloride

### EXAMPLE 22A

### 6-amino-1-methyl-2(11H)-quinolinone

A solution of 1-methyl-6-nitro-2(1H)-quinolinone in ethanol (5 mL) at room temperature was treated with Pd/C (10 mg), stirred under 1 atmosphere of hydrogen gas for 16 hours, filtered through a pad of diatomaceous earth (Celite®), and concentrated to provide the desired product of sufficient purity for subsequent use without further purification.

### EXAMPLE 22B

### 2′-methyl-5-((5-(((1-methyl-2-oxo-1,2-dihydro-6-quinolinyl)amino)methyl)-1H-imidazol-1-yl)methyl) (1,1′-biphenyl)-2-carbonitrile dihydrochloride

The desired product was prepared by substituting Example 21A and Example 22A for 4-nitrobenzaldehyde and Example 12A, respectively, in Example 12B. The purified concentrate was dissolved in dichloromethane, treated with 1M HCl in diethyl ether, and concentrated to provide the desired product.

MS (ESI(+)) m/z 460 (M+H)⁺; ¹H NMR (300 MHz, CDCl₃) δ 7.74-7.69 (m, 1H), 7.62-7.55 (m, 1H), 7.47 (d, 1H), 7.36-7.00 (m, 9H), 6.79 (dd, 1H), 6.67-6.65 (mn, 1H), 5.35-5.29 (m, 2H), 4.18 (d, 2H), 3.67 (s, 3H), 3.62-3.56 (m, 1H), 2.10 (m, 3H).

### EXAMPLE 23

### 5-((benzylamino)(1-methy-1H-imidazol-5-yl)methyl)-2′-methyl(1,1′-biphenyl)-2-carbonitrile

The desired product was prepared by substituting benzaldehyde for 4-nitrobenzaldehyde in Example 12B.

MS (ESI(+)) m/z 393 (M+H)⁺; ¹H NMR (300 MHz, CDCl₃) δ 7.75 (d, 1H), 7.46 (dd, 1H), 7.39-7.26 (m, 10H), 7.19 (d, 1H), 6.82 (s, 1H), 4.93 (s, 1H), 3.76 (abq, 2H), 3.52 (s, 3H), 2.18 (s, 3H).

### EXAMPLE 24

### 5-(((cyclohexylmethyl)amino)(1-methyl-1H-imidazol-5-yl)methyl)-2′-methyl(1,1′-biphenyl)-2-carbonitrile

The desired product was prepared by substituting cyclohexylcarboxaldehyde for 4-nitrobenzaldehyde in Example 12B.

MS (ESI(+)) m/z 399 (M+H)⁺; ¹H NMR (300 MHz, CDCl₃) δ 7.77-7.70 (m, 2H), 7.47 (d, 1H), 7.38-7.27 (m, 4H), 7.20 (d, 1H), 6.88 (s, 1H), 4.91 (s, 1H), 3.65 (s, 3H), 2.47-2.36 (m, 2H), 2.17 (s, 3H), 1.80-1.62 (m, 4H), 1.52-1.38 (m, 1H), 1.30-1.05 (m, 4H), 1.00-0.82 (m, 2H).

### EXAMPLE 25

### 5-(((4-cyanobenzyl)amino)(1-methyl-1H-imidazol-5-yl)methyl)-2′-methyl(1,1′-biphenyl)-2-carbonitrile

The desired product was prepared by substituting 4-formylbenzonitrile for 4-nitrobenzaldehyde in Example 12B.

MS (ESI(+)) m/z 418 (M+H)⁺; ¹H NMR (300 MHz, CDCl₃) δ 7.77 (d, 1H), 7.63 (d, 2H), 7.47-7.28 (m, 8H), 7.19 (d, 1H), 6.86 (s, 1H), 4.93 (s, 1H), 3.84 (abq, 2H), 3.52 (s, 3H), 2.17 (s, 3H), 2.00 (s, 1H).

### EXAMPLE 26

### 5-((((6-cyano-2′-methyl(1,1′-biphenyl)-3-yl)methyl)amino)(1-methyl-1H-imidazol-5-yl)methyl)-2-methyl (1,1′-biphenyl)-2-carbonitrile

The desired product was prepared by substituting Example 86I for 4-nitrobenzaldehyde in Example 12B.

MS (ESI(+)) m/z 508 (M+H)⁺; ¹H NMR (300 MHz, CDCl₃) δ 7.77-7.70 (m, 2H), 7.48-7.24 (m, 11H), 7.16 (dd, 2H), 6.85 (s, 1H), 4.97 (s, 1H), 3.92-3.82 (m, 2H), 3.54 (s, 3H), 2.16 (s, 3H), 2.14 (s, 3H), 2.02 (s, 1H).

### EXAMPLE 27

### 5-((ethyl(4-nitrobenzyl)amino)(1-methyl-1H-imidazol-5-yl)methyl)-2′-methyl(1,1′-biphenyl)-2-carbonitrile dihydrochloride

The free base of the desired product was obtained as a byproduct in Example 12B. The purified concentrate was dissolved in dichloromethane, treated with 1M HCl in diethyl ether, and concentrated to provide the desired product.

MS (ESI(+)) m/z 466 (M+H)⁺; ¹H NMR (400 MHz, CDCl₃) δ 8.17 (d, 2H), 7.74 (d, 1H), 7.46-7.43 (m, 4H), 7.38-7.26 (m, 4H), 7.17 (m, 1H), 6.99 (s, 1H), 5.06 (s, 1H), 3.80 (abq, 2H), 3.47 (s, 3H), 2.77-2.68 (m, 1H), 2.64-2.55 (m, 1H), 2.16 (s, 3H), 1.08 (t, 3H).

### EXAMPLE 28

### 5-(((4-cyanobenzyl)(ethyl)amino)(1-methyl-1H-imidazol-5-yl)methyl)-2′-methyl(1,1′-biphenyl)-2-carbonitrile dihydrochloride

The free base of the desired product was obtained as a byproduct in Example 25. The purified concentrate was dissolved in dichloromethane, treated with 1M HCl in diethyl ether, and concentrated to provide the desired product.

MS (ESI(+)) m/z 446 (M+H)⁺; ¹H NMR (400 MHz, CDCl₃) δ 8.17 (d, 2H), 7.74 (d, 1H), 7.46-7.27 (m, 8H), 7.17 (d, 1H), 6.99 (s, 1H), 5.06 (s, 1H), 3.80 (abq, 2H), 3.47 (s, 3H), 2.77-2.68 (m, 1H), 2.64-2.55 (m, 1H), 2.16 (s, 3H), 1.08 (t, 3H).

### EXAMPLE 29

### 4-(((4-cyanobenzyl)(methyl)amino)(1-methyl-1H-imidazol-5-yl)methyl)-2-(1-naphthyl)benzonitrile

A solution of Example 13B (42 mg, 0.09 mmol) in formic acid (5 mL) was treated with 37% aqueous formaldehyde(3 mL), heated to 95° C. for 4 hours, cooled to room temperature, and extracted with ethyl acetate. The extract was washed sequentially with saturated NaHCO₃, water, and brine, dried (MgSO₄), filtered, and concentrated. The concentrate was purified by flash column chromatography on silica gel with dichloromethane then 98:2/dichloromethane:methanol to provide the desired product.

MS (ESI(+)) m/z 468 (M+H)⁺; ¹H NMR (300 MHz, CDCl₃) δ 7.97 (d, 1H), 7.96-7.93 (m, 1H), 7.85 (d, 1H), 7.66-7.35 (m, 12H), 7.05 (d, 1H), 4.89 (d, 1H), 3.66 (d, 2H), 3.62 (d, 3H), 2.20 (d, 3H).

### EXAMPLE 30

### 4-((butyl(4-cyanobenzyl)amino)(1-methyl-1H-imidazol-5-yl)methyl)-2-(1-naphthyl)benzonitrile dihydrochloride

The desired product was prepared by substituting butyraldehyde and Example 13B for 4-nitrobenzaldehyde and Example 12A, respectively, in Example 12B. The purified concentrate was dissolved in dichloromethane, treated with 1M HCl in diethyl ether, and concentrated to provide the desired product.

MS (ESI(+)) m/z 510 (M+H)⁺; ¹H NMR (400 MHz, CDCl₃) δ 7.98-7.93 (m, 2H), 7.82 (dd, 1H), 7.61-7.35 (m, 12H), 7.02 (d, 1H), 5.06 (s, 1H), 3.85-3.70 (m, 2H), 3.44 (d, 3H), 2.69-2.62 (m, 1H), 2.58-2.50 (m, 1H), 1.50-1.41 (m, 2H), 1.26-1.16 (m, 2H), 0.80 (t, 3H).

### EXAMPLE 31

### 4-((1-methyl-1H-imidazol-5-yl)(phenethylamino)methyl)-2-(1-naphthyl)benzonitrile (31-A) and

### 4-(((2-hydroxy-2-phenylethyl)(2-phenylethyl)amino)(1-methyl-1H-imidazol-5-yl)methyl)-2-(1-naphthyl)benzonitrile (31-B)

The desired product was prepared by substituting a 9:1 mixture of phenylacetaldehyde/styrene oxide and Example 13A for 4-nitrobenzaldehyde and Example 12A, respectively, in Example 12B to provide a 9:1 mixture of Example 31-A and Example 31-B.

31-A: MS (ESI(+)) m/z 443 (M+H)⁺; ¹H NMR (400 MHz, CDCl₃) δ 7.94 (dd, 2H), 7.78, (d, 1H), 7.58-7.08 (m, 13H), 6.74 (d, 1H), 4.93 (d, 1H), 3.47 (d, 3H), 2.90-2.86 (m, 2H), 2.82-2.78 (m, 2H); 31-B: MS (ESI(+)) m/z 563 (M+H)⁺; ¹H NMR (400 MHz, CDCl₃) δ 6.69-6.66 (m, 1H), 4.86 (dd, 1H), 4.20-4.12 (m, 1H), 3.38 (d, 3H), 3.23-3.14 (m, 1H), 3.09-3.02 (m, 2H), 2.73-2.69 (m, 1H), 2.69-2.51 (m, 2H).

### EXAMPLE 32

### 5-((benzyl((1-methyl-1H-imidazol-5-yl)methyl)amino)methyl)-2′-methyl(1,1′-biphenyl)-2-carbonitrile dihydrochloride

### EXAMPLE 32A

### (1l-methyl-2-sulfanyl-1H-imidazol-5-yl)methanol

A solution of 1,3-dihydroxyacetone dimer (25 g, 0.28 mol) in n-butanol (115 mL) at room temperature was treated with acetic acid (56 mL), potassium thiocyanate (80.75 g, 0.83 mol) and methylarnine hydrochloride (41.15 g. 0.61 mol), stirred at room temperature for 3 days, treated with a 1:1 mixture of diethyl ether:hexanes (100 mL), washed with water, and concentrated to provide the desired product of sufficient purity for subsequent use without further purification.

¹H NMR (400 MHz, DMSO-d₆) δ 12.0 (s, 1H), 6.81 (s, 1H), 5.21 (t, 1H), 4.32 (d, 2H), 3.45 (s, 3H).

### EXAMPLE 32B

### (1-methyl-1H-imidazol-5-yl)methanol

A solution of Example 32A (50 g, 0.35 mol) in ethanol (500 mL) was treated with Raney® nickel (500 g), heated to reflux for 1 hour, cooled to room temperature, filtered, and concentrated to provide the desired product of sufficient purity for subsequent use without further purification.

MS (DCI/NH₃) m/z 113 (M+H)⁺; ¹H NMR (400 MHz, DMSO-d₆) δ 7.50 (s, 1H), 6.75 (s, 1H), 5.01 (s, 1H), 4.41 (s, 2H), 3.59 (s, 3H).

### EXAMPLE 32C

### 1-methyl-1H-imidazole-5-carbaldehyde

A solution of Example 32B (2.3 g, 20 mmol) in dioxane (100 mL), was treated with manganese dioxide (17.3 g, 200 mmol), heated to reflux for 16 hours, cooled to room temperature, filtered through a pad of diatomaceous earth (Celite®), and concentrated to provide the desired product of sufficient purity for subsequent use without further purification.

¹H NMR (300 MHz, CDCl₃) δ 9.77 (d, 1H), 7.79 (s, 1H), 7.62 (s, 1H), 3.95 (d, 3H).

### EXAMPLE 32D

### 5-((benzylamino)methyl)-2′-methyl(1,1′-biphenyl)-2-carbonitrile

The desired product was prepared by substituting Example 86I and benzylamine for 4-nitrobenzaldehyde and Example 12A, respectively, in Example 12B.

¹H NMR (300 MHz, CDCl₃) δ 7.75-7.68 (m, 2H), 7.47-7.14 (m, 10H), 3.90 (s, 2H), 3.83 (s, 2H), 2.19 (s, 3H), 1.65, (s, 1H).

### EXAMPLE 32E

### 5-((benzyl((1-methyl-1H-imidazol-5-yl)methyl)amino)methyl)-2′-methyl(1,1′-biphenyl)-2-carbonitrile dihydrochloride

The desired product was prepared by substituting Example 32C and Example 32D for nitrobenzaldehyde and Example 12A, respectively, in Example 12B.

MS (ESI(+)) m/z 407 (M+H)⁺; ¹H NMR (300 MHz, CDCl₃) δ 7.69 (d, 1H), 7.40-7.27 (m, 11H), 7.18 (d, 1H), 6.97 (s, 1H), 3.63 (s, 2H), 3.57 (s, 2H), 3.54 (s, 2H), 3.47 (s, 3H), 2.18 (s, 3H).

### EXAMPLE 33

### 4-(((3-bromo-4-cyanobenzyl)amino)(1-methyl-1H-imidazol-5-yl)methyl)-2-(1-naphthyl)benzonitrile dihydrochloride

### EXAMPLE 33A

### 4-amino-3-bromobenzaldehyde

A solution of 4-aminobenzaldehyde (3.0 g, 25 mmol) in methanol (50 mL), acetone (100 mL), and water (30 mL) was treated with p-toluenesulfonic acid monohydrate (1.0 g, 5.26 mmol), heated to reflux for 8 hours, cooled to 0° C., treated with N-bromosuccinimide (4.4 g, 25 mmol), stirred for 30 minutes, and concentrated. The concentrate was treated with ethyl acetate, washed sequentially with saturated Na₂CO₃, water, and brine, dried (Na₂SO₄), filtered, and concentrated to provide the desired product of sufficient purity for subsequent use without further purification.

¹H NMR (300 MHz, CDCl₃) δ 9.72 (s, 1H), 7.95 (d, 1H), 7.64 (dd, 1H), 6.80 (d, 1H), 4.72 (s, 2H).

### EXAMPLE 33B

### 2-bromo-4-formylbenzonitrile

A solution of Example 33A (1.0 g, 5 mmol) in acetone (5 mL) at 0° C. was added to 4.5M HCl (8 mL). The mixture was treated with 40% sodium nitrite (1 mL), warmed to room temperature, and stirred for 1 hour. The mixture was added to a 0° C. solution of copper(I) cyanide (0.45 g, 5 mmol) and potassium cyanide (0.65 g, 10 mmol) in water (20 mL) and toluene (50 mL), warmed to room temperature, stirred for 16 hours, and concentrated. The concentrate was extracted with ethyl acetate, washed sequentially with saturated Na₂CO₃, water, and brine, dried (Na₂SO₄), filtered, and concentrated to provide the desired product of sufficient purity for subsequent use without further purification.

¹H NMR (300 MHz, CDCl₃) δ 10.04 (s, 1H), 8.18 (d, 1H), 7.93 (dd, 1H), 7.85 (d, 1H).

### EXAMPLE 33C

### 4-(((3-bromo-4-cyanobenzyl)amino)(1-methyl-1H-imidazol-5-yl)methyl)-2-(1-naphthyl)benzonitrile dihydrochloride

The desired product was prepared by substituting Example 33B and Example 13A for 4-nitrobenzaldehyde and Example 12A, respectively, in Example 12B. The concentrate was purified by flash column chromatography on silica gel with dichloromethane then 98:2/dichloromethane:methanol. The concentrate was dissolved in dichloromethane, treated with 1M HCl in diethyl ether, and concentrated to provide the desired product.

MS (ESI(+)) m/z 534 (M+2H)⁺; ¹H NMR (300 MHz, CDCl₃) δ 7.97 (t, 2H), 7.87 (d, 1H), 7.67 (s, 1H), 7.61-7.43 (m, 9H), 7.36 (d, 1H), 6.91 (d, 1H), 4.98 (s, 1H), 3.91-3.82 (m, 2H), 3.56 (d, 3H).

### EXAMPLE 34

### 4-(((4-cyanobenzyl)((1-methyl-1H-imidazol-5-yl)methyl)amino)methyl)-2-(1-naphthyl)benzonitrile

### EXAMPLE 34A

### 4-(aminomethyl)benzonitrile

A solution of 4-(bromomethyl)benzonitrile (27.5 g, 0.14 mol) in DMF (125 mL) was treated with potassium phthalimide (27.8 g, 0.15 mol), heated to 130° C. for 2.5 hours, cooled to room temperature, poured over ice, filtered, rinsed with water and a 1:1 mixture of hexanes:diethyl ether, and dried for 16 hours in a vacuum oven at 50° C. The compound was treated with ethanol (200 mL) and 35% aqueous hydrazine (8 mL), heated to reflux for 3 hours, cooled to room temperature, filtered, and concentrated. The concentrate was purified by vacuum distillation to provide the desired product.

¹H NMR (300 MHz, CDCl₃) δ 7.63 (d, 2H), 7.45 (d, 2H), 3.96 (s, 2H), 1.48 (s, 2H).

### EXAMPLE 34B

### 4-(((4-cyanobenzyl)amino)methyl)-2-(1-naphthyl)benzonitrile

The desired product was prepared by substituting Example 89C and Example 34A for 4-nitrobenzaldehyde and Example 12A, respectively, in Example 12B.

MS (ESI(+)) m/z 374 (M+H)⁺.

### EXAMPLE 34C

### 4-(((4-cyanobenzyl)((1-methyl-1H-imidazol-5-yl)methyl)amino)methyl)-2-(1-naphthyl)benzonitrile

The desired product was prepared by substituting Example 32C and Example 34B for 4-nitrobenzaldehyde and Example 12A, respectively, in Example 12B.

¹H NMR (300 MHz, CDCl₃) δ 7.92-7.88 (m, 2H), 7.74-7.71 (m, 1H), 7.54-7.29 (m, 12H), 6.90 (s, 1H), 3.68-3.39 (m, 6H), 3.37 (s, 3H).

### EXAMPLE 35

### 4-(((3-chloro4-cyanobenzyl)amino)(1-methyl-1H-imidazol-5-yl)methyl)-2-(1-naphthyl)benzonitrile dihydrochloride

### EXAMPLE 35A

### 2-chloro-4-iodobenzonitrile

The desired product was prepared by substituting 4-amino-2-chlorobenzonitrile for 5-aminoquinoline in Example 43A.

¹H NMR (300 MHz, CDCl₃) δ 7.92 (d, 1H), 7.74 (dd, 1H), 7.36 (d, 1H).

### EXAMPLE 35B

### methyl 3-chloro-4-cyanobenzoate

A solution of Example 35A (39.9 g, 0.15 mol) in methanol (1 L) was treated with (1,1′-bis(diphenylphosphino)ferrocene)dichloropalladium(II) complex with dichloromethane (1:1) (1.25 g, 1.53 mmol) and triethylamine (24 mL), heated to 97° C. under 500 psi CO pressure for 20 hours, cooled to room temperature, filtered, and concentrated to provide the desired product of sufficient purity for subsequent use without further purification.

¹H NMR (300 MHz, CDCl₃) δ 8.17 (d, 1H), 8.02 (dd, 1H), 7.77 (d, 1H), 3.97 (s, 3H).

### EXAMPLE 35C

### 2-chloro-4-(hydroxymethyl)benzonitrile

The desired product was prepared by substituting Example 35B for Example 5A in Example 5B.

### EXAMPLE 35D

### 2-chloro-4-formylbenzonitrile

A solution of Example 35C (5.49 g, 32.76 mmol) in dichloromethane (100 mL) at room temperature was treated with Dess-Martin periodinane (25 g, 58.9 mmol), stirred for 20 minutes, treated with saturated NaHCO₃ and saturated Na₂S₂O₃, stirred for 5 minutes, concentrated, and extracted with diethyl ether. The extracts were washed with brine, dried (Na₂SO₄), filtered, and concentrated to provide the desired product.

MS (DCI(+)) m/z 183 (M+NH₄)⁺; ¹H NMR (300 MHz, CDCl₃) δ 10.06 (s, 1H), 8.03-8.02 (m, 1H), 7.88 (d, 2H).

### EXAMPLE 35E

### 4-(((3-chloro-4-cyanobenzyl)amino)(1-methyl-1H-imidazol-5-yl)methyl)-2-(1-naphthyl)benzonitrile dihydrochloride

The desired product was prepared by substituting Example 35D and Example 13A for 4-nitrobenzaldehyde and Example 12A, respectively, in Example 12B. The purified concentrate was dissolved in dichloromethane, treated with 1M HCl in diethyl ether, and concentrated to provide the desired product.

MS (ESI(+)) m/z 488 (M+H)⁺; ¹H NMR (400 MHz, CDCl₃) δ 7.96 (t, 2H), 7.86 (d, 1H) 7.62-7.42 (m, 10H), 7.31 (d, 1H), 6.91 (d, 1H), 4.98 (s, 1H), 3.90 -3.81 (m, 2H), 3.55 (d, 3H).

### EXAMPLE 36

### 4-(((1-(4-cyanophenyl)ethyl)amino)(1-methyl-1H-imidazol-5-yl)methyl)-2-(1-naphthyl)benzonitrile dihydrochloride

The desired product was prepared by substituting 4-acetylbenzonitrile and Example 13A for 4-nitrobenzaldehyde and Example 12A, respectively, in Example 12B. The purified concentrate was dissolved in dichloromethane, treated with 1M HCl in diethyl ether, and concentrated to provide the desired product.

MS (ESI(+)) m/z 468 (M+H)⁺; ¹H NMR (300 MHz, CDCl₃) δ 7.98-7.35 (m, 15H), 7.00-6.72 (m, 1H), 4.81-4.58 (m, 1H), 3.93-3.70 (m, 1H), 3.56-3.47 (m, 3H), 1.41-1.36 (m, 3H).

### EXAMPLE 37

### 4-(((4-cyano-3-iodobenzyl)amino)(1-methyl-1H-imidazol-5-yl)methyl)-2-(1-naphthyl)benzonitrile dihydrochloride

### EXAMPLE 37A

### 4-(hydroxymethyl)-2-iodobenzonitrile

A suspension of Example 63A (296 mg, 1.0 mmol) in water (10 mL) was treated with diatomaceous earth (Celite®) (296 mg), heated to reflux for 2 hours, cooled to room temperature, and filtered. The filtrate was extracted with ethyl acetate, dried (MgSO₄), filtered, and concentrated to provide the desired product of sufficient purity for subsequent use without further purification.

MS (DCI/NH₃) m/z 277 (M+NH₄)⁺; ¹H NMR (300 MHz, CDCl₃) δ 7.96 (s, 1H), 7.60 (d, 1H), 7.44 (dt, 1H), 4.75 (d, 2H), 1.86 (t, 1H).

### EXAMPLE 37B

### 4-formyl-2-iodobenzonitrile

A solution of Example 37A (70 mg, 0.27 mmol) in DMSO (2 mL) and triethylamine (190 μL, 1.35 mmol) at room temperature was treated with small portions of pyridine sulfur trioxide (107 mg, 0.68 mmol), stirred for 16 hours, treated with ethyl acetate, washed sequentially with 1M HCl, water, and brine, dried (Na₂SO₄), and filtered. The filtrate was treated with activated charcoal, stirred for 45 minutes, filtered through a pad of diatomaceous earth (Celite®) with 9:1/dichloromethane:methanol, and concentrated to provide the desired product of sufficient purity for subsequent use without further purification.

MS (DCI/NH₃) m/z 257 (M)⁺; ¹H NMR (300 MHz, CDCl₃) δ 10.01 (s, 1H), 8.41 (s, 1H), 7.96 (d, 1H), 7.80 (d, 1H).

### EXAMPLE 37C

### 4-(((4-cyano-3-iodobenzyl)amino)(1-methyl-1H-imidazol-5-yl)methyl)-2-(1-naphthyl)benzonitrile dihydrochloride

A solution of 13A (32 mg, 0.09 mmol) and molecular sieves (100 mg) in 1,2-dichloroethane (2 mL) at room temperature was treated with Example 37B (34 mg, 0.57 mmol) and acetic acid, stirred for 30 minutes, treated with sodium triacetoxyborohydride (60 mg, 0.28 mmol), and stirred for 16 hours. The mixture was treated with ethyl acetate, washed sequentially with saturated NaHCO₃, water, and brine, dried (MgSO₄), filtered, and concentrated. The concentrate was dissolved in dichloromethane (5 mL), treated with 4M HCl in dioxane (1 mL), stirred for 30 minutes, and concentrated. The concentrate was dissolved in ethyl acetate, washed sequentially with saturated NaHCO₃, water, and brine, dried (MgSO₄), filtered, and concentrated. The concentrate was purified by flash column chromatography on silica gel with dichloromethane then 98:2/dichloromethane:methanol. The concentrate was dissolved in dichloromethane, treated with 1M HCl in diethyl ether, and concentrated to provide the desired product.

MS (ESI(+)) m/z 580 (M+H)⁺; ¹H NMR (400 MHz, CDCl₃) δ 7.87 (t, 2H), 7.81-7.76 (m, 2H) 7.52-7.29 (m, 10H), 6.81 (d, 1H), 4.87 (s, 1H), 3.78-3.69 (m, 2H), 3.47 (d, 3H), 1.97 (s, 1H).

### EXAMPLE 38

### methyl 4-((((4-cyano-3-(1-naphthyl)phenyl)(1-methyl-1H-imidazol-5-yl)methyl)amino)methyl)benzoate

The desired product was prepared by substituting methyl 4-formylbenzoate for Example 37B in Example 37C. The concentrate was purified by flash column chromatography on silica gel with dichloromethane then 98:2/dichloromethane:methanol to provide the desired product.

MS (ESI(+)) m/z 487 (M+H)⁺; ¹H NMR (300 MHz, CDCl₃) δ 8.01-7.89 (m, 5H), 7.62-7.32 (m, 10H), 7.10-7.00 (m, 1H), 5.00-4.93 (m, 1H), 3.92 (s, 3H), 3.90-3.64 (m, 2H), 3.75-3.60 (m, 3H).

### EXAMPLE 39

### lithium 4-((((4-cyano-3-(1-naphthyl)phenyl)(1-methyl-1H-imidazol-5-yl)methyl)amino)methyl)benzoate

A solution of Example 38 (55 mg, 0.113 mmol) in methanol (2 mL) and water (0.5 mL) at room temperature was treated with lithium hydroxide monohydrate (4.7 mg, 0.112 mmol), stirred for 16 hours, treated with a second portion of lithium hydroxide monohydrate (2.4 mg, 0.057 mmol), stirred for 8 hours, and concentrated. The concentrate was treated with THF (1 mL) and water (1.0 mL), stirred for 16 hours, treated with a third portion of lithium hydroxide monohydrate (3.0 mg, 0.07 mmol), stirred for 16 hours, and concentrated. The concentrate was dissolved in water (3 mL), washed with diethyl ether, and lyophilized to provide the desired product.

MS (ESI(+)) m/z 473 (M+H)⁺; ¹H NMR (300 MHz, CDCl₃) δ 8.08-8.01 (m, 3H), 7.76-7.42 (m, 10H), 7.16 (d, 2H), 6.53 (d, 1H), 4.98 (s, 1H), 3.72-3.60 (m, 2H), 3.54 (d, 3H).

### EXAMPLE 40

### 4-(((4-chlorobenzyl)((1-methyl-1H-imidazol-5-yl)methyl)amino)methyl)-2-(1-naphthyl)benzonitrile dihydrochloride

### EXAMPLE 40A

### 4-(((4-chlorobenzyl)amino)methyl)-2-(1-naphthyl)benzonitrile

The desired product was prepared by substituting Example 89C and (4-chlorophenyl)methylamine for 4-nitrobenzaldehyde and Example 12A, respectively, in Example 12B.

MS (ESI(+)) m/z 383 (M+H)⁺; ¹H NMR (400 MHz, CDCl₃) δ 7.94 (dd, 2H), 7.79 (d, 1H), 7.58-7.42 (m, 8H), 7.30-7.24 (m, 3H), 3.91 (s, 2H), 3.81 (s, 2H).

### EXAMPLE 40B

### 4-(((4-chlorobenzyl)((1-methyl-1H-imidazol-5-yl)methyl)amino)methyl)-2-(1-naphthyl)benzonitrile dihydrochloride

The desired product was prepared by substituting Example 32C and Example 40A for 4-nitrobenzaldehyde and Example 12A, respectively, in Example 12B. The purified concentrate was dissolved in dichloromethane, treated with 1M HCl in diethyl ether, and concentrated to provide the desired product.

MS (ESI(+)) m/z 479 (M+2H)⁺; ¹H NMR (300 MHz, CDCl₃) δ 7.96 (dd, 2H), 7.80-7.77 (m, 1H), 7.58-7.19 (m, 12H), 7.10-7.00 (m, 1H), 3.70-3.48 (m, 6H), 1.90 (m, 3H).

### EXAMPLE 41

### 4-(((( 1-methyl-1H-imidazol-5-yl)methyl)(4-(trifluoromethoxy)benzyl)amino)methyl)-2-(1-naphthyl)benzonitrile dihydrochloride

### EXAMPLE 41A

### 4-(((4-trifluoromethoxybenzyl)amino)methyl)-2-(1-naphthyl)benzonitrile

The desired product was prepared by substituting Example 89C and 4-trifluoromethoxybenzylamine for 4-nitrobenzaldehyde and Example 12A, respectively, in Example 12B.

MS (ESI(+)) m/z 433 (M+H)⁺; ¹H NMR (400 MHz, CDCl₃) δ 7.94 (dd, 2H), 7.82-7.78 (m, 1H), 7.58-7.34 (m, 9H), 7.16 (d, 2H), 3.93 (s, 2H), 3.84 (s, 2H).

### EXAMPLE 41B

### 4-((((1-methyl-1H-imidazol-5-yl)methyl)(4-(trifluoromethoxy)benzyl)amino)methyl)-2-(1-naplhthyl)benzonitrile dihydrochloride

The desired product was prepared by substituting Example 32C and Example 41A for 4-nitrobenzaldehyde and Example 12A, respectively, in Example 12B. The purified concentrate was dissolved in dichloromethane, treated with 1M HCl in diethyl ether, and concentrated to provide the desired product.

MS (ESI(+)) m/z 527 (M+H)⁺; ¹H NMR (400 MHz, CDCl₃) δ 7.97-7.94 (m, 2H), 7.77 (d, 1H), 7.60-7.28 (m, 10H), 7.14 (d, 2H), 6.96 (s, 1H), 3.74-3.49 (m, 6H), 3.43 (s, 3H).

### EXAMPLE 42

### 4-(((4-chlorobenzyl)amino)(1-methyl-1H-imidazol-5-yl)methyl)-2-(1-naphthyl)benzonitrile

The desired product was prepared by substituting 4-chlorobenzaldehyde and Example 13A for 4-nitrobenzaldehyde and Example 12A, respectively, in Example 12B.

MS (ESI(+)) m/z 463 (M+H)⁺; ¹H NMR (400 MHz, CDCl₃) δ 7.97-7.92 (m, 2H), 7.84 (d, 1H), 7.60-7.39 (m, 8H), 7.29-7.20 (m, 4H), 6.85 (d, 1H), 4.94 (d, 1H), 3.82-3.70 (m, 2H), 3.43 (d, 3H).

### EXAMPLE 43

### 4-(((4-cyanobenzyl)oxy)(1-methyl-1H-imidazol-5-yl)methyl)-2-(5-quinolinyl)benzonitrile dihydrochloride

### EXAMPLE 43A

### 5-iodoquinoline

A solution of 5-aminoquinoline (5.5 g, 38.1 mmol) in 3M HCl (100 mL) at 0° C. was treated dropwise with a solution of sodium nitrite (3.65 g, 52.9 mmol) in water (25 mL), then with a solution of potassium iodide (13.0 g, 78.3 mmol) in water (25 mL) with periodic treatment with acetone to prevent foaming. The reaction was warmed to room temperature, stirred for 16 hours, treated with saturated sodium thiosulfate, and extracted with ethyl acetate. The extract was dried (Na₂SO₄), filtered, and concentrated. The concentrate was purified by flash column chromatography on silica gel with 8:1 to 4:1/hexanes:ethyl acetate to provide the desired product.

¹H NMR (400 MHz, CDCl₃) δ 8.89 (d, 1H), 8.39 (d, 1H), 8.14-8.10 (m, 2H), 7.51-7.41 (m, 2H).

### EXAMPLE 43B

### 5-quinolinylboronic acid

A solution of 1.6M n-butyllithium in diethyl ether (15.6 mL, 25 mmol) in diethyl ether (40 mL) at −78° C. was treated with a solution of Example 43A (2.55 g, 10 mmol) in diethyl ether (30 mL), stirred for 40 minutes, treated with a solution of tributyl borate (6.9 g, 17.4 mmol) in diethyl ether (10 mL), warmed to room temperature, and stirred for 16 hours. The mixture was cooled to 0° C., and adjusted to pH 2 with 1M HCl. The aqueous layer was cooled to 0° C., adjusted to pH 7 with saturated NaHCO₃, and the resulting precipitate was filtered and dried to provide the desired product of sufficient purity for subsequent use without further purification.

¹H NMR (300 MHz, DMSO-d₆) δ 8.88-8.82 (m, 1H), 8.46 (s, 1H), 8.04-8.00 (dd, 1H), 7.88 (dd, 1H), 7.72 (dd, 1H), 7.51 (dd, 1H).

### EXAMPLE 43C

### 4-(((4-cyanobenzyl)oxy)(1-methyl-1H-imidazol-5-yl)methyl)-2-(5-quinolinyl)benzonitrile dihydrochloride

A solution of Example 60C (45 mg, 0.1 mmol) in toluene (1 mL) and ethanol (1 mL) was treated with Example 43B (35 mg, 0.2 mmol), 2M Na₂CO₃ (0.15 mL, 0.3 mmol), lithium chloride (13 mg, 0.3 mmol), and Pd(PPh₃)₄ (5.8 mg, 0.005 mmol), heated to reflux for 16 hours, and cooled to room temperature. The mixture was treated with ethyl acetate, washed with water and brine, dried (Na₂SO₄), filtered, and concentrated. The concentrate was purified by flash column chromatography on silica gel with dichloromethane then 99:1 to 90:10/dichloromethane/methanol. The concentrate was dissolved in dichloromethane, treated with 1M HCl in diethyl ether, and concentrated to provide the desired product.

MS (ESI(+)) m/z 456 (M+H)⁺; ¹H NMR (400 MHz, CDCl₃) δ 8.98-8.97 (m, 1H), 8.24 (d, 1H), 7.90-7.78 (m, 3H), 7.66-7.40 (m, 9H), 6.98-6.97 (m, 1H), 5.70-5.69 (m, 1H), 4.69-4.58 (m, 2H), 3.49-3.44 (m, 3H).

### EXAMPLE 44

### 4-(((4-cyanobenzyl)oxy)(1-methyl-1H-imidazol-5-yl)methyl)-2-(5-isoquinolinyl)benzonitrile

### EXAMPLE 44A

### 5-iodoisoquinoline

The desired product was prepared by substituting 5-aminoisoquinoline for 5-aminoquinoline in Example 43A.

¹H NMR (300 MHz, CDCl₃) δ 9.15 (s, 1H), 8.64 (d, 1H), 8.28 (d, 1H), 7.99 (d, 1H), 7.85 (d, 1H), 7.37 (t, 1H).

### EXAMPLE 44B

### 5-isoquinolinylboronic acid

The desired product was prepared by substituting Example 44A for Example 43A in Example 43B.

### EXAMPLE 44C

### 4-(((4-cyanobenzyl)oxy)(1-methyl-1H-imidazol-5-yl)methyl)-2-(5-isoquinolinyl)benzonitrile

The desired product was prepared by substituting Example 44B for Example 43B in Example 43C. The concentrate was purified by flash column chromatography on silica gel with dichloromethane then 99:1 to 90:10/dichloromethane:methanol to provide the desired product.

MS (ESI(+)) m/z 456 (M+H)⁺; ¹H NMR (400 MHz, CDCl₃) δ 9.36 (s, 1H), 8.50 (dd, 1H), 8.12-8.08 (m, 1H), 7.89 (d, 1H), 7.73-7.41 (m, 9H), 7.27 (dd, 1H), 6.98 (d, 1H), 5.70 (d, 1H), 5.69-4.59 (m, 2H), 3.47 (s, 3H).

### EXAMPLE 45

### 4-(((4-cyanobenzyl)(1H-imidazol-5-ylmethyl)amino)methyl)-2-(1-naphthyl)benzonitrile dihydrochloride

A solution of Example 34B (25 mg, 0.067 mmol) in 1,2-dichloroethane (1 mL) at room temperature was treated with 1H-imidazole-5-carbaldehyde (9.6 mg, 0.1 mmol) and acetic acid (2 mL, 35 mmol), stirred for 30 minutes, treated with sodium triacetoxyborohydride (140 mg, 0.66 mmol), stirred for 72 hours, treated with additional 1H-imidazole-5-carbaldehyde (20 mg, 0.21 mmol), and stirred for 2 days. The mixture was treated with ethyl acetate, washed sequentially with saturated NaHCO₃, water, and brine, dried (MgSO₄), filtered, and concentrated. The concentrate was dissolved in dichloromethane (5 mL), treated with 4M HCl in dioxane (1 mL), stirred for 30 minutes, and concentrated. The concentrate was dissolved in ethyl acetate, washed sequentially with saturated NaHCO₃, water, and brine, dried (MgSO₄), filtered, and concentrated. The concentrate was purified by flash column chromatography on silica gel with dichloromethane then 98:2 to 95:5/dichloromethane/methanol. The concentrate was dissolved in dichloromethane, treated with 1M HCl in diethyl ether, and concentrated to provide the desired product.

MS (ESI(+)) m/z 454 (M+H)⁺; ¹H NMR (400 MHz, CDCl₃) δ 7.92 (dd, 2H), 7.75 (d, 1H), 7.64 (s, 1H), 7.57-7.32 (m, 12H), 6.89 (s, 1H), 3.79-3.61 (m, 6H).

### EXAMPLE 46

### 4-(((4-cyanobenzyl)oxy)(1H-imidazol-5-yl)methyl)-2-(1-naphthyl)benzonitrile hydrochloride

A solution of Example 49C (50 mg, 0.07 mmol) in 80% aqueous acetic acid (5 mL), at room temperature was stirred for 16 hours and concentrated. The concentrate was purified by flash column chromatography on silica gel with 9:1/dichloromethane:methanol to provide the desired product.

MS (ESI(+)) m/z 441 (M+H)⁺; ¹H NMR (500 MHz, CDCl₃) δ 7.94-7.91 (m, 2H), 7.82 (dd, 1H), 7.68-7.36 (m, 13H), 6.93 (d, 1H), 5.66 (d, 1H), 4.68 (abq, 2H).

### EXAMPLE 47

### 4-(((3,4-dichlorobenzyl)amino)(1-methyl-1H-imidazol-5-yl)methyl)-2-(1-naphthyl)benzonitrile

The desired product was prepared by substituting 3,4-dichlorobenzaldehyde and Example 13A for 4-nitrobenzaldehyde and Example 12A, respectively, in Example 12B.

MS (ESI(+)) m/z 497 (M)⁺; ¹H NMR (300 MHz, CDCl₃) δ 7.98-7.93 (m, 2H), 7.86 (d, 1H), 7.61-7.36 (m, 10H), 7.12 (d, 1H), 6.87 (s, 1H), 4.94 (s, 1H), 3.82-3.69 (m, 2H), 3.56 (d, 3H).

### EXAMPLE 48

### 4-((((4-cyano-3-(1-naphthyl)phenyl)(1-methyl-1H-imidazol-5-yl)methyl)amino)methyl)-N-methylbenzamide dihydrochloride

A solution of Example 39 (20 mg, 0.04 mmol) in DMF (1 mL) at room temperature was treated with 1-(3-dimethylaminopropyl)-3-ethylcarbodiimide hydrochloride (12 mg, 0.06 mmol), 1-hydroxybenzotriazole (8.5 mg, 0.06 mmol), methylamine hydrochloride (28.4 mg, 0.42 mmol), and 4-methylmorpholine (46 μL, 0.42 mmol), stirred for 16 hours, treated with ethyl acetate, washed sequentially with saturated NaHCO₃, water, and brine, dried (MgSO₄), filtered and concentrated. The concentrate was purified by flash column chromatography on silica gel with dichloromethane then 98:2 to 95:5/dichloromethane:methanol. The concentrate was dissolved in dichloromethane, treated with 1M HCl in diethyl ether, and concentrated to provide the desired product.

MS (ESI(+)) m/z 486 (M+H)⁺; ¹H NMR (400 MHz, CDCl₃) δ 7.96-7.92 (m, 2H), 7.83 (d, 1H), 7.71 (d, 2H), 7.59-7.33 (m, 10H), 6.86 (d, 1H), 6.14 (m, 1H), 4.94 (d, 1H), 3.89-3.67 (m, 2H), 3.53 (d, 3H), 3.00 (d, 3H).

### EXAMPLE 49

### 4-(((4-cyanobenzyl)oxy)(1-trityl-1H-imidazol-4-yl)methyl)-2-(1-naphthyl)benzonitrile

### EXAMPLE 49A

### 4-iodo-1-trityl-1H-imidazole

A suspension of 4-iodoimidazole (3.38 g, 17.4 mmol) and triphenylmethyl chloride (5.56 g, 19.9 mmol) in DMF (15 mL) at 0° C. was treated with triethylamine (1.5 mL, 10.8 mmol), warmed to room temperature, stirred for 16 hours, poured into ice water, filtered, and dried in a vacuum oven at 50° C. to provide the desired product of sufficient purity for subsequent use without further purification.

### EXAMPLE 49B

### 4-(hydroxy(1-trityl-1H-imidazol-4-yl)methyl)-2-(1-naphthyl)benzonitrile

A solution of Example 49A (873 mg, 2.0 mmol) in dichloromethane (8 mL) at room temperature was treated with 3M ethyl magnesium bromide in diethyl ether (0.73 mL, 2.2 mmol), stirred for 30 minutes, and cooled to −20° C. The mixture was treated with a solution of Example 89C (514 mg, 2 mmol) in dichloromethane (2 mL), warmed to room temperature, stirred for 16 hours, treated with saturated ammonium chloride, and concentrated. The concentrate was extracted with ethyl acetate, the extracts were washed with water and brine, dried (MgSO₄), filtered, and concentrated to provide the desired product.

¹H NMR (300 MHz, CDCl₃) δ 7.94 (dd, 2H), 7.79 (dd, 1H), 7.68-7.23 (m, 17H), 7.08-7.05 (m, 6H), 6.64-6.62 (m, 1H), 5.87 (d, 1H).

### EXAMPLE 49C

### 4-(((4-cyanobenzyl)oxy)(1-trityl-1H-imidazol-4-yl)methyl)-2-(1-naphthyl)benzonitrile

A solution of Example 49B (113 mg, 0.2 mmol) in dichloromethane (1 mL) at room temperature was treated with 4-(bromomethyl)benzonitrile (50 mg, 0.25 mmol) and silver (I) oxide (140 mg, 0.6 mmol), and stirred for 72 hours. The mixture was purified by flash column chromatography on silica gel with 6:1 to 4:1/hexanes:ethyl acetate to provide the desired product.

MS (ESI(+)) m/z 382 (M)⁺; ¹H NMR (400 MHz, CDCl₃) δ 7.94 (dd, 2H), 7.80 (dd, 1H), 7.65 (dq, 1H), 7.58-7.23 (m, 20H), 7.09-7.06 (m, 6H), 6.73 (dd, 1H), 5.56 (s, 1H), 4.69-4.60 (m, 2H).

### EXAMPLE 50

### ethyl 4-(((4-cyano-3-(1-naphthyl)phenyl)(1-methyl-1H-imidazol-5-yl)methyl)amino)-1-piperidinecarboxylate dihydrochloride

The desired product was prepared by substituting ethyl 4-oxo-1-piperidinecarboxylate and Example 13A for 4-nitrobenzaldehyde and Example 12A, respectively, in Example 12B. The purified concentrate was dissolved in dichloromethane, treated with 1M HCl in diethyl ether, and concentrated to provide the desired product.

MS (ESI(+)) m/z 494 (M+H)⁺; ¹H NMR (300 MHz, CDCl₃) δ 7.96 (dd, 2H), 7.85 (d, 1H), 7.61-7.42 (m, 8H), 6.74 (d, 1H), 5.15 (s, 1H), 4.16-4.00 (m, 2H), 4.11 (q, 2H), 3.62 (d, 3H), 2.87-2.78 (m, 2H), 2.70-2.61 (m, 1H), 1.97-1.73 (m, 2H), 1.40-1.20 (m, 5H).

### EXAMPLE 51

### 6-((((4-cyano-3-(1-naphthyl)phenyl)(1-methyl-1H-imidazol-5-yl)methyl)amino)methyl)nicotinonitrile trihydrochloride

### EXAMPLE 51A

### 6-formylnicotinonitrile

A solution of 6-methylnicotinonitrile (590 mg, 5.0 mmol) in dioxane (10 mL) and water (0.5 mL) was treated with selenium dioxide (555 mg, 5.0 mmol), heated to reflux for 16 hours, cooled to room temperature, and concentrated. The concentrate was purified by flash column chromatography on silica gel with hexanes then 9:1/hexanes:ethyl acetate to provide the desired product.

¹H NMR (300 MHz, CDCl₃) δ 10.13-10.12 (m, 1H), 9.06-9.05 (m, 1H), 8.19-8.16 (m, 1H), 8.09-8.05 (m, 1H).

### EXAMPLE 51B

### 6-((((4-cyano-3-(1-naphthyl)phenyl)(1-methyl-1H-imidazol-5-yl)methyl)amino)methyl)nicotinonitrile trihydrochloride

The desired product was prepared by substituting Example 51A and Example 13A for 4-nitrobenzaldehyde and Example 12A, respectively, in Example 12B. The concentrate was purified by flash column chromatography on silica gel with dichloromethane then 99:1 to 98:2/dichloromethane:methanol. The purified concentrate was dissolved in dichloromethane, treated with 1M HCl in diethyl ether, and concentrated to provide the desired product.

MS (ESI(+)) m/z 455 (M+H)⁺; ¹H NMR (300 MHz, CDCl₃) δ 8.80 (s, 1H), 7.97-7.82 (m, 4H), 7.59-7.36 (m, 9H), 6.94 (s, 1H), 5.07 (s, 1H), 3.99 (s, 2H), 3.56 (d, 3H), 2.67 (s, 1H).

### EXAMPLE 52

### methyl 6-((((4-cyano-3-(1-naphthyl)phenyl)(1-methyl-1H-imidazol-5-yl)methyl)amino)methyl)nicotinate trihydrochloride

### EXAMPLE 52A

### methyl 6-(hydroxymethyl)nicotinate

The desired product was prepared by substituting dimethyl 2,5-pyridinedicarboxylate for Example 5A in Example 5B.

### EXAMPLE 52B

### methyl 6-formylnicotinate

The desired product was prepared by substituting Example 52A for Example 37A in Example 37B.

### EXAMPLE 52C

### methyl 6-((((4-cyano-3-(1-naphthyl)phenyl)(1-methyl-1H-imidazol-5-yl)methyl)amino)methyl)nicotinate trihydrochloride

The desired product was prepared by substituting Example 52B and Example 13A for 4-nitrobenzaldehyde and Example 12A, respectively, in Example 12B. The purified concentrate was dissolved in dichloromethane, treated with 1M HCl in diethyl ether, and concentrated to provide the desired product.

MS (ESI(+)) m/z 488 (M+H)⁺; ¹H NMR (300 MHz, CDCl₃) δ 9.14 (s, 1H), 8.23 (dd, 1H), 7.97-7.92 (m, 2H), 7.83 (d, 1H), 7.61-7.40 (m, 7H), 7.32-7.24 (m, 2H), 6.93 (s, 1H), 5.05 (s, 1H), 3.98-3.92 (m, 5H), 3.56 (s, 3H), 2.71 (s, 1H).

### EXAMPLE 53

### N-(4-((((4-cyano-3-(1-naphthyl)phenyl)(1-methyl-1H-imidazol-5-yl)methyl)amino)methyl)phenyl)acetamide dihydrochloride

The desired product was prepared by substituting N-(4-formylphenyl)acetamide and Example 13A for 4-nitrobenzaldehyde and Example 12A, respectively, in Example 12B. The purified concentrate was dissolved in dichloromethane, treated with 1M HCl in diethyl ether, and concentrated to provide the desired product.

MS (ESI(+)) m/z 486 (M+H)⁺; ¹H NMR (400 MHz, CDCl₃) δ 7.93 (dd, 2H), 7.82 (d, 1H), 7.58-7.38 (m, 10H), 7.21 (d, 2H), 6.84 (d, 1H), 4.94 (s, 1H), 3.79-3.67 (m, 2H), 3.52 (d, 3H), 2.13 (s, 3H).

### EXAMPLE 54

### benzyl 4-(((4-cyano-3-(1-naphthyl)phenyl)(1-methyl-1H-imidazol-5-yl)methyl)amino)-1-piperidinecarboxylate dihydrochloride

The desired product was prepared by substituting benzyl-4-oxo-1-piperidinecarboxylate and Example 13A for 4-nitrobenzaldehyde and Example 12A, respectively, in Example 12B. The concentrate was dissolved in dichloromethane, treated with 1M HCl in diethyl ether, and concentrated to provide the desired product.

MS (ESI(+)) m/z 556 (M+H)⁺; ¹H NMR (400 MHz, CDCl₃) δ 7.94 (dd, 2H), 7.83 (d, 1H), 7.59-7.28 (m, 13H), 6.74 (d, 1H), 5.13-5.11 (m, 3H), 4.07 (m, 2H), 3.60 (d, 3H), 2.90-2.83 (m, 2H), 2.69-2.61 (m, 1H), 1.95-1.72 (m, 2H), 1.36-1.30 (m, 2H).

### EXAMPLE 55

### 4-(((1-benzyl-4-piperidinyl)amino)(1-methyl-1H-imidazol-5-yl)methyl)-2-(1-naphthyl)benzonitrile trihydrochloride

The desired product was prepared by substituting 1-benzyl-4-piperidinone and Example 13A for 4-nitrobenzaldehyde and Example 12A, respectively, in Example 12B. The concentrate was dissolved in dichloromethane, treated with 1M HCl in diethyl ether, and concentrated to provide the desired product.

MS (ESI(+)) m/z 512 (M+H)⁺; ¹H NMR (400 MHz, CDCl₃) δ 7.96-7.91 (m, 2H), 7.82 (d, 1H), 7.59-7.22 (m, 13H), 6.71 (d, 1H), 5.12 (s, 1H), 3.60 (d, 3H), 3.51-3.45 (m, 2H), 2.82-2.74 (m, 2H), 2.52-2.43 (m, 1H), 2.05-1.43 (m, 6H).

### EXAMPLE 56

### tert-butyl 4-(((4-cyano-3-(1 -naphthyl)phenyl)(1-methyl-1H-imidazol-5-yl)methyl)amino)-1-piperidinecarboxylate

A solution of Example 13A (20 mg, 0.06 mmol) in 1,2-dichloroethane (1 mL) at room temperature was treated with tert-butyl 4-oxo-1-piperidinecarboxylate (11.8 mg, 0.06 mmol) and acetic acid (21 mg, 0.35 mmol), stirred for 30 minutes, treated with sodium triacetoxyborohydride (37.5 mg, 0.18 mmol), stirred for 16 hours, treated with ethyl acetate, washed sequentially with saturated NaHCO₃, water, and brine, dried (MgSO₄), filtered, and concentrated. The concentrate was dissolved in methanol (3 mL), heated to 60° C. for 1 hour, cooled to room temperature, and concentrated. The concentrate was purified by flash column chromatography on silica gel with dichloromethane then 99:1 to 97:3/dichloromethane:methanol to provide the desired product.

MS (ESI(+)) m/z 522 (M+H)⁺; ¹H NMR (500 MHz, CDCl₃) δ 7.96-7.92 (m, 2H), 7.83 (d, 1H), 7.59-7.39 (m, 8H), 6.71 (d, 1H), 5.14 (s, 1H), 3.99-3.98 (m, 2H), 3.61 (d, 3H), 2.80-2.75 (m, 2H), 2.67-2.60 (m, H1H), 1.88-1.60 (m, 3H), 1.45 (s, 9H), 1.33-1.25 (m, 2H).

### EXAMPLE 57

### 4-(((1-benzoyl4-piperidinyl)amino)(1-methyl-1H-imidazol-5-yl)methyl)-2-(1-naphthyl)benzonitrile dihydrochloride

The desired product was prepared by substituting 1-benzoyl-4-piperidinone for tert-butyl 4-oxo-1-piperidinecarboxylate in Example 56.

MS (ESI(+)) m/z 526 (M+H)⁺; ¹H NMR (300 MHz, CDCl₃) δ 7.98-7.93 (m, 2H), 7.85 (d, 1H), 7.61-7.36 (m, 13H), 6.76 (d, 1H), 5.15 (s, 1H), 4.56 (s, 1H), 3.73-3.59 (m, 1H), 3.62 (d, 3H), 2.95-2.72 (m, 3H), 2.10-0.80 (m, 5H).

### EXAMPLE 58

### 4-((((4-cyano-3-(1-naphthyl)phenyl)(1-methyl-1H-imidazol-5-yl)methyl)amino)methyl)benzamide dihydrochloride

A solution of Example 39 (20 mg, 0.04 mmol) in DMF (0.5 mL) at room temperature was treated sequentially with PyBOP (33 mg, 0.06 mmol), 0.5M ammonia in dioxane (1 mL, 0.5 mmol), and HOBt, stirred for 16 hours, treated with ammonia, stirred for 16 hours, treated with ethyl acetate, washed sequentially with saturated NaHCO₃, water, and brine, dried (MgSO₄), filtered, and concentrated. The concentrate was purified by flash column chromatography on silica gel with dichloromethane then 95:5 to 90:10/dichloromethane:methanol. The concentrate was dissolved in dichloromethane, treated with 1M HCl in diethyl ether, and concentrated to provide the desired product.

MS (ESI(+)) m/z 472 (M+H)⁺; ¹H NMR (500 m/z, CDCl₃), δ 8.39 (d, 1H), 8.00-7.96 (m, 3H), 7.82 (dd, 2H), 7.75-7.72 (m, 1H), 7.68-7.42 (m, 8H), 7.11 (s, 1H), 5.17 (d, 1H), 3.92-3.82 (m, 2H), 3.77 (d, 3H).

### EXAMPLE 59

### 4-((1-methyl-1H-imidazol-5-yl)((4-nitrobenzyl)oxy)methyl)-2-(1,1-naphthyl)benzonitrile hydrochloride

The desired product was prepared by substituting Example 89D and 4-nitrobenzyl bromide for Example 5D and (bromomethyl)benzene, respectively, in Example 5E.

MS (DCI/NH₃) m/z 475 (M+H)⁺; ¹H NMR (300 MHz, DMSO-d₆) δ 9.09 (s, 1H), 8.22-8.02 (m, 6H), 7.79 (m, 1H), 7.69-7.43 (m, 9H), 6.19 (s, 1H), 4.8 (m, 2H), 3.79 (d, 3H).

### EXAMPLE 60

### 4-(((4-cyanobenzyl)oxy)(1-methyl-1H-imidazol-5-yl)methyl)-2-iodobenzonitrile hydrochloride

### EXAMPLE 60A

### 4-(hydroxymethyl)-2iodobenzonitrile

The desired product was prepared by substituting Example 93C for Example 5A in Example 1B.

### EXAMPLE 60B

### 4-formyl-2-iodobenzonitrile

The desired product was prepared by substituting Example 60A for Example 5B in Example 5C.

### EXAMPLE 60C

### 4-(hydroxy( 1-methyl-1H-imidazol-5-yl)methyl)-2-iodobenzonitrile

The desired product was prepared by substituting Example 60B for Example 1A in Example 1B.

¹H NMR (300 MHz, DMSO-d₆) δ 8.04 (s, 1H), 7.85 (d, 1H), 7.58 (m, 2H), 6.39 (s, 1H), 6.22 (d, 1H), 5.88 (d, 1H), 3.55 (s, 3H).

### EXAMPLE 60D

### 4-(((4-cyanobenzyl)oxy)(1-methyl-1H-imidazol-5-yl)methyl)-2-iodobenzonitrile hydrochloride

The desired product was prepared by substituting Example 60C and 4-cyanobenzyl bromide for Example 5D and (bromomethyl)benzene, respectively, in Example 5E.

MS (DCI/NH₃) m/z 455 (M+H)⁺; ¹H NMR (300 MHz, CDCl₃) δ 9.1 (s, 1H), 8.0 (m, 1H), 7.7 (m, 4H), 7.5 (m, 3H), 5.68 (br s, 1H), 4.63 (m, 2H), 3.8 (br s, 3H).

### EXAMPLE 61

### 4-(((3-chloro-4-cyanobenzyl)oxy)(1-methyl-1H-imidazol-5-yl)methyl)-2-(1-naphthyl)benzonitrile hydrochloride

### EXAMPLE 61A

### 2-chloro-4-(hydroxymethyl)benzonitrile

The desired product was prepared by substituting Example 35B for Example 5A in Example 5B.

MS (DCI/NH₃) m/z 185 (M+NH₄)⁺; ¹H NMR (300 MHz, CDCl₃) δ 7.67 (d, 1H), 7.57 (s, 1H), 7.37 (d, 1H), 4.69 (d, 2H), 1.90 (t, 1H).

### EXAMPLE 61B

### 4-(bromomethyl)-2-chlorobenzonitrile

A solution of Example 61A (0.22 g, 1.31 mmol) and LiBr (0.13 g, 1.44 mmol) in DMF (2 mL) at 0° C. was treated with PBr₃ (0.38 g, 1.39 mmol), stirred for 30 minutes, treated with water, and extracted with diethyl ether. The extract was washed with water and brine, dried (Na₂SO₄), filtered, and concentrated to provide the desired product of sufficient purity to be used in subsequent steps without further purification.

¹H NMR (300 MHz, CDCl₃) δ 7.67 (d, 1H), 7.57 (d, 1H), 7.40 (dd, 1H), 4.44 (s, 2H).

### EXAMPLE 61C

### 4-(((3 -chloro-4-cyanobenzyl)oxy)(1-methyl-1H-imidazol-5-yl)methyl)-2-(1-naphthyl)benzonitrile hydrochloride

The desired product was prepared by substituting Example 89D and Example 61B for Example 5D and (bromomethyl)benzene, respectively, in Example 5E.

MS (DCI/NH₃) m/z 489 (M+H)⁺; ¹H NMR (300 MHz, DMSO-d₆) δ 8.08 (m, 3H), 7.94 (m, 1H), 7.6 (m, 1H), 6.62 (m, 1H), 5.98 (s, 1H), 4.65 (m, 2H), 4.05 (s, 3H).

### EXAMPLE 62

### 4-(((4-cyanobenzyl)sulfanyl)(1-methyl-1H-imidazol-5-yl)methyl)-2-(1-naphthyl)benzonitrile hydrochloride

### EXAMPLE 62A

### 4-(sulfanylmethyl)benzonitrile

A mixture of 4-cyanobenzyl bromide (10 g, 50 mmol) and thiourea (9.8 g, 100 mmol) in ethanol (70 mL) was refluxed for 1 hour, cooled, and concentrated. The concentrate was washed with ethyl acetate, treated with 1.6M NaOH (100 mL), stirred for 22 hours, adjusted to pH 4 with concentrated HCl, and extracted with diethyl ether. The extract was washed with water and brine, dried (Na₂SO₄), filtered, and concentrated to provide the desired product of sufficient purity to be used in subsequent steps without further purification.

### EXAMPLE 62B

### 4-(chloro(1-methyl-1H-imidazol-5-yl)methyl)-2-(1-naphthyl)benzonitrile

A solution of Example 89D (100 mg, 0.29 mmol) in dichloromethane (10 mL) at 0° C. was treated with thionyl chloride (70 mg, 0.59 mmol), stirred for 15 minutes, warmed to room temperature, stirred for 2 hours, and concentrated to provide the desired product of sufficient purity to be used in subsequent steps without further purification.

### EXAMPLE 62C

### 4-(((4-cyanobenzyl)sulfanyl)(1-methyl-1H-imidazol-5-yl)methyl)-2-(1-naphthyl)benzonitrile hydrochloride

A solution of Example 62B in dichloromethane (5 mL) at room temperature was treated with Example 62A (53 mg, 0.35 mmol) and diisopropylethylamine (5 mL, 0.71 mmol), stirred for 18 hours, and concentrated. The concentrate was purified by flash column chromatography on silica gel with 98:2/chloroform:methanol, treated with 1M HCl in diethyl ether, and filtered to provide the desired product.

MS (DCI/NH₃) m/z 471 (M+H)⁺; ¹H NMR (300 MHz, DMSO-d₆) δ 9.3 (br s, 1H), 7.9 (m, 4H), 7.5 (m, 13H), 4.9 (br s, 2H), 4.2 (br s, 1H), 3.8 (br s, 3H).

### EXAMPLE 63

### 4-(((4-cyano-3-iodobenzyl)oxy)(1-methyl-1H-imidazol-5-yl)methyl)-2-(1-naphthyl)benzonitrile hydrochloride

### EXAMPLE 63A

### 2-iodo-4-methylbenzonitrile

The desired product was prepared by substituting 2-iodo-4-methylaniline for Example 87A in Examples 87B and 87C.

### EXAMPLE 63B

### 4-(bromomethyl)-2-iodobenzonitrile

A mixture of Example 63A (11.6 g, 47.2 mmol), N-bromosuccinimide (9.2 g, 51.9 mmol), and benzoyl peroxide (57 mg, 0.24 mmol) in carbon tetrachloride (150 mL) was heated to reflux for 18 hours, cooled to room temperature, washed with water and brine, dried (Na₂SO₄), filtered, and concentrated. The concentrate was purified by flash column chromatography on silica gel with 9:1/hexanes:ethyl acetate to provide the desired product.

MS (DCI/NH₃) m/z 339 and 341 (M+NH₄)⁺; ¹H NMR (300 MHz, CDCl₃) δ 7.95 (d, 1H), 7.59 (d, 1H), 7.48 (dd, 1H), 4.40 (s, 2H).

### EXAMPLE 63C

### 4-(((4-cyano-3-iodobenzyl)oxy)(1-methyl-1H-imidazol-5-yl)methyl)-2-(1-naphthyl)benzonitrile hydrochloride

The desired product was prepared by substituting Example 89D and Example 63B for Example 5D and (bromomethyl)benzene, respectively, in Example 5E.

MS (DCI/NH₃) m/z 581 (M+H)⁺; ¹H NMR (300 MHz, DMSO-d₆) δ 9.05 (s, 1H), 8.1 (m, 4H), 7.8 (m, 2H), 7.6 (m, 9H), 6.13 (s, 1H), 4.7 (m, 2H), 3.79 (d, 3H).

### EXAMPLE 64

### methyl 4-(((4-cyano-3-(1-naphthyl)phenyl)(1-methyl-1H-imidazol-5-yl)methoxy)methyl)benzoate hydrochloride

The desired product was prepared by substituting Example 89D and 4-(bromomethyl)-benzoate for Example 5D and (bromomethyl)benzene, respectively, in Example 5E.

MS (DCI/NH₃) m/z 488 (M+H)⁺; ¹H NMR (300 MHz, DMSO-d₆) δ 9.02 (s, 1H), 8.17 (m, 1H), 8.09 (m, 2H), 7.93 (m, 2H), 7.79 (m, 1H), 7.55 (m, 9H), 6.12 (s, 1H), 4.7 (m, 2H), 3.85 (s, 3H), 3.79 (d, 3H).

### EXAMPLE 65

### 4-((1-methyl-1H-imidazol-5-yl)((4-(trifluoromethyl)benzyl)oxy)methyl)-2-(1-naphthyl)benzonitrile hydrochloride

The desired product was prepared by substituting Example 89D and 4-(trifluoromethyl)benzyl bromide for Example 5D and (bromomethyl)benzene, respectively, in Example 5E.

MS (DCI/NH₃) m/z 498 (M+H)⁺; ¹H NMR (300 MHz, DMSO-d₆) δ 9.09 (m, 1H), 8.17 (m, 1H), 8.08 (m, 2H), 7.79 (d, 1H), 7.6 (m, 12H), 6.18 (s, 1H), 4.75 (m, 2H), 3.80 (d, 3H).

### EXAMPLE 66

### 4-(((4-chlorobenzyl)oxy)(1-methyl-1H-imidazol-5-yl)methyl)-2-(1-naphthyl)benzonitrile hydrochloride

The desired product was prepared by substituting Example 89D and 4-chlorobenzyl bromide for Example 5D and (bromomethyl)benzene, respectively, in Example 5E.

MS (DCI/NH₃) m/z 464 (M+H)⁺; ¹H NMR (300 MHz, DMSO-d₆) δ 9.03 (s, 1H), 8.17 (m, 1H), 8.09 (m, 2H), 7.78 (m, 2H), 7.62 (m, 5H), 7.51 (m, 2H), 7.41 (m, 4H), 6.10 (s, 1H), 4.6 (m, 2H), 3.78 (d, 3H).

### EXAMPLE 67

### 4-((1-methyl-1H-imidazol-5-yl)((4-(trifluoromethoxy)benzyl)oxy)methyl)-2-(1-naphthyl)benzonitrile hydrochloride

The desired product was prepared by substituting Example 89D and 4-(trifluoromethoxy)benzyl bromide for Example 5D and (bromomethyl)benzene, respectively, in Example 5E.

MS (DCI/NH₃) m/z 514 (M+H)⁺; ¹H NMR (300 MHz, DMSO-d₆) δ 8.94 (s, 1H), 8.16 (m, 1H), 8.09 (m, 2H), 7.78 (m, 1H), 7.61 (m, 4H), 7.50 (m, 5H), 7.35 (m, 3H), 6.11 (s, 1H), 4.65 (m, 2H), 3.77 (d, 3H).

### EXAMPLE 68

### 4-((1-methyl-1H-imidazol-5-yl)((3-(trifluoromethyl)benzyl)oxy)methyl)-2-(1-naphthyl)benzonitrile hydrochloride

The desired product was prepared by substituting Example 89D and 3-(trifluoromethyl)benzyl bromide for Example 5D and (bromomethyl)benzene, respectively, in Example 5E.

MS (DCI/NH₃) m/z 498 (M+H)⁺; ¹H NMR (300 MHz, DMSO-d₆) δ 8.98 (s, 1H), 8.17 (m, 1H), 8.09 (m, 2H), 7.79 (m, 1H), 7.6 (m, 11H), 7.40 (m, 1H), 6.13 (s, 1H), 4.7 (m, 2H), 3.78 (d, 3H).

### EXAMPLE 69

### lithium 4-(((4-cyano-3-(1-naphthyl)phenyl)(1-methyl-1H-imidazol-5-yl)methoxy)methyl)benzoate

A solution of Example 64 (98 mg, 0.20 mmol) in methanol (2 mL) at room temperature was treated with 1M LiOH (0.21 mL, 0.21 mmol), stirred for 48 hours, and concentrated. The concentrate was treated with water, washed with diethyl ether, and lyophilized to provide the desired product of sufficient purity to be used in subsequent steps without further purification.

MS (DCI/NH₃) m/z 474 (M+H)⁺; ¹H NMR (300 MHz, DMSO-d₆) δ 8.09 (m, 3H), 7.78 (m, 3H), 7.6 (m, 7H), 7.20 (m, 2H), 6.55 (d, 1H), 5.89 (d, 1H), 4.51 (m, 2H), 3.54 (d, 3H).

### EXAMPLE 70

### 4-(((4-cyano-3-(1-naphthyl)phenyl)(1-methyl-1H-imidazol-5-yl)methoxy)methyl)-N,N-dimethylbenzamide hydrochloride

A solution of Example 69 (50 mg, 0.10 mmol) and oxalyl chloride (0.10 mmol) in dichloromethane (2 mL) was treated with DMF (1 drop), stirred for 1 hour, and concentrated. The concentrate was treated with ethyl acetate, washed with water and brine, dried (Na₂SO₄), filtered, and concentrated. The concentrate was purified by flash column chromatography on silica gel with 98:2/chloroform:methanol, treated with HCl, and concentrated to provide the desired product.

MS (DCI/NH₃) m/z 501 (M+H)⁺; ¹H NMR (300 MHz, DMSO-d₆) δ 8.61 (m, 1H), 8.1 (m, 3H), 7.78 (m, 1H), 7.5 (m, 11H), 7.17 (m, 1H), 6.08 (m, 1H), 4.65 (m, 2H), 3.71 (d, 3H), 2.97 (s, 3H), 2.88 (s, 3H).

### EXAMPLE 71

### 4-(((4-cyanobenzyl)sulfonyl)(1-methyl-1H-imidazol-5-yl)methyl)-2-(1-naphthyl)benzonitrile hydrochloride

A solution of Example 62C (31 mg, 0.07 mmol) in dichloromethane (2 mL) at room temperature was treated with 70% m-CPBA (100 mg), stirred for 48 hours, treated with ethyl acetate, washed with saturated NaHCO₃ and brine, dried (Na₂SO₄), filtered, and concentrated. The concentrate was purified by flash column chromatography on silica gel with 98:2/chloroform:methanol. The appropriate fractions were treated with HCl and concentrated to provide the desired product.

MS (DCI/NH₃) m/z 503 (M+H)⁺.

### EXAMPLE 72

### 4-(((2,4-dichlorobenzyl)oxy)(1-methyl-1H-imidazol-5-yl)methyl)-2-(1-naphthyl)benzonitrile hydrochloride

### EXAMPLE 72A

### 2,4-dichloro-1-(iodomethyl)benzene

A solution of 2,4-dichlorobenzyl chloride (65 mg, 0.33 mmol) and NaI (0.5 g, 3.3 mmol) in acetone (5 mL) was heated to 50° C., stirred for 18 hours, and concentrated. The concentrate was treated with dichloromethane (1.5 mL) and filtered to provide the desired product of sufficient purity for use in subsequent steps without further purification.

### EXAMPLE 72B

### 4-(((2,4-dichlorobenzyl)oxy)(1-methyl-1H-imidazol-5-yl)methyl)-2-(1-naphthyl)benzonitrile hydrochloride

The desired product was prepared by substituting Example 89D and Example 72A for Example 5D and (bromomethyl)benzene, respectively, in Example 5E.

MS (DCI/NH₃) m/z 498 (M+H)⁺; ¹H NMR (300 MHz, DMSO-d₆) δ 9.09 (s, 1H), 8.16 (m, 1H), 8.09 (m, 2H), 7.78 (m, 1H), 7.55 (m, I1H), 6.18 (s, 1H), 4.7 (m, 2H), 3.79 (d, 3H).

### EXAMPLE 73

### 4-((1-methyl-1H-imidazol-5-yl)((4-(methylsulfonyl)benzyl)oxy)methyl)-2-(1-naphthyl)benzonitrile hydrochloride

### EXAMPLE 73A

### 1-(iodomethyl)-4-(methylsulfonyl)benzene

The desired product was prepared by substituting 4-(methylsulfonyl)benzyl chloride for 2,4-dichlorobenzyl chloride in Example 72A.

MS (DCI/NH₃) m/z 314 (M+H)⁺.

### EXAMPLE 73B

### 4-((1-methyl-1H-imidazol-5-yl)((4-(methylsulfonyl)benzyl)oxy)methyl)-2-(1-naphthyl)benzonitrile hydrochloride

The desired product was prepared by substituting Example 89D and Example 73A for Example 5D and (bromomethyl)benzene, respectively, in Example 5E.

MS (DCI/NH₃) m/z 508 (M+H)⁺; ¹H NMR (300 MHz, DMSO-d₆) δ 9.01 (m, 1H), 8.17 (m, 1H), 8.09 (m, 2H), 7.90 (m, 4H), 7.80 (m, 1H), 7.6 (m, 8), 6.16 (s, 1H), 4.75 (m, 2H), 3.79 (d, 3H), 3.20 (s, 3H).

### EXAMPLE 74

### 4-(((2,6-dichloro-4-pyridinyl)methoxy)(1-methyl-1H-imidazol-5-yl)methy)-2-(1-naphthyl)benzonitrile dihydrochloride

The desired product was prepared by substituting Example 89D and 4-(bromomethyl)-2,6-dichloropyridine for Example 5D and (bromomethyl)benzene, respectively, in Example 5E.

MS (DCI/₃) m/z 499 (M+H)⁺; ¹H NMR (300 m/z, DMSO-d₆) δ 9.04 (s, 1H), 8.17 (m, 1H), 8.08 (m, 2H), 7.81 (m, 1H), 7.58 (m, 11H), 6.16 (m, 1H), 4.7 (m, 2H), 3.79 (d, 3H).

### EXAMPLE 75

### 4-(((3-bromo-4-cyanobenzyl)oxy)(1-methyl-1H-imidazol-5-yl)methyl)-2-(1-naphthyl)benzonitrile hydrochloride

### EXAMPLE 75A

### 2-bromo-4-(hydroxymethyl)benzonitrile

The desired product was prepared by substituting Example 87C for Example 5A in Example 5B.

MS (DCI/NH₃) m/z 229 and 231 (M+NH₄)⁺; ¹H NMR (300 MHz, CDCl₃) δ 7.74 (s, 1H), 7.65 (m, 1H), 7.41 (d, 4.30 (s, 2H), 1.89 (br s, 1H).

### EXAMPLE 75B

### 2-bromo-4-(bromomethyl)benzonitrile

The desired product was prepared by substituting Example 75A for Example 61A in Example 61B.

MS (DCI/NH₃) m/z 293 (M+NH₄)⁺; ¹H NMR (300 MHz, CDCl₃) δ 7.73 (m, 1H), 7.64 (d, 1H), 7.44 (dd, 1H), 4.42 (s, 2H).

### EXAMPLE 75C

### 4-(((3-bromo-4-cyanobenzyl)oxy)(1-methyl-1H-imidazol-5-yl)methyl)-2-(1-naphthyl)benzonitrile hydrochloride

The desired product was prepared by substituting Example 89D and Example 75B for Example 5D and (bromomethyl)benzene, respectively, in Example 5E.

MS (DCI/NH₃) m/z 533 and 535 (M+H)⁺; ¹H NMR (300 MHz, DMSO-d₆) δ 9.07 (s, 1H), 8.17 (m, 1H), 8.09 (m, 2H), 7.91 (m, 2H), 7.80 (m, 1H), 7.55 (m, 9H), 6.15 (s, 1H), 4.73 (m, 2H), 3.79 (d, 3H).

### EXAMPLE 76

### 6-(((4-cyano-3-(1-naphthyl)phenyl)(1-methyl-1H-imidazol-5-yl)methoxy)methyl)nicotinonitrile dihydrochloride

### EXAMPLE 76A

### 6-(bromomethyl)nicotinonitrile

The desired product was prepared by substituting 6-methylnicotinonitrile for Example 63A in Example 63B.

MS (DCI/NH₃) m/z 197 (M+H)⁺; ¹H NMR (300 MHz, CDCl₃) δ 8.86 (s, 1H), 7.99 (dd, 1H), 7.60 (d, 1H), 4.58 (s, 2H).

### EXAMPLE 76B

### 6-(((4-cyano-3-(1-naphthyl)phenyl)(1-methyl-1H-imidazol-5-yl)methoxy)methyl)nicotinonitrile dihydrochloride

The desired product was prepared by substituting Example 89D and Example 76A for Example 5D and (bromomethyl)benzene, respectively, in Example 5E.

MS (DCI/NH₃) m/z 456 (M+H)⁺; ¹H NMR (300 MHz, DMSO-d₆) δ 9.12 (s, 1H), 8.98 (m, 1H), 8.32 (m, 1H), 8.11 (m, 3H), 7.80 (m, 1H), 7.6 (m, 10H), 6.23 (m, 1H), 4.72 (m, 2H), 3.81 (d, 3H).

### EXAMPLE 77

### 4-(((4-cyano-3-fluorobenzyl)oxy)(1-methyl-1H-imidazol-5-yl)methyl)-2-(1-naphthyl)benzonitrile hydrochloride

### EXAMPLE 77A

### 2-fluoro-4-methylbenzonitrile

The desired product was prepared by substituting 2-fluoro-4-methylaniline for Example 87A in Examples 87B and 87C.

MS (DCI/NH₃) m/z 153 (M+NH₄)⁺; ¹H NMR (300 MHz, CDCl₃) δ 7.50 (m, 1H), 7.16 (m, 2H), 2.44 (s, 3H).

### EXAMPLE 77B

### 4-(bromomethyl)-2-fluorobenzonitrile

The desired product was prepared by substituting Example 77A for Example 63A in Example 63B.

### EXAMPLE 77C

### 4-(((4-cyano-3-fluorobenzyl)oxy)(1-methyl-1H-imidazol-5-yl)methyl)-2-(1-naphthyl)benzonitrile hydrochloride

The desired product was prepared by substituting Example 89D and Example 77B for Example 5D and (bromomethyl)benzene, respectively, in Example 5E.

MS (DCI/NH₃) m/z 473 (M+H)⁺; ¹H NMR (300 MHz, DMSO-d₆) δ 8.88 (br s, 1H), 8.16 (m, 1H), 8.08 (m, 2H), 7.92 (m, 1H), 7.79 (m, 1H), 7.55 (m, 10H), 6.12 (m, 1H), 4.75 (m, 2H), 3.75 (d, 3H).

### EXAMPLE 78

### 5-((benzyloxy)(1-methyl-1H-1,2,4-triazol-5-yl)methyl)-2′-methyl(1,1′-biphenyl)-2-carbonitrile hydrochloride

### EXAMPLE 78A

### 5-(hydroxy(1-methyl-1H-1,2,4-triazol-5-yl)methyl)-2′-methyl(1,1′-biphenyl)-2-carbonitrile

A solution of 1-methyl-1H-1,2,4-triazole (68 mg, 0.82 mmol) in THF (3 mL) at −78° C. was treated with n-butyllithium (2.5M, 0.33 mL, 0.82 mmol), stirred for 1 hour, treated with a solution of 86I (150 mg, 0.68 mmol) in THF (2 mL), stirred for 16 hours while warming to room temperature, and treated with 5.5M ammonium chloride to provide two layers. The aqueous layer was adjusted to a pH greater than 7 with sodium bicarbonate and extracted with dichloromethane. The extract was dried (MgSO₄), filtered, and concentrated. The concentrate was purified by flash column chromatography on silica gel with 3:1/ethyl acetate:hexanes to provide the desired product.

MS (ESI(+)) m/z 305 (M+H)⁺; ¹H NMR (300 MHz, CDCl₃) δ 7.88 (s, 1H), 7.77 (d, 1H), 7.52 (m, 1H), 7.42 (s, 1H), 7.38-7.25 (m, 3H), 7.17 (d, 1H), 6.26 (s, 1H), 3.81 (s, 3H), 2.15 (s, 3H).

### EXAMPLE 78B

### 5-((benzyloxy)(1-methyl-1H-1,2,4-triazol-5-yl)methyl)-2′-methyl(1,1′-biphenyl)-2-carbonitrile hydrochloride

A solution of 78A (126 mg, 0.41 mmol) in dichloromethane (8 mL) at room temperature was treated with silver(I) oxide (115 mg, 0.5 mmol) and benzyl bromide (59 μL, 0.5 mmol), stirred for 48 hours in darkness, filtered, and concentrated. The concentrate was purified by flash column chromatography on silica gel with 60:40/hexanes:ethyl acetate. The appropriate fractions were dissolved in acetonitrile, treated with 1M HCl, and lyopholized to provide the desired product.

MS (ESI(+)) m/z 395 (M+H)⁺; ¹H NMR (300 MHz, DMSO-d₆) δ 8.01 (d, 1H), 7.91 (s, 1H), 7.66 (dd, 1H), 7.50 (s, 1H), 7.42-7.28 (m, 8H), 7.24 (m, 1H), 6.14 (s, 1H), 4.57 (s, 2H), 3.84 (s, 3H), 2.11 (s, 3H); Anal. calcd for C₂₅H₂₂N₄O·HCl0.5 H₂O: C, 68.25; H, 5.50; N, 12.73; Cl, 8.06. Found: C, 67.89; H, 5.61; N, 12.90; Cl, 8.34.

### EXAMPLE 79

### 5-((benzyloxy)(1-methyl-1H-pyrazol-5-yl)methyl)-2′-methyl(1,1′-biphenyl)-2-carbonitrile hydrochloride

### EXAMPLE 79A

### 5-(hydroxy(1,1-methyl-1H-pyrazol-5-yl)methyl)-2′-methyl(1,1′-biphenyl)-2-carbonitrile

The desired product was prepared by substituting 1-methyl-1H-pyrazole for 1-methyl-1H-1,2,4-triazole in Example 78A.

MS (ESI(+)) m/z 304 (M+H)⁺; ¹H NMR (300 MHz, CDCl₃) δ 7.78 (d, 1H), 7.50 (d, 1H), 7.40 (m, 2H), 7.38-7.25 (m, 3H), 7.19 (d, 1H), 6.05 (d, 1H), 6.04 (s, 1H), 3.84 (s, 3H), 2.17 (s, 3H).

### EXAMPLE 79B

### 5-((benzyloxy)(1-methyl-1H-pyrazol-5-yl)methyl)-2′-methyl(1,1′-biphenyl)-2-carbonitrile hydrochloride

The desired product was prepared by substituting Example 79A for Example 78A in Example 78B.

MS (DCI/NH₃) m/z 394 (M+H)⁺; ¹H NMR (300 MHz, DMSO-d₆) δ 8.01 (d, 1H), 7.64 (dd, 1H), 7.46 (d, 1H), 7.42-7.27 (m, 5H), 6.00 (d, 1H), 5.98 (s, 1H), 4.54 (q, 2H), 3.75 (s, 3H), 2.11 (s, 3H); Anal. calcd for C₂₆H₂₃N₃O·HCl: C, 72.63; H, 5.63; N, 9.77. Found: C, 72.61; H, 5.64; N, 9.62.

### EXAMPLE 80

### 5-((benzyloxy)(3-thienyl)methyl)-2′-methyl(1,1′-biphenyl)-2-carbonitrile

### EXAMPLE 80A

### 5-(hydroxy(3-thienyl)methyl)-2′-methyl(1,1′-biphenyl)-2-carbonitrile

A solution of 3-bromothiophene (70 μL, 0.75 mmol) in hexanes (3 mL) at −40° C. was treated with n-butyllithium (2.5M, 0.33 mL, 0.82 mmol), stirred for 20 minutes, added to a solution of 86I (150 mg, 0.68 mmol) in THF (3 mL) at −78° C., stirred for 16 hours while warming to room temperature, treated with 5.5M ammonium chloride, and extracted with dichloromethane. The extract was dried (MgSO₄), filtered, and concentrated. The concentrate was purified by flash column chromatography on silica gel with 9:1 to 4:1/hexanes:ethyl acetate to provide the desired product.

MS (ESI(+)) m/z 323 (M+NH₄)⁺; ¹H NMR (300 MHz, CDCl₃) δ 7.66 (d, 1H), 7.44 (dd, 1H), 7.36 (s, 1H), 7.29-7.11 (m, 6H), 6.92 (d, 1H), 5.91 (s, 1H), 2.10 (s, 3H).

### EXAMPLE 80B

### 5-((benzyloxy)(3-thienyl)methyl)-2′-methyl(1,1′-biphenyl)-2-carbonitrile

The desired product was prepared by substituting Example 80A for Example 78A in Example 78B.

¹H NMR (300 MHz, CDCl₃) δ 7.72 (d, 1H), 7.49 (dd, 1H), 7.40-7.12 (m, 12H), 6.98 (m, 1H), 5.56 (s, 1H), 4.55 (m, 2H), 2.16 (s, 3H); HRMS (FAB) calcd m/z for C₂₆H₂₂NO₅: 396.1422 (M+H)⁺. Found: 396.1419.

### EXAMPLE 81

### 5-((benzyloxy)(1-methyl-1H-1,2,3-triazol-5-yl)methyl)-2′-methyl(1,1′-biphenyl)-2-carbonitrile hydrochloride

### EXAMPLE 81A

### 5-(hydroxy(1-methyl-1H-1,2,3-triazol-5-yl)methyl)-2′-methyl(1,1′-biphenyl)-2-carbonitrile

The desired product was prepared by substituting 1-methyl-1H-1,2,3-triazole for 1-methyl-1H-1,2,4-triazole in Example 78A.

MS (ESI(+)) m/z 305 (M+H)⁺.

### EXAMPLE 81B

### 5-((benzyloxy)(1-methyl-1H-1,2,3-triazol-5-yl)methyl)-2′-methyl(1,1′-biphenyl)-2-carbonitrile hydrochloride

The desired product was prepared by substituting Example 81A for Example 78A in Example 78B.

MS (ESI(+)) m/z 395 (M+H)⁺; ¹H NMR (300 MHz, CD₃OD) δ 7.93 (d, 1H), 7.72 (s, 1H), 7.64 (dd, 1H), 7.51 (d, 1H), 7.37-7.29 (m, 8H), 7.22 (m, 1H), 6.02 (s, 1H), 4.64 (s, 2H), 4.04 (s, 3H), 2.16 (s, 3H); Anal. calcd for C₂₅H₂₂N₄O·HCl: C, 69.68; H, 5.38; N, 13.00. Found: C, 70.01; H, 5.37; N, 13.08.

### EXAMPLE 82

### 5-(((2-cyclohexylethyl)((1-methyl-1H-imidazol-5-yl)methyl)amino)methyl)-2′-methyl(1,1′-biphenyl)-2-carbonitrile hydrochloride

### EXAMPLE 82A

### 5-(((2-cyclohexylethyl)amino)methyl)-2′-methyl(1,1′-biphenyl)-2-carbonitrile

A solution of 861 and 2-(cyclohexyl)ethylamine (153 mg, 1.21 mmol) in dichloromethane (15 mL) at room temperature was treated with acetic acid (3 drops), stirred for 1 hour, treated with sodium triacetoxyborohydride (384 mg, 1.81 mmol), stirred for three hours, treated with ethyl acetate, washed with saturated NaHCO₃, dried (MgSO₄), filtered, and concentrated. The concentrate was purified by flash column chromatography on silica gel with 0.2% concentrated ammonium hydroxide/ethyl acetate to provide the desired product.

MS (ESI(+)) m/z 333 (M+H)⁺; ¹H NMR (300 MHz, CDCl₃) δ 7.70 (d, 1H), 7.46 (m, 1H), 7.35 (m, 1H), 7.31 (m, 2H), 7.26 (m, 1H), 7.20 (d, 1H), 3.89 (s, 2H), 2.66 (dd, 2H), 2.19 (s, 3H), 1.67 (m, 5H), 1.43 (m, 2H), 1.32-1.10 (m, 4H), 0.95-0.83 (m, 2H).

### EXAMPLE 82B

### 5-(((2-cyclohexylethyl)((1-methyl-1H-imidazol-5-yl)methyl)amino)methyl)-2′-methyl(1,1′-biphenyl)-2-carbonitrile hydrochloride

The free base of the desired product was prepared by substituting Example 82A and Example 32C for 2-(cyclohexyl)ethylamine and Example 861, respectively, in Example 82A. The purified concentrate was dissolved in acetonitrile, treated with 1M HCl, and lyopholized to provide the desired product.

MS (ESI(+)) m/z 427 (M+H)⁺, ¹H NMR (300 MHz, CD₃OD) δ 9.04 (s, 1H), 7.96 (m, 2H), 7.84 (m, 1H), 7.74 (m, 1H), 7.37 (m, 2H), 7.35-7.28 (m, 1H), 7.22 (m, 1H), 4.56 (br s, 4H), 3.94 (s, 3H), 3.18 (m, 2H), 2.19 (s, 3H), 1.73-1.64 (m, 7H), 1.31-1.14 (m, 4H), 1.01-0.89 (m, 2H); Anal. calcd for C₂₈H₃₄N₄·3.01 HCl·0.48 H₂O: C, 61.71; H, 7.02; N, 10.28. Found: C, 61.77; H, 7.02; N, 9.91.

### EXAMPLE 83

### 4-(((2-cyclohexylethyl)((1-methyl-1H-imidazol-5-yl)methyl)amino)methyl)-2-(1-naphthyl)benzonitrile dihydrochloride

### EXAMPLE 83A

### 4-(((2-cyclohexylethyl)amino)methyl)-2-(1-naphthyl)benzonitrile

The desired product was prepared by substituting Example 89C for Example 86I in Example 82A.

MS (ESI(+)) m/z 369 (M+H)⁺; ¹H NMR (300 MHz, CDCl₃) δ 7.96 (d, 1H), 7.93 (d, 1H), 7.79 (d, 1H), 7.59-7.41 (m, 7H), 3.92 (s, 2H), 2.66 (t, 2H), 1.67 (m, 5H), 1.51 (s, 1H), 1.40 (m, 2H), 1.35-1.06 (m, 4H), 0.96-0.83 (m, 2H).

### EXAMPLE 83B

### 4-(((2-cyclohexylethyl)((1-methyl-1H-imidazol-5-yl)methyl)amino)methyl)-2-(1-naphthyl)benzonitrile dihydrochloride

The desired product was prepared by substituting Example 83A for Example 82A in Example 82B.

MS (ESI(+)) m/z 463 (M+H)⁺; ¹H NMR (300 MHz, CD₃OD) δ 9.02 (s, 1H), 8.02 (m, 3H), 7.94 (m, 2H), 7.84 (m, 1H), 7.62 (m, 1H), 7.56 (m, 1H), 7.49 (m, 3H), 4.55 (br s, 4H), 3.95 (s, 3H), 3.18 (br s, 2H), 1.66 (m, 7H), 1.29-0.91 (m, 6H); Anal. calcd for C₃₇H₃₄N₄·2.18 HCl·1.58 H₂O: C, 65.26; H, 6.95; N, 9.82. Found: C, 65.30; H, 6.95; N, 9.56.

### EXAMPLE 84

### 4-(((cyclohexylmethyl)((1-methyl-1H-imidazol-5-yl)methyl)amino)methyl)-2-(1-naphthyl)benzonitrile dihydrochloride

### EXAMPLE 84A

### 4-(((cyclohexylmethyl)amino)methyl)-2-(1-naphthyl)benzonitrile

The desired product was prepared by substituting cyclohexylmethylamine for 2-(cyclohexyl)ethylamine in Example 83A.

### EXAMPLE 84B

### 4-(((cyclohexylmethyl)((1-methyl-1H-imidazol-5-yl)methyl)amino)methyl)-2-(1-naphthyl)benzonitrile dihydrochloride

The desired product was prepared by substituting Example 84A for Example 83A in Example 83B.

¹H NMR (300 MHz, CD₃OD) δ 9.01 (s, 1H), 8.09-7.75 (m, 6H), 7.64-7.47 (m, 5H), 4.5 (br s, 4H), 3.96 (s, 3H), 3.0 (br s, 2H), 1.85-1.68 (m, 6H), 1.29-1.13 (m, 3H), 0.92-0.88 (m, 2H);

HRMS (FAB) calcd m/z for C₃₀H₃₃N₄: 449.2705 (M+H)⁺. Found: 449.2715; Anal. calcd for C₃₀H₃₂N₄·2.31 HCl 2.02 H₂O: C, 63.30; H, 6.79; N, 9.84. Found: C, 63.28; H, 6.79; N, 9.94.

### EXAMPLE 85

### N-(4-cyano-3-(1-naphthyl)benzyl)-N-(2-cyclohexylethyl)-2-(1H-imidazol-1-yl)acetamide

### EXAMPLE 85A

### 2-chloro-N-(4-cyano-3-(-1-naphthyl)benzyl)-N-(2-cyclohexylethyl)acetamide

A solution of Example 83A (103 mg, 0.28 mmol) in dichloromethane (3 mL) and pyridine (0.05 mL) at 0° C. was treated with chloroacetic anhydride (53 mg, 0.31 mmol), stirred for 1 hour, poured into 1M NaHSO₄, and extracted with dichloromethane. The extract was dried (MgSO₄), filtered, and concentrated to provide the desired product of sufficient purity for subsequent use without further purification.

### EXAMPLE 85B

### N-(4-cyano-3-(1-naphthyl)benzyl)-N-(2-cyclohexylethyl)-2-(1H-imidazol-1-yl)acetamide

A solution of Example 85A in DMSO (3 mL) at room temperature was treated with imidazole (57 mg, 0.83 mmol), stirred for 2 hours, heated to 50° C., and stirred for 16 hours. The mixture was treated with saturated NaHCO₃, extracted with dichloromethane, dried (MgSO₄), filtered, and concentrated. The concentrate was purified by flash column chromatography on silica gel with 98.8:1:0.2/ethyl acetate:methanol:concentrated ammonium hydroxide to provide the desired product.

MS (ESI(+)) m/z 477 (M+H)⁺, ¹H NMR (300 m/z, DMSO-d₆) δ 8.09-7.99 (m, 3H), 7.68-7.41 (m, 8H), 7.03 (m, 1H), 6.84 (s, 1H), 5.06 (m, 2H), 4.80-4.68 (m, 2H), 3.40-3.25 (m, 2H), 2.54 (s, 2H), 1.63-0.83 (m, 11H).

### EXAMPLE 86

### 5-(hydroxy(1-methyl-1H-imidazol-5-yl)methyl)-2′-methyl(1,1′-biphenyl)-2-carbonitrile

### EXAMPLE 86A

### dimethyl 2′-methyl(1,1′-biphenyl)-2,5-dicarboxylate

The desired product was prepared by substituting 2-methylphenylboronic acid for 2-chlorophenylboronic acid in Example 10A.

### EXAMPLE 86B

### 6-(methoxycarbonyl)-2′-methyl(1,1′-biphenyl)-3-carboxylic acid

The desired product was prepared by substituting Example 86A for Example 10A in Example 10B.

### EXAMPLE 86C

### methyl 5-(hydroxymethyl)-2′-methyl(1,1′-biphenyl)-2-carboxylate

The desired product was prepared by substituting Example 86B for Example 10B in Example 10C.

MS (DCI/NH₃) m/z 257 (M+H)⁺; ¹H NMR (300 MHz, CDCl₃) δ 7.98 (d, 1H), 7.43 (dd, 1H), 7.28-7.16 (m, 4H), 7.07 (br d, 1H), 4.77 (s, 2H), 3.62 (s, 3H), 2.05 (s, 3H), 1.78 (br s, 1H).

### EXAMPLE 86D

### 4-(hydroxymethyl)-2-(1-naphthyl)benzonitrile

A solution of Example 86C (6.0 g, 23.4 mmol) in dichloromethane (25 mL) at room temperature was treated with chloromethyl ethyl ether (4.4 mL, 4.5 g, 47 mmol) and diisopropylethyl amine (8.3 mL, 6.1 g, 47 mmol), stirred for 1.5 hours, treated with water and diethyl ether, and extracted with diethyl ether. The extract was washed with brine, dried (Na₂SO₄), filtered, and concentrated to provide the desired product of sufficient purity for subsequent use without further purification.

MS (DCI/NH₃) m/z 315 (M+H)⁺; ¹H NMR (300 MHz, CDCl₃) δ 7.98 (d, 1H), 7.40 (dd, 1H), 7.28-7.16 (m, 4H), 7.07 (br d, 1H), 4.78 (s, 2H), 4.67 (s, 2H), 3.65 (q, 2H), 3.60 (s, 3H), 2.05 (s, 3H), 1.21 (t, 3H).

### EXAMPLE 86E

### 5-((ethoxymethoxy)methyl)-2′-methyl(1,1′-biphenyl)-2-carboxylic acid

The desired product was prepared by substituting Example 86D for Example 10F in Example 10G.

### EXAMPLE 86F

### 5-((ethoxymethoxy)methyl)-2′-methyl(1,1′-biphenyl)-2-carboxamide

The desired product was prepared by substituting Example 86E for Example 10G in Example 10H.

MS (DCI/NH₃) m/z 300 (M+H)⁺; ¹H NMR (300 MHz, DMSO-d₆) δ 7.50 (d, 1H), 7.37 (dd, 1H), 7.31 (br s, 1H), 7.25-7.08 (m, 6H), 4.72 (s, 2H), 4.59 (s, 2H), 3.55 (q, 2H), 2.05 (s, 3H), 1.21 (t, 3H).

### EXAMPLE 86G

### 5-(hydroxymethyl)-2′-methyl(1,1′-biphenyl)-2-carboxamide

A solution of Example 86F (0.37 g, 1.2 mmol) in methanol (5 mL) at room temperature was treated with concentrated HCl (0.1 mL), stirred for 16 hours, and concentrated. The concentrate was treated with toluene, concentrated, and dried under vacuum with P₂O₅ to provide the desired product of sufficient purity for subsequent use without further purification.

### EXAMPLE 86H

### 5-(hydroxymethyl)-2′-methyl(1,1′-biphenyl)-2-carbonitrile

The desired product was prepared by substituting Example 86G for Example 10H in Example 101.

MS (DCI/NH₃) m/z 241 (M+NH₄)⁺; ¹H NMR (300 MHz, CDCl₃) δ 7.73 (d, 1H), 7.45 (m, 1H), 7.37 (m, 1H), 7.30 (m, 2H), 7.25 (m, 1H), 7.18 (br d, 1H), 4.80 (br d, 2H), 2.20 (s, 3H), 1.93 (br t, 1H).

### EXAMPLE 86I

### 5-formyl-2′-methyl(1,1′-biphenyl)-2-carbonitrile

The desired product was prepared by substituting Example 86H for Example 5B in Example 5C.

MS (DCI/NH₃) m/z 239 (M+NH₄)⁺; ¹H NMR (300 MHz, CDCl₃) δ 10.12 (s, 1H), 7.95 (m, 2H), 7.89 (s, 1H), 7.42-7.30 (m, 3H), 7.22 (br d, 1H), 2.24 (s, 3H).

### EXAMPLE 86J

### 5-(hydroxy(1-methyl-1H-imidazol-5-yl)methyl)-2′-methyl(1,1′-biphenyl)-2-carbonitrile

The desired product was prepared by substituting Example 86I for Example 1A in Example 1B.

MS (DCI/NH₃) m/z 304 (M+H)⁺; ¹H NMR (300 MHz, DMSO-d₆) δ 7.95 (d, 1H), 7.60 (br d, 1H), 7.55 (s, 1H), 7.44 (br s, 1H), 7.38 (m, 2H), 7.30 (m, 1H), 7.22 (br d, 1H), 6.42 (s, 1H), 6.18 (d, 1H), 5.94 (d, 1H), 3.58 (s, 3H), 2.13 (br s, 3H). Anal. calcd for C₁₉H₁₇N₃O·00.20 H₂O: C, 74.34; H, 5.71; N, 13.69. Found: C, 74.26; H, 5.74; N, 13.68.

### EXAMPLE 87

### 5-((benzyloxy)(1-methyl-1H-imidazol-5-yl)methyl)(1,1′-biphenyl)-2-carbonitrile hydrochloride

### EXAMPLE 87A

### ethyl 4-amino-3-bromobenzoate

A solution of ethyl 4-aminobenzoate (5.5 g, 33 mmol) in dichloromethane (48 mL), at −12° C., was treated with pyridine (5.5 mL, 5.4 g, 68 mmol) and a solution of bromine (1.75 mL, 5.4 g, 34 mmol) in dichloromethane (15 mL), warmed to room temperature, stirred for 16 hours, and treated with diethyl ether and 0.5M H₃PO₄ to provide two layers. The organic layer was washed with brine, dried (Na₂SO₄), filtered, and concentrated. The concentrate was purified by flash column chromatography on silica gel with 85:15/hexanes:ethyl acetate to provide the desired product.

MS (DCI/NH₃) m/z 244 and 246 (M+H)⁺, 261 and 263 (M+NH₄)⁺; ¹H NMR (300 MHz, CDCl₃) δ 8.11 (d, 1H), 7.80 (dd, 1H), 6.72 (d, 1H), 4.50 (br s, 2H), 4.33 (q, 2H), 1.38 (t, 3H).

### EXAMPLE 87B

### 2-bromo-4-(ethoxycarbonyl)benzenediazonium tetrafluoroborate

A solution of Example 87A (1.2 g, 4.8 mmol) in dichloromethane (10 mL) at −8° C., was treated with a −8° C. solution of BF₃.OEt2 (0.9 mL, 1.0 g, 7.3 mmol) and tert-butyl nitrite (0.7 mL, 0.6 g, 5.9 mmol) and warmed to room temperature. The mixture was treated with hexanes and the resulting solid was removed by filtration to provide the desired product.

### EXAMPLE 87C

### ethyl 3-bromo-4-cyanobenzoate

A solution of copper(I) cyanide (520 mg, 5.8 mmol) and sodium cyanide (710 mg, 14.5 mmol) in water (3.5 mL) at 5° C. was treated with toluene (1.5 mL) and Example 87B, stirred for 30 minutes, warmed to room temperature, heated to 60° C. for 25 minutes, cooled to room temperature, and treated with water and ethyl acetate. The organic layer was washed with brine, dried (Na₂SO₄), filtered, and concentrated. The concentrate was treated with hexanes (22 mL), heated to 60° C., decanted, cooled to room temperature, cooled to 4° C. for 16 hours, and filtered to provide the desired product.

MS (DCI/NH₃) m/z 271 and 273 (M+NH₄)⁺; ¹ H NMR (300 MHz, CDCl₃) δ 8.33 (d, 1H), 8.07 (dd, 1H), 7.74 (d, 1H), 4.43 (q, 2H), 1.42 (t, 3H).

### EXAMPLE 87D

### ethyl 6-cyano(1,1′-biphenyl)-3-carboxylate

The desired product was prepared by substituting Example 87C and phenylboronic acid for 3-bromo-4-fluorobenzaldehyde and 2-methylphenylboronic acid, respectively, in Example 1A.

MS (DCI/NH₃) m/z 269 (M+NH₄)⁺; ¹H NMR (300 MHz, CDCl₃) δ 8.19 (d, 1H), 8.10 (dd, 1H), 7.84 (d, 1H), 7.59 (m, 2H), 7.50 (m, 3H), 4.43 (q, 2H), 1.42 (t, 3H).

### EXAMPLE 87E

### 5-(hydroxymethyl)(1,1′-biphenyl)-2-carbonitrile

The desired product was prepared by substituting Example 87D for Example 5A in Example 5B.

MS (DCI/NH₃) m/z 227 (M+NH)⁺; ¹H NMR (300 MHz, CDCl₃) δ 7.77 (d, 1H), 7.50 (m, 7H), 4.82 (d, 2H), 1.91 (t, 1H).

### EXAMPLE 87F

### 1-methyl-2-(triethylsilyl)-1H-imidazole

A solution of 1-methylimidazole (23 mL, 23.7 g, 288 mmol) in THF (700 mL) at −73° C. was treated dropwise with 2.5M n-butyllithium in hexanes (125 mL, 312 mmol), warmed to 0° C., stirred for 30 minutes, cooled to −73° C., treated with chlorotriethylsilane (50 g, 330 mmol), warmed to room temperature, stirred for 16 hours, and concentrated. The concentrate was treated with ethyl acetate and water, the organic layer was washed with brine, dried (Na₂SO₄), filtered, and concentrated. The concentrate was purified by vacuum distillation (0.5-0.6 mmHg, 98-100° C.) with a 6 inch Vigeraux column to provide the desired product.

¹H NMR (300 MHz, CDCl₃) δ 7.19 (s, 1H), 6.96 (s, 1H), 3.75 (s, 3H), 1.00 (m, 9H), 0.93 (m, 6H).

### EXAMPLE 87G

### 5-formyl(1,1′-biphenyl)-2-carbonitrile

The desired product was prepared by substituting Example 87E for Example 5B in Example 5C.

MS (DCI/NH₃) m/z 225 (M+NH₄)⁺; ¹H NMR (300 MHz, CDCl₃) δ 10.12 (s, 1H), 8.02 (m, 1H), 7.95 (m, 2H), 7.60 (m, 2H), 7.54(m,3H).

### EXAMPLE 87H

### 5-(hydroxy(1-methyl-1H-imidazol-5-yl)methyl)(1,1′-biphenyl)-2-carbonitrile

The desired product was prepared by substituting Example 87G for Example 1A in Example 1B.

MS (DCI/NH₃) m/z 290 (M+H)⁺; ¹H NMR (300 MHz, DMSO-d₆) δ 7.95 (d, 1H), 7.63 (s, 1H), 7.55 (s, 1H), 7.55 (m, 7H), 6.46 (s, 1H), 6.18 (d, 1H), 5.95 (d, 1H), 3.58 (s, 3H).

### EXAMPLE 87I

### 5-((benzyloxy)(1-methyl-1H-imidazol-5-yl)methyl)(1,1′-biphenyl)-2-carbonitrile hydrochloride

The desired product was prepared by substituting Example 87H for Example 2B in Example 2C.

MS (APCI(+)) m/z 380 (M+H)⁺; ¹H NMR (300 MHz, DMSO-d₆) δ 9.10 (s, 1H), 8.06 (d, 1H), 7.69 (m, 2H), 7.60 (m, 2H), 7.55 (m, 3H), 7.35 (m, 6H), 6.10 (s, 1H), 4.65 (dd, 1H), 4.55 (dd, 1H), 3.78 (s, 3H).

### EXAMPLE 88

### (2′-methyl-5-((1-methyl-1H-imidazol-5-yl)carbonyl)(1,1′-biphenyl)-2-carbonitrile hydrochloride

A solution of Example 86F (50 mg, 0.16 mmol) in dioxane (2 mL), at 85° C. was treated with MnO₂ (105 mg, 1.2 mmol), stirred for 1 hour, cooled to room temperature, filtered through diatomaceous earth (Celite(®), and concentrated. The concentrate was purified by flash column chromatography on silica gel with chloroform then 98:2/chloroform:methanol to provide the desired product.

MS (DCI/NH₃) m/z 302 (M+H)⁺; ¹H NMR (300 MHz, DMSO-d₆) δ 8.93 (br s, 1H), 8.17 (d, 1H), 8.08 (br s, 1H), 8.00 (dd, 1H), 7.82 (d, 1H), 7.40 (m, 2H), 7.35 (m, 2H), 4.03 (s, 3H), 2.19 (s, 3H).

### EXAMPLE 89

### 4-((benzyloxy)(1-methyl-1H-imidazol-5-yl)methyl)-2-(1-naphthyl)benzonitrile hydrochloride

### EXAMPLE 89A

### ethyl 4-cyano-3-(1-naphthyl)benzoate

The desired product was prepared by substituting Example 87C and 1-naphthylboronic acid for 3-bromo-4-fluorobenzaldehyde and 2-methylphenylboronic acid, respectively, in Example 1A.

MS (DCI/NH₃) m/z 319 (M+NH₄)⁺; ¹H NMR (300 MHz, CDCl₃) δ 8.20 (m, 2H), 7.96 (dd, 2H), 7.90 (d, 1H), 7.55 (m, 2H), 7.47 (m, 3H), 4.43 (q, 2H), 1.39 (t, 3H).

### EXAMPLE 89B

### 4-(hydroxymethyl)-2-(1-naphthyl)benzonitrile

The desired product was prepared by substituting Example 89A for Example 5A in Example 5B.

MS (DCI/NH₃) m/z 277 (M+NH₄)⁺; ¹H NMR (300 MHz, CDCl₃) δ 7.94 (m, 2H), 7.82 (d, 1H), 7.50 (m, 7H), 4.87 (s, 2H).

### EXAMPLE 89C

### 4-formyl-2-(1-naphthyl)benzonitrile

The desired product was prepared by substituting Example 89B for Example 5B in Example 5C.

MS (DCI/NH₃) m/z 275 (M+NH₄)⁺; ¹H NMR (300 MHz, CDCl₃) δ 10.15 (s, 1H), 8.02 (m, 5H), 7.62-7.46 (m, 5H).

### EXAMPLE 89D

### 4-(hydroxy(1-methyl-1H-imidazol-5-yl)methyl)-2-(1-naphthyl)benzonitrile

The desired product was prepared by substituting Example 89C for Example 1A in Example 1B.

¹H NMR (300 MHz, CDCl₃) δ 7.95 (m, 2H), 7.84 (d, 1H), 7.62-7.38 (envelope, 8H), 6.74 and 6.72 (both s, total 1H), 6.02 (s, 1H), 3.61 and 3.59 (both s, total 3H).

### EXAMPLE 89E

### 4-((benzyloxy)(1-methyl-1H-imidazol-5-yl)methyl)-2-(1-naphthyl)benzonitrile hydrochloride

The desired product was prepared by substituting Example 89D for Example 5D in Example 5E.

MS (APCI(+)) m/z 430 (M+H)⁺; ¹H NMR (300 MHz, DMSO-d₆) δ 9.10 (s, 1H), 8.16 (m, 1H), 8.09 (m, 2H), 7.79 (d, 1H), 7.64 (m, 4H), 7.51 (m, 2H), 7.44 (s, 1H), 7.35 (m, 5H), 6.11 (s, 1H), 4.64 (m, 2H), 3.80 and 3.78 (both s, total 3H); Anal. calcd for C₂₉H₂₄ClN₃O·0.95 H₂O: C, 72.10; H, 5.40; N, 8.70. Found: C, 72.17; H, 5.43; N, 8.70.

### EXAMPLE 90

### 4-((benzyloxy)(1-methyl-1H-imidazol-5-yl)methyl)-2-(3-thienyl)benzonitrile hydrochloride

### EXAMPLE 90A

### ethyl 4-cyano-3-(3-thienyl)benzoate

The desired product was prepared by substituting Example 87C and 3-thienylboronic acid for 3-bromo-4-fluorobenzaldehyde and 2-methylphenylboronic acid, respectively, in Example 1A.

MS (DCI/NH₃) m/z 275 (M+N)⁺; ¹H NMR (300 MHz, CDCl₃) δ 8.23 (m, 1H), 8.03 (dd, 1H), 7.80 (d, 1H), 7.73 (m, 1H), 7.47 (m, 3H), 7.46 (m, 2H), 4.43 (q, 2H), 1.42 (t, 3H).

### EXAMPLE 90B

### 4-(hydroxymethyl)-2-(3-thienyl)benzonitrile

The desired product was prepared by substituting Example 90A for Example 5A in Example 5B.

MS (DCI/NH₃) m/z 233 (M+NH₄)⁺.

### EXAMPLE 90C

### 4-formyl-2-(3-thienyl)benzonitrile

The desired product was prepared by substituting Example 90B for Example 5B in Example 5C.

MS (DCI/NH₃) m/z 231 (M+NH₄)⁺.

### EXAMPLE 90D

### 4-(hydroxy(1-methyl-1H-imidazol-5-yl)methyl)-2-(3-thienyl)benzonitrile

The desired product was prepared by substituting Example 90C for Example 1A in Example 1B.

MS (APCI(+)) m/z 296 (M+H)⁺.

### EXAMPLE 90E

### 4-((benzyloxy)(1-methyl-1H-imidazol-5-yl)methyl)-2-(3-thienyl)benzonitrile hydrochloride

The desired product was prepared by substituting Example 90D for Example 5D in Example 5E.

MS (APCI(+)) m/z 386 (M+H)⁺; ¹H NMR (300 MHz, DMSO-d₆) δ 9.10 (s, 1H), 8.02 (d, 1H), 7.95 (m, 1H), 7.77 (m, 2H), 7.62 (dd, 1H), 7.48 (dd, 1H), 7.37 (m, 6H), 6.04 (s, 1H), 4.58 (dd, 2H), 3.78 (s, 3H).

### EXAMPLE 91

### 5-((benzyloxy)(1-methyl-1H-imidazol-5-yl)methyl)-3′-methyl(1,1′-biphenyl)-2-carbonitrile hydrochloride

### EXAMPLE 91A

### ethyl 6-cyano-3′-methyl(1,1′-biphenyl)-3-carboxylate

The desired product was prepared by substituting Example 87C and 3-methylphenylboronic acid for 3-bromo4-fluorobenzaldehyde and 2-methylphenylboronic acid, respectively, in Example 1A.

MS (APCI(+)) m/z 283 (M+NH₄)⁺; ¹H NMR (300 MHz, CDCl₃) δ 8.18 (d, 1H), 8.08 (dd, 1H), 7.82 (d, 1H), 7.40 (m, 3H), 7.30 (m, 1H), 4.43 (q, 2H), 2.45 (s, 3H), 1.42 (t, 3H).

### EXAMPLE 91B

### 5-(hydroxymethyl)-3′-methyl(1,1′-biphenyl)-2-carbonitrile

The desired product was prepared by substituting Example 91A for Example 5A in Example 5B.

MS (DCI/NH₃) m/z 241 (M+NH₄)⁺; ¹H NMR (300 MHz, CDCl₃) δ 7.75 (d, 1H), 7.51 (s, 1H), 7.43 (d, 1H), 7.37 (m, 3H), 7.27 (m, 1H), 4.82 (s, 2H), 2.44 (s, 3H).

### EXAMPLE 91C

### 5-formyl-3′-methyl(1,1′-biphenyl)-2-carbonitrile

The desired product was prepared by substituting Example 91B for Example 5B in Example 5C.

MS (DCI/NH₃) m/z 239 (M+NH₄)⁺; ¹H NMR (300 MHz, CDCl₃) δ 10.12 (s, 1H), 8.00 (m, 1H), 7.92 (m, 2H), 7.40 (m, 3H), 7.30 (m, 1H), 2.46 (s, 3H).

### EXAMPLE 91D

### 5-(hydroxy(1-methyl-1H-imidazol-5-yl)methyl)-3′-methyl(1,1′-biphenyl)-2-carbonitrile

The desired product was prepared by substituting Example 91C for Example 1A in Example 1B.

MS (DCI/NH₃) m/z 304 (M+H)⁺; ¹H NMR (300 MHz, DMSO-d₆) δ 7.94 (d, 1H), 7.62 (s, 1H), 7.57 (m, 2H), 7.42 (m, 1H), 7.38 (m, 2H), 7.30 (m, 1H), 6.45 (s, 1H), 6.20 (d, 1H), 5.95 (d, 1H), 3.58 (s, 3H), 2.40 (s, 3H).

### EXAMPLE 91E

### 5-((benzyloxy)(1-methyl-1H-imidazol-5-yl)methyl)-3′-methyl(1,1′-biphenyl)-2-carbonitrile hydrochloride

The desired product was prepared by substituting Example 91D for Example 5D in Example 5E.

MS (APCI(+)) m/z 394 (M+H)⁺; ¹H NMR (300 MHz, DMSO-d₆) δ 9.10 (s, 1H), 8.07 (m, 1H), 7.68 (m, 2H), 7.38 (m, 10H), 6.09 (s, 1H), 4.60 (dd, 2H), 3.78 (s, 3H), 2.40 (m, 3H); Anal. calcd for C₂₆H₂₃ClN₃O·H₂O: C, 69.71; H, 5.85; N, 9.38. Found: C, 69.75; H, 5.70; N, 9.38.

### EXAMPLE 92

### 4-((benzyloxy)(1-methyl-1H-imidazol-5-yl)methyl)-2-(2-naphthyl)benzonitrile hydrochloride

### EXAMPLE 92A

### ethyl 4-cyano-3-(2-naphthyl)benzoate

The desired product was prepared by substituting Example 87C and 2-naphthylboronic acid for 3-bromo-4-fluorobenzaldehyde and 2-methylphenylboronic acid, respectively, in Example 1A.

MS (DCI/NH₃) m/z 319 (M+NH₄)⁺; ¹H NMR (300 MHz, CDCl₃) δ 8.30 (d, 1H), 8.13 (dd, 1H), 8.07 (d, 1H), 8.00 (d, 1H), 7.95 (m, 2H), 7.90 (d, 1H), 7.70 (dd, 1H), 7.58 (m, 2H), 4.44 (q, 2H), 1.43 (t, 3H).

### EXAMPLE 92B

### 4-(hydroxymethyl)-2-(2-naphthyl)benzonitrile

The desired product was prepared by substituting Example 92A for Example 5A in Example 5B.

MS (DCI/NH₃) m/z 277 (M+N₄)⁺; ¹H NMR (300 MHz, CDCl₃) δ 8.05 (d, 1H), 7.96 (d, 1H), 7.90 (m, 2H), 7.80 (d, 1H), 7.67 (dd, 1H), 7.63 (s, 1H), 7.53 (m, 2H), 7.47 (dd, 1H), 4.85 (d, 2H), 1.88 (t, 1H).

### EXAMPLE 92C

### 4-formyl-2-(2-naphthyl)benzonitrile

The desired product was prepared by substituting Example 92B for Example 5B in Example 5C.

MS (DCI/NH₃) m/z 275 (M+NH₄)⁺; ¹H NMR (300 MHz, CDCl₃) δ 10.18 (s, 1H), 8.14 (s, 1H), 8.09 (s, 1H), 8.00 (m, 3H), 7.94 (m, 2H), 7.70 (dd, 1H), 7.59 (m, 2H).

### EXAMPLE 92D

### 4-(hydroxy(1-methyl-1H-imidazol-5-yl)methyl)-2-(2-naphthyl)benzonitrile

The desired product was prepared by substituting Example 92C for Example 1A in Example 1B.

MS (DCI/NH₃) m/z 340 (M+H₄)⁺; ¹H NMR (300 MHz, CDCl₃) δ 8.13 (s, 1H), 8.08 (d, 1H), 8.02 (m, 2H), 7.98 (d, 1H), 7.77 (s, 1H), 7.70 (dd, 1H), 7.60 (m, 3H), 7.57 (s, 1H), 6.48 (s, 1H), 6.21 (d, 1H), 5.98 (d, 1H), 3.60 (s, 3H).

### EXAMPLE 92E

### 4-((benzyloxy)(1-methyl-1H-imidazol-5-yl)methyl)-2-(2-naphthyl)benzonitrile hydrochloride

The desired product was prepared by substituting Example 92D for Example 5D in Example 5E.

MS (APCI(+)) m/z 430 (M+H)⁺; ¹H NMR (300 MHz, DMSO-d₆) δ 9.12 (s, 1H), 8.15 (m, 2H), 8.11 (m, 2H), 8.03 (m, 2H), 7.81 (s, 1H), 7.73 (m, 2H), 7.62 (m, 2H), 7.38 (m, 5H), 6.12 (s, 1H), 4.62 (dd, 2H), 3.80 (s, 3H); Anal. calcd for C₂₉H₂₄ClN₃O·1.00 H₂O: C, 71.87; H, 5.41; N,8.68. Found: C, 71.87; H, 5.39; N, 8.65.

### EXAMPLE 93

### 5-((benzyloxy)(1-methyl-1H-imidazol-5-yl)methyl)-4′-methyl(1,1′-biphenyl)-2-carbonitrile hydrochloride

### EXAMPLE 93A

### ethyl 4-amino-3-iodobenzoate

A solution of ethyl 4-aminobenzoate (6.0 g, 36 mmol) in dichloromethane (1 10 mL) and methanol (65 mL) at room temperature was treated with calcium carbonate (10.8 g, 108 mmol) and benzyltrimethylammonium dichloroiodate (25 g, 72 mmol), stirred for 16 hours, and filtered. The filtrate was washed with 5% NaHSO₃, dried (Na₂SO₄), filtered, and concentrated to provide a solid. The solid was recrystallized from ethanol and water, treated with diethyl ether, stirred for 30 minutes, and filtered. The filtrate was concentrated to provide the desired product.

MS (DCI/NH₃) m/z 292 (M+H)⁺ and 309 (M+NH₄)⁺; ¹H NMR (300 MHz, CDCl₃) δ 8.33 (d, 1H), 7.82 (dd, 1H), 6.70 (d, 1H), 4.51 (br s, 2H), 4.42 (q, 2H), 1.38 (t, 3H).

### EXAMPLE 93B

### 4-(ethoxycarbonyl)-2-iodobenzenediazonium tetrafluoroborate

The desired product was prepared by substituting Example 93A for Example 87A in Example 87B.

### EXAMPLE 93C

### ethyl 4-cyano-3-iodobenzoate

The desired product was prepared by substituting Example 93B for Example 87B in Example 87C.

¹H NMR (300 MHz, CDCl₃) δ 8.56 (d, 1H), 8.10 (dd, 1H), 6.70 (d, 1H), 4.42 (q, 2H), 1.41 (t, 3H).

### EXAMPLE 93D

### methyl 4-amino-3-iodobenzoate

The desired product was prepared by substituting methyl-4-aminobenzoate for ethyl-4-aminobenzoate in Example 93A.

### EXAMPLE 93E

### 2-iodo-4-(methoxycarbonyl)benzenediazonium tetrafluoroborate

The desired product was prepared by substituting Example 93D for Example 93A in Example 93B.

### EXAMPLE 93F

### ethyl 4-cyano-3-iodobenzoate

The desired product was prepared by substituting Example 93E for Example 93B in Example 93C.

### EXAMPLE 93G

### methyl 6-cyano-4′-methyl(1,1′-biphenyl)-3-carboxylate

The desired product was prepared by substituting Example 93F and 4-methylphenylboronic for 3-bromo-4-fluorobenzaldehyde and 2-methylphenylboronic acid in Example 1A.

MS (DCI/NH₃) m/z 269 (M+N₄)⁺; ¹H NMR (300 MHz, CDCl₃) δ 8.18 (d, 1H), 8.06 (dd, 1H), 7.82 (d, 1H), 7.49 (d, 2H), 7.32 (d, 2H), 3.98 (s, 3H), 2.4 (s, 3H).

### EXAMPLE 93H

### 5-(hydroxymethyl)-4′-methyl(1,1′-biphenyl)-2-carbonitrile

The desired product was prepared by substituting Example 93G for Example 5A in Example 5B.

MS (DCI/NH₃) m/z 241 (M+NH₄)⁺; ¹H NMR (300 MHz, CDCl₃) δ 7.75 (d, 1H), 7.51 (s, 1H), 7.49 (d, 2H), 7.43 (d, 1H), 7.32 (d, 2H), 4.82 (s, 2H), 2.44 (s, 3H).

### EXAMPLE 93I

### 5-formyl-4′methyl(1,1′-biphenyl)-2-carbonitrile

The desired product was prepared by substituting Example 93H for Example 5B in Example 5C.

MS (DCI/NH₃) m/z 239 (M+NH₄)⁺; ¹H NMR (300 MHz, CDCl₃) δ 10.12 (s, 1H), 8.00 (s, 1H), 7.92 (s, 2H), 7.49 (d, 2H), 7.33 (d, 2H), 2.44 (s, 3H).

### EXAMPLE 93J

### 5-(hydroxy( 1-methyl-1H-imidazol-5-yl)methyl)-4′methyl(1,1′-biphenyl)-2-carbonitrile

The desired product was prepared by substituting Example 93I for Example 1A in Example 1B.

MS (DCI/NH₃) m/z 304 (M+H)⁺; ¹H NMR (300 MHz, DMSO-d₆) δ 7.93 (d, 1H), 7.62 (s, 1H), 7.55 (m, 3H), 7.46 (m, 2H), 7.35 (m, 2H), 6.45 (s, 1H), 6.20 (d, 1H), 5.95 (d, 1H), 3.58 (s, 3H), 2.40 (s, 3H).

### EXAMPLE 93K

### 5-((benzyloxy)(1-methyl-1H-imidazol-5-yl)methyl)-4′-methyl(1′-biphenyl)-2-carbonitrile hydrochloride

The desired product was prepared by substituting Example 93J for Example 5D in Example 5E.

MS (APCI(+)) m/z 394 (M+H)⁺; ¹H NMR (300 MHz, DMSO-d₆) δ 9.10 (s, 1H), 8.07 (m, 1H), 7.65 (m, 2H), 7.50 (d, 2H), 7.38 (m, 8H), 6.09 (s, 1H), 4.60 (dd, 2H), 3.78 (s, 3H), 2.40 (m, 3H); Anal. calcd for C₂₆H₂₃ClN₃O·0.80 H₂O: C, 70.28; H, 5.81; N, 9.46. Found: C, 70.21; H, 5.82; N, 9.46.

### EXAMPLE 94

### 5-((benzyloxy)(1-methyl-1H-imidazol-5-yl)methyl)-2′-phenyl(1,1′-biphenyl)-2-carbonitrile hydrochloride

### EXAMPLE 94A

### 2-(dihydroxyboryl)-1,1′-biphenyl

The desired product was prepared by substituting 2-bromobiphenyl for 3-bromo-1,1′-biphenyl in Example 6A.

MS (DCI/NH₃) m/z 216 (M+NH₄)⁺.

### EXAMPLE 94B

### ethyl 6-cyano-2′-phenyl(1,1′-biphenyl)-3-carboxylate

The desired product was prepared by substituting Example 93C and Example 94A for 3-bromo-4-fluorobenzaldehyde and 2-methylphenylboronic acid, respectively, in Example 1A.

MS (DCI/NH₃) m/z 345 (M+NH₄)⁺; ¹H NMR (300 MHz, CDCl₃) δ 7.95 (m, 2H), 7.62 (m, 1H), 7.55-7.40 (m, 4H), 7.20 (m, 2H), 7.10 (m, 2H), 4.35 (q, 2H), 1.38 (t, 3H).

### EXAMPLE 94C

### 5-(hydroxymethyl)-2′-phenyl(1,1′-biphenyl)-2-carbonitrile

The desired product was prepared by substituting Example 94B for Example 5A in Example 5B.

MS (DCI/NH₃) m/z 303 (M+NH₄)⁺; ¹H NMR (300 MHz, CDCl₃) δ 7.57 (d, 1H), 7.50 (m, 2H), 7.43 (m, 2H), 7.30 (m, 1H), 7.19 (m, 3H), 7.10 (m, 3H), 4.63 (s, 2H).

### EXAMPLE 94D

### 5-formyl-2′-phenyl(1,1′-biphenyl)-2-carbonitrile

The desired product was prepared by substituting Example 94C for Example 5B in Example 5C.

MS (DCI/NH₃) m/z 301 (M+NH₄)⁺; ¹H NMR (300 MHz, CDCl₃) δ 9.92 (s, 1H), 7.82 (m, 1H), 7.73 (m, 2H), 7.55-7.40 (m, 4H), 7.20 and 7.10 (both m, total 5H).

### EXAMPLE 94E

### 5-(hydroxy(1-methyl-1H-imidazol-5-yl)methyl)-2′-phenyl(1,1′-biphenyl)-2-carbonitrile

The desired product was prepared by substituting Example 94D for Example 1A in Example 1B.

MS (DCI/NH₃) m/z 366 (M+H)⁺; ¹H NMR (300 MHz, DMSO-d₆) δ 7.76 (d, 1H), 7.60-7.40 (m, 5H), 7.40-6.90 (envelope, 7H), 6.30-6.05 (envelope, 2H), 5.80 (d, 1H).

### EXAMPLE 94F

### 5-((benzyloxy)(1-methyl-1H-imidazol-5-yl)methyl)-2′-phenyl(1,1′-biphenyl)-2-carbonitrile hydrochloride

The desired product was prepared by substituting Example 94E for Example 5D in Example 5E.

MS (APCI(+)) m/z 456 (M+H)⁺; ¹H NMR (300 MHz, DMSO-d₆) δ 9.05 (s, 1H), 7.96 (d, 1H), 7.55 (m, 5H), 7.40-6.90 (envelope, 12H), 5.87 (s, 1H), 4.38 (dd, 2H), 3.50 (s, 3H); Anal. calcd for C₃₁H₂₆CIN₃O·1.00 H₂O: C, 73.00; H, 5.53; N, 8.24. Found: C, 72.97; H, 5.54; N, 8.37.

### EXAMPLE 95

### 5-((benzyloxy)(1-methyl-1H-imidazol-5-yl)methyl)-2′,5′-dimethyl(1,1′-biphenyl)-2-carbonitrile hydrochloride

### EXAMPLE 95A

### 2,5-dimethylphenylboronic acid

The desired product was prepared by substituting 2-bromo-p-xylene for 3-bromo-1,1′-biphenyl in Example 6A.

MS (DCI/NH₃) m/z 168 (M+NH₄)⁺.

### EXAMPLE 95B

### ethyl 6-cyano-2′,5′-dimethyl(1,1′-biphenyl)-3-carboxylate

The desired product was prepared by substituting Example 93C and Example 95A for 3-bromo-4-fluorobenzaldehyde and 2-methylphenylboronic acid, respectively, in Example 1A.

MS (DCI/NH₃) m/z 297 (M+NH₄)⁺; ¹H NMR (300 MHz, CDCl₃) δ 8.10 (dd, 1H), 8.05 (d, 1H), 7.82 (d, 1H), 7.20 (m, 2H), 7.02 (s, 1H), 4.41 (q, 2H), 2.39 (s, 3H), 2.16 (s, 3H), 1.41 (t, 3H).

### EXAMPLE 95C

### 5-(hydroxymethyl)-2′,5′-dimethyl(1,1′-biphenyl)-2-carbonitrile

The desired product was prepared by substituting Example 95B for Example 5A in Example 5B.

MS (DCI/NH₃) m/z 255 (M+NH₄)⁺; ¹H NMR (300 MHz, CDCl₃) δ 7.72 (d, 1H), 7.45 (m, 1H), 7.36 (s, 1H), 7.17 (m, 2H), 7.00 (s, 1H), 4.80 (s, 2H), 2.35 (s, 3H), 2.13 (s, 3H).

### EXAMPLE 95D

### 5-formyl-2′,5′-dimethyl(1,1′-biphenyl)-2-carbonitrile

The desired product was prepared by substituting Example 95C for Example 5B in Example 5C.

MS (DCI/NH₃) m/z 253 (M+NH₄)⁺; ¹H NMR (300 MHz, CDCl₃) δ 10.11 (s, 1H), 7.95 (dd, 1H), 7.91 (d, 1H), 7.86 (m, 1H), 7.20 (m, 2H), 7.00 (s, 1H), 2.38 (s, 3H), 2.14 (s, 3H).

### EXAMPLE 95E

### 5-(hydroxy(1-methyl-1H-imidazol-5-yl)methyl)-2′,5′-dimethyl(1,1′-biphenyl)-2-carbonitrile

The desired product was prepared by substituting Example 95D for Example 1A in Example 1B.

MS (DCI/NH₃) m/z 318 (M+H)⁺; ¹H NMR (300 MHz, DMSO-d₆) δ 7.93 (d, 1H), 7.58 (m, 2H), 7.42 (br s, 1H), 7.20 (m, 2H), 7.02 (br s, 1H), 6.40 (s, 1H), 6.18 (d, 1H), 5.93 (d, 1H), 3.58 (s, 3H), 2.32 (s, 3H), 2.09 (s, 3H).

### EXAMPLE 95F

### 5-((benzyloxy)(1-methyl-1H-imidazol-5-yl)methyl)-2′,5′-dimethyl(1,1′-biphenyl)-2-carbonitrile hydrochloride

The desired product was prepared by substituting Example 95E for Example 5D in Example 5E.

MS (APCI(+)) m/z 408 (M+H)⁺; ¹H NMR (300 MHz, DMSO-d₆) δ 9.09 (s, 1H), 8.04 (d, 1H), 7.67 (dd, 1H), 7.49 (d, 1H),7.38 (m, 6H), 7.22 (m, 2H), 7.07 (br s, 1H), 6.07 (s, 1H), 4.60 (dd, 2H), 3.76 (s, 3H), 2.32 (s, 3H), 2.09 (s, 3H); Anal. calcd for C₂₇H₂₆ClN₃O·0.70 H₂O: C, 71.03; H, 6.05; N, 9.20. Found: C, 71.03; H, 6.20; N, 9.26.

### EXAMPLE 96

### 4-(((4-cyanobenzyl)oxy)(1-methyl-1H-imidazol-5-yl)methyl)-2-(1-naphthyl)benzonitrile hydrochloride

The desired product was prepared by substituting Example 89D and 4-cyanobenzyl bromide for Example 5D and (bromomethyl)benzene, respectively in Example 5E.

MS (APCI(+)) m/z 455 (M+H)⁺; ¹H NMR (300 MHz, DMSO-d₆) δ 9.02 (s, 1H), 8.15 (dd, 1H), 8.09 (m, 2H), 7.80 (m, 3H), 7.65 (m, 2H), 7.58 (m, 5H), 7.45 (m, 2H), 6.15 (s, 1H), 4.73 (m, 2H), 3.79 and 3.77 (both s, total 3H); Anal. calcd for C₃₀H₂₃ClN₄O·1.00 HO: C, 70.79; H, 4.95; N, 11.01. Found: C, 70.99; H, 4.99; N, 10.93.

### EXAMPLE 97

### 4-(((2-methoxy-5-nitrobenzyl)oxy)(1-methyl-1H-imidazol-5-yl)methyl)-2-(1-naphthyl)benzonitrile hydrochloride

The desired product was prepared by substituting Example 89D and 2-methoxy-5-nitrobenzyl bromide for Example 5D and (bromomethyl)benzene, respectively, in Example 5E.

MS (APCI(+)) m/z 505 (M+H)⁺; ¹H NMR (300 MHz, DMSO-d₆) δ 9.08 (s, 1H), 8.25 (m, 2H), 8.15 (dd, 1H), 8.09 (m, 2H), 7.76 (d, 1H), 7.70-7.50 (m, SH), 7.46 (d, 1H), 7.40 (d, 1H), 7.20 (m, 1H), 6.18 and 6.17 (both s, total 1H), 4.70 (m, 2H), 3.82 and 3.80 (both s, total 6H); Anal. calcd for C₃₀H₂₅ClN₄O₄·0.85 H₂O: C, 64.77; H, 4.84; N, 10.07. Found: C, 64.74; H, 4.81; N, 10.01.

### EXAMPLE 98

### 5-((benzyloxy)(1-methyl-1H-imidazol-5-yl)methyl)-2′-ethyl(1,1′-biphenyl)-2-carbonitrile hydrochloride

### EXAMPLE 98A

### 2-ethylphenylboronic acid

The desired product was prepared by substituting 2-bromoethylbenzene for 3-bromo-1,1′-biphenyl in Example 6A.

MS (DCI/NH₃) m/z 168 (M+NH₄)⁺.

### EXAMPLE 98B

### ethyl 6-cyano-2′-ethyl(1,1′-biphenyl)-3-carboxylate

The desired product was prepared by substituting Example 93C and Example 98A for 3-bromo-4-fluorobenzaldehyde and 2-methylphenylboronic acid, respectively, in Example 1A.

MS (DCI/NH₃) m/z 297 (M+NH₄)⁺; ¹H NMR (300 MHz, CDCl₃) δ 8.12 (d, 1H), 8.06 (s, 1H), 7.82 (d, 1H), 7.40 (m, 2H), 7.30 (m, 1H), 7.17 (d, 1H), 4.40 (q, 2H), 2.50 (m, 2H), 1.40 (t, 3H), 1.19 (t, 3H).

### EXAMPLE 98C

### 2′-ethyl-5-(hydroxymethyl)(1,1′-biphenyl)-2-carbonitrile

The desired product was prepared by substituting Example 98B for Example 5A in Example 5B.

MS (DCI/NH₃) m/z 255 (M+NH₄)⁺; ¹H NMR (300 MHz, CDCl₃) δ 7.72 (d, 1H), 7.46 (m, 1H), 7.38 (m, 3H), 7.17 (m, 2H), 4.82 (s, 2H), 2.50 (m, 2H), 1.09 (t, 3H).

### EXAMPLE 98D

### 2′-ethyl-5-formyl(1,1′-biphenyl)-2-carbonitrile

The desired product was prepared by substituting Example 98C for Example 5B in Example 5C.

MS (DCI/NH₃) m/z 253 (M+NH₄)⁺; ¹H NMR (300 MHz, CDCl₃) δ 10.11 (s, 1H), 7.97 (dd, 1H), 7.92 (d, 1H), 7.89 (m, 1H), 7.40 (m, 2H), 7.30 (m, 1H), 7.18 (d, 1H), 2.50 (m, 2H), 1.10 (t, 3H).

### EXAMPLE 98E

### 2′-ethyl-5-(hydroxy(1-methyl-1H-imidazol-5-yl)methyl)(1,1′-biphenyl)-2-carbonitrile

The desired product was prepared by substituting Example 98D for Example 1A in Example 1B.

MS (DCI/NH₃) m/z 318 (M+H)⁺; ¹H NMR (300 MHz, DMSO-d₆) δ 7.93 (d, 1H), 7.57 (m, 2H), 7.40 (m, 3H), 7.30 (m, 1H), 7.20 (m, 1H), 6.40 (m, 1H), 6.19 (m, 1H), 5.93 (d, 1H), 3.55 (s, 3H), 2.40 (m, 2H), 1.00 (m, 3H).

### EXAMPLE 98F

### 5-((benzyloxy)(1-methyl-1H-imidazol-5-yl)methyl)-2′-ethyl(1,1′-biphenyl)-2-carbonitrile hydrochloride

The desired product was prepared by substituting Example 98E for Example 5D in Example 5E.

MS (APCI(+)) m/z 408 (M+H)⁺; ¹H NMR (300 MHz, DMSO-d₆) δ 9.12 (s, 1H), 8.07 (d, 1H), 7.70 (d, 1H), 7.51 (s, 1H), 7.40 (m, 3H), 7.35 (m, 7H), 6.09 (s, 1H), 4.60 (m, 2H), 3.76 (s, 3H), 2.40 (m, 2H), 1.00 (m, 3H); Anal. calcd for C₂₇H₂₆ClN₃O·0.90 H₂O: C, 70.47; H, 6.09; N, 9.130. Found: C, 70.68; H, 5.85; N, 9.24.

### EXAMPLE 99

### 5-((benzyloxy)(1-methyl-1H-imidazol-5-yl)methyl)-2′,3′-dimethyl(1,1′-biphenyl)-2-carbonitrile hydrochloride

### EXAMPLE 99A

### 2,3-dimethylphenylboronic acid

The desired product was prepared by substituting 3-bromoOxylene for 3-bromo-1,1′-biphenyl in Example 6A.

MS (DCI/NH₃) m/z 168 (M+NH₄)⁺.

### EXAMPLE 99B

### methyl 6-cyano-2′,3′-dimethyl(1,1′-biphenyl)-3-carboxylate

The desired product was prepared by substituting Example 93F and Example 99A for 3-bromo-4-fluorobenzaldehyde and 2-methylphenylboronic acid, respectively, in Example 1A.

MS (DCI/NH₃) m/z 283 (M+NH₄)⁺; ¹H NMR (300 MHz, CDCl₃) δ 8.10 (dd, 1H), 8.04 (m, 1H), 7.30 (d, 1H), 7.27 (d, 1H), 7.20 (dd, 1H), 7.04 (d, 1H), 3.96 (s, 3H), 2.09 (s, 3H).

### EXAMPLE 99C

### 5-(hydroxymethyl)-2′,3′-dimethyl(1,1′-biphenyl)-2-carbonitrile

The desired product was prepared by substituting Example 99B for Example 5A in Example 5B.

MS (DCI/NH₃) m/z 255 (M+NH₄); ¹H NMR (300 MHz, CDCl₃) δ 7.72 (d, 1H), 7.45 (m, 1H), 7.36 (s, 1H), 7.23 (d, 1H), 7.17 (dd, 1H), 7.03 (d, 1H), 4.81 (d, 2H), 2.35 (s, 3H), 2.09 (s, 3H), 1.85 (t, 1H).

### EXAMPLE 99D

### 5-formyl-2′,3′-dimethyl(1,1′-biphenyl)-2-carbonitrile

The desired product was prepared by substituting Example 99C for Example 5B in Example 5C.

MS (DCI/NH₃) m/z 253 (M+NH₄)⁺; ¹H NMR (300 MHz, CDCl₃) δ 10.11 (s, 1H), 7.95 (dd, 1H), 7.91 (d, 1H), 7.87 (s, 1H), 7.27 (d, 1H), 7.20 (d, 1H), 7.05 (d, 1H), 2.38 (s, 3H), 2.10 (s, 3H).

### EXAMPLE 99E

### 5-(hydroxy( 1-methyl-1H-imidazol-5-yl)methyl)-2′,3′-dimethyl(1,1′-biphenyl)-2-carbonitrile

The desired product was prepared by substituting Example 99D for Example 1A in Example 1B.

MS (DCI/NH₃) m/z 318 (M+H)⁺; ¹H NMR (300 MHz, DMSO-d₆) δ 7.93 (dd, 1H), 7.56 (m, 2H), 7.40 (d, 1H), 7.27 (d, 1H), 7.20 (m, 1H), 7.06 (m, 1H), 6.41 and 6.40 (both s, total 1H), 6.18 (m, 1H), 5.93 (d, 1H), 3.58 and 3.56 (both s, total 3H), 2.32 and 2.30 (both s, total 3H), 2.03 and 1.97 (both s, total 3H).

### EXAMPLE 99F

### 5-((benzyloxy)(1-methyl-1H-imidazol-5-yl)methyl)-2′,3′-dimethyl(1,1′-biphenyl)-2-carbonitrile hydrochloride

The desired product was prepared by substituting Example 99E for Example 5D in Example 5E.

MS (APCI(+)) 408 m/z (M+H)⁺; ¹H NMR (300 MHz, DMSO-d₆) δ 9.09 (s, 1H), 8.04 (m, 1H), 7.67 (m, 1H), 7.48 (s, 1H), 7.37 (m, 7H), 7.20 (m, 1H), 7.14 and 7.05 (both d, total 1H), 6.07 (s, 1H), 4.60 (m, 2H), 3.76 (s, 3H), 2.32 and 2.30 (both s, total 3H), 2.03 and 1.97 (both s, total 3H); Anal. calcd for C₂₇H₂₆ClN₃O·0.90 H₂O: C, 70.47; H, 6.09; N, 9.13. Found: C, 70.54; H, 5.88; N, 8.86.

### EXAMPLE 100

### 4-((benzyloxy)(1-methyl-1H-imidazol-5-yl)methyl)-2-cyclohexylbenzonitrile hydrochloride

### EXAMPLE 100A

### methyl 4-cyano-3-cyclohexylbenzoate

A mixture of 93F (400 mg, 1.4 mmol) and Pd(PPh₃)₄ (247 mg, 0.2 mmol) was treated with 0.33M cyclohexylzinc bromide in THF (5.5 mL, 1.8 mmol), heated to reflux, stirred for 1 hour, cooled to room temperature, treated with water, diethyl ether, and 2M HCl (3 drops), washed with brine, dried (Na₂SO₄), filtered, and concentrated. The concentrate was purified by flash column chromatography on silica gel with 95:5/hexanes:ethyl acetate to provide the desired product.

MS (DCI/NH₃) m/z 261 (M+NH₄)⁺; ¹H NMR (300 MHz, CDCl₃) δ 8.04 (d, 1H), 7.92 (dd, 1H), 7.69 (d, 1H), 3.96 (s, 3H), 3.01 (m, 1H), 1.90 (m, 4H), 1.80 (m, 1H), 1.50 (m, 4H), 1.30 (m, 1H).

### EXAMPLE 100B

### 2-cyclohexyl-4-(hydroxymethyl)benzonitrile

The desired product was prepared by substituting Example 100A for Example 5A in Example 5B.

MS (DCI/NH₃) m/z 233 (M+NH₄)⁺; ¹H NMR (300 MHz, CDCl₃) δ 7.60 (d, 1H), 7.36 (s, 1H), 7.27 (m, 1H), 4.77 (d, 2H), 3.00 (m, 1H), 1.90 (m, 5H), 1.80 (m, 1H), 1.50 (m, 4H), 1.30 (m, 1H).

### EXAMPLE 100C

### 2-cyclohexyl-4-formylbenzonitrile

The desired product was prepared by substituting Example 100B for Example 5B in Example 5C.

MS (DCI/NH₃) m/z 231 (M+NH₄)⁺; ¹H NMR (300 MHz, CDCl₃) δ 10.08 (s, 1H), 7.88 (s, 1H), 7.78 (s, 2H), 3.00 (m, 1H), 1.90 (m, 4H), 1.80 (m, 1H), 1.50 (m, 4H), 1.30 (m, 1H).

### EXAMPLE 100D

### 2-cyclohexyl-4-(hydroxy(1-methyl-1H-imidazol-5-yl)methyl)benzonitrile

The desired product was prepared by substituting Example 100C for Example 1A in Example 1B.

MS (DCI/NH₃) m/z 296 (M+H)⁺; ¹H NMR (300 MHz, DMSO-d₆) δ 7.75 (d, 1H), 7.55 (m, 2H), 7.39 (d, 1H), 6.33 (s, 1H), 6.11 (d, 1H), 5.87 (d, 1H), 3.58 (s, 3H), 2.86 (m, 1H), 1.80 (m, 5H), 1.40 (m, 5H).

### EXAMPLE 100E

### 4-((benzyloxy)(1-methyl-1H-imidazol-5-yl)methyl)-2-cyclohexylbenzonitrile hydrochloride

The desired product was prepared by substituting Example 100D for Example 5D in Example 5E.

MS (APCI(+)) m/z 386 (M+H)⁺; ¹H NMR (300 MHz, DMSO-d₆) δ 9.10 (s, 1H), 7.90 (d, 1H), 7.59 (s, 1H), 7.48 (dd, 1H), 7.37 (m, 5H), 7.27 (s, 1H), 6.00 (s, 1H), 4.62 (d, 1H), 4.46 (d, 1H), 3.76 (s, 3H), 2.90 (m, 1H), 1.85 (m, 4H), 1.74 (m, 1H), 1.45 (m, 4H), 1.25 (m, 1H); Anal. calcd for C₂₅H₂₈ClN₃O·0.65 H₂O: C, 69.24; H, 6.81; N, 9.69. Found: C, 69.29; H, 6.79; N, 9.79.

### EXAMPLE 101

### 4-((benzyloxy)(1-methyl-1H-imidazol-5-yl)methyl)-2-(5,6,7,8-tetrahydro-1-naphthalenyl)benzonitrile hydrochloride

### EXAMPLE 101A

### 5-bromo-1,2,3,4-tetrahydronaphthalene

A solution of copper(II) bromide (10.4 g, 46.7 mmol) and tert-butyl nitrite (7.0 mL, 6.1 g, 58.5 mmol) in acetonitrile (150 mL) at 65° C., was treated dropwise with a solution of 1-amino-5,6,7,8-tetrahydronaphthalene (6.1 mL, 6.5 g, 44 mmol) in acetonitrile (10 mL), stirred for 10 minutes, cooled to room temperature, treated with 3M HCl, and extracted with diethyl ether. The extract was washed with 3M HCl and brine, dried (Na₂SO₄), filtered, and concentrated. The concentrate was distilled under vacuum (0.3 mm Hg, 77-86° C.) and purified by flash column chromatography on silica gel with hexanes to provide the desired product.

¹H NMR (300 MHz, CDCl₃) δ 7.38 (d, 1H), 7.01 (d, 1H), 6.95 (dd, 1H), 2.75 (m, 4H), 1.80 (m, 4H).

### EXAMPLE 101B

### 5,6,7,8-tetrahydro-1-naphthalenylboronic acid

The desired product was prepared by substituting Example 101A for 3-bromo-1,1′-biphenyl in Example 6A.

MS (DCI/NH₃) m/z 194 (M+NH₄)⁺.

### EXAMPLE 101C

### ethyl 4-cyano-3-(5,6,7,8-tetrahydro-1-naphthalenyl)benzoate

The desired product was prepared by substituting Example 93C and Example 101B for 3-bromo-4-fluorobenzaldehyde and 2-methylphenylboronic acid, respectively, in Example 1A.

MS (DCI/NH₃) m/z 323 (M+NH₄)⁺; ¹H NMR (300 MHz, CDCl₃) δ 8.10 (dd, 1H), 8.03 (d, 1H), 7.80 (d, 1H), 7.20 (m, 2H), 7.00 (m, 1H), 4.41 (q, 2H), 2.86 (m, 2H), 2.42 (m, 2H), 1.80 (m, 4H), 1.40 (t, 3H).

### EXAMPLE 101D

### 4-(hydroxymethyl)-2-(5,6,7,8-tetrahydro-1-naphthalenyl)benzonitrile

The desired product was prepared by substituting Example 101C for Example 5A in Example 5B.

MS (DCI/NH₃) m/z 281 (M+NH₄)⁺; ¹H NMR (300 MHz, CDCl₃) δ 7.73 (d, 1H), 7.44 (d, 1H), 7.38 (s, 1H), 7.16 (m, 2H), 7.00 (m, 1H), 4.81 (d, 2H), 2.88 (m, 2H), 2.45 (m, 2H), 1.80 (m, 5H).

### EXAMPLE 101E

### 4-formyl-2-(5,6,7,8-tetrahydro-1-naphthalenyl)benzonitrile

The desired product was prepared by substituting Example 101D for Example 5B in Example 5C.

MS (DCI/NH₃) m/z 279 (M+NH₄)⁺; ¹H NMR (300 MHz, CDCl₃) δ 10.11 (s, 1H), 7.95 (dd, 1H), 7.91 (d, 1H), 7.87 (s, 1H), 7.20 (m, 2H), 7.00 (m, 1H), 2.88 (m, 2H), 2.45 (m, 2H), 1.80 (m, 4H).

### EXAMPLE 101F

### 4-(hydroxy(1-methyl-1H-imidazol-5-yl)methyl)-2-(5,6,7,8-tetrahydro-1-naphthalenyl)benzonitrile

The desired product was prepared by substituting Example 101E for Example 1A in Example 1B.

MS (DCI/NH₃) m/z 344 (M+H)⁺; ¹H NMR (300 MHz, DMSO-d₆) δ 7.90 (dd, 1H), 7.55 (m, 2H), 7.40 (d, 1H), 7.20 (m, 2H), 7.00 (m, 1H), 6.42 and 6.38 (both s, total 1H), 6.14 (m, 1H), 5.92 (d, 1H), 3.57 and 3.55 (both s, total 3H), 2.80 (m, 2H), 2.35 (m, 2H), 1.70 (m, 4H).

### EXAMPLE 101G

### 4-((benzyloxy)(1-methyl-1H-imidazol-5-yl)methyl)-2-(5,6,7,8-tetrahydro-1-naphthalenyl)benzonitrile hydrochloride

The desired product was prepared by substituting Example 101F for Example 5D in Example 5E.

MS (APCI(+)) m/z 434 (M+H)⁺; ¹H NMR (300 MHz, DMSO-d₆) δ 9.10 (s, 11H), 8.05 (dd, 11H), 7.65 (dd, 11H), 7.48 (s, 11H), 7.37 (m, 6H), 7.20 (m, 2H), 7.10 and 7.00 (both m, total 1H), 6.06 (s, 1H), 4.60 (m, 2H), 3.76 and 3.74 (both s, total 3H), 2.81 (m, 2H), 2.37 (m, 2H), 1.70 (m, 4H); Anal. calcd for C₂₉H₂₈ClN₃·1.20 H₂O: C, 70.85; H, 6.23; N, 8.55. Found: C, 70.90; H, 6.17; N, 8.55.

### EXAMPLE 102

### 4-((benzyloxy)(1-methyl-1H-imidazol-5-yl)methyl)-2-(2-methyl-1-naphthyl)benzonitrile hydrochloride

### EXAMPLE 102A

### 2-methyl-1-naphthylboronic acid

A slurry of Rieke® magnesium (0.5 g, 21 mmol) in THF (10 mL) at room temperature was treated with 1-bromo-2-methylnaphthalene (3.0 mL, 4.2 g, 19 mmol), stirred for 30 minutes, treated with a solution of trimethyl borate (10 mL, 9.1 g, 88 mmol) in diethyl ether (20 mL), stirred for 1 hour, treated sequentially with NaOH and concentrated HCl, and extracted with ethyl acetate. The extract was dried (Na₂SO₄), filtered, and concentrated. The concentrate was triturated with hexanes, and purified by flash column chromatography on silica gel with 4:1/hexanes:ethyl acetate to provide the desired product.

MS (DCI/NH₃) m/z 218 (M+NH₄)⁺.

### EXAMPLE 102B

### ethyl 4-cyano-3-(2-methyl-1-naphthyl)benzoate

The desired product was prepared by the method described in Synlett., 1992, page 207 using Examples 93C and 102A.

MS (DCI/NH₃) m/z 333 (M+NH₄)⁺.

### EXAMPLE 102C

### 4-(hydroxymethyl)-2-(methyl-1-naphthyl)benzonitrile

The desired product was prepared by substituting Example 102B for Example 5A in Example 5B.

### EXAMPLE 102D

### 4-formyl-2-(2-methyl-1-naphthyl)benzonitrile

A solution of Example 102C (45 mg, 0.16 mmol) in dichloromethane (1.7 mL) at room temperature was treated with Dess-Martin periodinane (87 mg, 0.2 mmol), stirred for 45 minutes, washed with saturated. NaHCO₃, dried (Na₂SO₄), filtered, and concentrated. The concentrate was purified by flash column chromatography on silica gel with 88:12/hexanes:ethyl acetate to provide the desired product.

MS (DCI/NH₃) m/z 289 (M+NH₄)⁺.

### EXAMPLE 102E

### 4-(hydroxy(1-methyl-1H-imidazol-5-yl)methyl)-2-(2-methyl-1-naphthyl)benzonitrile

The desired product was prepared by substituting Example 102D for Example 1A in Example 1B.

MS (DCI/NH₃) m/z 354 (M+H)⁺:

### EXAMPLE 102F

### 4-((benzyloxy)(1-methyl-1H-imidazol-5-yl)methyl)-2-(2-methyl-1-naphthyl)benzonitrile hydrochloride

The desired product was prepared by substituting Example 102E for example 5D in Example 5E.

MS (APCI(+)) m/z 444 (M+H)⁺; ¹H NMR (300 MHz, DMSO-d₆) δ 9.09 (s, 1H), 8.20 (d, 1H), 7.98 (d, 2H), 7.80 (m, 1H), 7.50 (m, 3H), 7.36 (m, 7H), 7.20 and 7.13 (both d, total 1H), 6.10 (s, 1H), 4.62 (m, 2H), 3.76 and 3.74 (both s, total 3H), 2.22 and 2.15 (both s, total 3H); Anal. calcd for C₃₀H₂₆ClN₃O·1.50 H₂O: C, 71.07; H, 5.76; N, 8.29. Found: C, 71.09; H, 5.57; N, 8.35.

### EXAMPLE 103

### 2-(1-anthryl)-4-((benzyloxy)(1-methyl-1H-imidazol-5-yl)methyl)benzonitrile hydrochloride

### EXAMPLE 103A

### 1-iodoanthracene

A solution of 1-aminoanthracene (5.0 g, 26 mmol) in acetone (500 mL) was treated with 2M HCl (50 mL), cooled to 3° C., treated dropwise with a solution of sodium nitrite (2.0 g, 29 mmol) in water (25 mL), stirred for 1 hour, treated with urea (10.6 g, 10 mmol) and a solution of KI (7.5 g, 45 mmol) in water (25 mL), stirred for 15 minutes, warmed to room temperature, stirred for 16 hours, heated to 60° C., stirred for 20 minutes, cooled to room temperature, and treated with 2M Na₂SO₃ to provide a precipitate. The precipitate was collected by filtration and dried under vacuum with P₂O₅. The filtrate was partially concentrated and extracted with diethyl ether. The extract was dried (Na₂SO₄), filtered, concentrated, and combined with the precipitate. The mixture was purified by flash column chromatography on silica gel with hexanes to provide the desired product.

MS (DCI/NH₃) m/z 305 (M+H)⁺.

### EXAMPLE 103B

### 1-anthrylboronic acid

The desired product was prepared by substituting Example 103A for 3-bromo-1,1′-biphenyl in Example 6A.

MS (DCI/NH₃) m/z 240 (M+NH₄)⁺.

### EXAMPLE 103C

### ethyl 3-(1-anthryl)-4-cyanobenzoate

The desired product was prepared by substituting Example 93C and Example 103B for 3-bromo-4-fluorobenzaldehyde and 2-methylphenylboronic acid, respectively, in Example 1A.

MS (DCI/NH₃) m/z 369 (M+NH₄)⁺; ¹H NMR (300 MHz, CDCl₃) δ 8.54 (s, 1H), 8.37 (m, 2H), 8.14 (d, 1H), 8.04 (d, 1H), 7.96 (m, 2H), 7.83 (d, 1H), 7.56 (m, 1H), 7.45 (m, 3H), 4.43 and 4.42 (both q, total 2H), 1.39 (t, 3H).

### EXAMPLE 103D

### 2-(1-anthryl)-4-(hydroxymethyl)benzonitrile

The desired product was prepared by substituting Example 103C for Example 5A in Example 5B.

MS (DCI/NH₃) m/z 327 (M+NH₄)⁺; ¹H NMR (300 MHz, CDCl₃) δ 8.51 (s, 1H), 8.10 (d, 1H), 8.03 (m, 2H), 7.85 (m, 2H), 7.60 (m, 2H), 7.55 (m, 2H), 7.45 (m, 2H), 4.89 (s, 2H).

### EXAMPLE 103E

### 2-(1-anthryl)-4-formylbenzonitrile

The desired product was prepared by substituting Example 103D for Example 102C in Example 102D.

MS (DCI/NH₃) m/z 325 (M+NH₄)⁺; ¹H NMR (300 MHz, CDCl₃) δ 10.18 (s, 1H), 8.55 (s, 1H), 8.18-7.97 (m, 5H), 7.83 (d, 1H), 7.57 (m, 1H), 7.47 (m, 3H).

### EXAMPLE 103F

### 2-(1-anthryl)-4-(hydroxy(1-methyl-1H-imidazol-5-yl)methyl)benzonitrile

The desired product was prepared by substituting Example 103E for Example 1A in Example 1B.

MS (DCI/NH₃) m/z 390 (M+H)⁺; ¹H NMR (300 MHz, DMSO-d₆) δ 8.72 (d, 1H), 8.24 (d, 1H), 8.10 (m, 3H), 7.95 (m, 1H), 7.73 and 7.63 (both m, total 3H), 7.52 (m, 4H), 6.57 (s, 1H), 6.22 (m, 1H), 6.03 (d, 1H), 3.54 and 3.52 (both s, total 3H).

### EXAMPLE 103G

### 2-(1-anthryl)-4-((benzyloxy)(1-methyl-1H-imidazol-5-yl)methyl)benzonitrile hydrochloride

The desired product was prepared by substituting Example 103F for Example 5D in Example 5E.

MS (APCI(+)) m/z 480 (M+H)⁺; ¹ H NMR (300 MHz, DMSO-d₆) δ 9.09 (s, 1H), 8.73 (s, 1H), 8.23 and 8.15 (both m, total 4H), 8.02 and 7.92 (both d, total 1H), 7.83 (m 1H), 7.73 (m, 1H), 7.64 and 7.53 (both m, total 5H), 7.37 and 7.30 (both m, total 5H), 6.14 (s, 1H), 4.65 (m, 2H), 3.61 and 3.59 (both s, total 3H); Anal. calcd for C₃₃H₂₆ClN₃O·1.30 H₂O: C, 73.47; H, 5.34; N, 7.79. Found: C, 73.53; H, 5.47; N, 7.79

### EXAMPLE 104

### 4-((benzyloxy)(1-methyl-1H-imidazol-5-yl)methyl)-2-(4-isoquinolinyl)benzonitrile dihydrochloride

### EXAMPLE 104A

### 4-(diethylboryl)isoquinoline

The desired product was prepared by the method described in Heterocycles 1984, Vol.22, p.2471.

MS (DCI/NH₃) m/z 198 (M+H)⁺.

### EXAMPLE 104B

### methyl 4-cyano-3-(4-isoquinolinyl)benzoate

The desired product was prepared by substituting Example 93G, Example 104A, and DMP for Example 3B, 2-methylphenylboronic acid, and DME, respectively, in Example 3C.

MS (DCI/NH₃) m/z 289 (M+H)⁺; ¹H NMR (300 MHz, CDCl₃) δ 9.39 (br s, 1H), 8.53 (br s, 1H), 8.26 (m, 1H), 8.22 (s, 1H), 8.12 (m, 1H), 7.96 (d, 1H), 7.71 (m, 2H), 7.52 (m, 1H), 3.98 (s, 3H).

### EXAMPLE 104C

### 4-(hydroxymethyl)-2-(4-isoquinolinyl)benzonitrile

The desired product was prepared by substituting Example 104B for Example 5A in Example 5B, and by adjusting the aqueous layer to pH >7 with saturated NaHCO₃ prior to extraction with diethyl ether.

MS (DCI/NH₃) m/z 261 (M+H)⁺.

### EXAMPLE 104D

### 4-formyl-2-(4-isoquinolinyl)benzonitrile

The desired product was prepared by substituting Example 104C for Example 102C in Example 102D.

MS (DCI/NH₃) m/z 259 (M+H)⁺.

### EXAMPLE 104E

### 4-(hydroxy(1-methyl-1H-imidazol-5-yl)methyl)-2-(4-isoquinolinyl)benzonitrile

The desired product was prepared by substituting Example 104D for Example 1A in Example 1B.

MS (DCI/NH₃) m/z 341 (M+H)⁺.

### EXAMPLE 104F

### 4-((benzyloxy)(1-methyl-1H-imidazol-5-yl)methyl)-2-(4-isoquinolinyl)benzonitrile dihydrochloride

The desired product was prepared by substituting Example 104E for Example 5D in Example 5E.

MS (APCI(+)) 431 m/z (M+H)⁺; ¹H NMR (300 MHz, DMSO-d₆) δ 9.17 and 9.13 (both s, total 1H), 9.14 (s, 1H), 8.18 and 8.10 (both s, total 1H), 8.40 (m, 1H), 8.23 (m 1H), 7.90 (m, 3H), 7.70 (m, 2H), 7.46 (s, 1H), 7.37 (m, 5H), 6.13 (s, 1H), 4.63 (m, 2H), 3.81 and 3.79 (both s, total 3H).

### EXAMPLE 105

### 4-((benzyloxy)(1-(ethoxymethyl)-1H-imidazol-5-yl)methyl)-2-(1-naphthyl)benzonitrile hydrochloride

### EXAMPLE 105A

### 1-(ethoxymethyl)-1H-imidazole

A solution of imidazole (13 g, 191 mmol) in THF (200 mL) at room temperature was treated with small portions of 60% NaH (7.6 g, 190 mmol), stirred for 30 minutes, treated with THF (100 mL) and chloromethyl ethyl ether (17.5 mL, 17.8 g, 189 mmol), and stirred for 16 hours, filtered through a pad of diatomaceous earth (Celite®) and concentrated. The concentrate was purified by vacuum distillation (5-5.5 mmHg, 96-98° C.) to provide the desired product.

¹H NMR (300 MHz, CDCl₃) δ 7.62 (s, 1H), 7.11 (s, 1H), 7.06 (s, 1H), 5.30 (s, 2H), 3.45 (q, 2H), 1.19 (t, 3H).

### EXAMPLE 105B

### 1-(ethoxymethyl)-2-(triethylsilyl)-1H-imidazole

The desired product was prepared substituting Example 105A for 1-methylimidazole in Example 87F.

¹H NMR (300 MHz, CDCl₃) δ 7.22 (s, 1H), 7.12 (s, 1H), 5.31 (s, 2H), 3.45 (q, 2H), 1.19 (t, 3H), 0.95 (m, 15H).

### EXAMPLE 105C

### 4-((1-(ethoxymethyl)-1H-imidazol-5-yl)(hydroxy)methyl)-2-(1-naphthyl)benzonitrile

The desired product was prepared by substituting Example 105B and Example 89C for Example 87F and Example 1A, respectively, in Example 1B.

MS (DCI/NH₃) m/z 384 (M+H)⁺; ¹H NMR (300 MHz, DMSO-d₆) δ 8.06 (m, 3H), 7.77 (s, 1H), 7.70-7.40 (m, 7H), 6.51 and 6.50 (both s, total 1H), 6.28 (d, 1H), 6.00 (d, 1H), 5.40 (m, 2H), 3.35 (m, 2H), 1.08 and 0.93 (both m, total 3H).

### EXAMPLE 105D

### 4-((benzyloxy)(1-(ethoxymethyl)-1H-imidazol-5-yl)methyl)-2-(1-naphthyl)benzonitrile hydrochloride

The desired product was prepared by substituting Example 105C for Example 5D in Example 5E.

MS (APCI(+)) m/z 474 (M+H)⁺; ¹H NMR (300 MHz, DMSO-d₆) δ 9.11 (br s, 1H), 8.10 (m, 3H), 7.75 (d, 1H), 7.70-7.45 (m, 7H), 7,34 (m, 5H), 6.07 (s, 1H), 5.55 (m, 2H), 4.60 (m, 2H), 3.35 (m, 2H), 0.94 (m, 3H); Anal. calcd for C₃₁H₂₈ClN₃O₂·0.75 H₂O: C, 71.12; H, 5.68; N, 8.03. Found: C, 71.16; H, 5.69; N, 8.08.

### EXAMPLE 106

### 4-(((4-cyanobenzyl)oxy)(1-(ethoxymethyl)-1H-imidazol-5-yl)methyl)-2-(1-naphthyl)benzonitrile hydrochloride

The desired product was prepared by substituting Example 105C and 4-cyanobenzyl bromide for Example 5D and (bromomethyl)benzene, respectively, in Example 5E.

MS (APCI(+)) m/z 499 (M+H)⁺; ¹H NMR (300 MHz, DMSO-d₆) δ 9.10 (br s, 1H), 8.10 (m, 3H), 7.86 (m, 1H), 7.79 (m, 2H), 7.75-7.45 (m, 9H), 6.10 (s, 1H), 5.55 (m, 2H), 4.67 (m, 2H), 3.35 (m, 2H), 0.94 (m, 3H); Anal. calcd for C₃₂H₂₇ClN₄O₂·0.90 H₂O: C, 69.72; H, 5.27; N, 10.16. Found: C, 69.78; H, 5.28; N, 10.01.

### EXAMPLE 107

### 4-(((4-cyanobenzyl)oxy)(1-methyl-1H-imidazol-5-yl)methyl)-2′-phenyl(1,1′-biphenyl)-2-carbonitrile hydrochloride

The desired product was prepared by substituting Example 94E and 4-cyanobenzyl bromide for Example 5D and (bromomethyl)benzene, respectively, in Example 5E.

MS (APCI(+)) m/z 481 (M+H)⁺; ¹H NMR (300 MHz, DMSO-d₆) δ 8.95 (br s, 1H), 7.95 (d, 1H), 7.82 (d, 2H), 7.50 (m, 7H), 7.30-6.90 (br m, 7H), 5.90 (s, 1H), 4.48 (m, 2H), 3.50 (s, 3H); Anal. calcd for C₃₂H₂₅ClN₄O·1.30 H₂O: C, 71.12; H1 5.15; N, 10.37. Found: C, 71.13; H, 4.90; N, 10.35.

### EXAMPLE 108

### 4-(((4-cyanobenzyl)oxy)(1-(3-hydroxypropyl)-1H-imidazol-5-yl)methyl)-2-(1-naphthyl)benzonitrile hydrochloride

### EXAMPLE 108A

### 1-(3-((tert-butyl(dimethyl)silyl)oxy)propyl)-1H-imidazole

The desired product was prepared by substituting (3-chloropropoxy)-tert-butyldimethylsilane for chloromethyl ethyl ether in Example 105A, and purified by flash column chromatography on silica gel with ethyl acetate then 98:2:1/ethyl acetate:ethanol:concentrated ammonium hydroxide.

MS (DCI/NH₃) m/z 241 (M+H)⁺; ¹H NMR (300 MHz, CDCl₃) δ 7.47 (s, 1H), 7.05 (s, 1H), 6.91 (s, 1H), 4.07 (t, 2H), 3.56 (t, 2H), 1.94 (m, 2H), 0.91 (s, 9H), 0.05 (s, 6H).

### EXAMPLE 108B

### 1-(3-((tert-butyl(dimethyl)silyl)oxy)propyl)-2-(triethylsilyl)-1H-imidazole

The desired product was prepared by substituting Example 108A for imidazole in Example 87F.

### EXAMPLE 108C

### 4-((1-(3-((tert-butyl(dimethyl)silyl)oxy)propyl)-1H-imidazol-5-yl)(hydroxy)methyl)-2-(1-naphthyl)benzonitrile

The desired product was prepared by substituting Example 89C and Example 108B for Example 1A and Example 87F, respectively, and by eliminating TBAF in Example 1B.

MS (APCI(+)) m/z 498 (M+H)⁺; ¹H NMR (300 MHz, DMSO-d₆) δ 8.06 (m, 3H), 7.71-7.37 (m, 8H), 6.51 and 6.52 (both s, total 1H), 6.25 (m, 1H), 5.97 (d, 1H), 4.00 (m, 2H), 3.52 (m, 2H), 1.82 (m, 2H), 0.83 and 0.81 (both s, total 9H), 0.05 (m, 6H).

### EXAMPLE 108D

### 4-((1 -(3-((tert-butyl(dimethyl)silyl)oxy)propyl)-1H-imidazol-5-yl)((4-cyanobenzyl)oxy)methyl)-2-(1-naphthyl)benzonitrile

The desired product was prepared by substituting Example 108C and 4-cyanobenzyl bromide for Example 5D and (bromomethyl)benzene, respectively, in Example 5E.

MS (APCI(+)) m/z 613 (M+H)⁺.

### EXAMPLE 108E

### 4-(((4-cyanobenzyl)oxy)(1-(3-hydroxypropyl)-1H-imidazol-5-yl)methyl)-2-(1-naphthyl)benzonitrile hydrochloride

A solution of Example 108C (32 mg, 0.05 mmol) in THF (0.25 mL) at room temperature was treated with 1M tetrabutylammonium fluoride in 95:5/THF:water (0.1 mL), stirred for 2.5 hours, and treated with half-saturated NH₄Cl and ethyl acetate to provide two layers. The organic layer was washed with brine, dried (Na₂SO₄), filtered, and concentrated. The concentrate was purified by flash column chromatography using 97:3:1/ to 96:4:1/ethyl acetate:ethanol:concentrated ammonium hydroxide. The appropriate fractions were concentrated and converted to the HCl salt to provide the desired product.

MS (APCI(+)) m/z 499 (M+H)⁺; ¹H NMR (300 MHz, DMSO-d₆) δ 9.10 (s, 1H), 8.15 (m, 1H), 8.08 (m, 2H), 7.70 (m, 3H), 7.70-7.40 (m, 9H), 6.15 (s, 1H), 4.73 (m, 2H), 4.20 (m, 2H), 3.50 (m, 2H), 1.86 (m, 2H).

### EXAMPLE 109

### 5-(1-hydroxy-1-(1-methyl-1H-imidazol-5-yl)-3-phenyl-2-propynyl)-2′-methyl(1,1′-biphenyl)-2-carbonitrile

### EXAMPLE 109A

### 5-(1-oxo-1-(1-methyl-1H-imidazol-5-yl)-2′-methyl(1,1′-biphenyl)-2-carbonitrile

A solution of Example 4A (400 mg, 1.32 mmol) in dioxane (8 mL) at 45° C. was treated with manganese dioxide (400 mg, 4.6 mmol), refluxed for 5 hours, cooled to room temperature, filtered through a pad of diatomaceous earth (Celite®), and concentrated. The concentrate was purified by flash column chromatography on silica gel with 10:0.6:0.1/ethyl acetate:methanol:concentrated ammonium hydroxide to provide the desired product.

MS (DCI/NH₃) m/z 302 (M+H)⁺ and 319 (M+NH₄)⁺; ¹H NMR (300 MHz, CD₃OD) δ 8.02-7.95 (m, 3H), 7.80 (d, 1H), 7.61 (d, 1H), 7.42-7.25 (m, 4H), 4.03 (s, 3H), 2.21 (s, 3H).

### EXAMPLE 109B

### 5-(1-hydroxy-1-(1-methyl-1H-imidazol-5-yl)-3-phenyl-2-propynyl)-2′-methyl(1,1′-biphenyl)-2-carbonitrile

A solution of phenylacetylene (37 μL, 0.34 mmol) in THF (1 mL) at −78° C. was treated with 1.5M tert-butyllithium in pentane (0.27 mL, 0.34 mmol), stirred for 1 hour, treated with Example 109A (50 mg, 0.17 mmol) in THF (1 mL), stirred for 16 hours while warming to room temperature, treated with water, and extracted with ethyl acetate. The extract was dried (MgSO₄), filtered, and concentrated. The concentrate was purified by flash column chromatography on silica gel with 10:0.6:0.1/ethyl acetate:methanol:concentrated ammonium hydroxide to provide the desired product.

MS (DCI/NH₃) m/z 404 (M+H)⁺; ¹H NMR (300 MHz, CDCl₃) δ 7.81-7.68 (m, 3H), 7.46-7.21 (m, 10H), 6.96 (br s, 1H), 3.60 (s, 3H), 2.17 (s, 3H).

### EXAMPLE 110

### 5-(1-hydroxy-1-(1-methyl-1H-imidazol-5-yl)-3-phenylpropyl)-2′-methyl(1,1′-biphenyl)-2-carbonitrile

A mixture of Example 109B (25 mg, 0.062 mmol), palladium on barium sulfate (20 mg), and potassium hydroxide (20 mg) in methanol (2 mL) was stirred under hydrogen (1 atm) for 16 hours, filtered through a pad of diatomaceous earth (Celite®), and concentrated. The concentrate was purified by flash column chromatography on silica gel with 10:0.6:0.1/ethyl acetate:methanol:concentrated ammonium hydroxide to provide the desired product.

MS (APCI(+)) m/z 408 (M+H)⁺; ¹H NMR (300 MHz, CDCl₃) δ 7.74 (d, 1H), 7.48-7.11 (m, 13H), 3.30 (s, 3H), 2.84 (m, 1H), 2.58-2.52 (m, 2H), 2.35 (m, 1H), 2.13 (s, 3H).

### EXAMPLE 111

### 4-(1-hydroxy- 1-(1-methyl-1H-imidazol-5-yl)-3-phenyl-2-propynyl)-2-(1-naphthyl)benzonitrile

### EXAMPLE 111A

### 4-(1-oxo-1-(1-methyl-1H-imidazol-5-yl))-2-(1 -naphthyl)benzonitrile

The desired product was prepared by substituting Example 89D for Example 4A in Example 109A.

MS (DCI/NH₃) m/z 338 (M+H)⁺ and 355 (M+NH₄)⁺; ¹H NMR (300 MHz, CDCl₃) δ 8.00-7.95 (m, 4H), 7.77-7.46 (m, 8H), 4.13 (s, 3H).

### EXAMPLE 111B

### 4-(1-hydroxy-1-(1-methyl-1H-imidazol-5-yl)-3-phenyl-2-propynyl)-2-(1-naphthyl)benzonitrile

The desired product was prepared by substituting Example 111A for Example 109A in Example 109B.

MS (DCI/NH₃) m/z 440 (M+H)⁺; ¹H NMR (300 MHz, CDCl₃) δ 7.98-7.80 (m, 3H), 7.71-7.26 (m, 13H), 6.98 (d, 1H), 3.64 (d, 3H).

### EXAMPLE 112

### 4-(((4-fluoro-3-(1-naphthyl)phenyl)(1-methyl-1H-imidazol-5-yl)methoxy)methyl)benzonitrile para-toluenesulfonic acid salt

### EXAMPLE 112A

### 4-fluoro-3-(1-naphthyl)benzaldehyde

The desired product was prepared by substituting 2-naphthylboronic acid and tetrakis(triphenylphosphine)palladium(0) for 2-methylphenylboronic acid and palladium(II) acetate, respectively in Example 1A.

MS (DCI/NH₃) m/z 250 (M+H)⁺; ¹H NMR (300 MHz, CDCl₃) δ 10.04 (s, 1H), 8.06-7.9 (m, 4H), 7.59-7.32 (m, 6H).

### EXAMPLE 112B

### (4-fluoro-3-(1-naphthyl)phenyl)(1-methyl-1H-imidazol-5-yl)methanol

The desired product was prepared by substituting Example 112A for Example 1A in Example 1B and chromatographed on silica gel with 10:0.6:0.1/ethyl acetate:methanol:concentrated ammonium hydroxide to provide the desired product.

MS (DCI/NH₃) m/z 333 (M+H)⁺; ¹H NMR (300 MHz, CD₃OD) δ 7.93 (d, 2H), 7.59-7.39 (m, 7H), 7.28 (dd, 1H), 7.00 (dt, 1H), 6.61 (s, 1H), 5.96 (s, 1H), 3.69 (s, 3H).

### EXAMPLE 112C

### 4-(((4-fluoro-3-(1-naphthyl)phenyl)(1-methyl-1H-imidazol-5-yl)methoxy)methyl)benzonitrile para-toluenesulfonic acid salt

The desired product was prepared by substituting Example 112B and 4-(bromomethyl)benzonitrile for Example 5D and (bromomethyl)benzene, respectively, in Example 5E, and chromatographed on silica gel with 10:0.6:0.1/ethyl acetate:methanol:concentrated ammonium hydroxide. The appropriate fractions were concentrated, and the free base was dissolved in ethanol, treated with para-toluenesulfonic acid, and concentrated to provide the desired product.

MS (DCI/NH₃) m/z 448 (M+H)⁺; ¹H NMR (300 MHz, CD₃OD) δ 8.89 (s, 1H), 7.94 (d, 2H), 7.70-7.62 (m, 4H), 7.60-7.46 (m, 6H), 7.39 (t, 2H), 7.27 (m, 1H), 7.17 (d, 2H), 5.95 (s, 1H), 4.77 (s, 2H), 4.73 (m, 1H), 3.86 (s, 3H), 2.33 (s, 3H); Anal. calcd. for C₂₉H₂₂N₃FO·(CH₃)C₆H4SO₃H·H₂O: C, 67.80; H, 5.06; N, 6.59. Found: C, 67.97; H, 5.09; N, 6.47.

### EXAMPLE 113

### 5-(((3-cyanobenzyl)oxy)(1-methyl-1H-imidazol-5-yl)methyl)-2′-methyl(1,1′-biphenyl)-2-carbonitrile hydrochloride

A suspension of silver(I) oxide (45 mg, 0.196 mmol) in dichloromethane(2 mL) at room temperature was treated with Example 86J (20 mg, 0.066 mmol) and 3-(bromomethyl)benzonitrile (15 mg, 0.076 mmol), and stirred for 16 hours, treated with methanol (2 mL), centrifuged, decanted, and concentrated. The concentrate was dissolved in 1:1/DMSO:methanol (1 mL) and purified by preparative HPLC. The concentrate was dissolved in dichloromethane (1 mL), treated with 1M HCl in diethyl ether (1 mL), and concentrated to provide the desired product.

MS (APCI(+)) m/z 419 (M+H)⁺; MS (APCI(−)) m/z 453 (M+Cl)⁻; ¹H NMR (500 MHz, DMSO-d₆) δ 9.04 (s, 1H), 8.05 (d, 1H), 7.85 (s, 1H), 7.79 (dt, 1H), 7.71 (dt, 1H), 7.68 (dd, 1H), 7.57 (t, 1H), 7.52 (d, 1H), 7.42 (s, 1H), 7.40-7.37 (m, 2H), 7.32 (m, 1H), 7.26 (br s, 1H), 6.09 (s, 1H), 4.71 (d, 1H), 4.61 (d, 1H), 3.75 (s, 3H), 2.12 (s, 3H).

### EXAMPLE 114

### 4-(((4-cyanobenzyl)((1-methyl-1H-imidazol-5-yl)methyl)amino)methyl)-2-(8-quinolinyl)benzonitrile

### EXAMPLE 114A

### 4-formyl-2-(8-quinolinyl)benzonitrile

The desired product can be prepared by substituting Example 200A and 8-quinolinylboronic acid for 3-bromo-4-fluorobenzaldehyde and 2-methylphenylboronic acid, respectively in Example 1A.

### EXAMPLE 114B

### 4-(((4-cyanobenzyl)amino)methyl)-2-(8-quinolinyl)benzonitrile

The desired product can be prepared by substituting Example 114A for Example 89C in Example 34B.

### EXAMPLE 114C

### 4-(((4-cyanobenzyl)((1-methyl-1H-imidazol-5-yl)methyl)amino)methyl)-2-(8-quinolinyl)benzonitrile

The desired product can be prepared by substituting Example 114B for Example 34B in Example 34C.

### EXAMPLE 115

### 5-(((4-(tert-butyl)benzyl)oxy)(1-methyl-1H-imidazol-5-yl)methyl)-2′-methyl(1,1′-biphenyl)-2-carbonitrile hydrochloride

The desired product was prepared by substituting 1-(bromomethyl)-4-tert-butylbenzene for 3-(bromomethyl)benzonitrile in Example 113.

MS (APCI(+)) m/z 450 (M+H)⁺; MS (APCI(−)) m/z 484 (M+Cl)⁻; ¹H NMR (500 MHz, DMSO-d₆) δ 9.10 (s, 1H), 8.04 (d, 1H), 7.65 (dd, 1H), 7.50 (d, 1H), 7.40-7.26 (m, 5H), 7.36 (d, 2H), 7.28 (d, 2H), 6.06 (s, 1H), 4.61 (d, 1H), 4.51 (d, 1H), 2.13 (s, 3H), 1.26 (s, 9H).

### EXAMPLE 116

### 5-(((4-cyanobenzyl)oxy)(1-methyl-1H-imidazol-5-yl)methyl)-2′-methyl(1,1′-biphenyl)-2-carbonitrile hydrochloride

The desired product was prepared by substituting 4-(bromomethyl)benzonitrile for 3-(bromomethyl)benzonitrile in Example 113.

MS (APCI(+)) m/z 419 (M+H)⁺; MS (APCI(−)) m/z 453 (M+Cl)⁻; ¹H NMR (500 MHz, DMSO-d₆) δ 9.07 (s, 1H), 8.05 (d, 1H), 7.82 (d, 2H), 7.68 (dd, 1H), 7.57 (d, 2H), 7.52 (d, 1H), 7.44 (s, 1H), 7.42-7.25 (m, 4H), 6.11 (s, 1H), 4.75 (d, 1H), 4.65 (d, 1H), 3.75 (s, 3H), 2.12 (s, 3H).

### EXAMPLE 117

### 5-(((3-iodobenzyl)oxy)(1-methyl-1H-imidazol-5-yl)methyl)-2′-methyl(1,1′-biphenyl)-2-carbonitrile hydrochloride

The desired product was prepared by substituting 1-(bromomethyl)-3-iodobenzene for 3-(bromomethyl)benzonitrile in Example 113.

MS (APCI(+)) m/z 520 (M+H)⁺; MS (APCI(−)) m/z 554 (M+Cl)⁻; ¹H NMR (500 MHz, DMSO-d₆) δ 9.12 (s, 1H), 8.05 (d, 1H), 7.73 (t, 1H), 7.69-7.66 (m, 2H), 7.51 (d, 1H), 7.42 (s, 1H), 7.40-7.26 (m, 5H), 7.16 (t, 1H), 6.08 (s, 1H), 4.63 (d, 1H), 4.53 (d, 1H), 3.76 (s, 3H), 2.13 (s, 3H).

### EXAMPLE 118

### 5-(((4-fluorobenzyl)oxy)(1-methyl-1H-imidazol-5-yl)methyl)-2′-methyl(1,1′-biphenyl)-2-carbonitrile hydrochloride

The desired product was prepared by substituting 1-(bromomethyl)-4-fluorobenzene for 3-(bromomethyl)benzonitrile in Example 113.

MS (APCI(+)) m/z 412 (M+H)⁺; MS (APCI(−)) m/z 446 (M+Cl)⁻; ¹H NMR (500 MHz, DMSO-d₆) δ 9.10 (s, 1H), 8.05 (d, 1H), 7.67 (dd, 1H), 7.50 (d, 1H), 7.43-7.26 (m, 7H), 7.18 (t, 2H), 6.06 (s, 1H), 4.63 (d, 1H), 4.54 (d, 1H), 3.75 (s, 3H), 2.13 (s, 3H).

### EXAMPLE 119

### 5-(((4-bromobenzyl)oxy)(1-methyl-1H-imidazol-5-yl)methyl)-2′-methyl(1,1′-biphenyl)-2-carbonitrile hydrochloride

The desired product was prepared by substituting 1-bromo-4-(bromomethyl)benzene for 3-(bromomethyl)benzonitrile in Example 113.

MS (APCI(+)) m/z 474 (M+H)⁺; MS (APCI(−)) m/z 508 (M+Cl)⁻; ¹H NMR (500 MHz, DMSO-d₆) δ 9.05 (s, 1H), 8.05 (d, 1H), 7.66 (dd, 1H), 7.55 (d, 2H), 7.50 (d, 1H), 7.41-7.26 (m, 5H), 7.33 (d, 2H), 6.06 (s, 1H), 4.62 (d, 1H), 4.52 (d, 1H), 3.74 (s, 3H), 2.12 (s, 3H).

### EXAMPLE 120

### 5-(((3-chlorobenzyl)oxy)(1-methyl-1H-imidazol-5-yl)methyl)-2′-methyl(1,1′-biphenyl)-2-carbonitrile hydrochloride

The desired product was prepared by substituting 1-(bromomethyl)-3-chlorobenzene for 3-(bromomethyl)benzonitrile in Example 113.

MS (APCI(+)) m/z 428 (M+H)⁺; MS (APCI(−)) m/z 462 (M+Cl)⁻; ¹H NMR (500 MHz, DMSO-d₆) δ 9.13 (s, 1H), 8.05 (d, 1H), 7.68 (dd, 1H), 7.52 (d, 1H), 7.43 (br s, 2H), 7.40-7.26 (m, 7H), 6.10 (s, 1H), 4.67 (d, 1H), 4.56 (d, 1H), 3.76 (s, 3H), 2.13 (s, 3H).

### EXAMPLE 121

### (2′-methyl-5-((1-methyl-1H-imidazol-5-yl)((4-nitrobenzyl)oxy methyl)(1,1′-biphenyl)-2-carbonitrile hydrochloride

The desired product was prepared by substituting 1-(bromomethyl)-4-nitrobenzene for 3-(bromomethyl)benzonitrile in Example 113.

MS (APCI(+)) m/z 439 (M+H)⁺; MS (APCI(−)) m/z 473 (M+Cl)⁻; ¹H NMR (500 MHz, DMSO-d₆) δ 9.12 (s, 1H), 8.21 (d, 2H), 8.06 (d, 1H), 7.70 (dd, 1H), 7.66 (d, 2H), 7.53 (d, 1H), 7.47 (s, 1H), 7.42-7.25 (m, 4H), 6.15 (s, 1H), 4.82 (d, 1H), 4.71 (d, 1H), 3.77 (s, 3H), 2.12 (s, 3H).

### EXAMPLE 122

### 5-(((2-methoxy-5-nitrobenzyl)oxy)(1-methyl-1H-imidazol-5-yl)methyl)-2′-methyl(1,1′-biphenyl)-2-carbonitrile hydrochloride

The desired product was prepared by substituting 2-(bromomethyl)-1-methoxy-4-nitrobenzene for 3-(bromomethyl)benzonitrile in Example 113.

MS (APCI(+)) m/z 469 (M+H)⁺; MS (APCI(−)) m/z 503 (M+Cl)⁻; ¹H NMR (500 MHz, DMSO-d₆) δ 9.11 (s, 1H), 8.26-8.24 (m, 2H), 8.06 (d, 1H), 7.66 (dd, 1H), 7.55 (d, 1H), 7.42-7.20 (m, 6H), 6.14 (s, 1H), 4.72 (d, 1H), 4.63 (d, 1H), 3.86 (s, 3H), 3.79 (s, 3H), 2.13 (s, 3H).

### EXAMPLE 123

### (2′-methyl-5-((1-methyl-1H-imidazol-5-yl)((3-(trifluoromethyl)benzyl)oxy)methyl)(1,1′-biphenyl)-2-carbonitrile hydrochloride

The desired product was prepared by substituting 1-(bromomethyl)-3-(trifluoromethyl)benzene for 3-(bromomethyl)benzonitrile in Example 113.

MS (APCI(+)) m/z 462 (M+H)⁺; MS (APCI(−)) m/z 496 (M+Cl)⁻; ¹H NMR (500 MHz, DMSO-d₆) δ 8.93 (s, 1H), 8.05 (d, 1H), 7.70-7.66 (m, 4H), 7.60 (t, 11H), 7.51 (d, 1H), 7.42-7.25 (m, 5H), 6.09 (s, 1H), 4.76 (d, 1H), 4.66 (d, 1H), 3.74 (s, 3H), 2.12 (s, 3H).

### EXAMPLE 124

### 5-(((2,6-dichlorobenzyl)oxy)(1-methyl-1H-imidazol-5-yl)methyl)-2′-methyl(1,1′-biphenyl)-2-carbonitrile hydrochloride

The desired product was prepared by substituting 2-(bromomethyl)-1,3-dichlorobenzene for 3-(bromomethyl)benzonitrile in Example 113.

MS (APCI(+)) m/z 462 (M+H)⁺; MS (APCI(−)) m/z 496 (M+Cl)⁻; ¹H NMR (500 MHz, DMSO-d₆) δ 9.00 (s, 1H), 8.06 (d, 1H), 7.69 (dd, 1H), 7.56 (br s, 1H), 7.50 (d, 1H), 7.49-7.25 (m, 7H), 6.14 (s, 1H), 4.85 (d, 1H), 4.73 (d, 1H), 3.81 (s, 3H), 2.14 (s, 3H).

### EXAMPLE 125

### 5-(((3,4-dichlorobenzyl)oxy)(1-methyl-1H-imidazol-5-yl)methyl)-2′-methyl(1,1′-biphenyl)-2-carbonitrile hydrochloride

The desired product was prepared by substituting 4-(bromomethyl)-1,2-dichlorobenzene for 3-(bromomethyl)benzonitrile in Example 113.

MS (APCI(+)) m/z 462 (M+H)⁺; MS (APCI(−)) m/z 469 (M+Cl)⁻; ¹H NMR (500 MHz, DMSO-d₆) δ 8.77 (s, 1H), 8.05 (d, 1H), 7.65 (dd, 1H), 7.62-7.60 (m, 3H), 7.48 (d, 1H), 7.42-7.25 (m, 5H), 6.03 (s, 1H), 4.64 (d, 1H), 4.55 (d, 1H), 3.70 (s, 3H), 2.12 (s, 3H).

### EXAMPLE 126

### 5-(((2-cyanobenzyl)oxy)(1-methyl-1H-imidazol-5-yl)methyl)-2′-methyl(1,1′-biphenyl)-2-carbonitrile hydrochloride

The desired product was prepared by substituting 2-(bromomethyl)benzonitrile for 3-(bromomethyl)benzonitrile in Example 113.

MS (APCI(+)) m/z 419 (M+H)⁺; MS (APCI(−)) m/z 453 (M+Cl)⁻; ¹H NMR (500 MHz, DMSO-d₆) δ 8.87 (s, 1H), 8.04 (d, 1H), 7.85 (dd, 1H), 7.72 (td, 1H), 7.68-7.65 (m, 2H), 7.55 (dd, 1H), 7.52 (d, 1H), 7.42-7.25 (m, 5H), 6.13 (s, 1H), 4.81 (d, 1H), 4.71 (d, 1H), 3.72 (s, 3H), 2.12 (s, 3H).

### EXAMPLE 127

### (2′-methyl-5-(((4-methylbenzyl)oxy)(1-methyl-1H-imidazol-5-yl)methyl)(1,1′-biphenyl)-2-carbonitrile hydrochloride

The desired product was prepared by substituting 1-(bromomethyl)-4-methylbenzene for 3-(bromomethyl)benzonitrile in Example 113.

MS (APCI(+)) m/z 408 (M+H)⁺; MS (APCI(−)) m/z 442 (M+Cl)⁻; ¹H NMR (500 MHz, DMSO-d₆) δ 8.87 (s, 1H), 8.04 (d, 1H), 7.65 (dd, 1H), 7.48 (d, 1H), 7.40-7.20 (m, 5H), 7.23 (d, 2H), 7.16 (d, 2H), 6.00 (s, 1H), 4.57 (d, 1H), 4.49 (d, 1H), 3.71 (s, 3H), 2.29 (s, 3H), 2.12 (s, 3H).

### EXAMPLE 128

### (2′-methyl-5-(((3-methylbenzyl)oxy)(1-methyl-1H-imidazol-5-yl)methyl)(1,1′-biphenyl)-2-carbonitrile hydrochloride

The desired product was prepared by substituting 1-(bromomethyl)-3-methylbenzene for 3-(bromomethyl)benzonitrile in Example 113.

MS (APCI(+)) m/z 408 (M+H)⁺; MS (APCI(−)) m/z 442 (M+Cl)⁻; ¹H NMR (500 MHz, DMSO-d₆) δ 8.91 (s, 1H), 8.10 (d, 1H), 7.72 (dd, 1H), 7.55 (d, 1H), 7.45-7.17 (m, 9H), 6.07 (s, 1H), 4.64 (d, 1H), 4.55 (d, 1H), 3.77 (s, 3H), 2.34 (s, 3H), 2.18 (s, 3H).

### EXAMPLE 129

### 5-(((2,5-difluorobenzyl)oxy)(1-methyl-1H-imidazol-5-yl)methyl)-2′-methyl(1,1′-biphenyl)-2-carbonitrile hydrochloride

The desired product was prepared by substituting 2-(bromomethyl)-1,4-difluorobenzene for 3-(bromomethyl)benzonitrile in Example 113.

MS (APCI(+)) m/z 430 (M+H)⁺; MS (APCI(−)) m/z 464 (M+Cl)⁻; ¹H NMR (500 MHz, DMSO-d₆) δ 8.83 (s, 1H), 8.04 (d, 1H), 7.65 (dd, 1H), 7.50 (d, 1H), 7.40-7.21 (m, 8H), 6.07 (s, 1H), 4.67 (d, 1H), 4.59 (d, 1H), 3.72 (s, 3H), 2.12 (s, 3H).

### EXAMPLE 130

### methyl 4-(((6-cyano-2′-methyl(1,1′-biphenyl)-3-yl)(1-methyl-1H-imidazol-5-yl)methoxy)methyl)benzoate hydrochloride

The desired product was prepared by substituting methyl 4-(bromomethyl)benzoate for 3-(bromomethyl)benzonitrile in Example 113.

MS (APCI(+)) m/z 452 (M+H)⁺; MS (APCI(−)) m/z 486 (M+Cl)⁻; ¹H NMR (500 MHz, DMSO-d₆) δ 8.78 (s, 1H), 8.04 (d, 1H), 7.94 (d, 2H), 7.67 (dd, 1H), 7.51 (d, 2H), 7.50 (d, 1H), 7.41-7.28 (m, 4H), 7.25 (s, 1H), 6.06 (s, 1H), 4.73 (d, 1H), 4.63 (d, 1H), 3.85 (s, 3H), 3.71 (s, 3H), 2.12 (s, 3H).

### EXAMPLE 131

### 5-(((3,5-difluorobenzyl)oxy)(1-methyl-1H-imidazol-5-yl)methyl)-2-methyl(1,1′-biphenyl)-2-carbonitrile hydrochloride

The desired product was prepared by substituting 1-(bromomethyl)-3,5-difluorobenzene for 3-(bromomethyl)benzonitrile in Example 113.

MS (APCI(+)) m/z 430 (M+H)⁺; MS (APCI(−)) m/z 464 (M+Cl)⁻; ¹H NMR (500 MHz, DMSO-d₆) δ 9.04 (s, 1H), 8.18 (d, 1H), 7.81 (dd, 1H), 7.65 (d, 1H), 7.54-7.22 (m, 8H), 6.20 (s, 1H), 4.81 (d, 1H), 4.71 (d, 1H), 3.86 (s, 3H), 2.26 (s, 3H).

### EXAMPLE 132

### 5-(((2-chlorobenzyl)oxy)(1-methyl-1H-imidazol-5-yl)methyl)-2′-methyl(1,1′-biphenyl)-2-carbonitrile hydrochloride

The desired product was prepared by substituting 1-(bromomethyl)-2-chlorobenzene for 3-(bromomethyl)benzonitrile in Example 113.

MS (APCI(+)) m/z 428 (M+H)⁺; MS (APCI(−)) m/z 462 (M+Cl)⁻; ¹H NMR (500 MHz, DMSO-d₆) δ 8.90 (s, 1H), 8.15 (d, 1H), 7.78 (dd, 1H), 7.67-7.34 (m, 10H), 6.21 (s, 1H), 4.83 (d, 1H), 4.73 (d, 1H), 3.83 (s, 3H), 2.23 (s, 3H).

### EXAMPLE 133

### 5-(((4-chlorobenzyl)oxy)(1-methyl-1H-imidazol-5-yl)methyl)-2′-methyl(1,1′-biphenyl)-2-carbonitrile hydrochloride

The desired product was prepared by substituting 1-(bromomethyl)-4-chlorobenzene for 3-(bromomethyl)benzonitrile in Example 113.

MS (APCI(+)) m/z 428 (M+H)⁺; MS (APCI(−)) m/z 462 (M+Cl)⁻; ¹H NMR (500 MHz, DMSO-d₆) δ 8.78 (s, 1H), 8.04 (d, 1H), 7.65 (dd, 1H), 7.48 (d, 1H), 7.44-7.22 (m, 9H), 6.02 (s, 1H), 4.62 (d, 1H), 4.53 (d, 1H), 3.70 (s, 3H), 2.12 (s, 3H).

### EXAMPLE 134

### 5-(((3-methoxybenzyl)oxy)(1-methyl-1H-imidazol-5-yl)methyl)-2′-methyl(1,1′-biphenyl)-2-carbonitrile hydrochloride

The desired product was prepared by substituting 1-(bromomethyl)-3-methoxybenzene for 3-(bromomethyl)benzonitrile in Example 113.

MS (APCI(+)) m/z 424 (M+H)⁺; MS (APCI(−)) m/z 458 (M+Cl)⁻; ¹H NMR (500 MHz, DMSO-d₆) δ 9.03 (s, 1H), 8.05 (d, 1H), 7.67 (dd, 1H), 7.51 (d, 1H), 7.40-7.22 (m, 5H), 6.94-6.85 (m, 4H), 6.05 (s, 1H), 4.61 (d, 1H), 4.52 (d, 1H), 3.75 (s, 3H), 3.72 (s, 3H), 2.13 (s, 3H).

### EXAMPLE 135

### (2′-methyl-5-(((2-methylbenzyl)oxy)(1-methyl-1H-imidazol-5-yl)methyl)(1,1′-biphenyl)-2-carbonitrile hydrochloride

The desired product was prepared by substituting 1-(bromomethyl)-2-methylbenzene for 3-(bromomethyl)benzonitrile in Example 113.

MS (APCI(+)) m/z 408 (M+H)⁺; MS (APCI(−)) m/z 442 (M+Cl)⁻; ¹H NMR (500 MHz, DMSO-d₆) δ 8.98 (s, 1H), 8.05 (d, 1H), 7.66 (dd, 1H), 7.51 (d, 1H), 7.42-7.15 (m, 9H), 6.06 (s, 1H), 4.64 (d, 1H), 4.56 (d, 1H), 3.71 (s, 3H), 2.24 (s, 3H), 2.12 (s, 3H).

### EXAMPLE 136

### 5-(((3-fluorobenzyl)oxy)(1-methyl-1H-imidazol-5-yl)methyl)-2′-methyl(1,1′-biphenyl)-2-carbonitrile hydrochloride

The desired product was prepared by substituting 1-(bromomethyl)-3-fluorobenzene for 3-(bromomethyl)benzonitrile in Example 113.

MS (APCI(+)) m/z 412 (M+H)⁺; MS (APCI(−)) m/z 446 (M+Cl)⁻; ¹H NMR (500 MHz, DMSO-d₆) δ 8.89 (s, 1H), 8.05 (d, 1H), 7.67 (dd, 1H), 7.51 (d, 1H), 7.42-7.12 (m, 9H), 6.05 (s, 1H), 4.66 (d, 1H), 4.56 (d, 1H), 3.72 (s, 3H), 2.12 (s, 3H).

### EXAMPLE 137

### 5-(((2,6-dichloro-4-pyridinyl)methoxy)(1-methyl-1H-imidazol-5-yl)methyl)-2′-methyl(1,1′-biphenyl)-2-carbonitrile hydrochloride

The desired product was prepared by substituting 4-(bromomethyl)-2,6-dichloropyridine for 3-(bromomethyl)benzonitrile in Example 113.

MS (APCI(+)) m/z 463 (M+H)⁺; MS (APCI(−)) m/z 497 (M+Cl)⁻; ¹H NMR (500 MHz, DMSO-d₆) δ 8.94 (s, 1H), 8.05 (d, 1H), 7.69 (dd, 1H), 7.57-7.54 (m, 3H), 7.42-7.25 (m, 5H), 6.10 (s, 1H), 4.74 (d, 1H), 4.63 (d, 1H), 3.73 (s, 3H), 2.12 (s, 3H).

### EXAMPLE 138

### 5-(((2-fluorobenzyl)oxy)(1-methyl-1H-imidazol-5-yl)methyl)-2′-methyl(1,1′-biphenyl)-2-carbonitrile hydrochloride

The desired product was prepared by substituting 1-(bromomethyl)-2-fluorobenzene for 3-(bromomethyl)benzonitrile in Example 113.

MS (APCI(+)) m/z 412 (M+H)⁺; MS (APCI(−)) m/z 446 (M+Cl)⁻; ¹H NMR (500 MHz, DMSO-d₆) δ 8.85 (s, 1H), 8.04 (d, 1H), 7.71-7.49 (m, 9H), 7.65 (dd, 1H), 7.50 (d, 1H), 6.06 (s, 1H), 4.68 (d, 1H), 4.60 (d, 1H), 3.72 (s, 3H), 2.13 (s, 3H).

### EXAMPLE 139

### (2′-methyl-5-((1-methyl-1H-imidazol-5-yl)((4-(trifluoromethyl)benzyl)oxy)methyl)(1,1′-biphenyl)-2-carbonitrile hydrochloride

The desired product was prepared by substituting 1-(bromomethyl)-4-(trifluoromethyl)benzene for 3-(bromomethyl)benzonitrile in Example 113.

MS (APCI(+)) m/z 462 (M+H)⁺; MS (APCI(−)) m/z 496 (M+Cl)⁻; ¹H NMR (500 MHz, DMSO-d₆) δ 8.88 (s, 1H), 8.05 (d, 1H), 7.72 (d, 2H), 7.67 (dd, 1H), 7.59 (d, 2H), 7.51 (d, 1H), 7.41-7.25 (m, 5H), 6.08 (s, 1H), 4.75 (d, 1H), 4.64 (d, 1H), 3.73 (s, 3H), 2.12 (s, 3H).

### EXAMPLE 140

### 5-(((3,5-dimethylbenzyl)oxy)(1-methyl-1H-imidazol-5-yl)methyl)-2′-methyl(1,1′-biphenyl)-2-carbonitrile hydrochloride

The desired product was prepared by substituting 1-(bromomethyl)-3,5-dimethylbenzene for 3-(bromomethyl)benzonitrile in Example 113.

MS (APCI(+)) m/z 422 (M+H)⁺; MS (APCI(−)) m/z 456 (M+Cl)⁻; ¹H NMR (500 MHz, DMSO-d₆) δ 8.86 (s, 1H), 8.05 (d, 1H), 7.65 (dd, 1H), 7.49 (d, 1H), 7.41-7.25 (m, 5H), 6.93 (s, 3H), 6.00 (s, 1H), 4.55 (d, 1H), 4.45 (d, 1H), 3.72 (s, 3H), 2.24 (s, 6H), 2.13 (s, 3H).

### EXAMPLE 141

### 5-(((4-fluoro-2-(trifluoromethyl)benzyl)oxy)(1-methyl-1H-imidazol-5-yl)methyl)-2′-methyl(1,1′-biphenyl)-2-carbonitrile hydrochloride

The desired product was prepared by substituting 1-(bromomethyl)-4-fluoro-2-(trifluoromethyl)benzene for 3-(bromomethyl)benzonitrile in Example 113.

MS (APCI(+)) m/z 480 (M+H)⁺; MS (APCI(−)) m/z 514 (M+Cl)⁻; ¹H NMR (500 MHz, DMSO-d₆) δ 8.73 (s, 1H), 7.86 (d, 1H), 7.59-7.42 (m, 1H), 7.47-7.28 (m, 4H), 7.22-7.03 (m, 5H), 5.97 (s, 1H), 4.59 (d, 1H), 4.47 (d, 1H), 3.53 (s, 3H), 1.91 (s, 3H).

### EXAMPLE 142

### (2′-methyl-5-((1-methyl-1H-imidazol-5-yl)((2-nitrobenzyl)oxy)methyl)(1,1′-biphenyl)-2-carbonitrile hydrochloride

The desired product was prepared by substituting 1-(bromomethyl)-2-nitrobenzene for 3-(bromomethyl)benzonitrile in Example 113.

MS (APCI(+)) m/z 439 (M+H)⁺; MS (APCI(−)) m/z 473 (M+Cl)⁻; ¹H NMR (500 MHz, DMSO-d₆) δ 8.12 (dd, 1H), 8.10 (d, 1H), 8.01 (s, 1H), 7.86 (dd, 1H), 7.83 (dd, 1H), 7.71 (dd, 1H), 7.67 (dd, 1H), 7.57 (d, 1H), 7.48 (s, 1H), 7.46-7.31 (m, 4H), 6.22 (s, 1H), 4.95 (d, 1H), 4.07 (d, 1H), 3.76 (s, 3H), 2.18 (s, 3H).

### EXAMPLE 143

### (2′-methyl-5-((1-methyl-1H-imidazol-5-yl)((3-(trifluoromethoxy)benzyl)oxy)methyl)(1,1′-biphenyl)-2-carbonitrile hydrochloride

The desired product was prepared by substituting 1-(bromomethyl)-3-(trifluoromethoxy)benzene for 3-(bromomethyl)benzonitrile in Example 113.

MS (APCI(+)) m/z 478 (M+H)⁺; MS (APCI(−)) m/z 512 (M+Cl)⁻; ¹H NMR (500 MHz, DMSO-d₆) δ 8.99 (s, 1H), 8.05 (d, 1H), 7.67 (dd, 1H), 7.52 (d, 1H), 7.50 (t, 1H), 7.41-7.25 (m, 8H), 6.08 (s, 1H), 4.71 (d, 1H), 4.61 (d, 1H), 3.74 (s, 3H), 2.12 (s, 3H).

### EXAMPLE 144

### 4-(((6-cyano-2′-methyl(1,1′-biphenyl)-3-yl)(1-methyl-1H-imidazol-5-yl)methoxy)methyl)-6-methylisophthalonitrile hydrochloride

The desired product was prepared by substituting 4-(bromomethyl)-6-methylisophthalonitrile for 3-(bromomethyl)benzonitrile in Example 113.

MS (APCI(+)) m/z 458 (M+H)⁺; MS (APCI(−)) m/z 492 (M+Cl)⁻; ¹H NMR (500 MHz, DMSO-d₆) δ 8.89 (s, 1H), 8.40 (s, 1H), 8.05 (d, 1H), 7.75 (s, 1H), 7.67 (dd, 1H), 7.52 (d, 1H), 7.41-7.25 (m, 5H), 6.16 (s, 1H), 4.86 (d, 1H), 4.74 (d, 1H), 3.72 (s, 3H), 2.55 (s, 3H), 2.12 (s, 3H).

### EXAMPLE 145

### 5-(((2′-cyano(1,1′-biphenyl)-4-yl)methoxy)(1-methyl-1H-imidazol-5-yl)methyl)-2′-methyl(1,1′-biphenyl)-2-carbonitrile hydrochloride

The desired product was prepared by substituting 4′-(bromomethyl)(1,1′-biphenyl)-2-carbonitrile for 3-(bromomethyl)benzonitrile in Example 113.

MS (APCI(+)) m/z 495 (M+H)⁺; MS (APCI(−)) m/z 529 (M+Cl)⁻; ¹H NMR (500 MHz, DMSO-d₆) δ 8.99 (s, 1H), 8.07 (d, 1H), 7.95 (dd, 1H), 7.80 (dd, 1H), 7.72 (dd, 1H), 7.62-7.53 (m, 7H), 7.41-7.25 (m, 5H), 6.12 (s, 1H), 4.73 (d, 1H), 4.64 (d, 1H), 3.77 (s, 3H), 2.14 (s, 3H).

### EXAMPLE 146

### methyl 3-(((6-cyano-2′-methyl(1,1′-biphenyl)-3-yl)(1-methyl-1H-imidazol-5-yl)methoxy)methyl)benzoate hydrochloride

The desired product was prepared by substituting methyl 3-(bromomethyl)benzoate for 3-(bromomethyl)benzonitrile in Example 113.

MS (APCI(+)) m/z 452 (M+H)⁺; MS (APCI(−)) m/z 486 (M+Cl)⁻; ¹H NMR (500 MHz, DMSO-d₆) δ 8.99 (s, 1H), 8.09 (d, 1H), 7.96-7.94 (m, 2H), 7.71-7.69 (m, 2H), 7.58-7.50 (m, 2H), 7.41-7.25 (m, 5H), 6.12 (s, 1H), 4.78 (d, 1H), 4.68 (d, 1H), 3.89 (s, 3H), 3.78 (s, 3H), 2.17 (s, 3H).

### EXAMPLE 147

### 5-(((3,4-difluorobenzyl)oxy)(1-methyl-1H-imidazol-5-yl)methyl)-2′-methyl(1,1′-biphenyl)-2-carbonitrile hydrochloride

The desired product was prepared by substituting 4-(bromomethyl)-1,2-difluorobenzene for 3-(bromomethyl)benzonitrile in Example 113.

MS (APCI(+)) m/z 430 (M+H)⁺; MS (APCI(−)) m/z 464 (M+Cl)⁻; ¹H NMR (500 MHz, DMSO-d₆) δ 8.95 (s, 1H), 8.05 (d, 1H), 7.67 (dd, 1H), 7.50 (d, 1H), 7.48-7.22 (m, 8H), 6.05 (s, 1H), 4.62 (d, 1H), 4.54 (d, 1H), 3.73 (s, 3H), 2.12 (s, 3H).

### EXAMPLE 148

### (2′-methyl-5-((1-methyl-1H-imidazol-5-yl)((3,4,5-trimethoxybenzyl)oxy)methyl)(1,1′-biphenyl)-2-carbonitrile hydrochloride

A solution of 5-(chloromethyl)-1,2,3-trimethoxybenzene (20 mg, 0.092 mmol) in acetone (3 mL) at 60° C. was treated with KI (166 mg, mmol), stirred for 16 hours, centrifuged, decanted, and concentrated. The concentrate was dissolved in dichloromethane (2 mL), treated with Example 86J (20 mg, 0.066 mmol) and silver(I) oxide (140 mg, 0.604 mmol), stirred for 16 hours, treated with methanol, centrifuged, decanted, and concentrated. The concentrate was treated with 1:1/:methanol/DMSO, purified by preparative HPLC, dissolved in dichloromethane, treated with 1M HCl in diethyl ether, and concentrated to provide the desired product.

MS (APCI(+)) m/z 484 (M+H)⁺; MS (APCI(−)) m/z 518 (M+Cl)⁻; ¹H NMR (500 MHz, DMSO-d₆) δ 8.94 (s, 1H), 8.04 (d, 1H), 7.65 (dd, 1H), 7.51 (d, 1H), 7.41-7.25 (m, 5H), 6.64 (s, 2H), 6.02 (s, 1H), 4.56 (d, 1H), 4.49 (d, 1H), 3.74 (s, 3H), 3.72 (s, 6H), 3.63 (s, 3H), 2.13 (s, 3H).

### EXAMPLE 149

### (2′-methyl-5-((1-methyl-1H-imidazol-5-yl)(8-quinolinylmethoxy)methyl)(1,1′-biphenyl)-2-carbonitrile hydrochloride

The desired product was prepared by substituting 8-(chloromethyl)quinoline for 5-(chloromethyl)-1,2,3-trimethoxybenzene in Example 148.

MS (APCI(+)) m/z 445 (M+H)⁺; MS (APCI(−)) m/z 479 (M+Cl)⁻; ¹H NMR (500 MHz, DMSO-d₆) δ 8.38 (d, 1H), 8.04 (dd, 1H), 7.98 (d, 1H), 7.93 (d, 1H), 7.76 (dd, 1H), 7.71 (br d, 1H), 7.64 (d, 1H), 7.61 (t, 1H), 7.56 (br s, 1H), 7.41-7.22 (m, 6H), 6.31 (br s, 1H), 4.90 (br d, 1H), 4.82 (br d, 1H), 3.76 (br s, 3H), 2.11 (s, 3H).

### EXAMPLE 150

### 5-(((3,5-dimethoxybenzyl)oxy)(1-methyl-1H-imidazol-5-yl)methyl)-2′-methyl(1,1′-biphenyl)-2-carbonitrile hydrochloride

The desired product was prepared by substituting 1-(chloromethyl)-3,5-dimethoxybenzene for 5-(chloromethyl)-1,2,3-trimethoxybenzene in Example 148.

MS (APCI(+)) m/z 454 (M+H)⁺; MS (APCI(−)) m/z 488 (M+Cl)⁻; ¹H NMR (500 MHz, DMSO-d₆) δ 8.94 (br s, 1H), 8.04 (d, 1H), 7.65 (d, 1H), 7.50 (s, 1H), 7.39-7.25 (m, 5H), 6.51-6.40 (m, 3H), 6.02 (br s, 1H), 4.56 (d, 1H), 4.47 (d, 1H), 3.73 (s, 3H), 3.70 (s, 6H), 2.12 (s, 3H).

### EXAMPLE 151

### (2′-methyl-5-((1-methyl-1H-imidazol-5-yl)((4-(methylsulfonyl)benzyl)oxy)methyl)(1,1′-biphenyl)-2-carbonitrile hydrochloride

The desired product was prepared by substituting 1-(chloromethyl)-4-(methylsulfonyl)benzene for 5-(chloromethyl)-1,2,3-trimethoxybenzene in Example 148.

MS (APCI(+)) m/z 472 (M+H)⁺; MS (APCI(−)) m/z 506 (M+Cl)⁻; ¹H NMR (500 MHz, DMSO-d₆) δ 9.09 (s, 1H), 8.06 (d, 1H), 7.91 (d, 2H), 7.68 (dd, 1H), 7.64 (d, 2H), 7.53 (d, 1H), 7.45 (s, 1H), 7.41-7.25 (m, 4H), 6.13 (s, 1H), 4.78 (d, 1H), 4.67 (d, 1H), 3.76 (s, 3H), 3.19 (s, 3H), 2.13 (br s, 3H).

### EXAMPLE 152

### 5-(((6-chloro-1,3-benzodioxol-5-yl)methoxy)(1-methyl-1H-imidazol-5-yl)methyl)-2′-methyl(1,1′-biphenyl)-2-carbonitrile hydrochloride

The desired product was prepared by substituting 5-chloro-6-(chloromethyl)-1,3-benzodioxole for 5-(chloromethyl)-1,2,3-trimethoxybenzene in Example 148.

MS (APCI(+)) m/z 472 (M+H)⁺; MS (APCI(−)) m/z 506 (M+Cl)⁻; ¹H NMR (500 MHz, DMSO-d₆) δ 8.85 (br s, 1H), 8.04 (d, 1H), 7.67 (br d, 1H), 7.50 (br s, 1H), 7.41-7.25 (m, 5H), 7.10 (s, 1H), 7.08 (s, 1H), 6.06 (s, 3H), 4.58 (d, 1H), 4.51 (d, 1H), 3.72 (s, 3H), 2.12 (s, 3H).

### EXAMPLE 153

### 5-(((4-isopropylbenzyl)oxy)(1-methyl-1H-imidazol-5-yl)methyl)-2′-methyl(1,1′-biphenyl)-2-carbonitrile hydrochloride

The desired product was prepared by substituting 1-(chloromethyl)-4-isopropylbenzene for 5-(chloromethyl)-1,2,3-trimethoxybenzene in Example 148.

MS (APCI(+)) m/z 436 (M+H)⁺; MS (APCI(−)) m/z 470 (M+Cl)⁻; ¹H NMR (500 MHz, DMSO-d₆) δ 9.03 (s, 1H), 8.04 (d, 1H), 7.65 (br d, 1H), 7.50 (br s, 1H), 7.41-7.25 (m, 5H), 7.27 (d, 2H), 7.21 (d, 2H), 6.05 (s, 1H), 4.59 (d, 1H), 4.50 (d, 1H), 3.74 (s, 3H), 2.87 (heplet, 1H), 2.13 (s, 3H), 1.18 (d, 6H).

### EXAMPLE 154

### 5-(((3,4-dimethylbenzyl)oxy)(1-methyl-1H-imidazol-5-yl)methyl)-2′-methyl(1,1′-biphenyl)-2-carbonitrile hydrochloride

The desired product was prepared by substituting 4-(chloromethyl)-1,2-dimethylbenzene for 5-(chloromethyl)-1,2,3-trimethoxybenzene in Example 148.

MS (APCI(+)) m/z 422 (M+H)⁺; MS (APCI(−)) m/z 456 (M+Cl)⁻; ¹H NMR (500 MHz, DMSO-d₆) δ 8.60 (br s, 1H), 8.03 (d, 1H), 7.63 (d, 1H), 7.46 (s, 1H), 7.41-7.25 (m, 5H), 7.15-7.02 (m, 3H), 6.00 (br s, 1H), 4.52 (d, 1H), 4.44 (d, 1H), 3.66 (s, 3H), 2.19 (s, 3H), 2.18 (s, 3H), 2.12 (s, 3H).

### EXAMPLE 155

### 5-(((4-(benzyloxy)benzyl)oxy)(1-methyl-1H-imidazol-5-yl)methyl)-2′-methyl(1,1′-biphenyl)-2-carbonitrile hydrochloride

The desired product was prepared by substituting 1-(benzyloxy)-4-(chloromethyl)benzene for 5-(chloromethyl)-1,2,3-trimethoxybenzene in Example 148.

MS (APCI(+)) m/z 500 (M+H)⁺; MS (APCI(−)) m/z 534 (M+Cl)⁻; ¹H NMR (500 MHz, DMSO-d₆) δ 8.93 (br s, 1H), 8.04 (d, 1H), 7.64 (dd, 1H), 7.48 (d, 1H), 7.41-7.24 (m, 10H), 7.28 (d, 2H), 6.98 (d, 2H), 6.00 (s, 1H), 5.10 (s, 2H), 4.54 (d, 1H), 4.46 (d, 1H), 3.71 (s, 3H), 2.12 (s, 3H).

### EXAMPLE 156

### 5-(((6-fluoro-4H-1,3-benzodioxin-8-yl)methoxy)(1-methyl-1H-imidazol-5-yl)methyl)-2′-methyl(1,1′-biphenyl)-2-carbonitrile hydrochloride

The desired product was prepared by substituting 8-(chloromethyl)-6-fluoro-4H-1,3-benzodioxine for 5-(chloromethyl)-1,2,3-trimethoxybenzene in Example 148.

MS (APCI(+)) m/z 470 (M+H)⁺; MS (APCI(−)) m/z 504 (M+Cl)⁻; ¹H NMR (500 MHz, DMSO-d₆) δ 8.95 (br s, 1H), 8.05 (d, 1H), 7.66 (d, 1H), 7.53 (s, 1H), 7.41-7.25 (m, 5H), 7.20 (dd, 1H), 6.95 ( dd, 1H), 6.08 (br s, 1H), 5.17 (d, 1H), 5.15 (d, 1H), 4.85 (s, 2H), 4.58 (d, 1H), 4.49 (d, 1H), 3.75 (s, 3H), 2.12 (s, 3H).

### EXAMPLE 157

### 5-(((2,4-dichlorobenzyl)oxy)(1-methyl-1H-imidazol-5-yl)methyl)-2′-methyl(1,1′-biphenyl)-2-carbonitrile hydrochloride

The desired product was prepared by substituting 2,4-dichloro-1-(chloromethyl)benzene for 5-(chloromethyl)-1,2,3-trimethoxybenzene in Example 148.

MS (APCI(+)) m/z 462 (M+H)⁺; MS (APCI(−)) m/z 496 (M+Cl)⁻; ¹H NMR (500 MHz, DMSO-d₆) δ 8.84 (br s, 1H), 8.04 (d, 1H), 7.65 (dd, 1H), 7.62 (d, 1H), 7.57 (d, 1H), 7.50 (d, 1H), 7.44 (dd, 1H), 7.41-7.25 (m, 5H), 6.10 (s, 1H), 4.70 (d, 1H), 4.60 (d, 1H), 3.72 (s, 3H), 2.12 (s, 3H).

### EXAMPLE 158

### 5-(((3,5-dimethylbenzyl)oxy)(1-methyl-1H-imidazol-5-yl)methyl)-2′-methyl(1,1′-biphenyl)-2-carbonitrile hydrochloride

The desired product was prepared by substituting 1,3-dichloro-5-(chloromethyl)benzene for 5-(chloromethyl)-1,2,3-trimethoxybenzene in Example 148.

MS (APCI(+)) m/z 462 (M+H)⁺; MS (APCI(−)) m/z 496 (M+Cl)^{-;} 1H NMR (500 MHz, DMSO-d₆) δ 8.45 (br s, 1H), 8.02 (d, 1H), 7.64 (d, 1H), 7.53 (t, 1H), 7.47 (s, 1H), 7.41 (d, 2H), 7.39-7.25 (m, 5H), 6.02 (br s, 1H), 4.64 (d, 1H), 4.54 (d, 1H), 3.65 (s, 3H), 2.12 (s, 3H).

### EXAMPLE 159

### 5-(((5-(tert-butyl)-1,2,4-oxadiazol-3-yl)methoxy)(1-methyl-1H-imidazol-5-yl)methyl)-2′-methyl(1,1′-biphenyl)-2-carbonitrile hydrochloride

The desired product was prepared by substituting 5-tert-butyl-3-(chloromethyl)-1,2,4-oxadiazole for 5-(chloromethyl)-1,2,3-trimethoxybenzene in Example 148.

MS (APCI(+)) m/z 442 (M+H)⁺; MS (APCI(−)) m/z 476 (M+Cl)⁻; ¹H NMR (500 MHz, DMSO-d₆) δ 8.00 (d, 1H), 7.59 (br d, 1H), 7.48 (br s, 1H), 7.41-7.25 (m, 4H), 7.24 (s, 1H), 7.14 (s, 1H), 7.03 (s, 1H), 4.68 (s, 2H), 3.63 (s, 3H), 2.13 (s, 3H), 1.34 (s, 9H).

### EXAMPLE 160

### 5-(((4-iodobenzyl)oxy)(1-methyl-1H-imidazol-5-yl)methyl)-2′-methyl(1,1′-biphenyl)-2-carbonitrile hydrochloride

The desired product was prepared by substituting 1-(bromomethyl)-4-iodobenzene for 3-(bromomethyl)benzonitrile in Example 113.

MS (APCI(+)) m/z 520 (M+H)⁺; MS (APCI(−)) m/z 554 (M+Cl)⁻; 1H NMR (500 MHz, DMSO-d₆) δ 8.78 (br s, 1H), 8.03 (d, 1H), 7.71 (d, 2H), 7.65 (dd, 1H), 7.47 (d, 1H), 7.41-7.25 (m, 5H), 7.17 (d, 2H), 6.01 (s, 1H), 4.58 (d, 1H), 4.49 (d, 1H), 3.70 (s, 3H), 2.12 (s, 3H).

### EXAMPLE 161

### 5-(((1,1′-biphenyl)-4-ylmethoxy)(1-methyl-1H-imidazol-5-yl)methyl)-2′-methyl(1,1′-biphenyl)-2-carbonitrile hydrochloride

The desired product was prepared by substituting 4-(chloromethyl)-1,1′-biphenyl for 5-(chloromethyl)-1,2,3-trimethoxybenzene in Example 148.

MS (APCI(+)) m/z 470 (M+H)⁺; MS (APCI(−)) m/z 504 (M+Cl)⁻; ¹H NMR (500 MHz, DMSO-d₆) δ 8.99 (s, 1H), 8.06 (d, 1H), 7.69 (dd, 1H), 7.66 (s, 2H), 7.64 (s, 2H), 7.52 (d, 1H), 7.49-7.25 (m, 10H), 6.09 (s, 1H), 4.69 (d, 1H), 4.59 (d, 1H), 3.76 (s, 3H), 2.13 (s, 3H).

### EXAMPLE 162

### 5-(((2-(4-chlorophenyl)-1,3-thiazol-4-yl)methoxy)(1-methyl-1H-imidazol-5-yl)methyl)-2′-methyl(1,1′-biphenyl)-2-carbonitrile hydrochloride

The desired product was prepared by substituting 4-(chloromethyl)-2-(4-chlorophenyl)-1,3-thiazole for 5-(chloromethyl)-1,2,3-trimethoxybenzene in Example 148.

MS (APCI(+)) m/z 511 (M+H)⁺; MS (APCI(−)) m/z 545 (M+Cl)⁻; ¹H NMR (500 MHz, DMSO-d₆) δ 9.12 (s, 1H), 8.04 (d, 1H), 7.89 (d, 2H), 7.78 (s, 1H), 7.67 (dd, 1H), 7.58 (d, 1H), 7.55 (d, 2H), 7.41-7.25 (m, 5H), 6.17 (s, 1H), 4.78 (d, 1H), 4.73 (d, 1H), 3.81 (s, 3H), 2.12 (s, 3H).

### EXAMPLE 163

### 5-(((5-(2-methoxyphenyl)-1,2,4-oxadiazol-3-yl)methoxy)(1-methyl-1H-imidazol-5-yl)methyl)-2′-methyl(1,1-biphenyl)-2-carbonitrile hydrochloride

The desired product was prepared by substituting 3-(chloromethyl)-5-(2-methoxyphenyl)-1,2,4-oxadiazole for 5-(chloromethyl)-1,2,3-trimethoxybenzene in Example 148.

MS (APCI(+)) m/z 492 (M+H)⁺; MS (APCI(−)) m/z 526 (M+Cl)⁻; ¹H NMR (500 MHz, DMSO-d₆) δ 8.01 (dd, 1H), 7.90 (dd, 1H), 7.67 (dd, 1H), 7.63 (d, 1H), 7.51 (br s, 1H), 7.41-7.25 (m, 6H), 7.30 (d, 1H), 7.14 (dd, 1H), 6.08 (br s, 1H), 4.78 (br s, 2H), 3.68 (br s, 3H), 2.11 (s, 3H).

### EXAMPLE 164

### 5-(((4-chloro-2-nitrobenzyl)oxy)(1-methyl-1H-imidazol-5-yl)methyl)-2′-methyl(1,1′-biphenyl)-2-carbonitrile hydrochloride

The desired product was prepared by substituting 4-chloro-1-(chloromethyl)-2-nitrobenzene for 5-(chloromethyl)-1,2,3-trimethoxybenzene in Example 148.

MS (APCI(+)) m/z 473 (M+H)⁺; MS (APCI(−)) m/z 509 (M+Cl)⁻; ¹H NMR (500 MHz, DMSO-d₆) δ 8.89 (br s, 1H), 8.15 (d, 1H), 8.04 (d, 1H), 7.85 (dd, 1H), 7.81 (d, 1H), 7.63 (d, 1H), 7.49 (s, 1H), 7.41-7.25 (m, 5H), 6.17 (s, 1H), 4.97 (d, 1H), 4.85 (d, 1H), 3.69 (s, 3H), 2.11 (s, 3H).

### EXAMPLE 165

### methyl 5-(((6-cyano-2′-methyl(1,1′-biphenyl)-3-yl)(1-methyl-1H-imidazol-5-yl)methoxy)methyl)-2-furoate hydrochloride

The desired product was prepared by substituting methyl 5-(chloromethyl)-2-furoate for 5-(chloromethyl)-1,2,3-trimethoxybenzene in Example 148.

MS (APCI(+)) m/z 442 (M+H)⁺; MS (APCI(−)) m/z 476 (M+Cl)^{-;} ¹H NMR (500 MHz, DMSO-d₆) δ 8.88 (br s, 1H), 8.03 (d, 1H), 7.61 (dd, 1H), 7.50 (d, 1H), 7.46 (s, 1H), 7.41-7.25 (m, 5H), 7.23 (d, 1H), 6.66 (d, 1H), 6.04 (s, 1H), 4.67 (s, 2H), 3.79 (s, 3H), 3.72 (s, 3H), 2.13 (s, 3H).

### EXAMPLE 166

### 2′-methyl-5-((1-methyl-1H-imidazol-5-yl)((5-(4-(trifluoromethyl)phenyl)-1,2,4-oxadiazol-3-yl)methoxy)methyl) (1,1′-biphenyl)-2-carbonitrile hydrochloride

The desired product was prepared by substituting 3-(chloromethyl)-5-(4-(trifluoromethyl)phenyl)-1,2,4-oxadiazole for 5-(chloromethyl)-1,2,3-trimethoxybenzene in Example 148.

MS (APCI(+)) m/z 530 (M+H)⁺; MS (APCI(−)) m/z 564 (M+Cl)⁻; ¹H NMR (500 MHz, DMSO-d₆) δ 8.98 (br s, 1H), 8.27 (d, 2H), 8.06 (d, 1H), 8.01 (d, 2H), 7.66 (dd, 1H), 7.57 (s, 1H), 7.41-7.25 (m, 5H), 6.22 (s, 1H), 4.87 (d, 1H), 4.70 (d, 1H), 3.81 (s, 3H), 2.13 (s, 3H).

### EXAMPLE 167

### methyl 8-(((6-cyano-2′-methyl(1,1′-biphenyl)-3-yl)(1-methyl-1H-imidazol-5-yl)methoxy)methyl)-4H-1,3-benzodioxine-6-carboxylate hydrochloride

The desired product was prepared by substituting methyl 8-(chloromethyl)-4H-1,3-benzodioxine-6-carboxylate for 5-(chloromethyl)-1,2,3-trimethoxybenzene in Example 148.

MS (APCI(+)) m/z 510 (M+H)⁺; MS (APCI(−)) m/z 544 (M+Cl)⁻; ¹H NMR (500 MHz, DMSO-d₆) δ8.93 (br s, 1H), 8.05 (d, 1H), 7.82 (d, 1H), 7.69 (d, 1H), 7.64 (d, 1H), 7.53 (s, 1H), 7.41-7.25 (m, 5H), 6.08 (s, 1H), 5.28 (d, 1H), 5.26 (d, 1H), 4.92 (s, 3H), 4.65 (d, 1H), 4.57 (d, 1H), 3.80 (s, 3H), 3.76 (s, 3H), 2.13 (s, 3H).

### Example 168

### (2′-methyl-5-((1-methyl-1H-imidazol-5-yl)((6-nitro-4H-1,3-benzodioxin-8-yl)methoxy)methyl)(1,1′-biphenyl)-2-carbonitrile hydrochloride

The desired product was prepared by substituting 8-(chloromethyl)-6-nitro-4H-1,3-benzodioxine for 5-(chloromethyl)-1,2,3-trimethoxybenzene in Example 148.

MS (APCI(+)) m/z 497 (M+H)⁺; MS (APCI(−)) m/z 531 (M+Cl)⁻; ¹H NMR (500 MHz, DMSO-d₆) δ 9.01 (s, 1H), 8.15 (d, 1H), 8.07 (d, 1H), 8.06 (d, 1H), 7.66 (dd, 1H), 7.54 (d, 1H), 7.41-7.25 (m, 5H), 6.13 (s, 1H), 5.35 (d, 1H), 5.33 (d, 1H), 4.98 (s, 2H), 4.70 (d, 1H), 4.61 (d, 1H), 3.77 (s, 3H), 2.13 (s, 3H).

### EXAMPLE 169

### 2′-methyl-5-((1,1-methyl-1H-imidazol-5-yl)((5-(3-(trifluoromethyl)phenyl)-1,2,4-oxadiazol-3-yl)methoxy)methyl)(1,1′-biphenyl)-2-carbonitrile hydrochloride

The desired product was prepared by substituting 3-(chloromethyl)-5-(3-(trifluoromethyl)phenyl)-1,2,4-oxadiazole for 5-(chloromethyl)-1,2,3-trimethoxybenzene in Example 148.

MS (APCI(+)) m/z 530 (M+H)⁺; MS (APCI(−)) m/z 564 (M+Cl)⁻; ¹H NMR (500 MHz, DMSO-d₆) δ 9.05 (s, 1H), 8.36 (d, 1H), 8.30 (s, 1H), 8.12 (d, 1H), 8.05 (d, 1H), 7.91 (t, 1H), 7.67 (dd, 1H), 7.56 (d, 1H), 7.41-7.25 (m, 5H), 6.23 (s, 1H), 4.92 (d, 1H), 4.87 (d, 1H), 3.83 (s, 3H), 2.12 (s, 3H).

### EXAMPLE 170

### 5-(((5-acetyl-2-methoxybenzyl)oxy)(1-methyl-1H-imidazol-5-yl)methyl)-2′-methyl(1,1′-biphenyl)-2-carbonitrile hydrochloride

The desired product was prepared by substituting 1-(3-(chloromethyl)-4-methoxyphenyl)ethanone for 5-(chloromethyl)-1,2,3-trimethoxybenzene in Example 148.

MS (APCI(+)) m/z 466 (M+H)⁺; MS (APCI(−)) m/z 500 (M+Cl)⁻; ¹H NMR (500 MHz, DMSO-d₆) δ 8.96 (s, 1H), 8.05 (d, 1H), 7.97 (d, 1H), 7.93 (s, 1H), 7.65 (d, 1H), 7.54 (s, 1H), 7.41-7.25 (m, 5H), 7.11(dd, 1H), 6.08 (s, 1H), 4.66 (d, 1H), 4.59 (d, 1H), 3.80 (s, 3H), 3.76 (s, 3H), 2.50 (s, 3H), 2.13 (s, 3H).

### EXAMPLE 171

### 2′-methyl-5-((1-methyl-1H-imidazol-5-yl)((5-phenyl-1,2,4-oxadiazol-3-yl)methoxy)methyl)(1,1′-biphenyl)-2-carbonitrile hydrochloride

The desired product was prepared by substituting 3-(chloromethyl)-5-phenyl-1,2,4-oxadiazole for 5-(chloromethyl)-1,2,3-trimethoxybenzene in Example 148.

MS (APCI(+)) m/z 462 (M+H)⁺; MS (APCI(−)) m/z 497 (M+Cl)⁻; ¹H NMR (500 MHz, DMSO-d₆) δ 8.95 (s, 1H), 8.07-8.04 (m, 3H), 7.73 (tt, 1H), 7.67-7.63 (m, 3H), 7.58 (d, 1H), 7.41-7.25 (m, 5H), 6.20 (s, 1H), 4.88 (d, 1H), 4.84 (d, 1H), 3.81 (s, 3H), 2.13 (s, 3H).

### EXAMPLE 172

### 5-(((5-(4-methoxyphenyl)- 1,2,4-oxadiazol-3-yl)methoxy)( 1-methyl-1H-imidazol-5-yl)methyl)-2′-methyl(1,1′-biphenyl)-2-carbonitrile hydrochloride

The desired product was prepared by substituting 3-(chloromethyl)-5-(4-methoxyphenyl)-1,2,4-oxadiazole for 5-(chloromethyl)-1,2,3-trimethoxybenzene in Example 148.

MS (APCI(+)) m/z 492 (M+H)⁺; MS (APCI(−)) m/z 526 (M+Cl)⁻; ¹H NMR (500 MHz, DMSO-d₆) δ 9.00 (s, 1H), 8.05 (d, 1H), 8.00 (d, 2H), 7.66 (dd, 1H), 7.57 (d, 1H), 7.41-7.25 (m, 5H), 7.16 (d, 2H), 6.20 (s, 1H), 4.85 (d, 1H), 4.81 (d, 1H), 3.88 (s, 3H), 3.82 (s, 3H), 2.13 (s, 3H).

### EXAMPLE 173

### 5-(((5-(3-methoxyphenyl)-1,2,4-oxadiazol-3-yl)methoxy)(1-methyl-1H-imidazol-5-yl)methyl)-2′-methyl(1,1′-biphenyl)-2-carbonitrile hydrochloride

The desired product was prepared by substituting 3-(chloromethyl)-5-(3-methoxyphenyl)-1,2,4-oxadiazole for 5-(chloromethyl)-1,2,3-trimethoxybenzene in Example 148.

MS (APCI(+)) m/z 492 (M+H)⁺; MS (APCI(−)) m/z 526 (M+Cl)⁻; ¹H NMR (500 MHz, DMSO-d₆) δ 9.07 (s, 1H), 8.05 (d, 1H), 7.68-7.63 (m, 2H), 7.58-7.53 (m, 3H), 7.41-7.25 (m, 6H), 6.22 (s, 1H), 4.89 (d, 1H), 4.86 (d, 1H), 3.86 (s, 3H), 3.83 (s, 3H), 2.13 (s, 3H).

### EXAMPLE 175

### 2′-methyl-5-((1-methyl-1H-imidazol-5-yl)((2-(4-(trifluoromethyl)phenyl)-1,3-thiazol-4-yl)methoxy)methyl)(1,1′-biphenyl)-2-carbonitrile hydrochloride

The desired product was prepared by substituting 4-(chloromethyl)-2-(4-(trifluoromethyl)phenyl)-1,3-thiazole for 5-(chloromethyl)-1,2,3-trimethoxybenzene in Example 148.

MS (APCI(+)) m/z 545 (M+H)⁺; MS (APCI(−)) m/z 579 (M+Cl)⁻; ¹H NMR (500 MHz, DMSO-d₆) δ 9.03 (s, 1H), 8.09 (d, 2H), 8.04 (d, 1H), 7.87 (s, 1H), 7.84 (d, 2H), 7.67 (dd, 1H), 7.57 (d, 1H), 7.41-7.25 (m, 5H), 6.16 (s, 1H), 4.81 (d, 1H), 4.76 (d, 1H), 3.80 (s, 3H), 2.12 (s, 3H).

### EXAMPLE 176

### 4-((1-methyl-1H-imidazol-5-yl)(((1-methyl-2-oxo-1,2-dihydro-4-pyridinyl)methyl)amino)methyl)-2-(1-naphthyl)benzonitrile

### EXAMPLE 176A

### 4-(methoxycarbonyl)-1-methylpyridinium iodide

A solution of 4-carbomethoxypyridine (5.6 g, 40 mmol) in toluene (20 mL) at 40 ° C. was treated dropwise with methyl iodide (2.5 mL, 5.7 g, 40 mmol), cooled to room temperature, stirred for 1.5 hours, heated to 80 ° C., stirred for 1 hour, treated with toluene (30 mL), and filtered to provide a solid was of sufficient purity for subsequent use without further purification.

### EXAMPLE 176B

### 1-methyl-2-oxo-1,2-dihydro-4-pyridinecarboxylic acid

A solution of Example 176A (4.0 g, 18 mmol) in water (20 mL) at room temperature was treated alternatively, at 45-minute intervals, with 2 mL and 3 mL portions of K₃Fe(CN)₆ (9.6 g, 29 mmol) in water (16 mL) at 50 ° C. and NaOH (3.5 g, 87 mmol) in water (6 mL) at room temperature. After the fourth addition (of the NaOH solution), the mixture was treated four times with 3 mL of K₃Fe(CN)₆ solution at 45 minute intervals, heated to 55° C., stirred for 1 hour, cooled to room temperature, adjusted to pH 3 with NaOH, and filtered to provide the desired product of sufficient purity for subsequent use without further purification.

MS (DCI/NH₃) m/z 154 (M+H)⁺ and 171 (M+NH₄)⁺; ¹H NMR (300 MHz, CD₃OD) δ 7.73 (d, 1H), 7.10 (d, 1H), 6.79 (dd, 1H), 3.60 (s, 3H).

### EXAMPLE 176C

### 4-(hydroxymethyl)-1-methyl-2(1H)-pyridinone

A solution of Example 176B (612 mg, 4.0 mmol) in THF (40 mL), at −8° C., was treated with isobutylchloroformate (0.57 mL, 0.60 g, 4.4 mmol) and N-methylmorpholine (0.48 mL, 0.44 g, 4.4 mmol), stirred for 1 hour, treated with sodium borohydride (930 mg, 24.6 mmol) and MeOH (12 mL), stirred for 2 hours, treated with concentrated HCl (2 mL), filtered, and concentrated. The concentrate was purified by flash column chromatography on silica gel with 85:15/hexanes:ethyl acetate to provide the desired product.

MS (DCI/NH₃) m/z 140 (M+H)⁺ and 157 (M+NH₄)⁺; ¹H NMR (300 MHz, CDCl₃) δ 7.23 (d, 1H), 6.57 (d, 1H), 6.18 (dd, 1H), 4.53 (s, 2H), 3.53 (s, 3H), 2.97 (br s, 1H).

### EXAMPLE 176D

### 1-methyl-2-oxo-1 ,2-dihydro-4-pyridinecarbaldehyde

The desired product was prepared by substituting Example 176C for Example 102C in Example 102D.

MS (DCI/NH₃) m/z 138 (M+H)⁺ and 155 (M+NH₄)⁺; ¹H NMR (300 MHz, CDCl₃) δ 9.89 (s, 1H), 7.42 (d, 1H), 7.00 (d, 1H), 6.56 (dd, 1H), 3.60 (s, 3H).

### EXAMPLE 176E

### 4-((1-methyl-1H-imidazol-5-yl)(((1-methyl-2-oxo-1,2-dihydro-4-pyridinyl)methyl)amino)methyl)-2-(1-naphthyl)benzonitrile

The desired product was prepared by substituting Example 13A and Example 176D for Example 12A and 4-nitrobenzaldehyde, respectively, in Example 12B.

MS (APCI(+)) m/z 460 (M+H)⁺; ¹H NMR (300 MHz, CDCl₃) δ 7.95 (m 2H), 7.84 (d, 1H), 7.54 (m, 4H), 7.45 (m, 4H), 7.21 (d, 1H), 6.89 (d, 1H), 6.52 (s, 1H), 6.12 (dd, 1H), 5.00 (d, 1H), 3.62 (d, 2H), 3.47 and 3.48 (both s, total 3H), 3.53 (s, 3H); Anal. calcd for C₂₉H₂₇Cl₂N₅O·0.65 H₂O: C, 64.01; H, 5.24; N, 12.87. Found: C, 64.11; H, 5.60; N, 12.50.

### EXAMPLE 177

### 2′-methyl-5-((1-methyl-1H-imidazol-5-yl)((5-methyl-3-isoxazolyl)methoxy)methyl)(1,1′-biphenyl)-2-carbonitrile hydrochloride

The desired product was prepared by substituting 3-(chloromethyl)-5-methylisoxazole for 5-(chloromethyl)-1,2,3-trimethoxybenzene in Example 148.

MS (APCI(+)) m/z 399 (M+H)⁺; MS (APCI(−)) m/z 433 (M+Cl)⁻; ¹H NMR (500 MHz, DMSO-d₆) δ 9.01 (s, 1H), 8.05 (d, 1H), 7.64 (dd, 1H), 7.49 (d, 1H), 7.41-7.25 (m, 5H), 6.31 (s, 1H), 6.08 (s, 1H), 4.65 (d, 1H), 4.59 (d, 1H), 3.74 (s, 3H), 2.38 (s, 3H), 2.13 (s, 3H).

### EXAMPLE 178

### (2′-methyl-5-((1-methyl-1H-imidazol-5-yl)((2-methyl-1-naphthyl)methoxy)methyl)(1,1′-biphenyl)-2-carbonitrile hydrochloride

The desired product was prepared by substituting 1-(chloromethyl)-2-methylnaphthalene for 5-(chloromethyl)-1,2,3-trimethoxybenzene in Example 148.

MS (APCI(+)) m/z 458 (M+H)⁺; MS (APCI(−)) m/z 492 (M+Cl)^{-;} ¹H NMR (500 MHz, DMSO-d₆) δ 8.96 (s, 1H), 8.06 (d, 1H), 8.05 (d, 1H), 7.88 (dd, 1H), 7.82 (d, 1H), 7.67 (dd, 1H), 7.54 (s, 1H), 7.49-7.22 (m, 8H), 6.20 (s, 1H), 5.12 (d, 1H), 4.99 (d, 1H), 3.68 (s, 3H), 2.43 (s, 3H), 2.13 (s, 3H).

### EXAMPLE 179

### (2′-methyl-5-((1-methyl-1H-imidazol-5-yl)((2,3,5,6-tetramethylbenzyl)oxy)methyl)(1,1′-biphenyl)-2-carbonitrile hydrochloride

The desired product was prepared by substituting 3-(chloromethyl)-1,2,4,5-tetramethylbenzene for 5-(chloromethyl)-1,2,3-trimethoxybenzene in Example 148.

MS (APCI(+)) m/z 450 (M+H)⁺; MS (APCI(−)) m/z 484 (M+Cl)⁻; ¹H NMR (500 MHz, DMSO-d₆) δ 8.98 (s, 1H), 8.06 (d, 1H), 7.67 (dd, 1H), 7.51 (s, 1H), 7.41-7.25 (m, 5H), 6.94 (s, 1H), 6.08 (s, 1H), 4.67 (d, 1H), 4.55 (d, 1H), 3.71 (s, 3H), 2.15 (s, 6H), 2.13 (s, 3H), 2.10 (s, 6H).

### EXAMPLE 180

### (2′-methyl-5-((1-methyl-1H-imidazol-5-yl)((4-(trifluoromethoxy)benzyl)oxy)methyl)(1,1′-biphenyl)-2-carbonitrile hydrochloride

The desired product was prepared by substituting 1-(chloromethyl)-4-(trifluoromethoxy)benzene for 5-(chloromethyl)-1,2,3-trimethoxybenzene in Example 148.

MS (APCI(+)) m/z 478 (M+H)⁺; MS (APCI(−)) m/z 512 (M+Cl)⁻; ¹H NMR (500 MHz, DMSO-d₆) δ 8.99 (s, 1H), 8.04 (d, 1H), 7.67 (dd, 1H), 7.51 (s, 1H), 7.50 (d, 2H), 7.41-7.25 (m, 5H), 7.34 (d, 2H), 6.08 (s, 1H), 4.68 (d, 1H), 4.58 (d, 1H), 3.74 (s, 3H), 2.12 (s, 3H).

### EXAMPLE 181

### 5-(((5,6-dichloro-3-pyridinyl)methoxy)(1-methyl-1H-imidazol-5-yl)methyl)-2′-methyl(1,1′-biphenyl)-2-carbonitrile hydrochloride

The desired product was prepared by substituting 2,3-dichloro-5-(chloromethyl)pyridine for 5-(chloromethyl)-1,2,3-trimethoxybenzene in Example 148.

MS (APCI(+)) m/z 463 (M+H)⁺; MS (APCI(−)) m/z 497 (M+Cl)⁻; ¹H NMR (500 MHz, DMSO-d₆) δ 9.01 (s, 1H), 8.41 (d, 1H), 8.16 (d, 1H), 8.04 (d, 1H), 7.67 (dd, 1H), 7.52 (d, 1H), 7.41-7.25 (m, 5H), 6.10 (s, 1H), 4.72 (d, 1H), 4.63 (d, 1H), 3.74 (s, 3H), 2.12 (s, 3H).

### EXAMPLE 182

### 5-(((3-chloro-5-(trifluoromethyl)-2-pyridinyl)methoxy)(1-methyl-1H-imidazol-5-yl)methyl)-2′-methyl(1,1′-biphenyl)-2-carbonitrile hydrochloride

The desired product was prepared by substituting 3-chloro-2-(chloromethyl)-5-(trifluoromethyl)pyridine for 5-(chloromethyl)-1,2,3-trimethoxybenzene in Example 148.

MS (APCI(+)) m/z 497 (M+H)⁺; MS (APCI(−)) m/z 531 (M+Cl)⁻; ¹H NMR (500 MHz, DMSO-d₆) δ 9.01 (s, 1H), 8.92 (d, 1H), 8.48 (d, 1H), 8.03 (d, 1H), 7.65 (dd, 1H), 7.52 (d, 1H), 7.41-7.25 (m, 5H), 6.18 (s, 1H), 4.92 (d, 1H), 4.85 (d, 1H), 3.80 (s, 3H), 2.13 (s, 3H).

### EXAMPLE 183

### 2′-methyl-5-((1-methyl-1H-imidazol-5-yl)(2-naphthylmethoxy)methyl)(1,1′-biphenyl)-2-carbonitrile hydrochloride

The desired product was prepared by substituting 2-(bromomethyl)naphthalene for 3-(bromomethyl)benzonitrile in Example 113.

MS (APCI(+)) m/z 444 (M+H)⁺; MS (APCI(−)) m/z 480 (M+Cl)⁻; ¹H NMR (500 MHz, DMSO-d₆) δ 8.95 (s, 1H), 8.05 (d, 1H), 7.92-7.87 (m, 4H), 7.71 (dd, 1H), 7.53-7.51 (m, 4H), 7.41-7.25 (m, 5H), 6.10 (s, 1H), 4.72 (d, 1H), 4.68 (d, 1H), 3.76 (s, 3H), 2.12 (s, 3H).

### EXAMPLE 184

### 5-(((3-bromobenzyl)oxy)(1-methyl-1H-imidazol-5-yl)methyl)-2′-methyl(1,1′-biphenyl)-2-carbonitrile hydrochloride

The desired product was prepared by substituting 1-bromo-3-(bromomethyl)benzene for 3-(bromomethyl)benzonitrile in Example 113.

MS (APCI(+)) m/z 474 (M+H)⁺; MS (APCI(−)) m/z 508 (M+Cl)⁻; ¹H NMR (500 MHz, DMSO-d₆) δ 8.94 (s, 1H), 8.05 (d, 1H), 7.66 (dd, 1H), 7.55 (t, 1H), 7.50 (d, 1H), 7.41-7.25 (m, 8H), 6.06 (s, 1H), 4.65 (d, 1H), 4.55 (d, 1H), 3.73 (s, 3H), 2.13 (s, 3H).

### EXAMPLE 185

### 5-(((2-bromobenzyl)oxy)(1-methyl-1H-imidazol-5-yl)methyl)-2′-methyl(1,1′-biphenyl)-2-carbonitrile hydrochloride

The desired product was prepared by substituting 1-bromo-2-(bromomethyl)benzene for 3-(bromomethyl)benzonitrile in Example 113.

MS (APCI(+)) m/z 473 (M+H)⁺; MS (APCI(−)) m/z 508 (M+Cl)⁻; ¹H NMR (500 MHz, DMSO-d₆) δ 8.97 (s, 1H), 8.04 (d, 1H), 7.68 (dd, 1H), 7.62 (dd, 1H), 7.55 (dd, 1H), 7.54 (d, 1H), 7.41-7.25 (m, 7H), 6.13 (s, 1H), 4.70 (d, 1H), 4.60 (d, 1H), 3.75 (s, 3H), 2.12 (s, 3H).

### EXAMPLE 186

### 5-(((2,6-difluorobenzyl)oxy)(1-methyl-1H-imidazol-5-yl)methyl)-2′-methyl(1,1′-biphenyl)-2-carbonitrile hydrochloride

The desired product was prepared by substituting 2-(bromomethyl)-1,3-difluorobenzene for 3-(bromomethyl)benzonitrile in Example 113.

MS (APCI(+)) m/z 430 (M+H)⁺; ¹H NMR (500 MHz, DMSO-d₆) δ 8.99 (s, 1H), 8.06 (d, 1H), 7.64 (dd, 1H), 7.50 (d, 1H), 7.48-7.15 (m, 7H), 7.12 (t, 1H), 6.10 (s, 1H), 4.70 (d, 1H), 4.61 (d, 1H), 3.77 (s, 3H), 2.14 (s, 3H).

### EXAMPLE 187

### 5-(((2-fluoro-4-(trifluoromethyl)benzyl)oxy)(1-methyl-1H-imidazol-5-yl)methyl)-2′-methyl(1,1′-biphenyl)-2-carbonitrile hydrochloride

The desired product was prepared by substituting 1-(bromomethyl)-2-fluoro-4-(trifluoromethyl)benzene for 3-(bromomethyl)benzonitrile in Example 113.

MS (APCI(+)) m/z 480 (M+H)⁺; MS (APCI(−)) m/z 514 (M+Cl)⁻; ¹H NMR (500 MHz, DMSO-d₆) δ 8.99 (s, 1H), 8.04 (d, 1H), 7.74-7.65 (m, 3H), 7.60 (d, 1H), 7.51 (d, 1H), 7.41-7.25 (m, 5H), 6.13 (s, 1H), 4.79 (d, 1H), 4.69 (d, 1H), 3.75 (s, 3H), 2.12 (s, 3H).

### EXAMPLE 188

### 4-(((6-cyano-2′-methyl(1,1′-biphenyl)-3-yl)(1-methyl-1H-imidazol-5-yl)methoxy)methyl)benzamide

A solution of Example 272 (12 mg, 0.028 mmol) in dichloromethane (1 mL) at room temperature was treated with PyBop (17.5 mg, 0.033 mmol, 1.2 eq) and 2M ammonia in methanol (100 μL), stirred for 16 hours, and concentrated. The concentrate was dissolved in 1:1/DMSO:methanol (1 mL) and purified by preparative HPLC to provide the desired product.

MS (APCI(+)) m/z 437 (M+H)⁺; MS (APCI(−)) m/z 471 (M+Cl)⁻; ¹H NMR (500 MHz, DMSO-d₆) δ 8.55 (br s, 1H), 8.04 (d, 1H), 7.93 (br s, 1H), 7.85 (d, 2H), 7.66 (dd, 1H), 7.49 (d, 1H), 7.42 (d, 2H), 7.41-7.25 (m, 5H), 7.11 (br s, 1H), 6.01 (s, 1H), 4.66 (d, 1H), 4.58 (d, 1H), 3.67 (s, 3H), 2.12 (s, 3H).

### EXAMPLE 189

### 4-(((6-cyano-2′-methyl(1,1′-biphenyl)-3-yl)(1-methyl-1H-imidazol-5-yl)methoxy)methyl)-N-methylbenzamide

A solution of Example 272 (12 mg, 0.028 mmol) in dichloromethane (1 mL) at room temperature was treated with PyBop (17.5 mg, 0.033 mmol, 1.2 eq) and 2M methylamine in methanol (100 μL), stirred for 16 hours, and concentrated. The concentrate was dissolved in 1:1/DMSO:methanol (1 mL) and purified by preparative HPLC to provide the desired product.

MS (APCI(+)) m/z 451 (M+H)⁺; MS (APCI(−)) m/z 485 (M+Cl)⁻; ¹H NMR (500 MHz, DMSO-d₆) δ 8.97 (s, 1H), 8.39 (q, 1H), 8.05 (d, 1H), 7.81 (d, 2H), 7.67 (dd, 1H), 7.51 (d, 1H), 7.43 (d, 2H), 7.41-7.25 (m, 5H), 6.06 (s, 1H), 4.68 (d, 1H), 4.59 (d, 1H), 3.73 (s, 3H), 2.78 (d, 3H), 2.12 (s, 3H).

### EXAMPLE 190

### 4-(((6-cyano-2′-methyl(1,1′-biphenyl)-3-yl)(1-methyl-1H-imidazol-5-yl)methoxy)methyl)-N,N-dimethylbenzamide

A solution of Example 272 (12 mg, 0.028 mmol) in dichloromethane (1 mL) at room temperature was treated with PyBop (17.5 mg, 0.033 mmol, 1.2 eq) and 2M dimethylamine in THF (100 EL), stirred for 16 hours, and concentrated. The concentrate was dissolved in 1:1/DMSO:methanol (1 mL) and purified by preparative HPLC to provide the desired product.

MS (APCI(+)) m/z 465 (M+H)⁺; MS (APCI(−)) m/z 499 (M+Cl)⁻; ¹H NMR (500 MHz, DMSO-d₆) δ 8.92 (s, 1H), 8.05 (d, 1H), 7.67 (dd, 1H), 7.51 (d, 1H), 7.41-7.25 (m, 9H), 6.06 (s, 1H), 4.68 (d, 1H), 4.58 (d, 1H), 3.74 (s, 3H), 2.97 (s, 3H), 2.88 (s, 3H), 2.13 (s, 3H).

### EXAMPLE 191

### 4-cyano-N-(4-cyanobenzyl)-N-((1-methy-1H-imidazol-5-yl)methyl)-3-(1-naphthyl)benzamide Example 191A

### 4-((((1-methyl-1H-imidazol-5-yl)methyl)amino)methyl)benzonitrile

The desired product was prepared by substituting 34A for 192C in Example 192D.

MS (APCI(+)) m/z 227 (M+H)⁺; ¹H NMR (500 MHz, CDCl₃) δ 7.61 (d, 1H), 7.44 (d, 1H), 7.41 (s, 1H), 6.91 (s, 1H), 3.86 (s, 2H), 3.76 (s, 2H), 3.66 (s, 3H).

### EXAMPLE 191B

### 4-carboxy-2-(1 -naphthyl)benzonitrile

A solution of Example 89A (0.20 g, 0.70 mmol) in THF (5.0 mL) and water (2.0 mL) at room temperature was treated with lithium hydroxide (0.040 g, 1.67 mmol), stirred for 2 hours, and concentrated. The concentrate was dissolved in water (10 mL) and adjusted to pH 3 with 10% HCl to provide a precipitate. The precipitate was filtered and washed with cold water to provide the desired product of sufficient purity for subsequent use without further purification.

MS (APCI(+)) m/z 291 (M+NH₄)⁺; ¹H NMR (500 MHz, DMSO-d₆) δ 8.18 (s, 2H), 8.11 (d, 1H), 8.08 (d, 1H), 8.02 (s, 1H), 7.69-7.51 (m, 4H), 7.45 (d, 1H).

### EXAMPLE 191C

### 4-cyano-N-(4-cyanobenzyl)-N-((1-methyl-1H-imidazol-5-yl)methyl)-3-(1-naphthyl)benzamide

The desired product was prepared by substituting Example 191A and Example 191B for Example 192D and 4-cyanobenzoic acid, respectively, in Example 196.

MS (APCI(+)) m/z 482 (M+H)⁺; ¹H NMR (500 MHz, DMSO-d₆) δ 8.61 (s, 1H), 8.06-8.01 (m, 3H), 7.74 (d, 1H), 7.70 (d, 2H), 7.62-7.59 (m, 2H), 7.55 (t, 1H), 7.44-7.37 (m, 5H), 7.29 (d, 1H), 4.75-4.69 (br s, 4H), 3.70 (s, 3H).

### EXAMPLE 192

### 4-((((1-methyl-1H-imidazol-5-yl)methyl)(4-trifluoromethylbenzyl)amino)methyl)-2-(1-naphthyl)benzonitrile dihydrochloride

### EXAMPLE 192A

### 4-(bromomethyl)-2-(1-naphthyl)benzonitrile

A solution of Example 89B (1.90 g, 7.34 mmol) in dioxane (35 mL) at room temperature was treated with N-bromosuccinimide (1.44 g, 8.09 mmol) and triphenylphosphine (2.12 g, 8.08 mmol), heated to 80° C. for 10 minutes, cooled to room temperature, and concentrated. The concentrate was treated with ethyl acetate (100 mL), washed with brine, dried (MgSO₄), filtered, and concentrated. The concentrate was purified by flash column chromatography on silica gel with 4:1/hexanes:ethyl acetate to provide the desired product.

MS (DCI/NH₃) m/z 339, 340, 341 and 342 (M+NH₄)⁺; ¹H NMR (500 MHz, CDCl₃) δ 7.92-7.8 (m, 2H), 7.83-7.80 (m, 1H), 7.60-7.44 (m, 7H), 4.53 (s, 2H).

### EXAMPLE 192B

### 4-(azidomethyl)-2-(1-naphthyl)benzonitrile

A solution of Example 192A (1.71 g, 5.31 mmol) in DMF (25 mL) at room temperature was treated with sodium azide (3.46 g, 53.1 mmol) and sodium iodide (80 mg, 0.53 mmol), stirred for 10 minutes, treated with ethyl acetate (100 mL), washed with brine (100 mL), dried (MgSO₄), filtered, and concentrated to provide the desired product of sufficient purity for subsequent use without further purification.

MS (DCI/NH₃) m/z 302 (M+NH₄)⁺; ¹H NMR (500 MHz, CDCl₃) δ 7.96 (d, 1H), 7.94 (d, 1H), 7.85 (d, 1H), 7.59-7.44 (m, 7H), 4.51 (s, 2H).

### EXAMPLE 192C

### 4-(aminomethyl)-2-(1-naphthyl)benzonitrile hydrochloride

A solution of Example 192B in THF (20 mL) at room temperature was treated with triphenylphosphine (1.39 g, 5.31 mmol), stirred for 30 minutes, treated with water (5 mL), heated to 60° C. for 30 minutes, and concentrated. The concentrate was treated with ethyl acetate (100 mL) and extracted with 2M HCl (100 mL). The aqueous extract was adjusted to pH 12 with sodium carbonate and extracted with diethyl ether (100 mL). The extract was dried (MgSO₄), filtered, and treated with 1M HCl in diethyl ether (10 mL) to provide a solid. The solid was collected by filtration and washed with diethyl ether to provide the desired product of sufficient purity for subsequent use without further purification.

MS (DCI/NH₃) m/z 259 (M+H)⁺ and 276 (M+NH)⁺; ¹H NMR (500 MHz, DMSO-d₆) δ 8.46 (br s, 2H), 8.13-8.07 (m, 3H), 7.79 (d, 1H), 7.75 (s, 1H), 7.70-7.52 (m, 4H), 7.50 (s, 1H), 4.22 (s, 2H).

### EXAMPLE 192D

### 4-((((1-methyl-1H-imidazol-5-yl)methyl)amino)methyl)-2-(1-naphthyl)benzonitrile dihydrochloride

A solution of Example 252A (0.68 g, 2.93 mmol) and Example 192C (0.82 g, 2.78 mmol) in 5% acetic acid/DMF (25 mL) at room temperature was treated with 4A molecular sieves, stirred for 1 hour, treated with sodium cyanoborohydride (0.26 g, 4.17 mmol), stirred for 16 hours, treated with ethyl acetate (100 mL), washed with saturated sodium carbonate and brine, dried (MgSO₄), filtered, and concentrated. The concentrate was treated with 1:1/methanol: 1M HCl (100 mL), stirred for 16 hours, and concentrated. The concentrate was adjusted to pH 12 with sodium carbonate and extracted with ethyl acetate. The extract was dried (MgSO₄), filtered, and concentrated to provide the desired product of sufficient purity for subsequent use without further purification.

MS (APCI(+)) m/z 353 (M+H)⁺; ¹H NMR (500 MHz, CDCl₃) δ 7.96-7.92 (m, 2H), 7.79 (d, 1H), 7.73 (s, 1H), 7.58-7.42 (m, 7H), 7.03 (s, 1H), 3.96 (s, 2H), 3.85 (s, 2H), 3.71 (s, 3H).

### EXAMPLE 192E

### 4-((((1-methyl-1H-imidazol-5-yl)methyl)(4-(trifluoromethyl)benzyl)amino)methyl)-2-(1-naphthyl)benzonitrile dihydrochloride

A solution of Example 192D in 5% acetic acid/DMF (1.0 mL) at room temperature was treated with 4-(trifluoromethyl)benzaldehyde (35 mg, 2.0 mmol) and anhydrous Na₂SO₄, stirred for 2 hours, treated with sodium cyanoborohydride (13 mg, 2.0 mmol), stirred for 16 hours, treated with ethyl acetate (1.0 mL), washed with saturated sodium carbonate and brine, filtered through a Chem Elut® CE1000M tube (Alltech, Northbrook, Ill.), and concentrated. The concentrate was treated with 1:1/methanol:2M HCl (1.0 mL), stirred for 16 hours, and concentrated. The concentrate was adjusted to pH 12 with sodium carbonate and extracted with ethyl acetate. The extract was dried (MgSO₄), filtered, and concentrated. The concentrate was purified by preparative HPLC, and the appropriate fractions were treated with dichloromethane (0.5 mL) and 1M HCl in diethyl ether (0.5 mL) and concentrated to provide the desired product.

MS (ESI(+)) m/z 511 and 512 (M+H)⁺; ¹H NMR (400 MHz, DMSO-d₆) δ 8.94 (s, 1H).8.08 (d, 1H), 8.06 (d, 1H), 7.94 (d, 1H), 7.67-7.46 (m, 9H), 7.50 (s, 1H), 7.46 (dd, 1H), 7.39 (d, 1H), 3.84-3.74 (m, 6H), 3.72 (s, 3H).

### EXAMPLE 193

### 4-(((4-cyano-3-(1-naphthyl)benzyl)((1-methyl-1H-imidazol-5-yl)methyl)amino)methyl)benzoic acid dihydrochloride

The desired product was prepared by substituting 4-formylbenzoic acid for 4-(trifluoromethyl)benzaldehyde in Example 192E.

MS (ESI(+)) m/z 487 (M+H)⁺; ¹H NMR (500 MHz DMSO-d₆) δ 8.94 (s, 1H), 8.08 (d, 1H), 8.06 (d, 1H), 7.95 (d, 1H), 7.85 (d, 2H), 7.66-7.58 (mn, 3H), 7.53 (s, 1H), 7.50-7.46 (m, 3H), 7.43 (d, 2H), 7.39 (dd, 1H), 3.82-3.70 (m, 6H), 3.68 (s, 3H).

### EXAMPLE 194

### N-(4-(((4-cyano-3-(1-naphthyl)benzyl)((1-methyl-1H-imidazol-5-yl)methyl)amino)methyl)phenyl)acetamide dihydrochloride

The desired product was prepared by substituting N-(4-formylphenyl)acetamide for 4-(trifluoromethyl)benzaldehyde in Example 192E.

MS (ESI(+)) m/z 500 (M+H)⁺; ¹H NMR (500 MHz, DMSO-d₆) δ 9.93 (s, 1H), 8.94 (s, 1H), 8.08 (d, 1H), 8.06 (d, 1H), 7.95 (d, 1H), 7.66-7.47 (m, 9H), 7.40 (d, 1H), 7.23 (d, 2H), 3.78-3.57 (m, 6H), 3.68 (s, 3H).

### EXAMPLE 195

### 4-((((1-methyl-1H-imidazol-5-yl)methyl)(4-(methylsulfonyl)benzyl)amino)methyl)-2-(1-naphthyl)benzonitrile dihydrochloride

The desired product was prepared by substituting 4-(methylsulfonyl)benzaldehyde for 4-(trifluoromethyl)benzaldehyde in Example 192E.

MS (ESI(+)) m/z 521 (M+H)⁺; ¹H NMR (500 MHz, DMSO-d₆) δ 8.80 (s, 1H), 8.09 (d, 1H), 8.06 (d, 1H), 7.95 (d, 1H), 7.83 (d, 2H), 7.67-7.47 (m, 9H), 7.40 (d, 1H), 3.84-3.76 (m, 6H), 3.69 (s, 3H), 3.17 (s, 3H).

### EXAMPLE 196

### 4-cyano-N-(4-cyano-3-(1-naphthyl)benzyl)-N-((1-methyl-1H-imidazol-5-yl)methyl)benzamide

A solution of Example 192D (35 mg, 0.10 mmol) in dichloromethane (0.5 mL) at room temperature was treated with a solution of 4-cyanobenzoic acid (15 mg, 1.0 mmol), PyBop (47 mg, 0.10 mmol), and N,N-diisopropylethylamine (39 mg, 0.30 mmol) in dichloromethane (0.5 mL), stirred for 72 hours, washed with brine, filtered through a Chem Elut® CE1000M tube, and concentrated. The concentrate was purified by preparative HPLC (CH₃CN/0.010M NH₄OAc) to provide the desired product.

MS (APCI(+)) m/z 482 (M+H)⁺; ¹H NMR (500 MHz, DMSO-d₆) δ 8.03 (d, 1H), 8.02 (d, 1H), 7.89 (d, 1H), 7.83 (d, 2H), 7.62 (t, 1H), 7.59-7.55 (m, 3H), 7.51 (dt, 1H), 7.46-7.42 (m, 3H), 7.38 (d, 1H), 7.27 (s, 1H), 6.80 (s, 1H), 4.65 (s, 4H), 3.43 (s, 3H).

### EXAMPLE 197

### 3,4-dichloro-N-(4-cyano-3-(1-naphthyl)benzyl)-N-((1-methyl-1H-imidazol-5-yl)methyl)benzamide

The desired product was prepared by substituting 3,4-dichlorobenzoic acid for 4-cyanobenzoic acid in Example 196.

MS (APCI(+)) m/z 525, 526, 527 and 528 (M+H)⁺; ¹H NMR (500 MHz, DMSO-d₆) δ 8.03 (d, 1H), 8.02 (d, 1H), 7.89 (d, 1H), 7.64-7.60 (m, 3H), 7.57 (dt, 1H), 7.51 (dt, 1H), 7.45-7.44 (m, 3H), 7.41-7.37 (m, 2H), 7.28 (s, 1H), 6.80 (s, 1H), 4.66 (s, 4H), 3.44 (s, 3H).

### EXAMPLE 198

### 4-chloro-N-(4-cyano-3-(1-naphthyl)benzyl)-3-fluoro-N-((1-methyl-1H-imidazol-5-yl)methyl)benzamide

The desired product was prepared by substituting 4-chloro-3-fluorobenzoic acid, for 4-cyanobenzoic acid in Example 196.

MS (APCI(+)) m/z 509, 510, 511 and 512 (M+H)⁺; ¹H NMR (500 MHz, DMSO-d₆) δ 8.03 (d, 1H), 8.02 (d, 1H), 7.89 (d, 1H), 7.63-7.55 (m, 3H), 7.50 (dt, 1H), 7.46-7.42 (m, 4H), 7.39 (d, 1H), 7.28 (s, 1H), 7.25 (dd, 1H), 6.80 (s, 1H), 4.66 (s, 2H), 4.65 (s, 2H), 3.44 (s, 3H).

### EXAMPLE 199

### 5,6-dichloro-N-(4-cyano-3-(1-naphthyl)benzyl)-N-((1-methyl-1H-imidazol-5-yl)methyl)nicotinamide

The desired product was prepared by substituting 5,6-dichloronicotinic acid for 4-cyanobenzoic acid in Example 196.

MS (APCI(+)) m/z 526, 527, 528 and 529 (M+H)⁺; ¹H NMR (500 MHz, DMSO-d₆) δ 8.42 (d, 1H), 8.14 (d, 1H), 8.03 (d, 1H), 8.02 (d, 1H), 7.89 (d, 1H), 7.62 (dt, 1H), 7.57 (dt, 1H), 7.49 (dt, 1H), 7.47-7.44 (m, 3H), 7.39 (d, 1H), 7.31 (s, 1H), 6.82 (s, 1H), 4.70 (s, 4H), 3.45 (s, 3H).

### EXAMPLE 200

### 5-(((4-cyanobenzyl)oxy)(1-methyl-1H-imidazol-5-yl)methyl)-2′-formyl(1,1′-biphenyl)-2-carbonitrile hydrochloride

### EXAMPLE 200A

### 2-bromo-4-formylbenzonitrile

A solution of compound 87C (5.1 g, 20.0 mmol) in dichloromethane (150 mL) at −100 ° C. was treated dropwise with 1M DIBAL-H in toluene (26.0 mL, 26.0 mmol), stirred for 30 minutes, treated with methanol (20 mL), stirred for 10 minutes, treated with saturated potassium sodium tartrate, warmed to room temperature, extracted with ethyl acetate, dried (MgSO₄), filtered, and concentrated. The concentrate was purified by flash column chromatography on silica gel with 4:1/hexanes:ethyl acetate to provide the desired product.

¹H NMR (300 MHz, CDCl₃) δ 10.04 (s, 1H), 8.17 (d, 1H), 7.93 (dd, 1H), 7.86 (d, 1H).

### EXAMPLE 200B

### 2-bromo-4-(hydroxy(1-methyl-1H-imidazol-5-yl)methyl)benzonitrile

A solution of Example 87F (2.59 g, 13.2 mmol) in THF (40 mL) at −78° C. was treated dropwise with 1.7M tert-butyllithium in pentane (7.06 mL, 12.0 mmol), stirred for 30 minutes, treated with a solution of Example 200A (2.1 g, 10.0 mmol) in THF (10 mL), stirred for 1 hour, treated with methanol (10 mL), stirred for 20 minutes, treated with saturated ammonium chloride (100 mL), warmed to room temperature, and extracted with ethyl acetate. The extract was dried (MgSO₄), filtered, and concentrated. The concentrate was purified by flash column chromatography on silica gel with 92:5:3/ethyl acetate:methanol: triethylamine to provide the desired product.

MS (APCI(+)) m/z 292 and 294 (M+H)⁺; ¹H NMR (300 MHz, CDCl₃) δ 7.94 (d, 1H), 7.86 (s, 1H), 7.57 (dd, 2H), 6.41 (s, 1H), 6.25 (d, 1H), 5.91 (d, 1H), 3.56 (s, 3H).

### EXAMPLE 200C

### 2-bromo-4-(((4-cyanobenzyl)oxy)(1-methyl-1H-imidazol-5-yl)methyl)benzonitrile

A solution of Example 200B (2.48 g, 8.5 mmol) and 4-cyanobenzyl bromide (2.50 g, 12.8 mmol) in dichloromethane (60 mL) at room temperature was treated with silver(I) oxide (7.8 g, 34 mmol), stirred for 16 hours in darkness, filtered through a pad of diatomaceous earth (Celite®) with methanol and concentrated. The concentrate was purified by flash column chromatography on silica gel with 92:5:3/ethyl acetate:methanol:triethylamine to provide the desired product.

MS (APCI(+)) m/z 407 and 409 (M+H)⁺; ¹H NMR (300 MHz, CDCl₃) δ 7.76 (s, 1H), 7.72-7.67 (m, 4H), 7.45-7.41 (m, 3H), 7.03 (br s, 1H), 5.61 (s, 1H), 4.65 (d, 1H), 4.57 (d, 1H), 3.44 (s, 3H).

### EXAMPLE 200D

### 5-(((4-cyanobenzyl)oxy)(1-methyl-1H-imidazol-5-yl)methyl)-2′-formyl(1,1′-biphenyl)-2-carbonitrile hydrochloride

A solution of Example 200C (30 mg, 0.074 mmol) and 2-formylphenylboronic acid (13 mg, 0.085 mmol) in n-propanol (0.5 mL) was treated with Pd(OAc)₂ (1.5 mg), triphenylphosphine (4.5 mg), 2.OM Na₂CO₃ (0.044 mL), and water (0.25 mL), heated to 100° C., stirred for 3 hours, and extracted with ethyl acetate. The extract was concentrated and the concentrate was purified by preparative HPLC to provide the desired product.

MS (APCI(+)) m/z 433 (M+H)⁺; ¹H NMR (300 MHz, DMSO-d₆) δ 9.87 (d, 1H), 9.08 (s, 1H), 8.10-8.03 (m, 2H), 7.86-7.75 (m, 3H), 7.75-7.73 (m, 2H), 7.60-7.50 (m, 4H), 7.41 (br s, 1H), 6.12 (s, 1H), 4.75 (d, 1H), 4.66 (d, 1H), 3.76 (s, 3H).

### EXAMPLE 201

### 5-(((4-cyanobenzyl)oxy)(1-methyl-1H-imidazol-5-yl)methyl)-2′-(trifluoromethyl)(1,1′-biphenyl)-2-carbonitrile hydrochloride

The desired product was prepared by substituting 2-trifluoromethylphenylboronic acid for 2-formylphenylboronic acid in Example 200D.

MS (APCI(+)) m/z 473 (M+H)⁺; ¹H NMR (300 MHz, DMSO-d₆) (rotamers) δ 9.07 and 9.05 (2s, 1H each), 8.08 (t, 1H), 7.92 (t, 1H), 7.84-7.82 (m, 2H), 7.77-7.40 (m, 2H), 7.60-7.50 (m, 4H), 7.38 (d, 1H), 6.13 (s, 1H), 4.74 (dd, 1H), 4.63 and 4.60 (2d, 1H each), 3.72 and 3.70 (2s, 3H each).

### EXAMPLE 202

### 2′,4′-dichloro-5-(((4-cyanobenzyl)oxy)(1-methyl-1H-imidazol-5-yl)methyl)(1,1′-biphenyl)-2-carbonitrile hydrochloride

The desired product was prepared by substituting with 2,4-dichlorophenylboronic acid for 2-formylphenylboronic acid in Example 200D.

MS (APCI(+)) m/z 473 (M+H)⁺; ¹H NMR (300 MHz, DMSO-d₆) (rotamers) δ 9.05 (s, 1H), 8.09 (d, 1H), 7.85-7.75 (m, 3H), 7.74 (dd, 1H), 7.65-7.45 (m, 5H), 7.41 (s, 1H), 6.13 (s, 1H), 4.75 (d, 1H), 4.65 and 4.61 (2d, 1H each), 3.73 (s, 3H).

### EXAMPLE 203

### 2-(1 -benzothien-2-yl)-4-(((4-cyanobenzyl)oxy)(1-methyl-1H-imidazol-5-yl)methyl)benzonitrile hydrochloride

The desired product was prepared by substituting benzothiophene-2-boronic acid for 2-formylphenylboronic acid in Example 200D.

MS (APCI(+)) m/z 461 (M+H)⁺; ¹H NMR (300 MHz, DMSO-d₆) δ 9.06 (s, 1H), 8.12 (d, 1H), 8.07 (t, 1H), 8.00 (d, 1H), 7.96 (s, 1H), 7.87 (s, 1H), 7.85 (d, 2H), 7.70 (d, 1H), 7.60 (d, 2H), 7.49-7.45 (m, 2H), 7.43 (s, 1H), 6.16 (s, 1H), 4.78 (d, 1H), 4.66 (d, 1H), 3.74 (s, 3H).

### EXAMPLE 204

### 5-(((4-cyanobenzyl)oxy)(1-methyl-1H-imidazol-5-yl)methyl)-2′-(hydroxymethyl)(1,1′-biphenyl)-2-carbonitrile

A solution of Example 200D (55 mg) in THF (1 mL) at room temperature was treated with a solution of CaCl₂ (30 mg) in ethanol (1 mL) and NaBH₄ (19 mg), stirred for 3 hours, and filtered. The filtrate was purified by preparative HPLC to provide the desired product.

MS (DCI/NH₃) m/z 435 (M+H)⁺; ¹H NMR (300 MHz, DMSO-d₆) δ 8.00 (d, 2H), 7.83 (d, 3H), 7.60-7.20 (m, 8H), 6.59 (s, 1H), 5.91 (s, 2H), 4.65 (d, 1H), 4.57 (d, 1H), 3.74 (s, 3H).

### EXAMPLE 205

### 2′-cyano-5′-(((4-cyanobenzyl)oxy)(1-methyl-1H-imidazol-5-yl)methyl)(1,1′-biphenyl)-2-carboxylic acid

A solution of Example 200D (50 mg) in acetone (2 mL) at room temperature was titrated with 2M CrO₃ in concentrated H₂SO₄ (Jones' reagent) until the orange endpoint, stirred for 16 hours, and concentrated. The concentrate was purified by preparative HPLC and lyophilized to provide the desired product.

MS (DCI/NH₃) m/z 449 (M+H)⁺; ¹H NMR (300 MHz, DMSO-d₆) δ 7.88-7.79 (m, 5H), 7.60-7.27 (m, 8H), 6.16 (s, 1H), 4.61 (d, 1H), 4.55 (d, 1H), 3.74 (s, 3H).

### EXAMPLE 206

### 4-cyano-N-(4-cyanobenzyl)-N-((1-methyl-1H-imidazol-5-yl)methyl)-3-(8-quinolinyl)benzamide

### EXAMPLE 206A

### 3-bromo-4-cyanobenzoic acid

A solution of Example 87C (150 mg) in methanol (3 mL) and water (1 mL) was treated with LiOH (80 mg) and stirred for 2 hours. The solution was adjusted to pH 2 with 1M HCl, then extracted with ethyl acetate. The extract was dried (MgSO₄), filtered, and concentrated to provide the desired product of sufficient purity for subsequent use without further purification.

MS (DCI/NH₃) m/z 243 and 245 (M+NH₄)⁺; ¹H NMR (300 MHz, CDCl₃) δ 8.40 (d, 1H), 8.13 (dd, 1H), 7.79 (d, 1H).

### EXAMPLE 206B

### 3-bromo-4-cyano-N-(4-cyanobenzyl)-N-((1-methyl-1H-imidazol-5-yl)methyl)benzamide

A solution of Example 206A (27 mg) and Example 191A (25 mg) in dichloromethane (1 mL) at room temperature was treated with diisopropylethylamnine (63 mL) and bromotris(pyrrolidino)phosphonium hexafluorophosphate (53.5 mg) and stirred for 16 hours. The mixture was purified by preparative HPLC and lyophilized to provide the desired product.

MS (APCI(+)) m/z 434 and 436 (M+H)⁺.

### EXAMPLE 206C

### 4-cyano-N-(4-cyanobenzyl)-N-((1-methyl-1H-imidazol-5-yl)methyl)-3-(8-quinolinyl)benzamide

A solution of Example 206B (10 mg) and 8-quinolinylboronic acid (8.0 mg) in n-propanol (0.8 mL) and water (0.4 mL) was treated with Pd(OAc)₂ (1.0 mg), triphenylphosphine (3.0 mg), and 2M Na₂CO₃ (15 mL), heated to 90° C., and stirred for 2 hours. The mixture was purified by preparative HPLC and lyophilized to provide the desired product.

MS (APCI(−)) m/z 517 (M+Cl)⁻; ¹H NMR (300 MHz, DMSO-d₆, at 90° C.) δ 8.92 (s, 1H), 8.79 (dd, 1H), 8.45 (dd, 1H), 8.12 (dd, 1H), 7.96 (d, 1H), 7.71-7.39 (m, 1OH), 4.77 (s, 2H), 4.74 (s, 2H), 3.74 (s, 3H).

### EXAMPLE 210

### 5-(1 -(benzyloxy)-2-(1H-imidazol-1-yl)ethyl)-2′-methyl(1,1′-biphenyl)-2-carbonitrile hydrochloride

### EXAMPLE 210A

### 2′-methyl-5-(2-oxiranyl)(1,1′-biphenyl)-2-carbonitrile

A solution of Example 86I (0.5 g, 2.26 mmol) in acetonitrile/water (30:1) was treated with trimethylsulfonium iodide (0.48 g, 2.32 mmol) and potassium hydroxide (0.226 g, 4.52 mmol), heated to 60° C., stirred for 4 hours, filtered, and concentrated. The concentrate was purified by flash column chromatography on silica gel with 9:1/hexanes:ethyl acetate to provide the desired product.

### EXAMPLE 210B

### 5-(1 -hydroxy-2-(1H-imidazol-1-yl)ethyl)-2′-methyl(1,1′-biphenyl)-2-carbonitrile

A solution of Example 210A (0.39 g, 1.65 mmol) in ethanol (15 mL) was treated with imidazole (0.121 g, 1.82 mmol) and catalytic pyridine, heated to reflux for 12 hours, and concentrated. The concentrate was purified by flash column chromatography on silica gel with 98:2/dichloromethane:methanol to provide the desired product.

### EXAMPLE 210C

### 5-(1-(benzyloxy)-2-(1H-imidazol-1-yl)ethyl)-2′-methyl(1,1′-biphenyl)-2-carbonitrile hydrochloride

The free base of the desired product was prepared by substituting Example 210B for Example 5D in Example 5E. The purified concentrate was treated with 1M HCl in diethyl ether and concentrated to provide the desired product.

MS (ESI(+)) m/z 394 (M+H)⁺; ¹H NMR (300 MHz, DMSO-d₆) δ 9.0 (s, 1H), 8.05 (d, 1H), 7.63 (s, 2H), 7.43 (s, 1H), 7.4-7.2 (m, 9H), 7.2-7.1 (m, 2H), 5.1-5.0 (m, 1H), 4.6-4.5 (m, 2H), 4.49 (d, 1H), 4.43 (m, 1H), 2.15 (s, 3H).

### EXAMPLE 211

### 5-(hydroxy(3-pyridinyl)methyl)-2′-methyl(1,1′-biphenyl)-2-carbonitrile

The desired product was prepared by substituting 3-bromopyridine for Example 87F in Example 1B.

MS (EST(+)) m/z 301 (M+H)⁺; ¹H NMR (300 MHz, DMSO-d₆) δ 8.64 (d, 1H), 8.45 (dd, 1H), 7.90 (d, 1H), 7.77 (dd, 1H), 7.61 (dd, 1H), 7.52 (s, 1H), 7.40-7.25 (m, 4H), 7.21 (d, 1H), 6.35 (d, 1H), 5.93 (d, 1H), 2.08 (s, 3H); Anal. calcd for C₂₀H₁₆N₂O·0.2 H₂O: C, 79.03; H, 5.44; N, 9.22. Found: C, 79.15; H, 5.55; N, 8.99.

### EXAMPLE 212

### 2′-methyl-5-((3-pyridinylamino)methyl)(1,1′-biphenyl)-2-carbonitrile

The desired product was prepared by substituting 3-aminopyridine for picolylamine in Example 215A.

MS (ESI(+)) m/z 300 (M+H)⁺; ¹H NMR (300 MHz, DMSO-d₆) δ 7.97 (d, 1H), 7.91 (d, 1H), 7.76 (dd, 1H), 7.43 (s, 1H), 7.40-7.25 (m, 3H), 7.20 (d, 1H), 7.04 (dd, 1H), 6.9-6.8 (m, 1H), 6.64 (t, 1H), 4.45 (d, 2H), 2.04 (s, 3H); Anal. calcd for C₂₀H₁₇N₃·0.3 H₂O): C, 78.82; H, 5.82; N, 13.79. Found: C, 79.19; H, 5.96; N, 13.41.

### EXAMPLE 213

### 5-((benzyloxy)(1,3-thiazol-5-yl)methyl)-2′-methyl(1,1′-biphenyl)-2-carbonitrile hydrochloride

The desired product was prepared by substituting Example 214 for Example 5D in Example 5E.

MS (ESI(+)) m/z 397 (M+H)⁺; ¹H NMR (300 MHz, DMSO-d₆) δ 9.11 (s, 1H), 8.0-7.9 (m, 2H), 7.66 (dd, 1H), 7.50 (s, 1H), 7.5-7.2 (m, 9H), 6.15 (s, 1H), 4.57 (s, 2H), 2.09 (s, 3H).

### EXAMPLE 214

### 5-(hydroxy(1,3-thiazol-5-yl)methyl)-2′-methyl(1,1′-biphenyl)-2-carbonitrile

The desired product was prepared by substituting 2-trimethylsilylthiazole for Example 87F in Example 1B.

MS (ESI(+)) m/z 307 (M+H)⁺; ¹H NMR (300 MHz, DMSO-d₆) δ 9.02 (s, 1H), 7.94 (d, 1H), 7.79 (s, 1H), 7.63 (dd, 1H), 7.50 (s, 1H), 7.40-7.25 (m, 3H), 7.22 (d, 1H), 6.69 (d, 1H), 6.23 (d, 1H), 2.10 (s, 3H); Anal. calcd for C₁₈H₁₄N₂SO·0.2 H₂O: C, 69.74; H, 4.68; N, 9.04. Found: C, 69.78; H, 4.79; N, 8.82.

### EXAMPLE 215

### 5-((benzyl(3-pyridinylmethyl)amino)methyl)-2′-methyl(1,1′-biphenyl)-2-carbonitrile hydrochloride

### EXAMPLE 215A

### 5-((3-pyridinylmethyl)amino)methyl-2′-methyl(1,1′-biphenyl)-2-carbonitrile

A solution of Example 86I (0.2 g, 0.9 mmol) in 1,2-dichloroethane (10 mL) at room temperature was treated with picolylamine (0.12 g, 1.0 mmol), acetic acid (3.6mmol), and sodium (triacetoxy)borohydride, stirred for 16 hours, treated with saturated NaHCO₃, and extracted with ethyl acetate. The extract was washed with water and brine, dried (MgSO₄), filtered, and concentrated. The concentrate was purified by flash column chromatography on silica gel with 97:3/dichloromethane:methanol to provide the desired product.

### EXAMPLE 215B

### 5-((benzyl(3-pyridinylmethyl)amino)methyl)-2′-methyl(1,1′-biphenyl)-2-carbonitrile hydrochloride

The free base of the desired product was prepared by substituting benzaldehyde and Example 215A for Example 86I and picolylamine, respectively, in Example 215A. The purified concentrate was treated with 1M HCl in diethyl ether and concentrated to provide the desired product.

MS (ESI(+)) m/z 404 (M+H)⁺; ¹H NMR (300 MHz, DMSO-d₆) δ 8.9 (br s, 1H), 8.80 (d, 1H), 8.57 (d, 1H), 7.90 (d, 2H), 7.4-7.3 (m, 8H), 7.17 (d, 1H), 3.7-3.5 (m, 6H), 2.10 (s, 3H).

### EXAMPLE 216

### 2′-methyl-5-((3-pyridinylmethyl)amino)(1,1′-biphenyl)-2-carbonitrile

The desired product was prepared by substituting 3-pyridinecarboxaldehyde and Example 225B for Example 86I and picolylamine, respectively, in Example 215A.

MS (ESI(−)) m/z 298 (M−H)⁻; MS (ESI(+)) m/z 300 (M+H)⁺; ¹H NMR (300 MHz, DMSO-d₆) δ 8.58 (s, 1H), 8.47 (d, 1H), 7.73 (d, 1H), 7.52 (d, 1H), 7.4-7.2 (m, 5H), 7.12 (d, 1H), 6.61 (d, 1H), 6.5 (s, 1H), 4.43 (d, 2H), 2.05 (s, 3H).

### EXAMPLE 217

### 5-(benzyl(3-pyridinylmethyl)amino)-2′-methyl( 1,1′-biphenyl)-2-carbonitrile

A solution of Example 216 (206 mg, 0.69 mmol) in THF at 0° C. was treated dropwise with 1M potassium tert-butoxide in THF (750 μL, 0.75 mmol), stirred for 30 minutes, treated with benzyl bromide (132 mg, 0.75 mmol), warmed to room temperature, stirred for 16 hours, treated with water, and extracted with ethyl acetate. The extract was washed with brine, dried (MgSO₄), filtered, and concentrated. The concentrate was purified by flash column chromatography on silica gel with 98:2/dichloromethane:methanol to provide the desired product.

MS (ESI(+)) m/z 390 (M+H)⁺; ¹H NMR (300 MHz, DMSO-d₆) δ 8.5-8. 4 (m, 2H), 7.65-7.55 (m, 2H), 7.4-7.2 (m, 8H), 7.08 (d, 1H), 6.83 (d, 1H), 6.55 (s, 1H), 4.9-4.8 (br m, 4H), 2.89 (s, 3H).

### EXAMPLE 218

### 4-((benzyloxy)(1-methyl-1H-imidazol-5-yl)methyl)benzomtrile hydrochloride

### EXAMPLE 218A

### 4-(hydroxy( l-methyl-1H-imidazol-5-yl)methyl)benzonitrile

The desired product was prepared by substituting 4-cyanobenzaldehyde for Example 1A in Example 1B.

### EXAMPLE 218B

### 4-((benzyloxy)(1-methyl-1H-imidazol-5-yl)methyl)benzonitrile hydrochloride

The desired product was prepared by substituting Example 218A for Example 210B in Example 210C.

MS (ESI(+)) m/z 304 (M+H)⁺; ¹ H NMR (300 MHz, DMSO-d₆) δ 9.15 (s, 1H), 7.97 (d, 2H), 7.67 (d, 2H), 7.4-7.3 (m, 6H), 6.05 (s, 1H), 4.55 (m, 2H), 3.74 (s, 3H); Anal. calcd for C₁₉H₁₇N₃O·0.8 H₂O: C, 64.42; H, 5.58; N, 11.86. Found: C, 64.44; H, 5.62; N, 11.01.

### EXAMPLE 219

### 4-(((1-methyl-1H-imidazol-5-yl)(phenyl)methoxy)methyl)benzonitrile hydrochloride

### EXAMPLE 219A

### (1-methyl-1H-imidazol-5-yl)(phenyl)methanol

The desired product was prepared by substituting benzaldehyde for Example 1A in Example 1B.

### EXAMPLE 219B

### 4-(((1-methyl-1H-imidazol-5-yl)(phenyl)methoxy)methyl)benzonitrile hydrochloride

The desired product was prepared by substituting Example 219A and 4-cyanobenzyl bromide for Example 210B and benzyl bromide, respectively, in Example 210C.

MS (ESI(+)) m/z 304 (M+H)⁺; ¹H NMR (300 MHz, DMSO-d₆) δ 9.12 (d, 1H), 7.84 (d, 2H), 7.57 (d, 2H), 7.5-7.4 (m, 5H), 7.34 (s, 1H), 5.95 (s, 1H), 4.63 (m, 2H), 3.74 (s, 3H); Anal. calcd for C₁₉H₁₇N₃O·1.0 H₂O: C, 63.77; H, 5.63; N, 11.74. Found: C, 63.99; H, 5.60; N, 10.68.

### EXAMPLE 220

### 5-(1-(benzyloxy)-2-(1-methyl-1H-imidazol-2-yl)ethyl)-2′-methyl(1,1′-biphenyl)-2-carbonitrile hydrochloride

### EXAMPLE 220A

### 5-(hydroxy(1-methyl-1H-imidazol-5-yl)methyl)-2′-methyl(1,1′-biphenyl)-2-carbonitrile

The desired product was prepared by substituting 1,2-dimethylimidazole for Example 87F in Example 1B.

### EXAMPLE 220B

### 5-(1-(benzyloxy)-2-(1-methyl-1H-imidazol-2-yl)ethyl)-2′-methyl(1,1′-biphenyl)-2-carbonitrile hydrochloride

The desired product was prepared by substituting Example 220A for Example 210B in Example 210C.

MS (ESI(+)) m/z 408 (M+H)⁺; ¹H NMR (300 MHz, DMSO-d₆) δ 8.05 (d, 1H), 7.7-7.1 (m, 14H), 5.05-4.95 (m, 1H), 4.38 (m, 2H), 3.71 (s, 3H), 2.13 (s, 3H), 14.43 (br s, 1H); Anal. calcd for C₂₇H₂₆N₃OCl·1.25 H₂O: C, 69.51; H, 6.15; N, 9.00. Found: C, 69.61; H, 5.96; N, 8.23.

### EXAMPLE 221

### 5-((benzyloxy)(1-methyl-1H-imidazol-2-yl)methyl)-2′-methyl(1,1′-biphenyl)-2-carbonitrile hydrochloride

### EXAMPLE 221A

### 5-(hydroxy(1-methyl-1H-imidazol-2-yl)methyl)-2′-methyl(1,1′-biphenyl)-2-carbonitrile

The desired product was prepared by substituting 1-methylimidazole for Example 87F in Example 1B.

### EXAMPLE 221B

### 5-((benzyloxy)(1-methyl-1H-imidazol-2-yl)methyl)-2′-methyl(1,1′-biphenyl)-2-carbonitrile hydrochloride

The desired product was prepared by substituting Example 221A for Example 210B in Example 210C.

MS (ESI(+)) m/z 394 (M+H)⁺; ¹H NMR (300 MHz, DMSO-d₆) δ 8.06 (d, 1H), 7.7-7.5 (m, 4H), 7.4-7.2 (m, 9H), 6.41 (s, 1H), 4.69 (s, 2H), 3.77 (s, 3H), 2.11 (s, 3H).

### EXAMPLE 222

### 5-(1H-imidazol-1-ylmethyl)-2′-methyl(1,1′-biphenyl)-2-carbonitrile hydrochloride

A suspension of Example 20A (200 mg, 0.7 mmol) in DMF (5 mL) was treated with imidazole (57 mg, 0.84 mmol) and K₂CO₃ (193 mg, 1.4 mmol), heated to 50° C., stirred for 2 hours, treated with ethyl acetate, washed with brine, dried (MgSO₄), filtered, and concentrated. The concentrate was purified by flash column chromatography on silica gel with 95:5/dichloromethane:methanol, treated with 1M HCl in diethyl ether, and concentrated to provide the desired product.

MS (ESI(+)) m/z 274 (M+H)⁺; ¹H NMR (300 MHz, DMSO-d₆) δ 9.35 (s, 1H), 8.02 (d, 1H), 7.86 (t, 1H), 7.73 (t, 1H), 7.65-7.55 (m, 2H), 7.5-7.3 (m, 3H), 7.23 (d, 1H), 5.59 (s, 2H), 2.11 (s, 3H); Anal. calcd for C₁₈_{H}₁₆N₃Cl·0.9 H₂O: C, 66.32; H, 5.50; N, 12.89. Found: C, 66.45; H, 5.67; N, 11.74.

### EXAMPLE 223

### 4-(((1-methyl-1H-imidazol-5-yl)(3-(1-naphthyl)phenyl)methoxy)methyl)benzonitrile

The desired product was prepared by substituting 4-(bromomethyl)benzonitrile for benzyl bromide in Example 224C.

MS (ESI(+)) m/z 430 (M+H)⁺; ¹H NMR (300 MHz, DMSO-d₆) δ 8.0-7.9 (m, 2H), 7.9-7.7 (m, 3H), 7.6-7.4 (m, 1 1H), 6.61 (s, 1H), 5.87 (s, 1H), 4.65 (m, 2H), 3.57 (s, 3H); Anal. calcd for C₂₉H₂₃N₃O·0.25 H₂O: C, 80.25; H, 5.45; N, 9.68. Found: C, 80.02; H, 5.56; N, 9.56.

### EXAMPLE 224

### benzyl (1-methyl-1H-imidazol-5-yl)(3-(1-naphthyl)phenyl)methyl ether hydrochloride

### EXAMPLE 224A

### 3-(1-naphthyl)benzaldehyde

The desired product was prepared by substituting 3-bromobenzaldehyde and 1-naphthylboronic acid for 3-bromo-4-fluorobenzaldehyde and 2-methylphenylboronic acid, respectively, in Example 1A.

### EXAMPLE 224B

### (1-methyl-1H-imidazol-5-yl)(3-(1-naphthyl)phenyl)methanol

The desired product was prepared by substituting Example 224A for Example 1 in Example 1B.

### EXAMPLE 224C

### benzyl (1-methyl-1H-imidazol-5-yl)(3-(1-naphthyl)phenyl)methyl ether hydrochloride

The desired product was prepared by substituting Example 224B for Example 5D in Example 5E.

MS (ESI(+)) m/z 405 (M+H)⁺; ¹H NMR (300 MHz, DMSO-d₆) δ 8.00 (d, 1H), 7.97 (d, 1H), 7.79 (d, 1H), 7.6-7.4 (m, 9H), 7.40-7.25 (m, 5H), 6.56 (s, 1H), 5.81 (s, 1H), 4.54 (m, 2H), 3.56 (s, 3H); Anal. calcd for C₂₈H₂₄N₂O·0.5 H₂O: C, 81.15; H, 5.89; N, 6.56. Found: C, 81.32; H, 6.09; N, 6.77.

### EXAMPLE 225

### 2′-methyl-5-(((1-methyl-1H-imidazol-5-yl)methyl)amino)(1,1′-biphenyl)-2-carbonitrile

### EXAMPLE 225A

### 6-cyano-2′-methyl(1,1′-biphenyl)-3-carboxylic acid

A solution of Example 86H (2.0 g, 8.9 mmol) in acetone (25 mL) at 0° C. was titrated with Jones' reagent, stirred for 30 minutes, treated with iso-propanol and concentrated to ⅓ its original volume treated with water (200 mL) while stirring vigorously, then filtered and dried in a vacuum oven to provide the desired product.

### EXAMPLE 225B

### 3-((tert-butoxycarbonyl)amino-6-cyano-2′-methyl-1,1′-biphenyl

A solution of Example 225A (2.16 g, 9.11 mmol) in tert-butanol (30 mL) was treated with diphenylphosphoryl azide (1.96 mL, 9.11 mmol) and triethylamine (1.3 mL, 9.11 mmol), heated to reflux, stirred for 21 hours, cooled to room temperature, and concentrated. The concentrate was treated with ethyl acetate (50 mL), washed sequentially with water, 5% citric acid, water, 5% NaHCO₃, and brine, dried (MgSO₄), filtered, and concentrated. The concentrate was purified by flash column chromatography on silica gel with 85:15/hexanes:ethyl acetate, to provide the desired product.

### EXAMPLE 225C

### 5-amino-2′-methyl(1,1′-biphenyl)-2-carbonitrile

A solution of Example 225B in dichloromethane (5 mL) was treated with trifluoroacetic acid (5 mL), stirred for 45 minutes, and concentrated under a nitrogen atmosphere. The concentrate was treated with ethyl acetate, washed with saturated NaHCO₃ and brine; dried (MgSO₄), filtered, and concentrated. The concentrate was purified by flash column chromatography on silica gel with 60:40/hexanes:ethyl acetate to provide the desired product.

### EXAMPLE 225D

### 1-methyl-2-triethylsilylimidazole-5-carboxaldehyde

A solution of Example 87F (1 g, 5.10 mmol) in THF (20 mL) at −78 ° C. was treated dropwise with 1.7M tert-butyllithium in hexanes (3 mL, 5.10 mmol), stirred for 10 minutes, treated slowly with N-formylmorpholine, stirred for 1 hour, treated with saturated NaHCO₃ and extracted with ethyl acetate. The extract was washed with brine, dried (MgSO₄), filtered, and concentrated to provide the desired product of sufficient purity for subsequent use without further purification.

### EXAMPLE 225E

### 2′-methyl-5-(((1-methyl-1H-imidazol-5-yl)methyl)amino)(1,1′-biphenyl)-2-carbonitrile

A solution of Example 225C (100 mg, 0.48 mmol) in 1,2-dichloroethane (5 mL) at room temperature was treated with Example 225D (215 mg, 0.96 mmol), (triacetoxy)borohydride (283 mg, 1.33 mmol), and acetic acid (136 μL, 2.38 mmol), stirred for 16 hours, treated with saturated NaHCO₃ and extracted with ethyl acetate. The extract was washed with saturated NaHCO₃ and brine, dried (MgSO₄), filtered, and concentrated. The concentrate was purified by flash column chromatography on silica gel with 95:5/dichloromethane: methanol to provide the desired product.

MS (ESI(+)) m/z 303 (M+H)⁺; ¹H NMR (300 MHz, DMSO-d₆) δ 7.6-7.5 (m, 2H), 7.4-7.2 (m, 3H), 7.16 (d, 1H), 7.06 (t, 1H), 6.85 (d, 1H), 6.76 (dd, 1H), 6.58 (d, 114), 4.32 (d, 2H), 3.60 (s, 3H), 2.13 (s, 3H); Anal. calcd for C₁₉H₁₉N₄·0.75 H₂O: C, 72.24; H, 6.22; N, 17.73. Found: C, 72.50; H, 5.97; N, 17.17.

### EXAMPLE 226

### 5-(benzyl((1-methyl-1H-imidazol-5-yl)methyl)amino)-2′-methyl(1,1-biphenyl)-2-carbonitrile

A solution of Example 225E (100 mg, 0.33 mmol) in THF (2 mL) at room temperature was treated dropwise with 1M potassium tert-butoxide in THF (500 μL, 0.50 mmol) and benzyl bromide (50 mL, 0.42 mmol), sealed in a screw-cap vial, heated to 50° C., stirred for 3 hours, cooled to room temperature, treated with ethyl acetate, washed with water and brine, dried (MgSO₄), filtered, and concentrated. The concentrate was purified by flash column chromatography on silica gel with 96:4/dichloromethane:methanol to provide the desired product.

MS (ESI(+)) m/z 393 (M+H)⁺; ¹H NMR (300 MHz, DMSO-d₆) δ 7.60 (d, 1H), 7.55 (d, 1H), 7.40-7.15 (m, 8H), 7.11 (d, 1H), 6.89 (dd, 1H), 6.7-6.6 (m, 2H), 4.9-4.7 (m, 4H), 3.56 (s, 3H), 1.94 (s, 3H); Anal. calcd for C₂₆H₂₄N₄·0.25 H₂O: C, 78.65; H, 6.22; N, 14.11. Found: C, 78.71; H, 6.24; N, 13.88.

### EXAMPLE 227

### 4-(methyl((1-methyl-1H-imidazol-5-yl)methyl)amino)-2-(1-naphthyl)benzonitrile

The desired product was prepared by substituting methyl iodide for benzyl bromide in Example 232.

MS (ESI(+)) m/z 353 (M+H)⁺; ¹H NMR (300 MHz, DMSO-d₆) δ 8.02 (d, 2H), 7.72 (d, 1H), 7.6-7.4 (m, 6H), 7.01 (dd, 1H), 6.88 (d, 1H), 6.66 (s, 1H), 4.67 (m, 2H), 3.54 (s, 3H), 3.02 (s, 3H); Anal. calcd for C₂₃H₂₀N₄·1.0 H₂O: C, 74.57; H, 5.98; N, 15.12. Found: C, 74.55; H, 5.85; N, 13.83.

### EXAMPLE 228

### 4-(allyl( (1-methyl-1H-imidazol-5-yl)methyl)amino)-2-(1-naphthyl)benzonitrile

The desired product was prepared by substituting allyl bromide for benzyl bromide in Example 232.

MS (ESI(+)) m/z 379 (M+H)⁺; ¹H NMR (300 MHz, DMSO-d₆) δ 8.01 (d, 2H), 7.70 (d, 1H), 7.6-7.4 (m, 6H), 7.00 (dd, 1H), 6.85 (d, 1H), 6.69 (s, 1H), 5.85-5.75 (m, 1H), 5.2-5.1 (m, 2H), 4.66 (m, 2H), 4.07 (dd, 2H), 3.54 (s, 3H); Anal. calcd for C₂₅H₂₂N₄·0.5 H₂O: C, 77.49; H, 5.98; N, 14.45. Found: C, 77.50; H, 6.00; N, 14.14.

### EXAMPLE 229

### 5-((4-cyanobenzyl)((1-methyl-1H-imidazol-5-yl)methyl)amino)-2′-methyl(1,1′-biphenyl)-2-carbonitrile

The desired product was prepared by substituting 4-(bromomethyl)benzonitrile for benzyl bromide in Example 226.

MS (ESI(+)) m/z 418 (M+H)⁺; ¹H NMR (300 MHz, DMSO-d₆) δ 7.81 (d, 2H), 7.63 (d, 1H), 7.55 (s, 1H), 7.38 (d, 2H), 7.35-7.20 (m, 3H), 7.12 (d, 1H), 6.88 (dd, 1H), 6.7-6.6 (m, 2H), 4.82 (br s, 4H), 3.55 (s, 3H), 1.94 (s, 3H); Anal. calcd for C₂₇H₂₃N₅·0.4 H₂O: C, 76.36; H, 5.65; N, 16.49. Found: C, 76.40; H, 5.58; N, 16.17.

### EXAMPLE 230

### 4-(((1-methyl-1H-imidazol-5-yl)methyl)(3-phenylpropyl)amino)-2-(1-naphthyl)benzonitrile

The desired product was prepared by substituting 1-bromo-3-phenylpropane for benzyl bromide in Example 232.

MS (ESI(+)) m/z 457 (M+H)⁺; ¹H NMR (300 MHz, DMSO-d₆) δ 8.02 (d, 2H), 7.7-7.4 (m, 7H), 7.2-7.0 (m, 5H), 6.94 (dd, 1H), 6.72 (d, 1H), 6.61 (s, 1H), 4.64 (m, 2H), 3.52 (s, 3H), 3.5-3.3 (m, 2H), 2.6-2.5 (m, 2H), 1.75-1.90 (m, 2H).

### EXAMPLE 231

### 4-((4-cyanobenzyl)((1-methyl-1H-imidazol-5-yl)methyl)amino)-2-(1-naphthyl)benzonitrile

The desired product was prepared by substituting 4-(bromomethyl)benzonitrile for benzyl bromide in Example 232.

MS (ESI(+)) m/z 454 (M+H)⁺; ¹H NMR (300 MHz, DMSO-d₆) δ 7.99 (d, 2H), 7.81 (d, 2H), 7.72 (d, 1H), 7.6-7.5 (m, 3H), 7.5-7.3 (m, 5H), 6.99 (dd, 1H), 6.80 (d, 1H), 6.71 (s, 1H), 5.0-4.7 (m, 4H), 3.54 (s, 3H); Anal. calcd for C₃₀H₂₃N₅·0.75 H₂O: C, 77.14; H, 5.28; N, 14.99. Found: C, 77.32; H, 5.31; N, 14.66.

### EXAMPLE 232

### 4-(benzyl((1-methyl-1H-imidazol-5-yl)methyl)amino)-2-(1-naphthyl)benzonitrile

The desired product was prepared by substituting Example 234 for Example 225E in Example 226.

MS (ESI(+)) m/z 429 (M+H)⁺; ¹H NMR (300 MHz, DMSO-d₆) δ 8.00 (d, 2H), 7.70 (d, 1H), 7.6-7.2 (m, 11H), 7.00 (dd, 1H), 6.84 (d, 1H), 6.71 (s, 1H), 4.83 (m, 2H), 4.74 (m, 2H), 3.54 (s, 3H); Anal. calcd for C₂₉H₂₄N₄·0.5 H₂O: C, 79.60; H, 5.75; N, 12.80. Found: C, 79.80; H, 5.79; N, 12.68.

### EXAMPLE 233

### 4-(hexyl((1-methyl-1H-imidazol-5-yl)methyl)amino)-2-(1-naphthyl)benzonitrile

The desired product was prepared by substituting hexyl iodide for benzyl bromide in Example 232.

MS (ESI(+)) m/z 423 (M+H)⁺; ¹H NMR (300 MHz, DMSO-d₆) δ 8.01 (d, 2H), 7.7-7.4 (m, 7H), 6.96 (dd, 1H), 6.81 (d, 1H), 6.64 (s, 1H), 4.63 (m, 2H), 3.53 (s, 3H), 3.5-3.3 (m, 2H), 1.6-1.4 (m, 2H), 1.3-1.2 (m, 6H), 0.9-0.7 (m, 3H); HRMS calcd m/z for C₂₈H₃₁N₄: 423.2549 (M+H)⁺. Found: 423.2551.

### EXAMPLE 234

### 4-(((1-methyl-1H-imidazol-5-yl)methyl)amino)-2-(1-naphthyl)benzonitrile

### EXAMPLE 234A

### tert-butyl 4-cyano-3-(1-naphthyl)phenylcarbamate

The desired product was prepared by substituting Example 191B for Example 225A in Example 225B.

### EXAMPLE 234B

### 4-amino-2-(1-naphthyl)benzonitrile

The desired product was prepared by substituting Example 234A for Example 225B in Example 225C.

### EXAMPLE 234C

### 4-(((1-methyl-1H-imidazol-5-yl)methyl)amino)-2-(1-naphthyl)benzonitrile

The desired product was prepared by substituting Example 234B for Example 225C in Example 225E.

MS (ESI(+)) m/z 339 (M+H)⁺; ¹H NMR (300 MHz, DMSO-d₆) δ 8.01 (d, 2H), 7.7-7.4 (m, 6H), 7.12 (t, 1H), 6.9-6.8 (m, 2H), 6.74 (d, 1H), 4.34 (d, 2H), 3.60 (s, 3H); Anal. calcd for C₂₀H₁₈N₄O₂·1.25 H₂O: C, 73.21; H, 5.72; N, 15.52. Found: C, 73.07; H, 5.43; N, 14.84.

### EXAMPLE 235

### N-(4-cyano-3-(1-naphthyl)phenyl)-N-((1-methyl-1H-imidazol-5-yl)methyl)benzamide

The desired product was prepared by substituting benzoyl chloride for benzyl bromide in Example 232.

MS (ESI(+)) m/z 443 (M+H)⁺; ¹H NMR (300 MHz, DMSO-d₆) δ 8.00 (dd, 2H), 7.95 (d, 1H), 7.6-7.3 (m, 10H), 7.24 (d, 1H), 7.13 (d, 1H), 6.72 (s, 1H), 6.64 (d, 1H), 5.24 (s, 2H), 3.59 (s, 3H); Anal. calcd for C₂₉H₂₂N₄O·0.75 H₂O: C, 76.38; H, 5.19; N, 12.28. Found: C, 76.58; H, 5.23; N, 12.08.

### EXAMPLE 236

### N-(6-cyano-2′-methyl(1,1′-biphenyl)-3-yl)-N-((1-methyl-1H-imidazol-5-yl)methyl)benzamide

The desired product was prepared by substituting benzoyl chloride for benzyl bromide in Example 226.

MS (ESI(+)) m/z 407 (M+H)⁺; ¹H NMR (300 MHz, DMSO-d₆) δ 7.82 (d, 1H), 7.51 (s, 1H), 7.4-7.2 (m, 9H), 7.05 (d, 1H), 6.92 (d, 1H), 6.68 (s, 1H), 5.21 (s, 2H), 3.58 (s, 3H), 1.73 (s, 3H); Anal. calcd for C₂₀H₁₈N₄O₂·0.5 H₂O: C, 75.16; H, 5.57; N, 13.48. Found: C, 75.40; H, 5.63; N, 13.40.

### EXAMPLE 237

### 5-((3-cyanobenzyl)((1-methyl-1H-imidazol-5-yl)methyl)amino)-2′-methyl(1,1′-biphenyl)-2-carbonitrile

The desired product was prepared by substituting 3-(bromomethyl)benzonitrile for benzyl bromide in Example 226.

MS (ESI(+)) m/z 418 (M+H)⁺; ¹H NMR (300 MHz, DMSO-d₆) δ 7.8-7.5 (m, 6H), 7.35-7.20 (m, 3H), 7.12 (d, 1H), 6.90 (dd, 1H), 6.7-6.6 (m, 2H), 4.9-4.7 (m, 4H), 3.85 (s, 3H), 1.94 (s, 3H); Anal. calcd for C₂₇H₂₃N₅·0.75 H₂O: C, 75.23; H, 5.72; N, 16.24. Found: C, 75.38; H, 5.56; N, 16.33.

### EXAMPLE 238

### 4-((1-methyl-1H-imidazol-5-yl)carbonyl)-2-(8-quinolinyl)benzonitrile

### EXAMPLE 238A

### 2-bromo-4-((1-methyl-1H-imidazol-5-yl)carbonyl)benzonitrile

A solution of Example 200B (250 mg) in dichloromethane(5.0 mL) at room temperature was treated with silver(I) oxide (0.79 g), stirred for 16 hours, filtered through a pad of diatomaceous earth (Celite®), and concentrated. The concentrate was purified by flash column chromatography on silica gel with 95:5/ethyl acetate:methanol to provide the desired product.

MS (APCI(+)) m/z 290 and 292 (M+H)⁺.

### EXAMPLE 238B

### 4-((1-methyl-1H-imidazol-5-yl)carbonyl)-2-(8-quinolinyl)benzonitrile

The desired product was prepared by substituting Example 238A and 8-quinolinylboronic acid for Example 200C and 2-formylphenylboronic acid, respectively, in Example 200D.

MS (APCI(+)) m/z 338 (M+H)⁺.

### EXAMPLE 240

### 4-(((3,4-dichlorobenzyl)oxy)(1-methyl-1H-imidazol-5-yl)methyl)-2-(8-quinolinyl)benzonitrile dihydrochloride

### EXAMPLE 240A

### 2-bromo-4-(((3,4-dichlorobenzyl)oxy)(1-methyl-1H-imidazol-5-yl)methyl)benzonitrile

The desired product was prepared by substituting 3, 4-dichlorobenzyl bromide for 4-cyanobenzyl bromide in Example 200C.

MS (APCI(+)) m/z 450 and 452 (M+H)⁺.

### EXAMPLE 240B

### 4-(((3,4-dichlorobenzyl)oxy)(1-methyl-1H-imidazol-5-yl)methyl)-2-(8-quinolinyl)benzonitrile dihydrochloride

The desired product was prepared by substituting Example 240A for Example 238A in Example 238B.

MS (APCI(−)) m/z 533, 535, and 537 (M+^{35/37}Cl)⁻; ¹H NMR (300 MHz, DMSO-d₆) (rotamers) δ 9.13 and 9.11 (2s, 1H each), 8.96 (d, 1H), 8.54 and 8.51 (2d, 1H each), 8.16 and 8.07 (2d, 1H each), 7.94-7.36 (m, 8H), 7.09 (d, 1H), 6.77 (br s, 0.5H), 6.36 (dd, 0.5H), 6.24 and 6.11 (2s, 1H each), 4.75-4.57 (m, 2H), 3.82 and 3.80 (2s, 3H each).

### EXAMPLE 241

### 4-(((3-fluoro-4-(trifluoromethyl)benzyl)oxy)(1-methyl-1H-imidazol-5-yI)methyl)-2-(8-quinolinyl)benzonitrile dihydrochloride

### EXAMPLE 241A

### 2-bromo-4-(((3-fluoro-4-(trifluoromethyl)benzyl)oxy)(1-methyl-1H-imidazol-5-yl)methyl)benzonitrile

The desired product was prepared by substituting 4-trifluoromethyl-3-fluoro-benzyl bromide for 4-cyanobenzyl bromide in Example 200C.

MS (APCI(+)) m/z 468 and 470 (M+H)⁺.

### EXAMPLE 241B

### 4-(((3-fluoro-4-(trifluoromethyl)benzyl)oxy)(1-methyl-1H-imidazol-5-yl)methyl)-2-(8-2quinolinyl)benzonitrile dihydrochloride

The desired product was prepared by substituting Example 241A and 8-quinolinylboronic acid for Example 200C and 2-formylphenylboronic acid, respectively, in Example 200D.

MS (APCI(+)) m/z 517 (M+H)⁺; MS (APCI(−)) m/z 551 (M+Cl)⁻; ¹H NMR (300 MHz, DMSO-d₆) (rotamers) δ 9.13 and 9.11 (2s, 1H each), 8.96 and 8.94 (2d, 1H each), 8.54 and 8.51 (2d, 1H each), 8.16 and 8.07 (2d, 1H each), 7.96-7.38 (m, 8H), 7.09 (d, 1H), 6.77 (br s, 0.5H), 6.36 (dd, 0.5H), 6.25 and 6.12 (2s, 1H each), 4.75-4.57 (m, 2H), 3.82 and 3.80 (2s, 3H each).

### EXAMPLE 242

### 4-(((4-fluoro-3-(trifluoromethyl)benzyl)oxy)(1-methyl-1H-imidazol-5-yl)methyl)-2-(8-quinolinyl)benzonitrile dihydrochloride

### EXAMPLE 242A

### 2-bromo-4-(((4-fluoro-3-(trifluoromethyl)benzyl)oxy)(1-methyl-1H-imidazol-5-yl)methyl)benzonitrile

The desired product was prepared by substituting 3-trifluoromethyl-4-fluoro-benzyl bromide for 4-cyanobenzyl bromide in Example 200C.

MS (APCI(+)) m/z 468 and 470 (M+H)⁺.

### EXAMPLE 242B

### 4-(((4-fluoro-3-(trifluoromethyl)benzyl)oxy)(1-methyl-1H-imidazol-5-yl)methyl)-2-(8-quinolinyl)benzonitrile dihydrochloride

The desired product was prepared by substituting Example 242A and 8-quinolinylboronic acid for Example 200C and 2-formylphenylboronic acid, respectively, in Example 200D.

MS (APCI(+)) m/z 517 (M+H)⁺; MS (APCI(−)) m/z 551 (M+Cl)⁻; ¹H NMR (300 MHz, DMSO-d₆) (rotamers) δ 9.16 and 9.11 (2s, 1H each), 8.96 and 8.94 (2d, 1H each), 8.56 and 8.51 (2d, 1H each), 8.16 and 8.07 (2d, 1H each), 7.95-7.20 (m, 8H), 7.09 (m, 1H), 6.79 (br s, 0.5H), 6.35 (dd, 0.5H), 6.28 and 6.15 (2s, 1H each), 4.82-4.64 (m, 2H), 3.83 and 3.81 (2s, 3H each).

### EXAMPLE 243

### 4-(((4-cyano-3-(8-quinolinyl)phenyl)(1-methyl-1H-imidazol-5-yl)methoxy)methyl)benzoic acid dihydrochloride

### EXAMPLE 243A

### methyl 4-(((3-bromo-4-cyanophenyl)(1-methyl-1H-imidazol-5-yl)methoxy)methyl)benzoate

The desired product was prepared by substituting methyl 4-(bromomethyl)benzoate for 4-cyanobenzyl bromide in Example 200C.

MS (APCI(+)) m/z 440 and 442 (M+H)⁺.

### EXAMPLE 243B

### 4-(((4-cyano-3-(8-quinolinyl)phenyl)(1-methyl-1H-imidazol-5-yl)methoxy)methyl)benzoic acid dihydrochloride

The desired product was prepared by substituting Example 243A and 8-quinolinylboronic acid for Example 200C and 2-formylphenylboronic acid, respectively, in

### EXAMPLE 200D.

MS (APCI(+)) m/z 475 (M+H)⁺; ¹H NMR (300 MHz, DMSO-d₆) (rotamers) δ 9.12 and 9.10 (2s, 1H each), 9.00-8.85 (m, 1H), 8.51 and 8.49 (2d, 1H each), 8.32 (d, 1H), 8.11 (d, 1H), 7.97-7.48 (m, 8H), 7.09 (m, 1H), 6.80 (br s, 0.5H), 6.38 (dd, 0.5H), 6.26 and 6.13 (2s, 1H each), 4.82-4.65 (m, 2H), 15 3.81 and 3.80 (2s, 3H each).

### EXAMPLE 244

### 6-(((4-cyano-3-(8-quinolinyl)phenyl)(1-methyl-1H-imidazol-5-yl)methoxy)methyl)nicotinamide trihydrochloride

### EXAMPLE 244A

### 6-(((3-bromo-4-cyanophenyl)(1-methyl-1H-imidazol-5-yl)methoxy)methyl)nicotinonitrile

The desired product was prepared by substituting 6-bromomethyl nicotinonitrile for 4-cyanobenzyl bromide in Example 200C.

MS (APCI(+)) m/z 408 and 410 (M+H)⁺.

### EXAMPLE 244B

### 6-(((4-cyano-3-(8-quinolinyl)phenyl)(1-methyl-1H-imidazol-5-yl)methoxy)methyl)nicotinamide trihydrochloride

The desired product was prepared by substituting Example 244A and 8-quinolinylboronic acid for Example 200C and 2-formylphenylboronic acid, respectively, in Example 200D.

MS (APCI(+)) m/z 475 (M+H)⁺; MS (APCI(−)) m/z 509 (M+Cl)⁻; ¹H NMR (300 MHz, DMSO-d₆) (rotamers) δ 9.12 (2s, 1H), 9.02-8.74 (m, 2H), 8.59 and 8.50 (2d, 1H each), 8.29-7.48 (m, 10H), 6.38 and 6.35 (2s, 1H each), 4.88-4.70 (m, 2H), 3.86 and 3.83 (2s, 3H each).

### EXAMPLE 245

### 6-(((4-cyano-3-(8-quinolinyl)phenyl)(1-methyl-1H-imidazol-5-yl)methoxy)methyl)nicotinic acid trihydrochloride

### EXAMPLE 245A

### methyl 6-(((3-bromo-4-cyanophenyl)(1-methyl-1H-imidazol-5-yl)methoxy)methyl)nicotinate

The desired product was prepared by substituting methyl 6-bromomethyl-nicotinate for 4-cyanobenzyl bromide in Example 200C.

MS (APCI(+)) m/z 441 (M+H)⁺.

### EXAMPLE 245B

### 6-(((4-cyano-3-(8-quinolinyl)phenyl)(1-methyl-1H-imidazol-5-yl)methoxy)methyl)nicotinic acid trihydrochloride

The desired product was prepared by substituting Example 245A and 8-quinolinylboronic acid for Example 200C and 2-formylphenylboronic acid, respectively, in Example 200D.

MS (APCI(+)) m/z 476 (M+H)⁺; MS (APCI(−)) m/z 510 (M+Cl)⁻; ¹H NMR (300 MHz, DMSO-d₆) (rotamers) δ 9.12 and 9.11 (2s, 1H each), 9.04-8.88 (m, 2H), 8.58 and 8.50 (2d, 1H each), 8.31 and 8.25 (2d, 1H each), 8.15 and 8.06 (2d, 1H each), 7.98-7.88 (m, 1H), 7.78-7.48 (m, 5H), 7.13-7.08 (m, 1H), 6.35 (s, 1H), 4.91-4.74 (m, 2H), 3.85 and 3.83 (2s, 3H each).

### EXAMPLE 247

### 6-(((4-cyano-3-(8-quinolinyl)phenyl)(1-methyl-1H-imidazol-5-yl)methoxy)methyl)nicotinonitrile trihydrochloride

A solution of Example 244A (34 mg, 0.084 mmol) and 8-quinolinylboronic acid (23 mg, 0.13 mmol) in 1,2-dimethoxyethane (1.5 mL) was treated with cesium fluoride (32 mg, 0.2 mmol) and Pd(Ph₃P)₄ (4 mg), purged with argon, heated to 100° C., stirred for 16 hours, and filtered. The filtrate was purified by HPLC on a C₁₈ reverse phase column with acetonitrile/10 mM ammonium acetate, concentrated, lyophilized, dissolved in dichloromethane, treated with 1M HCl in diethyl ether, and concentrated to provide the desired product.

MS (ESI(+)) m/z 457 (M+H)⁺ and 489 (M+Na)⁺; ¹H NMR (300 MHz, DMSO-d₆) δ 9.09 (s, 1H), 8.97 (dd, 1H), 8.87 (dd, 1H), 8.50 (dd, 1H), 8.31 (dd, 1H), 8.16 (dd, 1H), 8.06 (d, 1H), 7.87 (dd, 1H), 7.79-7.70 (m, 4H), 7.62 (dd, 1H), 7.56 (s, 1H), 6.22 (s, 1H), 4.80 (q, 2H), 3.80 (s, 3H).

### EXAMPLE 248

### 5-(((3,4-dichlorobenzyl)oxy)(1-methyl-1H-imidazol-5-yl)methyl)-2′-(trifluoromethyl)(1,1′-biphenyl)-2-carbonitrile hydrochloride

The desired product was prepared by substituting Example 240A and 2-(trifluoromethyl)phenylboronic acid for Example 200C and 2-formylphenylboronic acid, respectively, in Example 200D.

MS (APCI(−)) m/z 550, 552, and 554 (M+^{35/37} Cl)⁻; ¹H NMR (300 MHz, DMSO-d₆) (rotamers) δ 8.82 (2s, 1H), 8.08 (dd, 1H), 7.92 (t, 1H), 7.88-7.80 (m, 1H), 7.76-7.67 (m, 2H), 7.63-7.50 (m, 4H), 7.40-7.25 (m, 2H), 6.07 (s, 1H), 4.66-4.49 (m, 2H), 3.69 and 3.68 (2s, 3H each)

### EXAMPLE 249

### 5-(((3-fluoro-4-(trifluoromethyl)benzyl)oxy)(1-methyl-1H-imidazol-5-yl)methyl)-2′-(trifluoromethyl)(1,1′-biphenyl)-2-carbonitrile hydrochloride

The desired product was prepared by substituting Example 241A and 2-(trifluoromethyl)phenylboronic acid for Example 200C and 2-formylphenylboronic acid, respectively, in Example 200D.

MS (APCI(−)) m/z 568 (M+Cl)⁻; MS (APCI(+)) m/z 534 (M+H)⁺; ¹H NMR (300 MHz, DMSO-d₆) (rotamers) δ 9.03 and 9.01 (2s, 1H each), 8.08 (dd, 1H), 7.92 (t, 1H), 7.84-7.71 (m, 4H), 7.60-7.49 (m, 3H), 7.40-7.36 (m, 2H), 6.14 (s, 1H), 4.76 (d, 1H), 4.67 and 4.60 (2d, 1H each), 3.72 and 3.70 (2s, 3H each).

### EXAMPLE 250

### 5-(((4-fluoro-3-(trifluoromethyl)benzyl)oxy)(1-methyl-1H-imidazol-5-yl)methyl)-2′-(trifluoromethyl)(1,1′-biphenyl)-2-carbonitrile hydrochloride

The desired product was prepared by substituting Example 242A and 2-(trifluoromethyl)phenylboronic acid for Example 200C and 2-formylphenylboronic acid, respectively, in Example 200D.

MS (APCI(−)) m/z 568 (M+Cl)⁻; MS (APCI(+)) m/z 534 (M+H)⁺; ¹H NMR (300 MHz, DMSO-d₆) (rotamers) δ 9.03 and 9.01 (2s, 1H each), 8.08 (dd, 1H), 7.94-7.68 (m, 6H), 7.60-7.49 (m, 3H), 7.34 (d, 1H), 6.11 (s, 1H), 4.78-4.60 (m, 2H), 3.72 and 3.70 (2s, 3H each).

### EXAMPLE 251

### 6-(((6-cyano-2′-(trifluoromethyl)(1,1′-biphenyl)-3-yl)(1-methyl-1H-imidazol-5-yl)methoxy)methyl)nicotinonitrile dihydrochloride

The desired product was prepared by substituting Example 244A and 2-(trifluoromethyl)phenylboronic acid for Example 200C and 2-formylphenylboronic acid, respectively, in Example 200D.

MS (APCI(−)) m/z 508 (M+Cl)⁻; MS (APCI(+)) m/z 474 (M+H)⁺; ¹H NMR (300 MHz, DMSO-d₆) (rotamers) δ 9.09 and 9.08 (2s, 1H each), 8.97 (dd, 1H), 8.34-8.31 (m, 1H), 8.08 (dd, 1H), 7.92 (t, 1H), 7.84-7.68 (m, 4H), 7.60-7.59 (m, 1H), 7.55-7.52 (m, 1H), 7.43 (d, 1H), 6.21 (s, 1H), 4.84-4.68 (m, 2H), 3.74 and 3.72 (2s, 3H each).

### EXAMPLE 252

### 4-(2-((4-cyanobenzyl)oxy)-2-(1-methyl-1H-imidazol-5-yl)ethyl)-2-(1-naphthyl)benzonitrile hydrochloride

### EXAMPLE 252A

### 1-methyl-2-(triethylsilyl)-1H-imidazole-5-carbaldehyde

A solution of Example 87F (10 g, 51 mmol) in THF (150 mL) at −74 ° C., was treated dropwise with 1.7M tert-butyllithium in pentane (32 mL, 54 mmol), stirred for 20 minutes, treated with 4-formylmorpholine (5.5 mL, 6.3 g, 5.5 mmol), stirred for 1 hour, warmed to room temperature, and treated with ethyl acetate and water. The organic layer was washed with brine, dried (Na₂SO₄), filtered, and concentrated to provide the desired product of sufficient purity for subsequent use without further purification.

MS (DCI₃) m/z 225 (M+H)⁺; ¹H NMR (300 MHz, CDCl₃) δ 9.76 (s, 1H), 7.89 (s, 1H), 4.00 (s, 3H), 1.00 (m, 15H).

### EXAMPLE 252B

### 4-(bromomethyl)-2-(1-naphthyl)benzonitrile

A solution of Example 89B (1.3 g, 5.0 mmol) in DMF (10 mL) at 0° C. was treated with LiBr (0.44 g, 5.1 mmol) and PBr₃ (0.47 mL, 1.35 g, 5.0 mmol), warmed to room temperature, poured over ice and extracted with diethyl ether. The extract was washed with water and brine, dried (Na₂SO₄), filtered, and concentrated to provide the desired product of sufficient purity for subsequent use without further purification.

¹H NMR (300 MHz, CDCl₃) δ 7.96 (m, 3H), 7.82 (m, 1H), 7.55 (m, 7H), 4.56 (s, 2H).

### EXAMPLE 252C

### 4-(2-hydroxy-2-(1-methyl- 1H-imidazol-5-yl)ethyl)-2-(1-naphthyl)benzonitrile

The desired product was prepared by the method described in J. Org. Chem. 1988, Vol.53, page 5789 using Example 252A and Example 252B, then purified by flash column chromatography on silica gel with 95:4:1 to 90:9:1/ethyl acetate:ethanol:concentrated ammonium hydroxide.

MS (DCI/NH₃) m/z 354 (M+H)⁺.

### EXAMPLE 252D

### 4-(2-((4-cyanobenzyl)oxy)-2-(1-methyl-1H-imidazol-5-yl)ethyl)-2-(1-naphthyl)benzonitrile hydrochloride

The desired product was prepared by substituting Example 252C and 4-cyanobenzyl bromide for Example 5D and (bromomethyl)benzene, respectively, in Example 5E.

MS (APCI(+)) m/z 469 (M+H)⁺; ¹H NMR (300 MHz, DMSO-d₆) δ 9.09 (s, 1H), 8.06 (m, 2H), 8.00 (d, 1H), 7.75-7.22 (envelope, 12H), 5.20 (m 1H), 4.62 (m, 1H), 4.50 (m, 1H), 3.88 (s, 3H), 3.50 (m, 2H); Anal. calcd for C₃₁H₂₅ClN₄O·3.00 H₂O: C, 66.60; H, 5.59; N, 10.02. Found: C, 66.19; H, 5.46; N, 10.50.

### EXAMPLE 253

### 4-(((4-cyanophenyl)(1-methyl-1H-imidazol-5-yl)methoxy)methyl)-2-(1-naphthyl)benzonitrile hydrochloride

### EXAMPLE 253A

### 4-(hydroxy(1-methyl-1H-imidazol-5-yl)methyl)benzonitrile

Example 252A and 4-cyanophenylzinc iodide were processed as described in J. Org. Chem. 1988, Vol.53, page 5789, treated with NaBH4, and purified to provide the desired product.

MS (DCI/NH₃) m/z 214 (M+H)⁺; ¹H NMR (300 MHz, DMSO-d₆) δ 7.84 (m, 2H), 7.60 (s, 1H), 7.55 (m, 2H), 6.38 (s, 1H), 6.18 (d, 1H), 5.90 (d, 1H), 3.57 (s, 3H).

### EXAMPLE 253B

### 4-(((4-cyanophenyl)(1-methyl-1H-imidazol-5-yl)methoxy)methyl)-2-(1-naphthyl)benzonitrile hydrochloride

The desired product was prepared by substituting Example 253A and Example 252B for Example 5D and (bromomethyl)benzene, respectively, in Example 5E.

MS (APCI(+)) m/z 455 (M+H)⁺; ¹H NMR (300 MHz, DMSO-d₆) δ 9.07 (d, 1H), 8.06 (m, 3H), 7.93 (m, 2H), 7.66 (m, 4H), 7.60 (m, 2H), 7.50 (m, 2H), 7.43 (d, 1H), 7.35 (s, 1H), 6.12 (s, 1H), 4.82, (m, 1H), 4,68 (m, 1H), 3.75 and 3.74 (both s, total 3H); Anal. calcd for C₃₀H₂₃ClN₄O·1.75 H₂O: C, 68.96; H, 5.11; N, 10.72. Found: C, 68.65; H, 4.92; N, 11.17.

### EXAMPLE 254

### 4-((2-(4-cyanophenyl)-1-(1-methyl-1H-imidazol-5-yl)ethoxy)methyl)-2-(1-naphthyl)benzonitrile hydrochloride

### EXAMPLE 254A

### 4-(2-hydroxy-2-(1-methyl-1H-imidazol-5-yl)ethyl)benzonitrile

Example 252A and 4-cyanobenzylzinc bromide were processed as described in J. Org. Chem. 1988, Vol.53, page 5789, treated with NaBH4, and purified to provide the desired product.

MS (DCI/NH₃) m/z 228 (M+H)⁺; ¹H NMR (300 MHz, DMSO-d₆) δ 7.73 (m, 2H), 7.59 (m, 2H), 7.55 (s, 1H), 6.80 (s, 1H), 5.30 (d, 1H), 4.81(m, 1H), 3.60 (s, 3H), 3.15 (m, 2H).

### EXAMPLE 254B

### 4-((2-(4-cyanophenyl)-1-(1-methyl-1H-imidazol-5-yl)ethoxy)methyl)-2-(1-naphthyl)benzonitrile hydrochloride

The desired product was prepared by substituting Example 254A and Example 252B for Example 5D and (bromomethyl)benzene, respectively, in Example 5E, and by substituting 4:1 dichloromethane/DMF for dichloromethane.

MS (APCI(+)) m/z 469 (M+H)⁺; ¹H NMR (300 MHz, DMSO-d₆) δ 9.10 (s, 1H), 8.07 (m, 2H), 7.96 (d, 1H), 7.88 and 7.78 (both m, total 1H), 7.70-7.35 (m, 1 lH), 5.13 and 5.00 (both m, total 1H), 4.67 (m, 1H), 4.55 (m, 1H), 3.86 (s, 3H), 3.30 (m, 2H).

### EXAMPLE 255

### 4-((1-(1-methyl-1H-imidazol-5-yl)-3-phenylpropoxy)methyl)-2-(1-naphthyl)benzonitrile hydrochloride

### EXAMPLE 255A

### 1-(1-methyl-1H-imidazol-5-yl)-3-phenyl-1-propanol

The desired product was prepared by substituting phenethylmagnesium chloride for phenylmagnesium bromide in Example 256A.

MS (DCI/NH₃) m/z 217 (M+H)⁺; ¹H NMR (300 MHz, CDCl₃) δ 7.43 (s, 1H), 7.30 (m, 2H), 7.20 (m, 3H), 6.94 (s, 1H), 4.63 (t, 1H), 3.69 (s, 3H), 2.80 (m, 2H), 2.23 (m, 2H).

### EXAMPLE 255B

### 4-((1-(1-methyl-1H-imidazol-5-yl)-3-phenylpropoxy)methyl)-2-(1-naphthyl)benzonitrile hydrochloride

The desired product was prepared by substituting Example 255A and Example 252B for Example 5D and (bromomethylbenzene), respectively, in Example 5E.

MS (APCI(+)) m/z 458 (M+H)⁺; ¹H NMR (300 MHz, DMSO-d₆) δ 9.05 (s, 1H), 8.08 (m, 3H), 7.75 (m, 1H), 7.65 (m, 3H), 7.54 (m, 3H), 7.43 (m, 1H), 7.20 (m, 5H), 4.80 (m, 1H), 4.60 (m, 2H), 3.82 and 3.80 (both s, total 3H), 2.70 (m, 2H), 2.30 (m, 1H), 2.13 (m, 1H); Anal. calcd for C₃₁H₂₈ClN₃O·2.40 H20: C, 69.30; H, 6.15; N, 7.83. Found: C, 69.15; H, 5.59; N, 7.83.

### EXAMPLE 256

### 4-(((1-methyl-1H-imidazol-5-yl)(phenyl)methoxy)methyl)-2-(1-naphthyl)benzonitrile hydrochloride

### EXAMPLE 256A

### (1-methyl-1H-imidazol-5-yl)(phenyl)methanol

A solution of Example 252A (1.2 g, 5.4 mmol) in THF (11 mL) at −10° C., was treated with phenylmagnesium bromide (3.M, 1.8 mL, 5.4 mmol), stirred for 1 hour, treated with methanol, warmed to room temperature, stirred for 16 hours, concentrated, and treated with ethyl acetate and water. The organic layer was washed with brine, dried (Na₂SO₄), filtered, and concentrated to provide the desired product of sufficient purity for subsequent use without further purification.

MS (DCI/NH₃) m/z 189 (M+H)⁺; ¹H NMR (300 MHz, DMSO-d₆) δ 7.51 (s, 1H), 7.38 (m, 4H), 7.29 (m, 1H), 6.38 (s, 1H), 5.91 (d, 1H), 5.77 (d, iH), 3.55 (s, 3H).

### EXAMPLE 256B

### 4-(((1-methyl-1H-imidazol-5-yl)(phenyl)methoxy)methyl)-2-(1-naphthyl)benzonitrile hydrochloride

The desired product was prepared by substituting Example 256A and Example 252B for Example 5A and (bromomethyl)benzene, respectively, in Example 5E, and by substituting 4:1/dichloromethane:DMF for dichloromethane.

MS (APCI(+)) m/z 430 (M+H)⁺; ¹H NMR (300 MHz, DMSO-d₆) δ 9.07 (s, 1H), 8.05 (m, 3H), 7.65 (m, 5H), 7.50 (m, 7H), 7.30 (s, 1H), 5.99 (s, 1H), 4.80 (m, 1H), 4.66 (m, 1H), 3.75 and 3.74 (both s, total 3H); Anal. calcd for C₂₉H₂₄ClN₃O·1.70 H₂O: C, 70.14; H, 5.56; N, 8.46. Found: C, 70.14; H, 5.49; N, 8.49.

### EXAMPLE 257

### 4((1-methyl-1H-imidazol-5-yl)(phenyl)methyl)amino)methyl)-2-(1-naphthyl)benzonitrile dihydrochloride

### EXAMPLE 257A

### (1-methyl-1H-imidazol-5-yl)(phenyl)methanamine hydrochloride

The desired product was prepared by substituting Example 256A for Example 89D in Example 13A.

MS (DCI/NH₃) m/z 188 (M+H)⁺; ¹H NMR (300 MHz, DMSO-d₆) δ 7.45 (s, 1H), 7.35 (m, 5H), 6.55 (s, 1H), 5.09 (s, 1H), 3.50 (s, 3H), 2.26 (br s, 2H).

### EXAMPLE 257B

### 4((((1-methyl-1H-imidazol-5-yl)(phenyl)methyl)amino)methyl)-2-(1-naphthyl)benzonitrile dihydrochloride

Example 89C and Example 257A were processed as described in Example 12B, substituting dichloromethane for 1,2-dichloroethane. The mixture was treated with methanol and stirred for 4 hours prior to treatment with ethyl acetate to provide the desired product.

MS (APCI(+)) m/z 429 (M+H)⁺; ¹H NMR (300 MHz, DMSO-d₆) δ 9.05 (s, 1H), 8.05 (m, 3H), 7.82, 7.72, and 7.60 (envelope, 6H), 7.45 (m, 7H), 5.75 (br s, 1H), 4.15 (br m, 2H), 3.81 and 3.79 (both s, total 3H); Anal. calcd for C₂₉H₂₆Cl₂N₄·1.65 H₂O: C, 65.57; H, 5.56; N, 10.55. Found: C, 65.61; H, 5.54; N, 10.49.

### EXAMPLE 258

### 4-((1-(1-methyl-1H-imidazol-5-yl)-2-phenylethoxy)methyl)-2-(1-naphthyl)benzonitrile hydrochloride

### EXAMPLE 258A

### 1-(1-methyl-1H-imidazol-5-yl)-2-phenylethanol

The desired product was prepared by substituting benzylmagnesium chloride for phenylmagnesium bromide in Example 256A.

MS (DCI/NH₃) m/z 203 (M+H)⁺; ¹H NMR (300 MHz, DMSO-d₆) δ 7.46 (s, 1H), 7.24 (m, 5H), 6.80 (s, 1H), 5.23 (d, 1H), 4.77 (m, 1H), 3.55 (s, 3H), 3.05 (m, 2H).

### EXAMPLE 258B

### 4-((1-(1-methyl-1H-imidazol-5-yl)-2-phenylethoxy)methyl)-2-(1-naphthyl)benzonitrile hydrochloride

The desired product was prepared by substituting Example 257A and Example 252B for Example 5D and (bromomethyl)benzene, respectively, in Example 5E, and substituting 4:1 dichloromethane/DMF for dichloromethane.

MS (APCI(+)) m/z 444 (M+H)⁺; ¹H NMR (300 MHz, DMSO-d₆) δ 9.05 (s, 1H), 8.09 (m, 2H), 7.95 (d, 1H), 7.70-7.00 (envelope, 13H), 5.07 (m, 1H), 4.70 (m, 1H), 4.55 (m, 1H), 3.80 (m, 3H), 3.12 (m, 2H); Anal. calcd for C₃₀H₂₆ClN₃O·2.70 H₂O: C, 68.16; H, 5.99; N, 7.95. Found: C, 68.14; H, 5.89; N, 7.99.

### EXAMPLE 259

### 4-(((1-(1-methyl-1H-imidazol-5-yl)-2-phenylethyl)amino)methyl)-2-(1-naphthyl)benzonitrile dihydrochloride

### EXAMPLE 259A

### 1-(-1-methyl-1H-imidazol-5-yl)-2-phenylethanediazonium chloride

A solution of Example 258A (0.4 g, 2.0 mmol) in dichloromethane at 0° C. was treated with thionyl chloride, stirred for 30 minutes, warmed to room temperature, stirred for 1.5 hours, and concentrated. The concentrate was treated with DM (5 mL) and sodium azide (0.54 g, 8.2 mmol), heated to 55° C., stirred for 3.5 hours, and treated with ethyl acetate and 0.5M NaHCO₃. The organic layer was washed with brine, dried (Na₂SO₄), filtered, and concentrated to provide the desired product of sufficient purity for subsequent reaction without further purification.

### EXAMPLE 259B

### 1-(1-methyl-1H-imidazol-5-yl)-2-phenylethylamine

A solution of Example 259A in THF (5 mL) was treated with triphenylphosphine (0.75 g, 2.8 mmol), heated to reflux, stirred for 1 hour, cooled to room temperature, treated with water (0.5 mL), stirred for 16 hours, concentrated, and treated with 2M HCl and ethyl acetate. The aqueous layer was adjusted to pH 10 with 2M Na₂CO₃ and extracted with 3:1/chloroform:isopropanol. The extract was dried (Na₂SO₄), filtered, and concentrated to provide the desired product of sufficient purity for subsequent use without further purification.

MS (DCI/NH₃) m/z 202 (M+H)⁺; ¹H NMR (300 MHz, DMSO-d₆) δ 7.42 (s, 1H), 7.24 (m, 2H), 7.18 (m, 3H), 6.78 (s, 1H), 4.05 (m, 1H), 3.52 (s, 3H), 2.94 (m, 2H).

### EXAMPLE 259C

### 4-((1 -(1-methyl-1H-imidazol-5-yl)-2-phenylethyl)amino)methyl)-2-(1-naphthyl)benzonitrile dihydrochloride

The desired product was prepared by substituting Example 259A for Example 257A in Example 257B.

MS (APCI(+)) m/z 443 (M+H)⁺; ¹H NMR (300 MHz, DMSO-d₆) δ 8.95 (s, 1H), 8.20 (s, 1H), 8.10 (m, 3H), 7.95 (br d, 1H), 7.86 (br d, 1H), 7.66 (m, 1H), 7.60 (m, 1H), 7.53 (m, 3H), 7.25 (m, 3H), 7.14 (m, 2H), 4.91 (br s, 1H), 4.40 (br m, 2H), 3.80 (br m, 1H), 3.59 (s, 3H), 3.30 (m, 1H); Anal. calcd for C₃₀H₂₈Cl₂N₄·1.40 H₂O: C, 66.64; H, 5.74; N, 10.36. Found: C, 66.92; H, 5.83; N, 9.92.

### EXAMPLE 260

### 4(((1-(1-methyl-1H-imidazol-5-yl)-3-phenylpropyl)amino)methyl)-2-(1-naphthyl)benzonitrile dihydrochloride

### EXAMPLE 260A

### 1-(1-methyl-1H-imidazol-5-yl)-3-phenylpropyldiazonium chloride

The desired product was prepared by substituting Example 255A for Example 258A in Example 259A.

### EXAMPLE 260B

### 1-(1-methyl-1H-imidazol-5-yl)-3-phenylpropylamine

The desired product was prepared by substituting Example 260A for Example 259A in Example 259B.

MS (DCI/NH₃) m/z 216 (M+H)⁺.

### EXAMPLE 260C

### 4-((1-(1-methyl-1 H-imidazol-5-yl)-3-phenylpropyl)amino)methyl)-2-(1-naphthyl)benzonitrile dihydrochloride

The desired product was prepared by substituting Example 260B for Example 257A in Example 257B.

MS (APCI(+)) m/z 457 (M+H)⁺; ¹H NMR (300 MHz, DMSO-d₆) δ 9.05 (br s, 1H), 8.10 (m, 2H), 8.00 (br s, 1H), 7.90 (br m, 1H), 7.81 (s, 1H), 7.60 (m, 4H), 7.50 (m, 2H), 7.25 (m, 2H), 7.16 (m, 3H), 4.50, 4.35, and 4.20 (envelope, 3H), 3.79 and 3.75 (both s, total 3H), 2.60 (m, 2H), 2.40 and 2.20 (both br m, total 2H).

### EXAMPLE 261

### 4(((4-cyanophenyl)-1-(1-methyl-1H-imidazol-5-yl)ethyl)amino)methyl)-2-(1-naphthyl)benzonitrile ditrifluoroacetic acid salt

### EXAMPLE 261A

### 2-(4-cyanophenyl)-1-(1-methyl-1H-imidazol-5-yl)ethanediazonium chloride

The desired product was prepared by substituting Example 254A for Example 258A in Example 259A.

### EXAMPLE 261B

### 4-(2-amino -2-(1-methyl-1H-imidazol-5-yl)ethyl)benzonitrile

The desired product was prepared by substituting Example 261A for Example 259A in Example 259B.

MS (DCI/NH₃) m/z 227 (M+H)⁺.

### EXAMPLE 261C

### 4-(((2-(4-cyanophenyl)-1-(1-methyl-1H-imidazol-5-yl)ethyl)amino)methyl)-2-(1-naphthyl)benzonitrile ditrifluoroacetic acid

The desired product was prepared by substituting Example 261B for Example 257A in Example 257B, and purified by preparative HPLC with 0-70% acetonitrile/0.1% trifluoroacetic acid.

MS (APCI(+)) m/z 468 (M+H)⁺; ¹H NMR (300 MHz, DMSO-d₆) δ 8.86 (s, 1H), 8.10 (m, 3H), 7.77-7.56 (envelope, 7H), 7.55-7.35 (envelope, 5H), 4.60 (br s, 1H), 4.13 (br s, 2H), 3.25 (br m, 2H); Anal. calcd for C₃₅H₂₇F₆N₅O₄-1.70 H₂O: C, 57.89; H, 4.22; N, 9.64. Found: C, 57.85; H, 4.11; N, 9.71.

### EXAMPLE 262

### 4-(((3-cyanobenzyl)oxy)(1-methyl-1H-imidazol-5-yl)methyl)-2-(1-naphthyl)benzonitrile

The desired product was prepared by substituting Example 89D and 3-(bromoethyl)benzonitrile for Example 1B and (bromomethyl)benzene, respectively, in Example 1C.

MS (ESI(+)) m/z 455 (M+H)⁺; ¹H NMR (300 MHz, DMSO-d₆) δ 9.15 (s, 1H), 8.15 (dd, 1H), 8.1 (t, 2H), 7.9-7.45 (m, 11H), 6.15 (s, 1H), 4.7 (q, 2H), 3.8 (d, 3H), 3.6 (s, 1 H); Anal. calcd for C₃₀H₂₃ClN₄O·1.6H₂O: C, 69.32; H, 5.08; N, 10.78. Found: C, 69.40; H, 5.16; N, 10.21.

### EXAMPLE 263

### 4-(((4-bromobenzyl)oxy)(1-methyl-1H-imidazol-5-yl)methyl)-2-(1-naphthyl)benzonitrile

The desired product was prepared by substituting Example 89D and 1-bromo-4-(bromomethyl)benzene for Example 1B and (bromomethyl)benzene, respectively, in Example 1C.

MS (ESI(+)) m/z 508 (M+H)⁺; ¹H NMR (300 MHz, DMSO-d₆) δ 9.1 (s, 1H), 8.15 (dd, 1H), 8.05 (t, 2H), 7.8 (d, 1H), 7.7-7.4 (m, 8H), 7.35 (dd, 2H), 6.6 (s, 1), 4.6 (q, 2H), 3.8 (d, 3H); Anal. calcd for C₂₉H₂₃BrClN₃O·0.9H₂O: C, 62.08; H, 4.46; N, 7.49. Found: C, 62.13; H, 4.50; N, 7.43.

### EXAMPLE 264

### 4-((3 -chlorobenzyl)((1-methyl-1H-imidazol-5-yl)methyl)amino)-2-(1-naphthyl)benzonitrile

The desired product was prepared by substituting 3-(bromomethyl)-1-chlorobenzene for benzyl bromide in Example 232.

MS (ESI(+)) m/z 463 (M+H)⁺; H NMR (300 MHz, DMSO-d₆) δ 7.99 (d, 2H), 7.72 (d, 1H), 7.6-7.3 (m, 8H), 7.25-7.20 (m, 1H), 7.2-7.1 (m, 1H), 7.00 (dd, 1H), 6.83 (d, 1H), 6.70 (s, 1H), 4.84 (m, 2H), 4.75 (m, 2H), 3.55 (s, 3H); Anal. calcd for C₂₉H₂₃N₄Cl·0.5 H₂O: C, 73.79; H, 5.12; N, 11.87. Found: C, 73.74; H, 5.03; N, 11.72.

### EXAMPLE 265

### 4-(benzyl(1H-imidazol-5-ylmethyl)amino)-2-(1-naphthyl)benzonitrile

### EXAMPLE 265A

### 4-((1H-imidazol-5-ylmethyl)amino)-2-(1-naphthyl)benzonitrile

The desired product was prepared by substituting Example 270A for Example 234B in Example 234C.

### EXAMPLE 265B

### 4-(benzyl(1H-imidazol-5-ylmethyl)amino)-2-(1-naphthyl)benzonitrile

The desired product was prepared by substituting Example 265A for Example 234C in Example 234D.

MS (ESI(+)) m/z 415 (M+H)⁺; ¹H NMR (300 MHz, DMSO-d₆) δ 11.96 (br s, 1H), 7.98 (d, 2H), 7.7-7.5 (m, 4H), 7.5-7.2 (m, 8H), 7.1-7.0 (m, 2H), 6.83 (d, 1H), 4.9-4.7 (m, 2H), 4.59 (m, 2H); Anal. calcd for C₂₈H₂₂N₄·0.75 H₂O: C, 78.57; H, 5.53; N, 13.08. Found: C, 78.33; H, 5.21; N, 12.93.

### EXAMPLE 266

### 4-((3-cyanobenzyl)((1-methyl-1H-imidazol-5-yl)methyl)amino)-2-(1-naphthyl)benzonitrile

The desired product was prepared by substituting 3-(bromomethyl)benzonitrile for benzyl bromide in Example 232.

MS (ESI(+)) m/z 454 (M+H)⁺; ¹H NMR (300 MHz, DMSO-d₆) δ 7.99 (d, 2H), 7.8-7.3 (m, 11H), 7.01 (dd, 1H, 1H), 6.82 (d, 1H), 6.71 (s, 1H), 5.0-4.7 (m, 4H), 3.54 (s, 3H); Anal. calcd for C₃₀H₂₃N₅·1.0 H₂O: C, 76.41; H, 5.34; N, 14.85. Found: C, 76.47; H, 5.14; N, 14.46.

### EXAMPLE 267

### N-(4-cyano-3-(1-naphthyl)phenyl)-N-((1-methyl-1H-imidazol-5-yl)methyl)benzenesulfonamide

The desired product was prepared by substituting benzenesulfonyl chloride for benzyl bromide in Example 232.

MS (ESI(+)) m/z 479 (M+H)⁺; ¹H NMR (300 MHz, DMSO-d₆) δ 8.1-8.0 (m, 2H), 7.97 (d, 1H), 7.80-7.45 (m, 10H), 7.38 (dd, 1H), 7.27 (d, 1H), 7.09 (d, 1H), 6.59 (s, 1H), 4.93 (m, 2H), 3.66 (s, 3H); Anal. calcd for C₂₈H₂₂N₄O₂S·0.75 H₂0: C, 68.34; H, 4.81; N, 11.38. Found: C, 68.30; H, 4.73; N, 10.93.

### EXAMPLE 268

### methyl 4-((4-cyano((1-methyl-1H-imidazol-5-yl)methyl)-3-(1-naphthyl)anilino)methyl)benzoate

The desired product was prepared by substituting methyl 4-(bromomethyl)-benzoate for benzyl bromide in Example 232.

MS (ESI(+)) m/z 487 (M+H)⁺; ¹H NMR (300 MHz, DMSO-d₆) δ 7.98 (d, 2H), 7.92 (d, 2H), 7.70 (d, 1H), 7.6-7.5 (m, 3H), 7.40 (dd, 1H), 7.4-7.3 (m, 4H), 7.00 (dd, 1H), 6.81 (d, 1H), 6.72 (s, 1H), 5.0-4.7 (m, 4H), 3.85 (s, 3H), 3.54 (s, 3H); Anal. calcd for C₂₀H₁₈N₄O₂·1.0 H₂O: C, 73.79; H, 5.59; N, 11.10. Found: C, 73.87; H, 5.40; N, 10.60.

### EXAMPLE 269

### 4-((4-cyano((1-methyl-1H-imidazol-5-yl)methyl)-3-(1-naphthyl)anilino)methyl)benzoic acid

The desired product was prepared by substituting Example 268 for Example 10F in Example 10G.

MS (ESI(+)) m/z 473 (M+H)⁺; ¹H NMR (300 MHz, DMSO-d₆) δ 12.90 (br s, 1H), 7.97 (dd, 2H), 7.90 (d, 2H), 7.71 (d, 1H), 7.6-7.3 (m, 8H), 6.82 (d, 1H), 6.73 (s, 1H), 5.0-4.7 (m, 4H), 3.55 (s, 3H); HRMS calcd m/z for C₃₀H₂₅N₄O₂: 473.1978 (M+H)⁺. Found: 473.1984.

### EXAMPLE 270

### 5-(benzyl(1H-imidazol-5-ylmethyl)amino)-2′-methyl(1,1′-biphenyl)-2-carbonitrile

### EXAMPLE 270A

### 1-(triphenylmethyl)imidazole-4-carboxaldehyde

The desired product was prepared as described in J. Med. Chem., 1996, Vol.39, page 353.

### EXAMPLE 270B

### 2′-methyl-5-(((1-trityl-1H-imidazol-4-yl)methyl)amino)(1,1′-biphenyl)-2-carbonitrile

The desired product was prepared by substituting Example 270A for Example 225D in Example 225E.

### EXAMPLE 270C

### 5-(benzyl((1-trityl-1H-imidazol-4-yl)methyl)amino)-2′-methyl(1,1′-biphenyl)-2-carbonitrile

The desired product was prepared by substituting Example 270B for Example 225E in Example 226.

### EXAMPLE 270D

### 5-(benzyl(1H-imidazol-5-ylmethyl)amino)-2′-methyl(1,1′-biphenyl)-2-carbonitrile

A solution of Example 270C (380 mg, 0.61 mmol) in dichloromethane (10 mL) at room temperature was treated with trifluoroacetic acid (3 mL) and triethylsilane (1.5 mL), stirred for 2 hours, and concentrated under a nitrogen atmosphere. The concentrate was treated with ethyl acetate, washed with saturated NaHCO₃ and brine; dried (MgSO₄), filtered, and concentrated. The concentrate was purified by flash column chromatography on silica gel with 95:5/dichloromethane:methanol to provide the desired product.

MS (ESI(+)) m/z 379 (M+H)⁺; ¹H NMR (300 MHz, DMSO-d₆) δ 11.90 (br s, 1H), 7.6-7.5 (m, 2H), 7.4-7.1 (m, 9H), 7.1-7.0 (m, 2H), 6.95 (dd, 1H), 6.64 (d, 1H), 4.78 (br s, 2H), 4.57 (br s, 2H), 1.96 (s, 3H); Anal. calcd for C₂₅H₂₂N₄·0.5 H₂O: C, 77.49; H, 5.98; N, 14.45. Found: C, 77.20; H, 6.08; N, 13.96.

### EXAMPLE 271

### methyl 3-((4-cyano((1-methyl-1H-imidazol-5-yl)methyl)-3-(1-naphthyl)anilino)methyl)benzoate

The desired product was prepared by substituting methyl 3-(bromomethyl)benzoate for benzyl bromide in Example 232.

MS (ESI(+)) m/z 487 (M+H)⁺; ¹H NMR (300 MHz, DMSO-d₆) δ 8.0-7.9 (m, 2H), 7.9-7.8 (m, 1H), 7.77 (s, 1H), 7.71 (d, 1H), 7.60-7.45 (m, 5H), 7.41 (dd, 1H), 7.31 (d, 2H), 7.03 (dd, 1H), 6.85 (d, 1H), 6.72 (s, 1H), 5.0-4.7 (m, 4H), 3.83 (s, 3H), 3.56 (s, 3H); Anal. calcd for C₃₁H₂₆N₄O₂·0.75 H₂O: C, 74.45; H, 5.54; N, 11.20. Found: C, 74.58; H, 5.31; N, 10.83.

### EXAMPLE 272

### 4-(((6-cyano-2′-methyl(1,1′-biphenyl)-3-yl)(1-methyl-1H-imidazol-5-yl)methoxy)methyl)benzoic acid

A solution of Example 130 in THF (10 mL) and water (5 mL) at room temperature was treated with LiOH (100 mg ), stirred for 16 hours, adjusted to pH 7 with saturated ammonium chloride (20 mL) and extracted with ethyl acetate. The extract was dried (Na₂SO₄), filtered, and concentrated to provide the desired product of sufficient purity for subsequent use without further purification.

MS (APCI(+)) m/z 438 (M+H)⁺; MS (APCI(−)) m/z 472 (M+Cl)⁻; ¹H NMR (500 MHz, DMSO-d₆) δ 8.95 (br s, 1H), 8.06 (d, 1H), 7.93 (d, 2H), 7.68 (dd, 1H), 7.52 (s, 1H), 7.48 (d, 2H), 7.41-7.25 (m, 5H), 6.08 (s, 1H), 4.66 (d, 1H), 4.64 (d, 1H), 3.74 (s, 3H), 2.13 (s, 3H).

### EXAMPLE 273

### 4-((1-methyl-1H-imidazol-5-yl)((3-chlorobenzyl)oxy)methyl)-2-(1-naphthyl)benzonitrile hydrochloride

The desired product was prepared by substituting Example 89D and 3-chlorobenzyl bromide for Example 5D and (bromomethyl)benzene, respectively, in Example 5E.

MS (DCI/NH₃) m/z 464 (M+H)⁺; ¹H NMR (300 MHz, DMSO-d₆) δ 9.05 (s, 1H), 8.17 (m, 1H), 8.09 (m, 2H), 7.78 (m, 1H), 7.5 (m, 11H), 7.40 (m, 1H), 6.12 (s, 1H), 4.65 (m, 2H), 3.79 (d, 3H).

### EXAMPLE 274

### 5-(((4-cyano-3-(1-naphthyl)phenyl)(1-methyl-1H-imidazol-5-yl)methoxy)methyl)-2-pyridinecarbonitrile dihydrochlolide

### EXAMPLE 274A

### methyl 6-cyanonicotinate

A solution of 6-cyanonicotinic acid (5 g) in methanol (100 mL) was titrated to a yellow endpoint with 2M trimethylsilyldiazomethane in hexanes and concentrated. The concentrate was purified by flash column chromatography on silica gel with 3:1/hexanes:ethyl acetate to provide the desired product.

### EXAMPLE 274B

### 5-(hydroxymethyl)-2-pyridinecarbonitrile

The desired product was prepared by substituting Example 274A for Example 5A in Example 5B.

MS (DCI/NH₃) m/z 136 (M+H)⁺; ¹H NMR (300 MHz, CDCl₃) δ 8.71 (m, 1H), 7.88 (m, 1H), 7.71 (m, 1H), 4.86 (d, 2H).

### EXAMPLE 274C

### 5-(bromomethyl)-2-pyridinecarbonitrile

The desired product was prepared by substituting Example 274B for Example 61A in Example 61B.

MS (DCI/NH₃) m/z 197 and 199 (M+H)⁺; ¹H NMR (300 MHz, CDCl₃) δ 8.73 (d, 1H), 7.89 (dd, 1H), 7.70 (d, 1H), 4.50 (s, 2H).

### EXAMPLE 274D

### 5-(((4-cyano-3-(1-naphthyl)phenyl)(1-methyl-1H-imidazol-5-yl)methoxy)methyl)-2-pyridinecarbonitrile dihydrochloride

The desired product was prepared by substituting Example 89D and Example 274C for Example 5D and (bromomethyl)benzene, respectively, in Example 5E.

MS (DCI/NH₃) m/z 456 (M+H)⁺; ¹H NMR (300 MHz, CDCl₃) δ 8.69 (s, 1H), 7.99 (t, 2H), 7.89 (d, 1H), 7.79 (m, 2H), 7.68 (m, 1H), 7.5 (m, 7H), 7.08 (d, 1H), 5.75 (d, 1H), 4.70 (s, 2H), 3.55 (d, 3H).

### EXAMPLE 275

### 5-(((4-cyanobenzyl)oxy)(1-methyl-1H-imidazol-5-yl)methyl)-3-(1-naphthyl)-2-pyridinecarbonitrile dihydrochloride

### EXAMPLE 275A

### methyl 5,6-dichloronicotinate

A solution of 5,6-dichloronicotinic acid (19.2 g, 100 mmol) in methanol (150 mL) at 0° C. was treated with thionyl chloride (10.9 mL, 150 mmol), warmed to room temperature over 18 hours, treated with ethyl acetate, washed sequentially with half-saturated NaHCO₃, water, and brine, dried (Na₂SO₄), filtered, and concentrated to provide the desired product of sufficient purity for subsequent use without further purification.

### EXAMPLE 275B

### methyl 5-chloro-6-cyanonicotinate

A mixture of Example 275A (2.0 g, 10 mmol), potassium iodide (830 mg, 5 mmol), K₂CO₃ (6.91 g, 50 mmol), and potassium cyanide (3.26 g, 50 mmol) in DMSO (20 mL) at 80° C. was stirred for 6 hours, cooled, treated with ethyl acetate, washed with water and brine, and concentrated. The concentrate was purified by flash column chromatography on silica gel with 3:1/hexanes:ethyl acetate to provide the desired product.

### EXAMPLE 275C

### methyl 6-cyano-5-(1-naphthyl)nicotinate

A mixture of Example 275B (800 mg, 4 mmol), 1-naphthaleneboronic acid (1.5 g, 8.7 mmol), palladium(I) acetate (17 mg, 0.08 mmol), 2-dimethylamino-2′-dicyclohexylphosphino-biphenyl (44 mg, 0.11 mmol), and CsF (2 g, 13 mmol) in dioxane (20 mL) at room temperature was stirred for 48 hours, treated with ethyl acetate, washed with water and brine, dried (Na₂SO₄), filtered, and concentrated. The concentrate was purified by flash column chromatography on silica gel with 9:1/hexanes:ethyl acetate to provide the desired product.

### EXAMPLE 275D

### 5-(hydroxymethyl)-3-(1-naphthyl)-2-pyridinecarbonitrile

The desired product was prepared by substituting Example 275C for Example 5A in Example 5B.

### EXAMPLE 275E

### 5-formyl-3-(1-naphthyl)-2-pyridinecarbonitrile

The desired product was prepared by substituting Example 275D for Example 35C in Example 35D.

¹H NMR (300 MHz, CDCl₃) δ 10.28 (s, 1H), 9.25 (d, 1H), 8.36 (d, 1H), 8.02 (m, 2H), 7.57 (m, 4H), 7.42 (m, 1H), 4.50 (s, 2H).

### EXAMPLE 275F

### 5-(hydroxy(1-methyl-1H-imidazol-5-yl)methyl)-3-(1-naphthyl)-2-pyridinecarbonitrile

The desired product was prepared by substituting Example 275E for Example 1A in Example 1B.

MS (DCI/NH₃) m/z 341 (M+H)⁺; ¹H NMR (300 MHz, CDCl₃) δ 8.81 (dd, 1H), 7.98 (m, 3H), 7.5 (m, 4H), 6.78 (s, 1H), 6.11 (s, 1H), 4.10 (m, 2H), 3.65 (d, 3H).

### EXAMPLE 275G

### 5-(((4-cyanobenzyl)oxy)(1-methyl-1H-imidazol-5-yl)methyl)-3-(1-naphthyl)-2-pyridinecarbonitrile dihydrochloride

The desired product was prepared by substituting Example 275F and 4-cyanobenzyl bromide for Example 5D and (bromomethyl)benzene, respectively, in Example 5E.

MS (DCI/NH₃) m/z 456 (M+H)⁺; ¹H NMR (300 MHz, CDCl₃) δ 8.82 (dd, 1H), 7.98 (m, 3H), 7.5 (m, 4H), 7.09 (d, 1H), 5.80 (s, 1H), 4.66 (m, 2H), 3.54 (s, 3H).

### EXAMPLE 276

### 4-((1-methyl-1H-imidazol-5-yl)((4-azidobenzyl)oxy)methyl)-2-(1-naphthyl)benzonitrile hydrochloride

### EXAMPLE 276A

### 4-(hydroxymethyl)benzenediazonium tetrafluoroborate

The desired product was prepared by substituting 4-aminobenzyl alcohol for Example 87A in Example 87B.

### EXAMPLE 276B

### (4-azidophenyl)methanol

The desired product was prepared by substituting Example 276A and sodium azide for Example 87B and CuCN/NaCN, respectively, in Example 87C.

¹H NMR (300 MHz, CDCl₃) δ 7.48 (d, 2H), 7.02 (d, 2H), 4.69 (s, 2H).

### EXAMPLE 276C

### 1-azido-4-(bromomethyl)benzene

The desired product was prepared by substituting Example 276B for Example 61A in Example 61B.

### EXAMPLE 276D

### 4-((1-methyl-1H-imidazol-5-yl)((4-azidobenzyl)oxy)methyl)-2-(1-naphthyl)benzonitrile hydrochloride

The desired product was prepared by substituting Example 89D and Example 276C for Example 5D and (bromomethyl)benzene, respectively, in Example 5E.

MS (DCI/NH₃) m/z 471 (M+H)⁺; ¹H NMR (300 MHz, CDCl₃) δ 7.96 (m, 2H), 7.87 (d, 1H), 7.70 (s, 1H), 7.50 (m, 7H), 7.29 (m, 2H), 6.99 (m, 3H), 5.63 (s, 1H), 4.54 (s, 2H), 3.52 (d, 3H).

### EXAMPLE 277

### methyl 6-(((6-cyano-2′-(trifluoromethyl)(1,1′-biphenyl)-3-yl)(1-methyl-1H-imidazol-5-yl)methoxy)methyl)nicotinate dihydrochloride

The desired product was prepared by substituting Example 245A and 2-(trifluoromethyl)phenylboronic acid for Example 200C and 2-formylphenylboronic acid, respectively, in Example 200D.

MS (APCI(−)) m/z 541 (M+Cl)⁻; MS (APCI(+)) m/z 507 (M+H)⁺; ¹H NMR (300 MHz, DMSO-d₆) (rotamers) δ 9.13 and 9.12 (2s, 1H each), 9.02 (dd, 1H), 8.32-8.28 (m, 1H), 8.09 (dd, 1H), 7.92 (t, 1H), 7.83-7.51 (m, 6H), 7.60-7.49 (m, 1H), 7.41 (dd, 1H), 6.22 (s, 1H), 4.82 (d, 1H), 4.72 (dd, 1H), 3.76 and 3.74 (2s, 3H each).

### EXAMPLE 278

### 5-(((4-cyanobenzyl)oxy)(1-methyl-1H-imidazol-5-yl)methyl)-2′,3′-dimethyl(1,1′-biphenyl)-2-carbonitrile hydrochloride

The desired product was prepared by substituting 2,3-dimethylphenylboronic acid for 2-formylphenylboronic acid in Example 200D.

MS (APCI(−)) m/z 467 (M+Cl)⁻; MS (APCI(+)) m/z 433 (M+H)⁺; ¹H NMR (300 MHz, DMSO-d₆) (rotamers) δ 8.99 (s, 1H), 8.04 (dd, 1H), 7.82 (d, 2H), 7.68-7.66 (m, 1H), 7.57 (d, 2H), 7.47 (d, 1H), 7.40 (d, 1H), 7.28 (d, 1H), 7.21 (q, 1H), 7.13-7.02 (m, 1H), 6.09 (s, 1H), 4.76-4.62 (m, 2H), 3.73 (s, 3H), 2.32 (d, 3H), 2.03 and 1.97 (2s, 3H each).

### EXAMPLE 279

### 2′,3′-dichloro-5-(((4-cyanobenzyl)oxy)(1-methyl-1H-imidazol-5-yl)methyl)(1,1′-biphenyl)-2-carbonitrile hydrochloride

The desired product was prepared by substituting 2,3-dichlorophenylboronic acid for 2-formylphenylboronic acid in Example 200D.

MS (APCI(−)) m/z 507 and 509 (M+Cl)⁻; MS (APCI(+)) m/z 473 and 475 (M+H)⁺; ¹H NMR (300 MHz, DMSO-d₆) δ 8.99 (s, 1H), 8.04 (dd, 1H), 7.82 (d, 2H), 7.68-7.66 (m, 1H), 7.57 (d, 2H), 7.47 (d, 1H), 7.39 (d, 1H), 7.28 (d, 1H), 7.21 (q, 1H), 7.13-7.02 (m, 1H), 6.09 (s, 1H), 4.76-4.62 (m, 2H), 3.73 (s, 3H).

### EXAMPLE 280

### 6-(((2′,3′-dichloro-6-cyano(1,1′-biphenyl)-3-yl)(1-methyl-1H-imidazol-5-yl)methoxy)methyl)nicotinonitrile dihydrochloride

The desired product was prepared by substituting Example 244A and 2,3-dichlorophenylboronic acid for Example 200C and 2-formylphenylboronic acid, respectively, in Example 200D.

MS (ESI(+)) m/z 474 and 476 (M+H)⁺; ¹H NMR (300 MHz, DMSO-d₆) δ 9.08 (s, 1H), 8.98 (dd, 1H), 8.33 (dd, 1H), 8.10 (d, 1H), 7.82-7.46 (m, 7H), 6.21 (s, 1H), 4.85-4.71 (m, 2H), 3.76 (s, 3H).

### EXAMPLE 281

### 6-(((6-cyano-2′,3′-dimethyl(1,1′-biphenyl)-3-yl)(1-methyl-1H-imidazol-5-yl)methoxy)methyl)nicotinonitrile dihydrochloride

The desired product was prepared by substituting Example 244A and 2,3-dimethylphenylboronic acid for Example 200C and 2-formylphenylboronic acid, respectively, in Example 200D.

MS (ESI(+)) m/z 434 (M+H)⁺ and 456 (M+Na)⁺; ¹H NMR (300 MHz, DMSO-d₆) (rotamers) δ 9.11 (s, 1H), 8.33 (dd, 1H), 8.04 (d, 1H), 7.72-7.50 (mn, 6H), 7.29 (d, 1H), 7.21 (dd, 1H), 7.12 (d, 1H), 6.19 (s, 1H), 4.85-4.70 (m, 2H), 3.78 (s, 3H), 2.32 (d, 3H), 2.03 and 1.97 (2s, 3H each).

### EXAMPLE 282

### 4-cyano-N-((4-cyano-3-(1-naphthyl)phenyl)(1-methyl-1H-imidazol-5-yl)methyl)benzenesulfonamide

A solution of Example 13A (50 mg, 0.148 mmol) in dichloromethane (1 mL) at room temperature was treated with 4-cyanobenzenesulfonyl chloride (35 mg, 0.174 mmol), triethylamine (150 μL), and catalytic DMAP, stirred for 14 hours, and concentrated. The concentrate was purified by flash column chromatography on silica gel with 95:5:1/ethyl acetate:ethanol:concentrated ammonium hydroxide to provide the desired product.

MS (DCI/NH₃) m/z 504 (M+H)⁺; ¹H NMR (300 MHz, DMSO-d₆) δ 9.13 (br s, 1H), 8.06 (m, 2H), 7.89 (m, 3H), 7.72-7.45 (m, 9H), 7.22-7.10 (m, 1H), 6.31-6.26 (two s, 1H), 5.96 (br. 1H), 3.63-3.60 (two s, 3H);

### EXAMPLE 283

### 4-((4-cyanoanilino)(1-methyl-1H-imidazol-5-yl)methyl)-2-(1-naphthyl)benzonitrile

### EXAMPLE 283A

### 4-(hydroxy(1-methyl-1H-imidazol-5-yl)methyl)-2-(1-naphthyl)benzonitrile

A solution of Example 89D (1.13 g, 3.33 mmol) in dichloromethane (20 mL) at 0° C. was treated dropwise with thionyl chloride (1.4 mL, 19.2 mmol), warmed to room temperature, stirred for 2 hours, concentrated, treated with toluene, and concentrated to provide the desired product of sufficient purity for subsequent use without further purification.

### EXAMPLE 283B

### 4-((4-cyanoanilino)(1-methyl-1H-imidazol-5-yl methyl)-2-(1-naphthyl)benzonitrile

A solution of Example 283A (100 mg, 0.280 mmol) in DMF (2 mL) at room temperature was treated with 4-cyanoaniline (165 mg, 1.40 mmol) and diisopropylethylamine (100 μL, 0.574 mmol), stirred for 72 hours, treated with ethyl acetate, washed with water and brine, dried (MgSO₄), filtered, and concentrated. The concentrate was purified by flash column chromatography on silica gel with 95:5/dichloromethane:methanol to provide the desired product.

MS (DCI/NH₃) m/z 440 (M+H)⁺; ¹H NMR (300 MHz, DMSO-d₆) δ 8.08 (m, 3H), 7.5-7.20 (m, 10H), 6.78 (m, 2H), 6.40-6.36 (two s, 1H), 6.15-6.11 (two s, 1H), 3.61-3.59 (two s, 3H);

### EXAMPLE 284

### 4-((3-cyanoanilino)(1-methyl-1H-imidazol-5-yl)methyl)-2-(1-naphthyl)benzonitrile

The desired product was prepared by substituting 3-cyanoaniline for 4-cyanoaniline in Example 283B.

MS (DCI/NH₃) m/z 440 (M+H)⁺; ¹H NMR (300 MHz, CDCl₃) δ 7.90 (m, 3H), 7.52 (m, 7H), 7.25 (m, 2H), 7.07 (m. 1H), 6.80 (m, 2H), 6.62 (m, 1H), 5.68 (m, 1H), 4.72 (m, 1H), 3.69-3.67 (two s, 3H);

### EXAMPLE 285

### tert-butyl 1-((4-cyano-3-(1-naphthyl)phenyl)(1-methyl-1H-imidazol-5-yl)methyl)-4-piperidinylcarbamate

A solution of Example 283A (30 mg, 0.0838 mmol) in DMF (1 mL) at room temperature was treated with tert-butyl 4-piperidinylcarbamate (90 mg, 0.449 mmol) and diisopropylethylamine (80 μL, 0.46 mmol), stirred for 72 hours, heated to 60° C., stirred for 16 hours, treated with ethyl acetate, washed with water and brine, dried (MgSO₄), filtered, and concentrated. The concentrate was purified by flash column chromatography on silica gel with 95:5/dichloromethane:methanol to provide the desired product.

MS (ESI(+)) m/z 522 (M+H)⁺; ¹H NMR (300 MHz, DMSO-d₆) δ 8.05 (m, 1H), 7.94 (s, 1H), 7.50 (m, 7H), 6.89 (m, 1H), 6.75 (m, 1H), 4.02 (m, 2H), 3.60-3.50 (m, 3H), 3.31 (s, 3H), 3.05 (m, 1H), 2.70 (m, 2H), 1.72 (m, 2H), 1.38 (s, 9H).

### EXAMPLE 286

### 4-((4-cyanophenoxy)(1-methyl-1H-imidazol-5-yl)methyl)-2-(1-naphthyl)benzonitrile

A solution of Example 89D in THF (2 mL) at room temperature was treated with DEAD (60 μL, 0.38 mmol), 4-hydroxybenzonitrile (42 mg, 0.353 mmol), and triphenylphosphine (93 mg, 0.355 mmol), stirred for 16 hours, treated with diethyl ether, washed with 1M NaOH, water, and brine, dried (MgSO₄), filtered, and concentrated. The concentrate was purified by flash column chromatography on silica gel with 95:5:1/ethyl acetate:ethanol:concentrated ammonium hydroxide to provide the desired product.

MS (DCI/NH₃) m/z 441 (M+H)⁺; ¹H NMR (300 MHz, DMSO-d₆) δ 8.08 (m, 3H), 7.80-7.15 (m, 1lH), 7.07(s, 1H), 6.63-6.60 (two s, 1H), 3.62-3.60 (two s, 3H);

### EXAMPLE 287

### 4-(((4-cyanophenyl)(1-methyl-1H-imidazol-5-yl)methyl)amino)-2-(1-naphthyl)benzonitrile

### EXAMPLE 287A

### 2-(1-naphthyl)-4-nitrobenzonitrile

A solution of 1-naphthylboronic acid (2.58 g, 15.0 mmol) in toluene (20 mL) and dioxane (20 mL) was treated with 2-chloro-4-nitrobenzonitrile (1.83 g, 10.0 mmol), trans-dichloro(bis(tricyclohexylphosphino))palladium (370 mg, 0.50 mmol), and 2M Na₂CO₃ (20 mL), purged with nitrogen, heated to reflux, stirred for 19 hours, treated with ethyl acetate, washed with water and brine, dried (MgSO₄), filtered, and concentrated. The concentrate was triturated with ethyl acetate/hexanes to provide the desired product.

MS (DCI/NH₃) m/z 292 (M+NH₄)⁺; ¹H NMR (300 MHz, CDCl₃) δ 8.41(m, 2H), 8.00 (m, 3H), 7.53(m, 5H).

### EXAMPLE 287B

### 4-amino-2-(1-naphthyl)benzonitrile

A suspension of Example 287A (500 mg, 1.82 mmol) in ethanol (7 mL) at room temperature was treated with concentrated HCl (2 mL) and a solution of SnCl₂.2H₂O (1.25 g, 5.54 mmol) in ethanol (4 mL), stirred for 3 hours, and concentrated. The concentrate was treated with diethyl ether and 30% NaOH. The aqueous layer was extracted with diethyl ether, and the extract was washed sequentially with 1M NaOH, water, and brine, dried (MgSO₄), filtered, and concentrated. The concentrate was triturated with hexanes to provide the desired product.

MS (DCI/NH₃) m/z 262 (M+NH₄)⁺; ¹H NMR (300 MHz, CDCl₃) δ 7.91 (m, 1H), 7.55 (m, 8H), 6.70 (m, 2H), 4.2 (br s, 2H).

### EXAMPLE 287C

### 4-(hydroxy(1-methyl-1H-imidazol-5-yl)methyl)benzonitrile

A solution of Example 87F (3.35 g, 17.08 mmol) in THF (50 mL) at −78° C. was treated dropwise with 1.5M tert-butyllithium in pentane (11.4 mL, 17.1 mmol), stirred for 30 minutes, treated with a solution of 4-cyanobenzaldehyde (2.04 g, 15.56 mmol) in THF (10 mL) at −78° C., stirred for 1 hour, treated with methanol (4 mL), warmed to room temperature, stirred for 1 hour, treated with 1M HCl (40 mL), stirred for 1.5 hours, adjusted to pH 12 with 30% NaOH, and extracted with ethyl acetate. The extract was washed with brine, dried (MgSO₄), filtered, and concentrated. The concentrate was triturated with 4:1/hexanes:ethyl acetate to provide the desired product.

MS (DCI/NH₃) m/z 214 (M+H)⁺ and 231 (M+NH₄)⁺; ¹H NMR (300 MHz, CDCl₃) δ 7.67 (d, 2H), 7.52 (d, 2H), 7.40 (s, 1H), 6.67 (s, 1H), 5.95 (s, 1H), 3.53 (s, 3H).

### EXAMPLE 287D

### 4-(chloro(1-methyl-1H-imidazol-5-yl)methyl)benzonitrile

A solution of Example 286 in dichloromethane (40 mL) at 0° C. was treated with thionyl chloride (2.8 mL, 38.4 mmol), warmed to room temperature, stirred for 4 hours, and concentrated. The concentrate was treated with toluene and concentrated to provide the desired product of sufficient purity for subsequent use without further purification.

### EXAMPLE 287E

### 4-(((4-cyanophenyl)(1-methyl)amino)-2-(1-naphthyl)benzonitrile

A solution of Example 287D (143 mg, 0.534 mmol) in DMF (4 mL) at room temperature was treated with Example 287B (130 mg, 0.532 mmol) and diisopropylethylamine (470 μL, 2.70 mmol), stirred for 72 hours, treated with ethyl acetate, washed with water and brine, dried (MgSO₄), filtered, and concentrated. The concentrate was purified by flash column chromatography on silica gel with 95:5/dichloromethane:methanol to provide the desired product.

MS (DCI/NH₃) m/z 440 (M+H)⁺; ¹H NMR (300 MHz, CDCl₃) δ 7.90-7.50 (m, 11H), 6.60 (m, 3H), 5.66 (m, 1H), 5.05 (m, 1H), 3.68-3.62 (two s, 3H).

### EXAMPLE 288

### 6-(((4-cyano-3-(1-naphthyl)benzyl)((1-methyl-1H-imidazol-5-yl)methyl)amino)methyl)nicotinonitrile

A solution of Example 192D (35 mg, 0.10 mmol) in 5% acetic acid/DME (1.0 mL) at room temperature was treated with 6-formylnicotinonitrile (30 mg, 3.0 mmol) and 4A molecular sieves, stirred for 1 hour, treated with sodium triacetoxyborohydride (40 mg, 2.0 mmol), stirred for 16 hours, treated with ethyl acetate (1.0 mL), washed with saturated sodium bicarbonate and brine, filtered through a Chem Elut® CE1000M tube (Alltech, Northbrook, Ill.), and concentrated. The concentrate was purified by preparative HPLC (CH₃CN/0.010M NH₄OAc) to provide the desired product.

MS (APCI(+)) m/z 469 (M+H)⁺; ¹H NMR (300 MHz, DMSO-d₆) δ 9.78 (s, 1H), 8.88 (s, 1H), 8.17 (dd, 1H), 8.07 (d, 1H), 8.05 (d, 1H), 7.93 (d, 1H), 7.66-7.45 (m, 8H), 7.39 (d, 1H), 3.83 (s, 2H), 3.80 (s, 2H), 3.69 (s, 2H), 3.50 (s, 3H).

### EXAMPLE 289

### 6-(((4-cyano-3-(1-naphthyl)phenyl)(3-thienyl)methoxy)methyl)nicotinonitrile

### EXAMPLE 289A

### 4-(hydroxyl(3-thienyl)methyl)-2-(1-naphthyl)benzonitrile

The desired product was prepared by substituting Example 89C for Example 86I in Example 80A.

MS (DCI/NH₃) m/z 359 (M+NH₄)⁺; ¹H NMR (500 MHz, CDCl₃) δ 7.92 (m, 2H), 7.80 (dd, 1H), 7.60-7.41 (m, 7H), 7.31 (m, 1H), 7.23 (m, 1H), 7.01 (dd, 1H), 5.98 (d, 1H), 2.42 (d, 1H).

### EXAMPLE 289B

### 6-(((4-cyano-3-(1 -naphthyl)phenyl)(3-thienyl)methoxy)methyl)nicotinonitrile

Example 289A (360 mg, 1.06 mmol) and Example 76A (416 mg, 2.11 mmol) were dissolved in THF (5 mL). The solution was purged with nitrogen and cooled to −5° C. Sodium hydride (30 mg, 1.25 mmol) was added and the reaction was stirred for 1.5 hours. Aqueous ammonium chloride was added and the mixture was partitioned between ethyl acetate and water. The organic phase was dried (MgSO₄), filtered, and concentrated. Chromatography of the residue on silica gel with 4:1 hexanes:ethyl acetate provided the desired product.

MS (DCI/NH₃) m/z 458 (M+H)⁺; ¹H NMR (300 MHz, CDCl₃) 6 8.79 (dd, 1H), 7.94 (m, 3H), 7.83 (m, 1H), 7.65-7.44 (m, 8H), 7.35 (m, 1H), 7.30 (m, 1H), 7.02 (dd, 1H), 5.72 (s, 1H), 4.75 (d, 2H).

### EXAMPLE 290

### 4-(((4-cyanobenzyl)oxy)(1,3-thiazol-5-yl)methyl)-2-(1-naphthyl)benzonitrile

### EXAMPLE 290A

### 4-(hydroxy(1,3-thiazol-5-yl)methyl)-2-(1-naphthyl)benzonitrile

The desired product was prepared by substituting 2-triethylsilylthiazole and Example 89C for Example 87F and Example 1A, respectively, in Example 1B.

MS (ESI(+)) m/z 343 (M+H)⁺; ¹H NMR (500 MHz, DMSO-d₆) δ 9.11 (s, 1H), 8.03 (m, 3H), 7.87 (d, 1H), 7.73 (d, 1H), 7.63 (m, 2H), 7.57 (m, 1H), 7.50 (m, 2H), 7.41 (m, 1H), 6.28 (s, 1H).

### EXAMPLE 290B

### 4-(((4-cyanobenzyl)oxy)(1,3-thiazol-5-yl)methyl)-2-(1-naphthyl)benzonitrile

The desired product was prepared by substituting Example 290A for Example 289A and 4-cyanobenzyl bromide for example 76A in Example 289.

MS (ESI(+)) m/z 458 (M+H)⁺; ¹H NMR (500 MHz, CH₃OD) δ 9.69 (s, 1H), 8.14 (d, 1H), 7.98 (m, 3H), 7.79 (m, 1H), 7.68 (m, 3H), 7.60-7.51 (m, 4H), 7.49-7.41 (m, 3H), 6.20 (d, 1H), 4.78 (m, 2H); Anal. Calcd. for C₂₉H₁₉N₃OS·HCl: C, 70.51; H, 4.08; N, 8.51. Found: C, 70.42; H, 4.20; N, 8.61.

### EXAMPLE 291

### 6-(((4-cyano-3-(1-naphthyl)phenyl)(1,3-thiazol-5-yl)methoxy)methyl)nicotinonitrile

The desired product was prepared by substituting Example 290A for Example 289A in Example 289 and was converted to the trifluoroacetic acid salt.

MS (ESI(+)) m/z 459 (M+H)⁺; ¹H NMR (500 MHz, DMSO-d₆) δ 9.14 (s, 1H), 8.97 (d, 1H), 8.32 (m, 1H), 8.08 (m, 3H), 7.98 (d, 1H), 7.80 (dd, 1H), 7.70-7.37 (m, 7H), 6.33 (s, 1H), 4.77 (m, 2H); Anal. Calcd. for C₂₈H₁₈N₄OS·0.95 C₂BF₃O₂: C, 63.35; H, 3.37; N, 9.88. Found: C, 63.34; H, 3.22; N, 9.87.

### EXAMPLE 292

### 6-(((4-cyano-3-(1-naphthyl)phenyl)(3-pyridinyl)methoxy)methyl)nicotinonitrile

### EXAMPLE 292A

### 4-(hydroxy(3-pyridinyl)methyl)-2-(1 -naphthyl)benzonitrile

A solution of 3-iodopyridine (588 mg, 2.87 mmol) in THF (10 mL) at −50° C. was slowly treated with 0.8M isopropyl magnesium bromide (3.6 mL, 2.88 mmol). The mixture was stirred at <-25° C. for approximately 1 hour, treated with Example 89C (491 mg, 1.91 mmol), and stirred overnight while warming to room temperature. The reaction was quenched with aqueous NH₄Cl and extracted with ethyl acetate. The combined extracts were dried (MgSO₄), filtered, and concentrated. Chromatography of the residue on silica gel eluting with 70 to 80% ethyl acetate/hexanes provided the desired product.

MS (ESI(+)) m/z 337 (M+H)⁺; ¹H NMR (300 MHz, CDCl₃) δ 8.58 (s, 1H), 8.49 (d, 1H), 7.93 (m, 2H), 7.80 (dd, 1H), 7.70 (ddd, 1H), 7.59-7.44 (m, 4H), 7.41 (m, 3H), 7.28 (dd, 1H), 5.94 (s, 1H).

### EXAMPLE 292B

### 6-(((4-cyano-3-(1-naphthyl)phenyl)(3-pyridinyl)methoxy)methyl)nicotinonitrile trifluoroacetate salt

The desired product was prepared by substituting Example 292A for Example 289A in Example 289B and converting the product to the trifluoroacetate salt.

MS (ESI(+)) m/z 453 (M+H)⁺; ¹H NMR (500 MHz, CDCl₃) δ 8.98 (t, 1H), 8.93 (s, 1H), 8.71 (d, 1H), 8.34 (ddd, 1H), 8.25 (m, 1H), 8.07 (m, 3H), 7.86-7.77 (m, 3H), 7.70 (dd, 1H), 7.67-7.37 (m, 5H), 6.11 (d, 1H), 4.84-4.76 (m, 2H). Anal. Calcd. for C₃₀H₂₀N₄O·1.49 C₂HF₃O₂·0.25 H₂O: C, 63.19; H, 3.54; N, 8.94. Found: C, 63.17; H, 3.49; N, 9.04.

Following the schemes and the examples described above, the following compounds can be prepared:

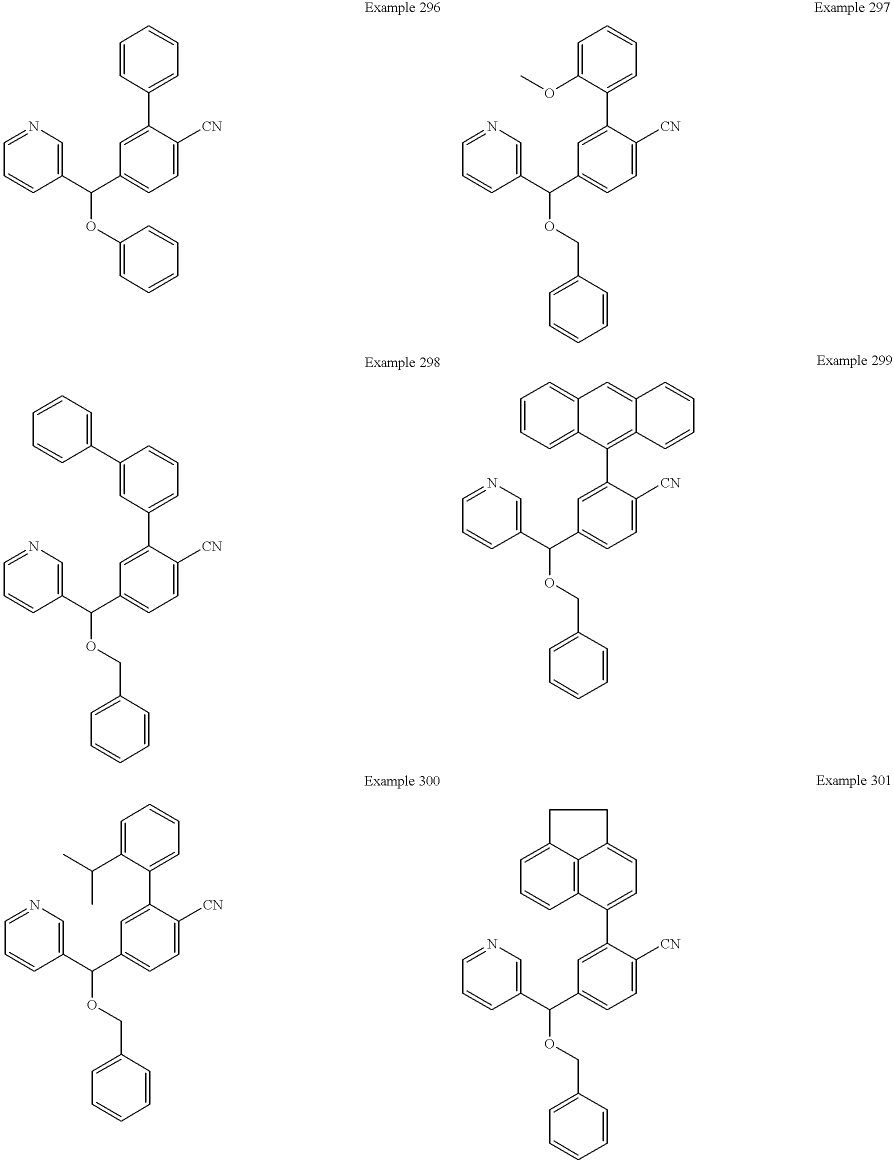

It will be evident to one skilled in the art that the invention is not limited to the foregoing illustrative examples, and that it can be embodied in other specific forms without departing from the essential attributes thereof. It is therefore desired that the examples be considered in all respects as illustrative and not restrictive, reference being made to the appended claims, and all changes which come within the meaning and range of equivalency of the claims are therefore intended to be embraced within.

What is claimed is:

1. A compound of formula (I) 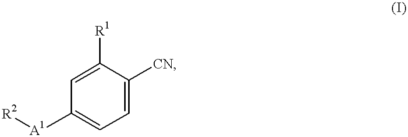
or pharmaceutically acceptable salts thereof, wherein
A¹ is L¹-M¹-L² or alkylene, wherein the alkylene can be optionally substituted with one, two, or three substituents independently selected from the group consisting of amino, hydroxyl, oxo, and -Q¹-Q²-R³;
with the proviso that when A¹ is methylene, the methylene is substituted;
L¹and L² are independently absent or alkylene, wherein the alkylenes defining L¹ and L² can be optionally substituted with one or two substituents independently selected from the group consisting of alkyl, cycloalkyl, cycloalkylalkyl, aryl, arylalkyl, heteroaryl, heteroarylalkyl, and oxo;
with the proviso that at least one of L¹ or L² is present;
M¹ is selected from the group consisting of O, N(R⁴), N(R⁵)SO₂, SO₂N(R⁵), N(R⁵)C(O), C(O)N(R⁵), OC(O), C(O)O, C(O), N(R⁵)C(O)O, OC(O)N(R⁵), OC(O)O, N(R⁵)C(O)N(R⁵), and S(O)ₜ, wherein t is zero, one, or two;
wherein, for the groups defining M¹, the left ends are attached to L¹ and the right ends are attached to L²;
Q¹ is absent or selected from the group consisting of O, N(R⁴), N(R⁵)C(O), N(R⁵)SO₂, and S(O)ₜ;
Q² is absent or selected from the group consisting of alkylene, alkenylene, and alkynylene;
R¹ is selected from the group consisting of halo, cycloalkyl, aryl, and heteroaryl;
R² is a heteroaryl selected from the group consisting of imidazolyl, pyrazolyl, pyrrolyl, thienyl, triazolyl, pyridyl, and thiazolyl;
R³ is selected from the group consisting of alkyl, cycloalkyl, aryl, heteroaryl, and heterocycloalkyl;
R⁴ is selected from the group consisting of hydrogen, alkyl, alkenyl, alkynyl, alkanoyl, alkylsulfonyl, a nitrogen protecting group, aminosulfonyl, aryl, arylalkyl, aryloyl, arylsulfonyl, cycloalkyl, cycloalkylalkyl, cycloalkyloyl, cycloalkylsulfonyl, heteroaryl, heteroarylalkyl, heteroaryloyl, heteroarylsulfonyl, heterocycloalkyl, heterocycloalkylalkyl, heterocycloalkyloyl, and heterocycloalkylsulfonyl; and
R⁵ is selected from the group consisting of hydrogen, alkyl, aryl, arylalkyl, cycloalkyl, cycloalkylalkyl, heteroaryl, heteroarylalkyl, heterocycloalkyl, and heterocycloalkylalkyl.

2. A compound according to claim 1 of formula (II) 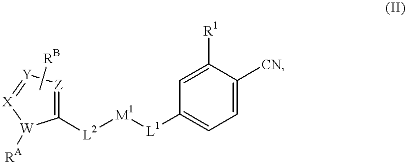
or a pharmaceutically acceptable salt thereof, wherein
L¹, L², M¹, and R¹ are defined above;
R^{A} is absent or selected from the group consisting of hydrogen, optionally substituted alkyl, alkoxycarbonyl, and a nitrogen protecting group;
R^{B} is absent or selected from the group consisting of optionally substituted alkyl, alkoxy, alkanoyl, alkanoyloxy, alkoxycarbonyl, alkylsulfonyl, amino, aminosulfonyl, azido, carboxamido, carboxyl, cyano, halo, hydroxyl, perfluoroalkyl, and perfluoroalkoxy;
W is C(H)═C(H), X is N, and Y and Z are C(H); or
W is C(H)═N or N═C(H), wherein each group is drawn with its left end attached to X and its right end attached to the carbon substituted with L²; and X, Y and Z are C(H); or
W is N or S, one of X, Y, or Z is C(H), and the remainder are C(H) or N;
with the proviso that R^{A} is present when and only when W is N.

3. A compound according to claim 2, wherein
M¹ is O;
L¹ is optionally substituted alkylene;
L² is optionally substituted alkylene;
W and Y are N; and
X and Z are C(H).

4. A compound according to claim 2 wherein,
M¹ is O;
L¹ is optionally substituted alkylene;
L² is optionally substituted alkylene;
W is N═C(H); and
X, Y, and Z are C(H).

5. A compound according to claim 2 wherein
M¹ is O;
L¹ is optionally substituted alkylene;
L² is optionally substituted alkylene;
W is S;
Y is N; and
X and Z are C(H).

6. A compound according to claim 2, wherein
M¹ is N(R⁴);
W is N;
Y is N; and
X and Z are C(H).

7. A compound according to claim 2, wherein
M¹ is N(R⁴);
W is N═C(H); and
X, Y and Z are C(H).

8. A compound according to claim 2 wherein
M¹is N(R⁴);
W is S;
Y is N; and
X and Z are C(H).

9. A compound according to claim 1 of formula (III) 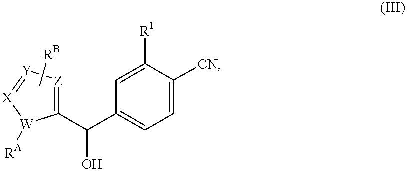
or a pharmaceutically acceptable salt thereof, wherein
R¹ is defined above;
R^{A} is absent or selected from the group consisting of hydrogen, optionally substituted alkyl, alkoxycarbonyl, and a nitrogen protecting group;
R^{B} is absent or selected from the group consisting of optionally substituted alkyl, alkoxy, alkanoyl, alkanoyloxy, alkoxycarbonyl, alkylsulfonyl, amino, aminosulfonyl, azido, carboxamido, carboxyl, cyano, halo, hydroxyl, perfluoroalkyl, and perfluoroalkoxy; and
W is C(H)═C(H), X is N, and Y and Z are C(H); or
W is C(H)=N or N=C(H), wherein each group is drawn with its left end attached to X and its right end attached to the carbon substituted with L²; and X, Y and Z are C(H); or
W is N or S, one of X, Y, or Z is C(H), and the remainder are C(H) or N;
with the proviso that R^{A} is present when and only when W is N.

10. A compound according to claim 9 wherein
W is N;
Y is N; and
X and Z are C(H).

11. A compound according to claim 9 wherein
W is S;
Y is N; and
X and Z are C(H).

12. A compound according to claim 9 wherein
W is N═C(H); and
X, Y, and Z are C(H).

13. A compound according to claim 1 of formula (IV) 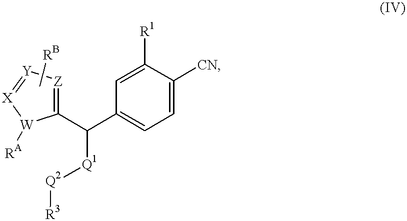
or a pharmaceutically acceptable salt thereof, wherein
Q¹, R¹, and R³ are defined above;
Q² is absent or alkylene;
R^{A} is absent or selected from the group consisting of hydrogen, optionally substituted alkyl, alkoxycarbonyl, and a nitrogen protecting group;
R^{B} is absent or selected from the group consisting of optionally substituted alkyl, alkoxy, alkanoyl, alkanoyloxy, alkoxycarbonyl, alkylsulfonyl, amino, aminosulfonyl, azido, carboxamido, carboxyl, cyano, halo, hydroxyl, perfluoroalkyl, and perfluoroalkoxy; and
W is C(H)═C(H), X is N, and Y and Z are C(H); or
W is C(H)═N or N═C(H), wherein each group is drawn with its left end attached to X and its right end attached to the carbon substituted with L²; and X, Y and Z are C(H); or
W is N or S, one of X, Y, or Z is C(H), and the remainder are C(H) or N;
with the proviso that R^{A} is present when and only when W is N.

14. A compound according to claim 13 wherein
Q¹ is O;
W is N;
Y is N; and
X and Z are C(H).

15. A compound according to claim 13 wherein
Q¹ is O;
W is N═C(H); and
X, Y, and Z are C(H).

16. A compound according to claim 13 wherein
Q¹ is O;
W is S;
Y is N; and
X and Z are C(H).

17. A compound according to claim 13 wherein
Q¹ is N(R⁴);
W is N;
Y is N; and
X and Z are C(H).

18. A compound according to claim 13 wherein
Q¹ is N(R⁴);
W is N═C(H); and
X, Y, and Z are C(H).

19. A compound according to claim 13 wherein
Q¹ is N(R⁴);
W is S;
Y is N; and
X and Z are C(H).

20. A compound according to claim 13 wherein
Q¹ is S(O)ₜ, wherein t is zero, one, or two;
W is N;
Y is N; and
X and Z are C(H).

21. A compound according to claim 13 wherein
Q¹ is S(O)ₜ, wherein t is zero, one, or two;
W is N═C(H); and
X, Y, and Z are C(H).

22. A compound according to claim 13 wherein
Q¹ is S(O)ₜ, wherein t is zero, one, or two;
W is S;
Y is N; and
X and Z are C(H).

23. A compound according to claim 13 wherein
Q¹ is N(R⁵)SO₂;
W is N;
Y is N; and
X and Z are C(H).

24. A compound according to claim 13 wherein
Q¹ is N(R⁵)SO₂;
W is N═C(H); and
X, Y, and Z are C(H).

25. A compound according to claim 13 wherein
Q¹ is N(R⁵)SO₂;
W is S;
Y is N; and
X and Z are C(H).

26. A compound according to claim 13 wherein
Q¹ is absent;
W is N;
Y is N; and
X and Z are C(H).

27. A compound according to claim 13 wherein
Q¹ is absent;
W is N═C(H); and
X, Y, and Z are C(H).

28. A compound according to claim 13 wherein
Q¹ is absent;
W is S;
Y is N; and
X and Z are C(H).

29. A compound according to claim 1 of formula (V) 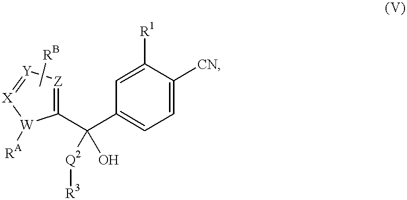
or pharmaceutically acceptable salts thereof, wherein
Q², R¹ , and R² are defined above;
R^{A} is absent or selected from the group consisting of hydrogen, optionally substituted alkyl, alkoxycarbonyl, and a nitrogen protecting group;
R^{B} is absent or selected from the group consisting of optionally substituted alkyl, alkoxy, alkanoyl, alkanoyloxy, alkoxycarbonyl, alkylsulfonyl, amino, aminosulfonyl, azido, carboxamido, carboxyl, cyano, halo, hydroxyl, perfluoroalkyl, and perfluoroalkoxy; and
W is C(H)═C(H), X is N, and Y and Z are C(H); or
W is C(H)═N or N═C(H), wherein each group is drawn with its left end attached to X and its right end attached to the carbon substituted with L²; and X, Y and Z are C(H); or
W is N or S, one of X, Y, or Z is C(H), and the remainder are C(H) or N;
with the proviso that R^{A} is present when and only when W is N.

30. A compound according to claim 29 wherein
W is N;
Y is N; and
X and Z are C(H).

31. A compound according to claim 29 wherein
W is N═C(H); and
X, Y, and Z are C(H).

32. A compound according to claim 29 wherein
W is S;
Y is N; and
X and Z are C(H).

33. A compound according to claim 1 of formula (XIV) 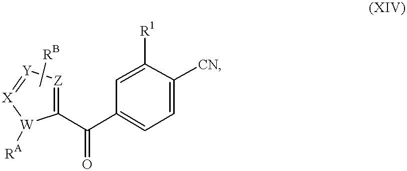
or a pharmaceutically acceptable salt thereof, wherein
R^{A} is absent or selected from the group consisting of hydrogen, optionally substituted alkyl, alkoxycarbonyl, and a nitrogen protecting group;
R^{B} is absent or selected from the group consisting of optionally substituted alkyl, alkoxy, alkanoyl, alkanoyloxy, alkoxycarbonyl, alkylsulfonyl, amino, aminosulfonyl, azido, carboxamido, carboxyl, cyano, halo, hydroxyl, perfluoroalkyl, and perfluoroalkoxy; and
W is C(H)═C(H), X is N, and Y and Z are C(H); or
W is C(H)═N or N═C(H), wherein each group is drawn with its left end attached to X and its right end attached to the carbon substituted with L²; and X, Y and Z are C(H); or
W is N or S, one of X, Y, or Z is C(H), and the remainder are C(H) or N;
with the proviso that R^{A} is present when and only when W is N.

34. A compound according to claim 33 wherein
W is N;
Y is N; and
X and Z are C(H).

35. A compound according to claim 33 wherein
W is N═C(H); and
X, Y, and Z are C(H).

36. A compound according to claim 33 wherein
W is S; and
X, Y, and Z are C(H).
